(19) United States
(12) Patent Application Publication
(10) Pub. No.: US 2023/0322656 A1
(43) Pub. Date: Oct. 12, 2023

(54) ORGANIC COMPOUND, AND ELECTRONIC COMPONENT AND ELECTRONIC DEVICE HAVING SAME

(71) Applicant: SHAANXI LIGHTE OPTOELECTRONICS MATERIAL CO., LTD., Xi?an (CN)

(72) Inventors: Linnan MA, Xi'an (CN); Peng NAN, Xi'an (CN); Youngkook KIM, Xi'an (CN); Yingwen LI, Xi'an (CN)

(21) Appl. No.: 18/003,141

(22) PCT Filed: Mar. 22, 2022
(86) PCT No.: PCT/CN2022/082291

§ 371 Date: Dec. 22, 2022

(30) Foreign Application Priority Data
Apr. 2, 2021 (CN) 202110362511.8

(51) Int. Cl. C07C 211/54; H10K 85/60; C07D 307/91; C07D 333/76; C07D 209/82; C07D 409/12; C07D 405/12; C07F 7/08; H10K 85/40; H10K 50/18; H01L 33/50

(52) U.S. Cl. CPC C07C 211/54; C07D 209/82; C07D 307/91; C07D 333/76; C07D 405/12; C07D 409/12; C07F 7/081; C07F 7/0812; H01L 33/50; H10K 50/181; H10K 85/40; H10K 85/615; H10K 85/626; H10K 85/633; H10K 85/636; H10K 85/6572; H10K 85/6574; H10K 85/6576; C07B 2200/05

(57) ABSTRACT

The present application relates to an organic compound, and an electronic component and electronic device having the same, and belongs to the technical field of organic electroluminescence. The organic compound of the present application has a structure shown in formula 1. When used in an electronic component, the organic compound of the present application can significantly improve the performance of the electronic component.

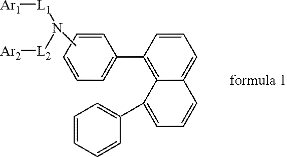

### CROSS REFERENCE TO RELATED APPLICATION

The present application claims priority to Chinese Patent Application 202110362511.8 filed on Apr. 2, 2021, which is incorporated into the present application by reference in its entirety.

### TECHNICAL FIELD

The present application relates to the technical field of organic electroluminescence, and in particular to an organic compound, and an electronic component and electronic device having the same.

### BACKGROUND

With the development of electronic technology and the progress of material science, electronic components for realizing electroluminescence or photoelectric conversion are more and more extensively used. Such an electronic component usually includes: a cathode and an anode that are arranged oppositely, and a functional layer arranged between the cathode and the anode. The functional layer includes a plurality of organic or inorganic film layers, and generally includes an energy conversion layer, a hole transport layer (HTL) arranged between the energy conversion layer and the anode, and an electron transport layer (ETL) arranged between the energy conversion layer and the cathode.

For example, an organic light-emitting device (OLED) generally includes an anode, an HTL, an organic light-emitting layer as an energy conversion layer, an ETL, and a cathode that are successively stacked. When a voltage is applied to the cathode and the anode, an electric field is generated at each of the two electrodes; and under the action of the electric field, both electrons at a cathode side and holes at an anode side move towards the organic light-emitting layer and are combined in the organic light-emitting layer to form excitons, and the excitons in an excited state release energy outwards, thereby causing the organic light-emitting layer to emit light.

In a structure of an OLED, an electron blocking layer (EBL) is arranged to block electrons transmitted from an organic light-emitting layer, thereby ensuring that an electron and a hole can be efficiently recombined in the organic light-emitting layer; the EBL can also block excitons diffused from the organic light-emitting layer to reduce the triplet-state quenching of the excitons, thereby ensuring the light-emitting efficiency of the OLED: and a material of the EBL has a relatively-high LUMO value, which can effectively block the transmission and diffusion of electrons and excitons from the organic light-emitting layer to an anode. With the continuous market development, the requirements for the light-emitting efficiency and service life of devices are increasingly high. The development of stable and efficient EBL materials to reduce the driving voltage, improve the light-emitting efficiency of a device, and prolong the life span of a device is of very important practical application values.

### SUMMARY

The present application is intended to provide an organic compound, and an electronic component and electronic device having the same. When used in an OLED, the organic compound can improve the light-emitting efficiency of the OLED and prolong the life span of the OLED.

In a first aspect of the present application, an organic compound with a structure shown in formula 1 is provided:

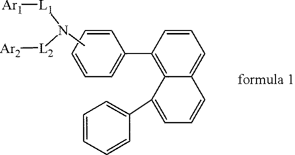

where Ar₁ is selected from the group consisting of formula I, formula II, and formula III:

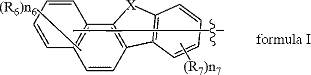

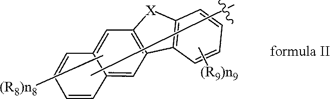

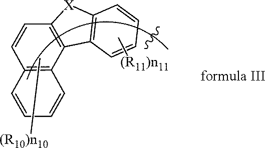

where

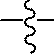

represents a chemical bond;
X is selected from the group consisting ofC(R₁R₂), O, S, N(R₃), and Si(R₄R₅).
R₁, R₂, R₃, R₄, and R₅ are the same or different, and are each independently selected from the group consisting of alkyl with 1 to 5 carbon atoms, aryl with 6 to 12 carbon atoms, and heteroaryl with 3 to 12 carbon atoms; or R₁ and R₂ form a saturated or unsaturated 5-13 membered ring; or R₄ and R₅ form a saturated or unsaturated 5-13 membered ring;
R₆, R₇, R₈, R₉, R₁₀, and R₁₁ are the same or different, and are each independently selected from the group consisting of deuterium, halogen, cyano, alkyl with 1 to 5 carbon atoms, trialkylsilyl with 3 to 12 carbon atoms, aryl with 6 to 12 carbon atoms, and heteroaryl with 3 to 12 carbon atoms,
R₆, R₇, R₈, R₉. R₁₀, and R₁₁, are collectively represented by Rᵢ: n₆, n₇, n₈, n₉, n₁₀, and n₁₁ are collectively represented by nᵢ; nᵢ indicates the number of Rᵢ, and i is a variable of 6, 7, 8, 9, 10, or 11; when i is 6, 8, or 10, nᵢ is selected from the group consisting of 0, 1, 2, 3, 4, 5, and 6; when i is 7, 9, or 11, iii is selected from the group consisting of 0, 1. 2, 3, and 4; and when nᵢ is greater than 1, any two Rᵢ are the same or different;
Ar₂ is selected from the group consisting of a substituted or unsubstituted aryl with 6 to 40 carbon atoms and a substituted or unsubstituted heteroaryl with 3 to 30 carbon atoms;
L₁ and L₂ are the same or different, and are each independently selected from the group consisting of a single bond, a substituted or unsubstituted arylene with 6 to 20 carbon atoms, and a substituted or unsubstituted heteroarylene with 3 to 20 carbon atoms;
substituents in L₁, L₂, and Ar₂ are the same or different, and are each independently selected from the group consisting of deuterium, halogen, cyano, alkyl with 1 to 10 carbon atoms, trialkylsilyl with 3 to 12 carbon atoms, aryl with 6 to 20 carbon atoms, heteroaryl with 3 to 20 carbon atoms, haloalkyl with 1 to 10 carbon atoms, and alkoxy with 1 to 10 carbon atoms; and optionally, any two adjacent substituents in Ar₂ form a saturated or unsaturated 3-15 membered ring.

The organic compound of the present application has a core structure formed by combining 1,8-diphenyl-substituted naphthyl with a fused aromatic hydrocarbon and a triarylamine structure. The 1,8-diphenyl-substituted naphthyl can make the transfer of holes from an HTL to an organic light-emitting layer smooth, such that the organic compound has excellent hole transport efficiency and the voltage and efficiency performance of an electronic component using the organic compound can be effectively improved. In addition, the introduction of the 1.8- diphenyl-substituted naphthyl changes a conjugate plane of the compound, which may cause molecular crystallization, thereby reducing a life span of a device. Therefore, in order to overcome the adverse effects of 1,8-diphenyl-substituted naphthyl, a fused aromatic hydrocarbon with large steric hindrance and conjugative effect is introduced into the organic compound, which effectively improves a crystallization effect of the organic compound and enhances a film-forming effect of the organic compound. The triarylamine structure has prominent hole transport performance, and the triarylamine structure can be bonded with 1,8-diphenyl-substituted naphthyl and a fused aromatic hydrocarbon to increase the molecular rigidity and significantly improve the thermal stability, such that the structural stability can be maintained at a high temperature for a long time, thereby improving the light-emitting efficiency of an OLED and the power generation efficiency of a photoelectric conversion device. The organic compound of the present application has excellent hole transport performance, a low intermolecular stacking effect, and excellent film-forming performance, which can improve the efficiency performance and life performance of electronic components such as photoelectric conversion devices and electroluminescent devices.

In a second aspect of the present application, an electronic component is provided, including an anode, a cathode, and at least one functional layer between the anode and the cathode, where the functional layer includes the organic compound described above.

In a third aspect of the present application, an electronic device is provided, including the electronic component described above.

Other features and advantages of the present application will be described in detail in the following DETAILED DESCRIPTION section.

### BRIEF DESCRIPTION OF THE DRAWINGS

The accompanying drawings are provided for further understanding the present application, and constitute a part of the specification. The accompanying drawings and the following specific embodiments are intended to explain the present application, but do not limit the present application.

FIG. 1 is a schematic structural diagram of an OLED according to an embodiment of the present application; and

FIG. 2 is a schematic diagram of an electronic device according to an embodiment of the present application.

### REFERENCE NUMERALS

100 represents an anode; 200 represents a cathode; 300 represents a functional layer; 3 10 represents a hole injection layer (HIL); 320 represents an HTL; 330 represents an EBL; 340 represents an organic electroluminescent layer; 350 represents an ETL; 360 represents an electron injection layer (EIL); and 400 represents an electronic device.

### DETAILED DESCRIPTION

Exemplary embodiments will be described below comprehensively with reference to the accompanying drawings. However, the exemplary embodiments can be implemented in various forms and should not be construed as being limited to examples described herein. On the contrary, these embodiments are provided such that the present application is comprehensive and complete, and fully conveys the concept of the exemplary embodiments to those skilled in the art. The described features, structures, or characteristics may be incorporated into one or more embodiments in any suitable manner. In the following description, many specific details are provided to give a full understanding of the embodiments of the present application.

In a first aspect, the present application provides an organic compound with a structure shown in formula I:

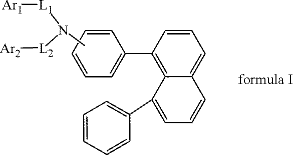

where An is selected from the group consisting of formula I, formula II, and formula III:

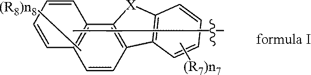

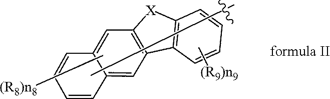

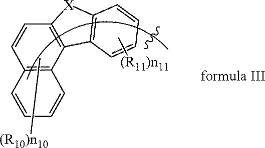

where

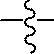

represents a chemical bond;
X is selected from the group consisting of C(R₁R₂)5 O, S, N(R₃), and Si(R₄R₅);
R₁, R₂, R₃, R₄, and R₅ are the same or different, and are each independently selected from the group consisting of alkyl with 1 to 5 carbon atoms, aryl with 6 to 12 carbon atoms, and heteroaryl with 3 to 12 carbon atoms; or R₁ and R₂ form a saturated or unsaturated 5-13 membered ring: or R₄ and R₅ form a saturated or unsaturated 5- 13 membered ring;
R₆, R₇, R₈, R₉, R₁₀, and R₁₁ are the same or different, and are each independently selected from the group consisting of deuterium, halogen, cyano, alkyl with 1 to 5 carbon atoms, trialkylsilyl with 3 to 12 carbon atoms, aryl with 6 to 12 carbon atoms, and heteroaryl with 3 to 12 carbon atoms;
R₆, R₇, R₈, R₉, R₁₀, and R₁₁ are collectively represented by Rᵢ: n₆, n₇, n₈, n₉, n₁₀, and n₁₁ are collectively represented by nᵢ; nᵢ indicates the number of Rᵢ, and i is a variable of 6, 7, 8, 9, 10, or 11; when i is 6, 8, or 10, nᵢ is selected from the group consisting of 0, 1, 2, 3, 4, 5, and 6; when i is 7, 9, or 11, nᵢ is selected from the group consisting of 0, 1, 2, 3, and 4; and when nᵢ is greater than 1, any two Rᵢ are the same or different;
Ar₂ is selected from the group consisting of substituted or unsubstituted aryl with 6 to 40 carbon atoms and substituted or unsubstituted heteroaryl with 3 to 30 carbon atoms;
L₁ and L₂ are the same or different, and are each independently selected from the group consisting of a single bond, substituted or unsubstituted arylene with 6 to 20 carbon atoms, and substituted or unsubstituted heteroarylene with 3 to 20 carbon atoms,
substituents in L₁, L₂, and Ar₂ are the same or different, and are each independently selected from the group consisting of deuterium, halogen, cyano, alkyl with I to 10 carbon atoms, trialkylsilyl with 3 to 12 carbon atoms, aryl with 6 to 20 carbon atoms, heteroaryl with 3 to 20 carbon atoms, haloalkyl with 1 to 10 carbon atoms, and alkoxy with 1 to 10 carbon atoms; and optionally, any two adjacent substituents in Ar₂ form a saturated or unsaturated 3-15 membered ring, for example, any two adjacent substituents form fluorenyl.

In the present application, “optionally, any two adjacent substituents in Ar₂ form a saturated or unsaturated 3-15 membered ring” means that any two adjacent substituents in Ar₂ may form a saturated or unsaturated 3-15 membered ring or may not form a saturated or unsaturated 3-150 membered ring.

In the present application,

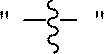

refers to a bond attached to other substituents or binding sites.

The description manners used in the present application such as “each are independently selected from the group consisting of” and “are each independently selected from the group consisting of” can be used interchangeably, and should be understood in a broad sense, which can mean that, in different groups, specific options expressed by the same symbol do not affect each other; or in the same group, specific options expressed by the same symbol do not affect each other. For example,

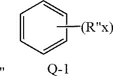

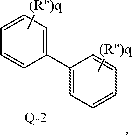

where q is independently 0, 1, 2, or 3 and substituents R″ each are independently selected from the group consisting of hydrogen, deuterium, fluorine, and chlorine” means that, in formula Q-1, there are q substituents R″ on the benzene ring, the substituents R″ can be the same or different, and options for each substituent R″ do not affect each other; and in formula Q-2, there are q substituents R″ on each benzene ring of the biphenyl, the numbers q of substituents R″ on the two benzene rings can be the same or different, the substituents R″ can be the same or different, and options for each substituent R″ do not affect each other.

In the present application, the term “substituted or unsubstituted” means that a functional group after the term may have or may not have a substituent (hereinafter, for ease of description, substituents are collectively referred to as Rc). For example, the “substituted or unsubstituted aryl” refers to Re-substituted aryl or unsubstituted aryl. The substituents Rc may include, for example, deuterium, halogen, cyano, heteroaryl, aryl, alkyl, haloalkyl, cycloalkyl, alkoxy, and trialkylsilyl; and optionally, any two of the substituents are linked to form a 3-15 membered saturated or unsaturated ring together with atoms attached to the two. In the present application, a substituted functional group may have one or more of the above-mentioned substituents Rc, where when two substituents Rc are attached to the same atom, these two substituents Rc may exist independently or are linked to form a ring with the atom; and when there are two adjacent substituents Rc on the functional group, the two adjacent substituents Rc may exist independently or are fused with the functional group to form a ring.

In the present application, the number of carbon atoms in a substituted or unsubstituted functional group refers to the number of all carbon atoms. For example, if L₁ is substituted arylene with 12 carbon atoms, the number of all carbon atoms in the arylene and substituents thereon is 12. For example, if Ar₁ is

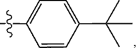

the number of carbon atoms in Ar₁ is 10; and if L₁ is,

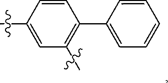

the number of carbon atoms in L₁ is 12.

In the present application, unless otherwise specifically defined, the term “hetero” means that a functional group includes at least one heteroatom such as B, N, O, S, P, Si, or Se, and the rest atoms in the functional group are carbon and hydrogen.

In the present application, the alkyl may include linear alkyl or branched alkyl. The alkyl may have 1 to 10 carbon atoms. In the present application, a numerical range such as “1 to 10” refers to any integer in the range. For example, alkyl with 1 to 10 carbon atoms refers to alkyl with 1, 2, 3, 4, 5, 6, 7, 8, 9, or 10 carbon atoms.

For example, the alkyl may be alkyl with 1 to 5 carbon atoms, and specific examples of the alkyl may include, but are not limited to, methyl, ethyl, propyl, isopropyl, n-butyl, isobutyl, sec-butyl, tert-butyl, and pentyl.

In the present application, the aryl refers to any functional group or substituent derived from an aromatic carbocyclic ring. The aryl may refer to a monocyclic aryl group (such as phenyl) or a polycyclic aryl group. In other words, the aryl may refer a monocyclic aryl group, a fused-ring aryl group, two or more monocyclic aryl groups that are conjugated through carbon-carbon bonds, a monocyclic aryl group and a fused-ring aryl group that are conjugated through carbon-carbon bonds, and two or more fused-ring aryl groups that are conjugated through carbon-carbon bonds. That is, unless otherwise specified, two or more aromatic groups that are conjugated through carbon-carbon bonds can also be regarded as the aryl of the present application. For example, the fused-ring aryl group may include a bicyclic fused aryl group (such as naphthyl) and a tricyclic fused aryl group (such as phenanthryl, fluorenyl, and anthracenyl). Examples of the aryl may include, but are not limited to, phenyl, naphthyl, fluorenyl, anthracenyl, phenanthryl, biphenyl, terphenyl, tetraphenyl, pentaphenyl, benzo[9,10]phenanthryl, pyrenyl, benzofluoranthenyl, and chrysenyl. In the present application, the biphenyl can be construed as phenyl-substituted aryl, and can also be construed as unsubstituted aryl.

The arylene involved in the present application refers to a divalent group obtained after one hydrogen atom is further removed from aryl.

In the present application, the substituted aryl may refer to aryl in which one or more hydrogen atoms are substituted by a group such as deuterium, halogen, cyano, trifluoromethyl, heteroaryl, aryl, trimethylsilyl, alkyl, cycloalkyl, or alkoxy. It should be understood that the number of carbon atoms in the substituted aryl refers to the total number of carbon atoms in the aryl and substituents thereon. For example, in substituted aryl with 18 carbon atoms, there are a total of 18 carbon atoms in the aryl and substituents thereon.

In the present application, the heteroaryl refers to a monovalent aromatic ring with 1, 2, 3, 4, 5, 6, 7, or more heteroatoms or a derivative thereof. The heteroatoms may be one or more selected from the group consisting of B, O, N, P, Si, Se, and S. The heteroaryl can be monocyclic heteroaryl or polycyclic heteroaryl. In other words, the heteroaryl may refer to a single aromatic ring system or multiple aromatic ring systems conjugated through carbon-carbon bonds, where each aromatic ring system is an aromatic monocyclic ring or an aromatic fused ring. For example, the heteroaryl may include, but is not limited to, thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolinyl, quinazolinyl, quinoxalinyl, phenoxazinyl, phthalazinyl, pyridopyrimidinyl, pyridopyrazinyl, pyrazinopyrazinyl, isoquinolinyl, indolyl, carbazolyl, benzoxazolyl, benzimidazolyl, benzothiazolyl, benzocarbazolyl, benzothienyl, dibenzothiophenyl, thienothienyl, benzofuranyl, phenanthrolinyl, isoxazolyl, thiadiazolyl, benzothiazolyl, phenothiazinyl, silylfluorenyl, dibenzofuranyl, N-phenylcarbazolyl, N-pyridylcarbazolyl, and N-methylcarbazolyl. The thienyl, furyl, phenanthrolinyl, and the like are heteroaryl with a single aromatic ring system; and the N-arylcarbazolyl, N-heteroarylcarbazolyl, and the like are heteroaryl with multiple ring systems conjugated through carbon-carbon bonds.

The heteroarylene involved in the present application refers to a divalent group obtained after one hydrogen atom is further removed from heteroaryl.

In the present application, substituted heteroaryl refers to heteroaryl in which one or more hydrogen atoms are substituted by groups such as deuterium, halogen, cyano, aryl, heteroaryl, trimethylsilyl, alkyl, cycloalky, and alkoxy. It should be understood that the number of carbon atoms in the substituted heteroaryl refers to the total number of carbon atoms in the heteroaryl and substituents thereon.

In the present application, specific examples of aryl as a substituent may include, but are not limited to, phenyl, biphenyl, naphthyl, anthracenyl, phenanthryl, and chrysenyl.

In the present application, specific examples of heteroaryl as a substituent may include, but are not limited to, pyridyl, carbazolyl, dibenzofuranyl, dibenzothiophenyl, quinolinyl, quinazolinyl, quinoxalinyl, and isoquinolinyl.

In the present application, the halogen may include fluorine, iodine, bromine, chlorine, or the like.

In the present application, specific examples of trialkylsilyl may include, but are not limited to, trimethylsilyl and triethylsilyl.

In the present application, specific examples of haloalkyl may include, but are not limited to, trifiuoromethyl.

In the present application, a non-positional bond refers to a single bond

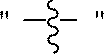

extending from a ring system, which means that one end of the bond can be attached to any position in the ring system through which the bond penetrates, and the other end is attached to the remaining part in the compound molecule.

For example, as shown in the following formula (f), the naphthyl represented by the formula (f) is attached to the remaining part in the molecule through two non-positional bonds that penetrate through the bicyclic ring, which indicates any possible attachment modes shown in formula (f-1) to formula (f-10):

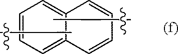

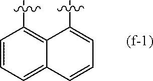

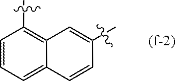

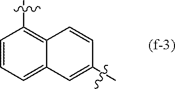

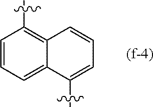

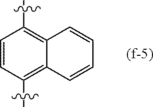

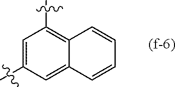

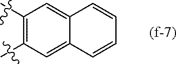

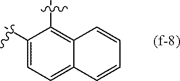

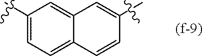

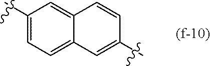

For example, as shown in the following formula (X′), the dibenzofuranyl represented by the formula (X′) is attached to the remaining part of the molecule through a non-positional bond extending from the middle of a benzene ring at a side, which indicates any possible attachment modes shown in formula (X′-1) to formula (X′-4):

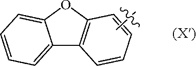

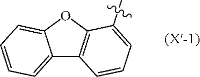

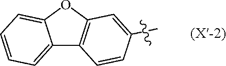

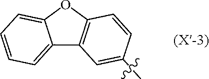

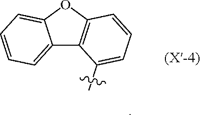

In the present application, in formula I, formula II, and formula III,

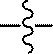

represents a chemical bond, where one end of the bond can be attached to any position of the benzodiphenyl five-membered ring shown in each formula, that is, the end can be attached to a carbon atom in a naphthalene ring or a carbon atom of a benzene ring, or may be attached to X. When an end of

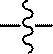

is attached to X and X is C(R₁R₂), N(R₃), or Si(R₄R₅), the end of

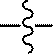

can be specifically attached to R₁, R₂, R₃, R₄, or R₅. For example, when Ar₁ is a group shown in formula I, both n₆ and n₇ in formula 1 are 0, and X is N(Ph). Ar₁ may have a structure shown as follows:

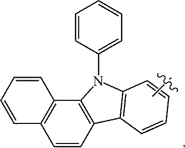

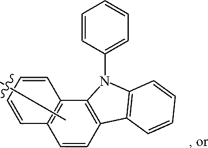

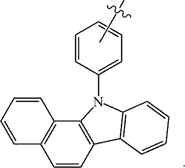

In an embodiment of the present application, L₁ and L₂ are each independently selected from the group consisting of a single bond and substituted or unsubstituted arylene with 6 to 12 carbon atoms. For example, L₁ and L₂ are each independently selected from the group consisting of a single bond and substituted or unsubstituted arylene with 6, 7, 8, 9, 10, 11, or 12 carbon atoms.

Optionally, L₁ and L₂ are each independently selected from the group consisting of a single bond, substituted or unsubstituted phenylene, substituted or unsubstituted naphthylene, and substituted or unsubstituted biphenylene.

Preferably, substituents in L₁ and L₂ are each independently selected from the group consisting of deuterium, fluorine, cyano, methyl, ethyl, isopropyl, tert-butyl, phenyl, naphthyl, and pyridyl.

Optionally, L₁ and L₂ are each independently selected from the group consisting of a single bond and a substituted or unsubstituted group V; an unsubstituted group V is selected from the group consisting of the following groups:

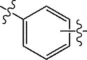

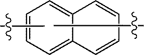

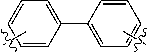

a substituted group V may have one or more substituents, and the one or more substituents are each independently selected from the group consisting of deuterium, fluorine, cyano, methyl, ethyl, isopropyl, tert-butyl, phenyl, naphthyl, and pyridyl; and when the substituted group V has two or more substituents, the two or more substituents are the same or different.

Optionally, L₁ and L₂ are each independently selected from the group consisting of a single bond and the following groups:

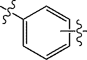

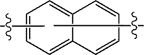

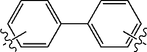

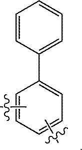

Further optionally, L₁ and L₂ are each independently selected from the group consisting of a single bond and the following groups:

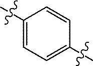

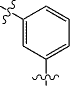

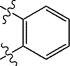

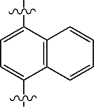

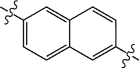

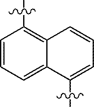

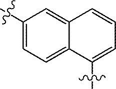

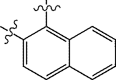

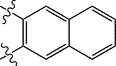

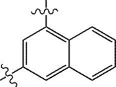

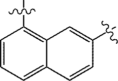

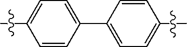

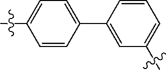

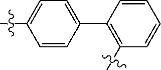

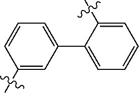

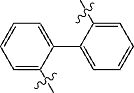

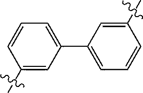

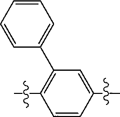

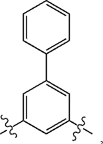

In an embodiment of the present application, Ar₂ is selected from the group consisting of substituted or unsubstituted aryl with 6 to 25 carbon atoms and substituted or unsubstituted heteroaryl with 3 to 20 carbon atoms. For example, Ar₂ is selected from the group consisting of substituted or unsubstituted aryl with 6, 7, 8, 9, 10, 11, 12, 13, 14, 15, 16, 17, 18, 19, 20, 21, 22, 23, 24, or 25 carbon atoms and substituted or unsubstituted heteroaryl with 3, 4, 5, 6, 7, 8, 9, 10, 11, 12, 13, 14, 15, 16, 17, 18, 19, or 20 carbon atoms.

Preferably, a substituent in Ar₂ is independently selected from the group consisting of deuterium, fluorine, cyano, trimethylsilyl, alkyl with 1 to 5 carbon atoms, aryl with 6 to 12 carbon atoms, and heteroaryl with 3 to 12 carbon atoms; and optionally, any two adjacent substituents in Ar₂ form a saturated or unsaturated 5-13 membered ring.

Optionally, Ar₂ is selected from the group consisting of substituted or unsubstituted phenyl, substituted or unsubstituted biphenyl, substituted or unsubstituted naphthyl, substituted or unsubstituted dibenzofuranyl, substituted or unsubstituted dibenzothiophenyl, substituted or unsubstituted fluorenyl, substituted or unsubstituted phenanthryl, and substituted or unsubstituted carbazolyl.

Preferably, a substituent in Ar₂ is independently selected from the group consisting of deuterium, cyano, fluorine, trimethylsilyl, methyl, ethyl, isopropyl, tert-butyl, phenyl, naphthyl, biphenyl, and pyridyl; and optionally, any two adjacent substituents in Ar₂ form a fluorene ring.

Optionally, Ar₂ is a substituted or unsubstituted group W; an unsubstituted group W is selected from the group consisting of the following groups:

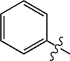

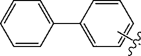

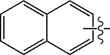

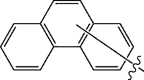

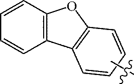

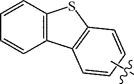

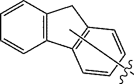

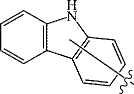

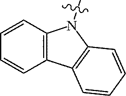

a substituted group W has one or more substituents, and the one or more substituents are each independently selected from the group consisting of deuterium, cyano, fluorine, trimethylsilyl, methyl, ethyl, isopropyl, tert-butyl, phenyl, naphthyl, biphenyl, and pyridyl; and when the substituted group W has two or more substituents, the two or more substituents are the same or different.

Optionally, Ar₂ is selected from the group consisting of the following groups:

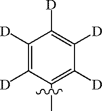

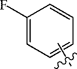

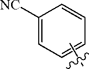

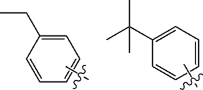

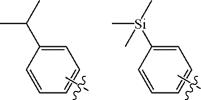

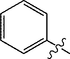

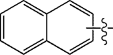

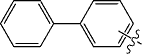

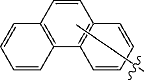

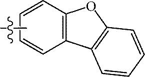

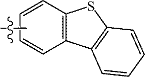

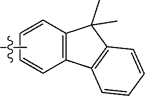

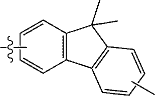

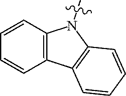

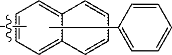

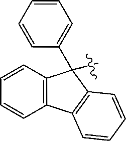

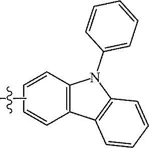

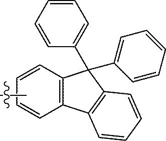

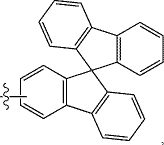

Further optionally, Ar₂ is selected from the group consisting of the following groups:

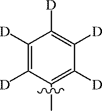

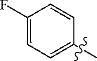

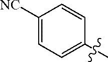

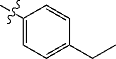

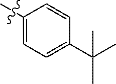

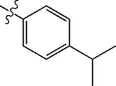

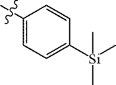

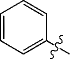

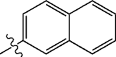

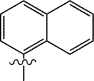

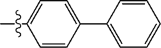

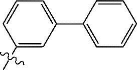

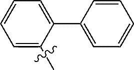

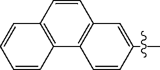

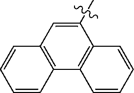

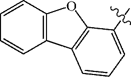

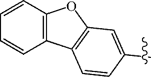

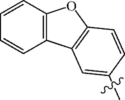

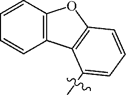

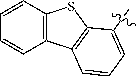

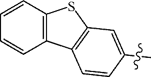

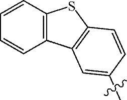

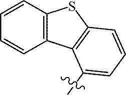

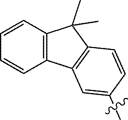

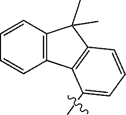

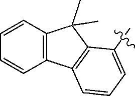

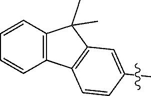

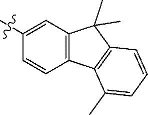

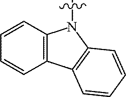

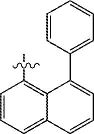

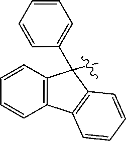

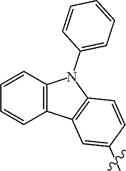

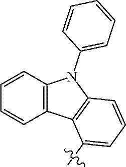

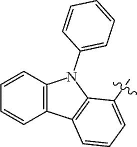

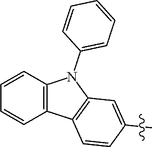

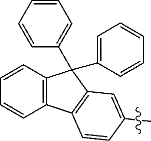

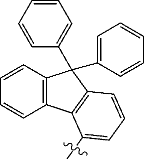

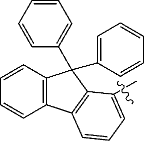

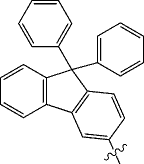

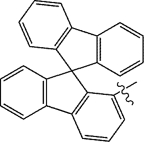

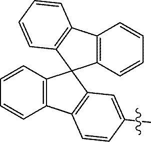

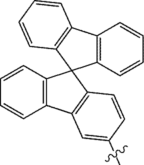

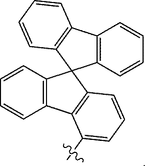

In an embodiment of the present application, R₁, R₂, R₃, R₄, and R₅ are each independently selected from the group consisting of methyl, ethyl, isopropyl, tert-butyl, phenyl, naphthyl, biphenyl, pyridyl, pyrimidinyl, quinolinyl, isoquinolinyl, carbazolyl, dibenzofuranyl, and dibenzothiophenyl; or, R₁ and R₂ form cyclopentane, cyclohexane, or a fluorene ring; or, R₄ and R₅ form cyclopentane, cyclohexane, or a fluorene ring.

Optionally, R₆, R₇, R₈, R₉, R₁₀, and R₁₁ are each independently selected from the group consisting of deuterium, fluorine, cyano, methyl, ethyl, isopropyl, tert-butyl, trimethylsilyl, phenyl, naphthyl, biphenyl, pyridyl, pyrimidinyl, quinolinyl, isoquinolinyl, carbazolyl, dibenzofuranyl, and dibenzothiophenyl.

In an embodiment of the present application. Ar₁ is a substituted or unsubstituted group Q; an unsubstituted group Q is selected from the group consisting of the following groups:

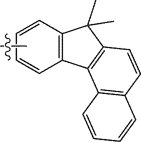

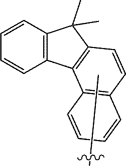

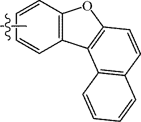

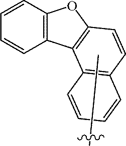

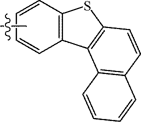

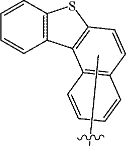

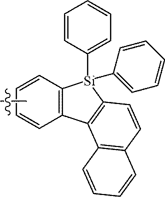

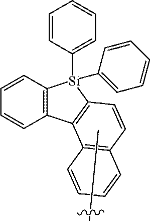

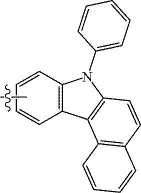

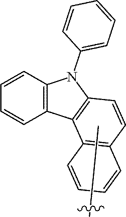

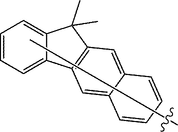

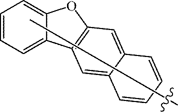

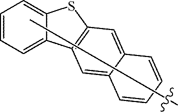

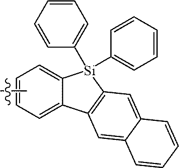

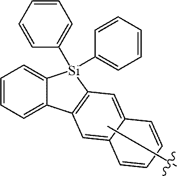

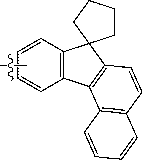

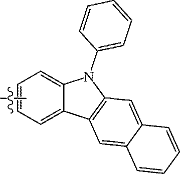

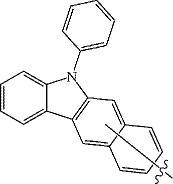

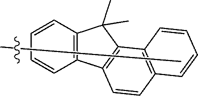

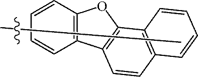

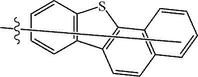

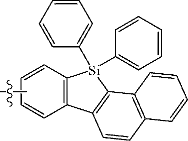

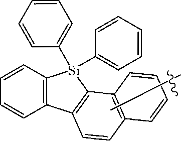

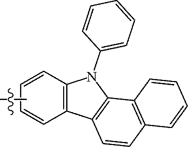

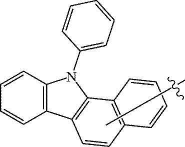

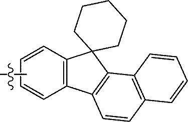

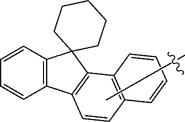

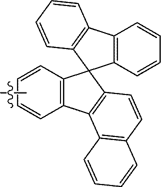

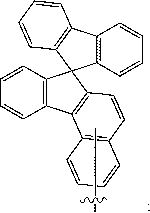

a substituted group Q has one or more substituents, and the one or more substituents are each independently selected from the group consisting of deuterium, cyano, fluorine, methyl, ethyl, isopropyl, tert-butyl, phenyl, naphthyl, biphenyl, pyridyl, cyclohexyl, carbazolyl, and trimethylsilyl; and when the substituted group Q has two or more substituents, the two or more substituents are the same or different.

Optionally, Ar₁ is selected from the group consisting of the following groups:

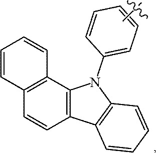

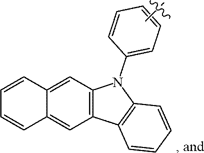

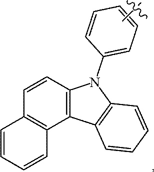

Optionally, the organic compound is selected from the group consisting of the following compounds:

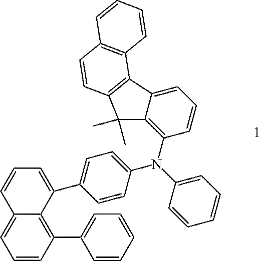 [SMILES: CC1(C)c2ccc3ccccc3c2-c2cccc(N(c3ccccc3)c3ccc(-c4cccc5cccc(-c6ccccc6)c45)cc3)c21]

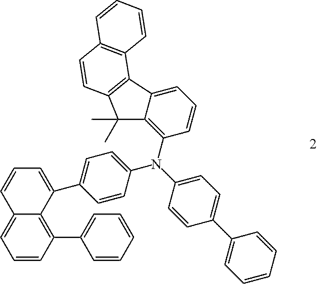 [SMILES: CC1(C)c2ccc3ccccc3c2-c2cccc(N(c3ccc(-c4ccccc4)cc3)c3ccc(-c4cccc5cccc(-c6ccccc6)c45)cc3)c21]

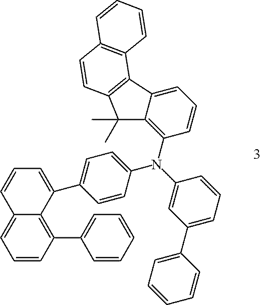 [SMILES: CC1(C)c2ccc3ccccc3c2-c2cccc(N(c3ccc(-c4cccc5cccc(-c6ccccc6)c45)cc3)c3cccc(-c4ccccc4)c3)c21]

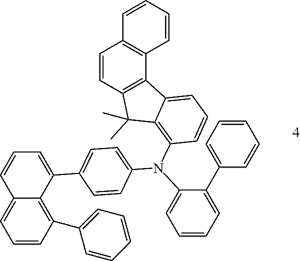 [SMILES: CC1(C)c2ccc3ccccc3c2-c2cccc(N(c3ccc(-c4cccc5cccc(-c6ccccc6)c45)cc3)c3ccccc3-c3ccccc3)c21]

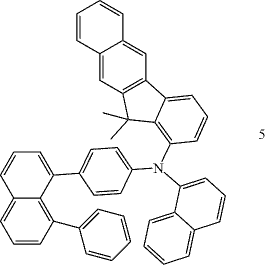 [SMILES: CC1(C)c2cc3ccccc3cc2-c2cccc(N(c3ccc(-c4cccc5cccc(-c6ccccc6)c45)cc3)c3cccc4ccccc34)c21]

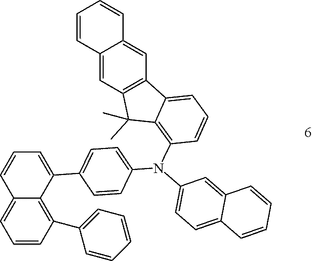 [SMILES: CC1(C)c2cc3ccccc3cc2-c2cccc(N(c3ccc(-c4cccc5cccc(-c6ccccc6)c45)cc3)c3ccc4ccccc4c3)c21]

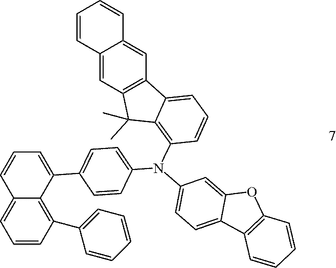 [SMILES: CC1(C)c2cc3ccccc3cc2-c2cccc(N(c3ccc(-c4cccc5cccc(-c6ccccc6)c45)cc3)c3ccc4c(c3)oc3ccccc34)c21]

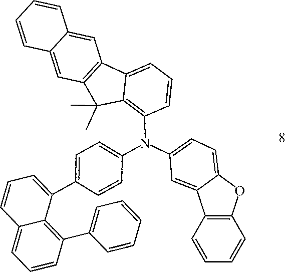 [SMILES: CC1(C)c2cc3ccccc3cc2-c2cccc(N(c3ccc(-c4cccc5cccc(-c6ccccc6)c45)cc3)c3ccc4oc5ccccc5c4c3)c21]

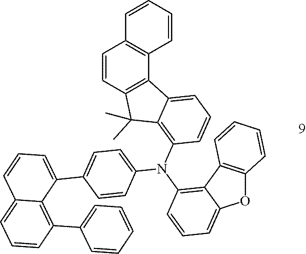 [SMILES: CC1(C)c2ccc3ccccc3c2-c2cccc(N(c3ccc(-c4cccc5cccc(-c6ccccc6)c45)cc3)c3cccc4oc5ccccc5c34)c21]

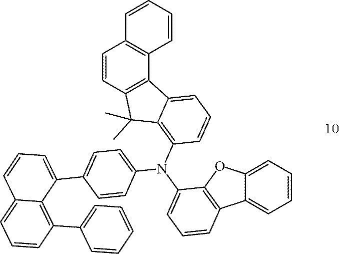 [SMILES: CC1(C)c2ccc3ccccc3c2-c2cccc(N(c3ccc(-c4cccc5cccc(-c6ccccc6)c45)cc3)c3cccc4c3oc3ccccc34)c21]

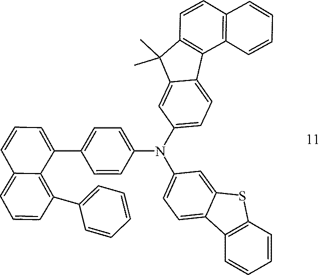 [SMILES: CC1(C)c2cc(N(c3ccc(-c4cccc5cccc(-c6ccccc6)c45)cc3)c3ccc4c(c3)sc3ccccc34)ccc2-c2c1ccc1ccccc21]

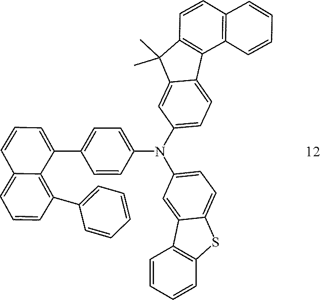 [SMILES: CC1(C)c2cc(N(c3ccc(-c4cccc5cccc(-c6ccccc6)c45)cc3)c3ccc4sc5ccccc5c4c3)ccc2-c2c1ccc1ccccc21]

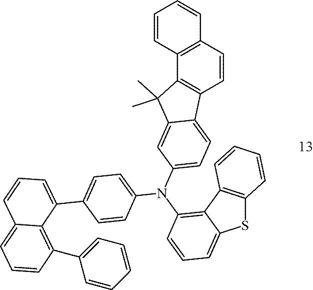 [SMILES: CC1(C)c2cc(N(c3ccc(-c4cccc5cccc(-c6ccccc6)c45)cc3)c3cccc4sc5ccccc5c34)ccc2-c2ccc3ccccc3c21]

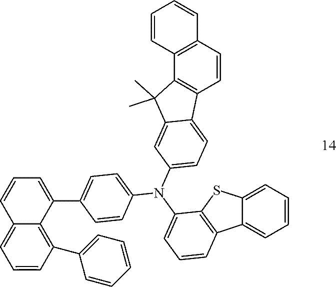 [SMILES: CC1(C)c2cc(N(c3ccc(-c4cccc5cccc(-c6ccccc6)c45)cc3)c3cccc4c3sc3ccccc34)ccc2-c2ccc3ccccc3c21]

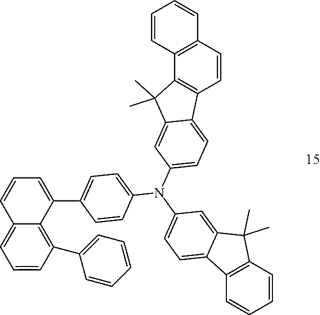 [SMILES: CC1(C)c2ccccc2-c2ccc(N(c3ccc(-c4cccc5cccc(-c6ccccc6)c45)cc3)c3ccc4c(c3)C(C)(C)c3c-4ccc4ccccc34)cc21]

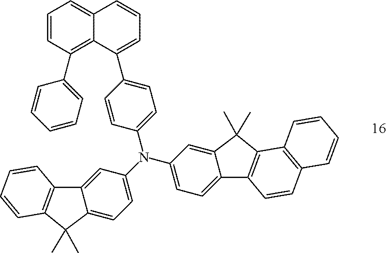 [SMILES: CC1(C)c2ccccc2-c2cc(N(c3ccc(-c4cccc5cccc(-c6ccccc6)c45)cc3)c3ccc4c(c3)C(C)(C)c3c-4ccc4ccccc34)ccc21]

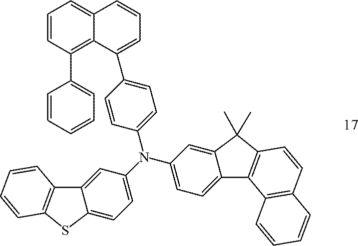 [SMILES: CC1(C)c2cc(N(c3ccc(-c4cccc5cccc(-c6ccccc6)c45)cc3)c3ccc4sc5ccccc5c4c3)ccc2-c2c1ccc1ccccc21]

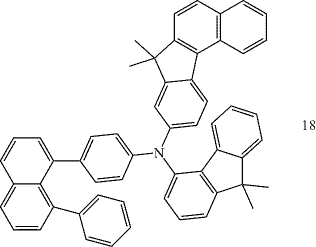 [SMILES: CC1(C)c2ccccc2-c2c(N(c3ccc(-c4cccc5cccc(-c6ccccc6)c45)cc3)c3ccc4c(c3)C(C)(C)c3ccc5ccccc5c3-4)cccc21]

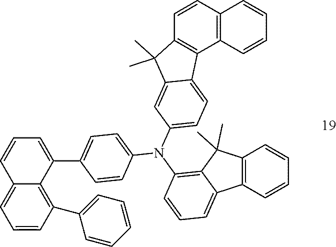 [SMILES: CC1(C)c2cc(N(c3ccc(-c4cccc5cccc(-c6ccccc6)c45)cc3)c3cccc4c3C(C)(C)c3ccccc3-4)ccc2-c2c1ccc1ccccc21]

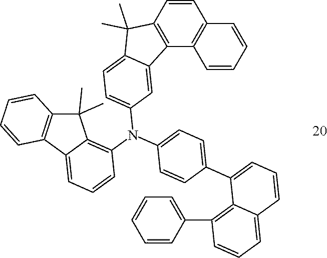 [SMILES: CC1(C)c2ccc(N(c3ccc(-c4cccc5cccc(-c6ccccc6)c45)cc3)c3cccc4c3C(C)(C)c3ccccc3-4)cc2-c2c1ccc1ccccc21]

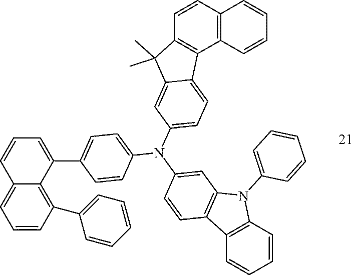 [SMILES: CC1(C)c2cc(N(c3ccc(-c4cccc5cccc(-c6ccccc6)c45)cc3)c3ccc4c5ccccc5n(-c5ccccc5)c4c3)ccc2-c2c1ccc1ccccc21]

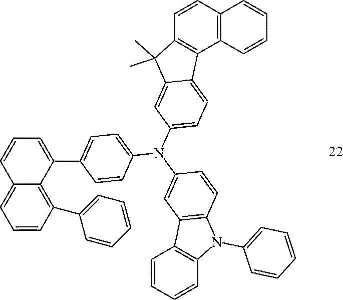 [SMILES: CC1(C)c2cc(N(c3ccc(-c4cccc5cccc(-c6ccccc6)c45)cc3)c3ccc4c(c3)c3ccccc3n4-c3ccccc3)ccc2-c2c1ccc1ccccc21]

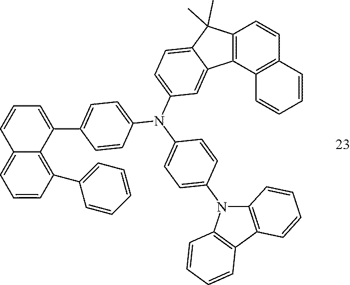 [SMILES: CC1(C)c2ccc(N(c3ccc(-c4cccc5cccc(-c6ccccc6)c45)cc3)c3ccc(-n4c5ccccc5c5ccccc54)cc3)cc2-c2c1ccc1ccccc21]

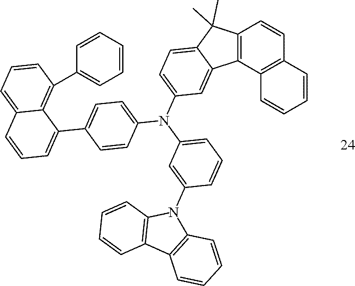 [SMILES: CC1(C)c2ccc(N(c3ccc(-c4cccc5cccc(-c6ccccc6)c45)cc3)c3cccc(-n4c5ccccc5c5ccccc54)c3)cc2-c2c1ccc1ccccc21]

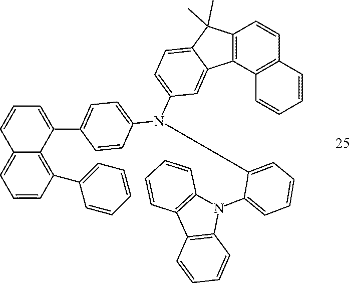 [SMILES: CC1(C)c2ccc(N(c3ccc(-c4cccc5cccc(-c6ccccc6)c45)cc3)c3ccccc3-n3c4ccccc4c4ccccc43)cc2-c2c1ccc1ccccc21]

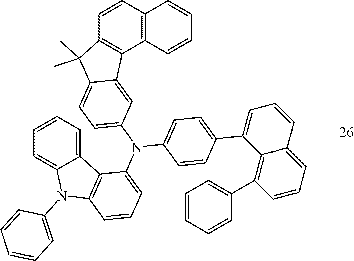 [SMILES: CC1(C)c2ccc(N(c3ccc(-c4cccc5cccc(-c6ccccc6)c45)cc3)c3cccc4c3c3ccccc3n4-c3ccccc3)cc2-c2c1ccc1ccccc21]

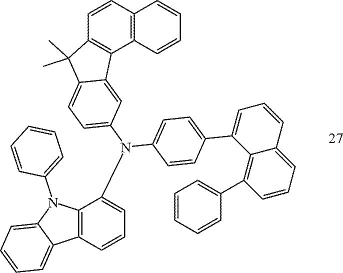 [SMILES: CC1(C)c2ccc(N(c3ccc(-c4cccc5cccc(-c6ccccc6)c45)cc3)c3cccc4c5ccccc5n(-c5ccccc5)c34)cc2-c2c1ccc1ccccc21]

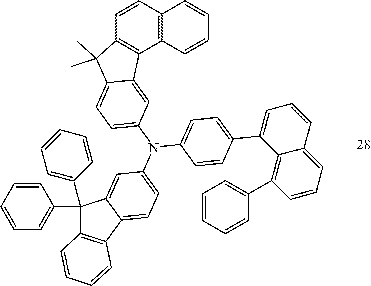 [SMILES: CC1(C)c2ccc(N(c3ccc(-c4cccc5cccc(-c6ccccc6)c45)cc3)c3ccc4c(c3)C(c3ccccc3)(c3ccccc3)c3ccccc3-4)cc2-c2c1ccc1ccccc21]

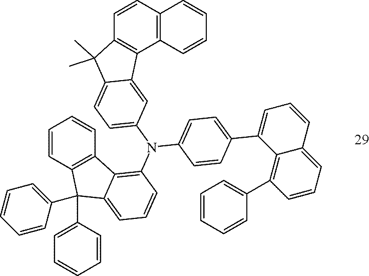 [SMILES: CC1(C)c2ccc(N(c3ccc(-c4cccc5cccc(-c6ccccc6)c45)cc3)c3cccc4c3-c3ccccc3C4(c3ccccc3)c3ccccc3)cc2-c2c1ccc1ccccc21]

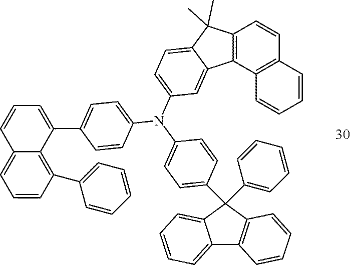 [SMILES: CC1(C)c2ccc(N(c3ccc(-c4cccc5cccc(-c6ccccc6)c45)cc3)c3ccc(C4(c5ccccc5)c5ccccc5-c5ccccc54)cc3)cc2-c2c1ccc1ccccc21]

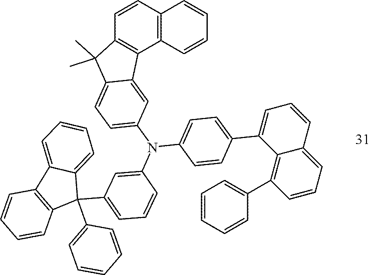 [SMILES: CC1(C)c2ccc(N(c3ccc(-c4cccc5cccc(-c6ccccc6)c45)cc3)c3cccc(C4(c5ccccc5)c5ccccc5-c5ccccc54)c3)cc2-c2c1ccc1ccccc21]

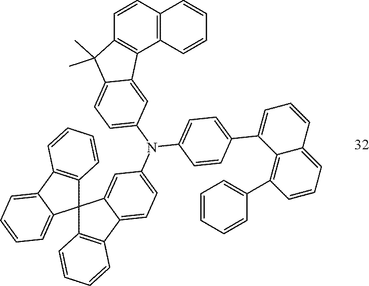 [SMILES: CC1(C)c2ccc(N(c3ccc(-c4cccc5cccc(-c6ccccc6)c45)cc3)c3ccc4c(c3)C3(c5ccccc5-c5ccccc53)c3ccccc3-4)cc2-c2c1ccc1ccccc21]

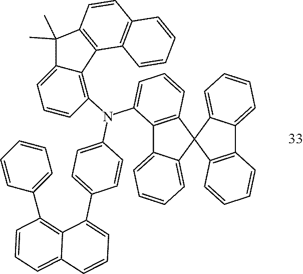 [SMILES: CC1(C)c2cccc(N(c3ccc(-c4cccc5cccc(-c6ccccc6)c45)cc3)c3cccc4c3-c3ccccc3C43c4ccccc4-c4ccccc43)c2-c2c1ccc1ccccc21]

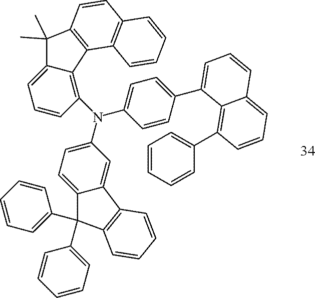 [SMILES: CC1(C)c2cccc(N(c3ccc(-c4cccc5cccc(-c6ccccc6)c45)cc3)c3ccc4c(c3)-c3ccccc3C4(c3ccccc3)c3ccccc3)c2-c2c1ccc1ccccc21]

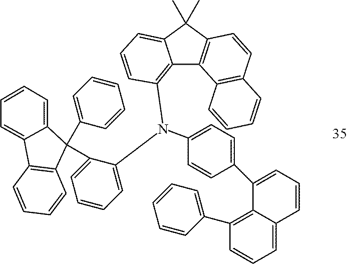 [SMILES: CC1(C)c2cccc(N(c3ccc(-c4cccc5cccc(-c6ccccc6)c45)cc3)c3ccccc3C3(c4ccccc4)c4ccccc4-c4ccccc43)c2-c2c1ccc1ccccc21]

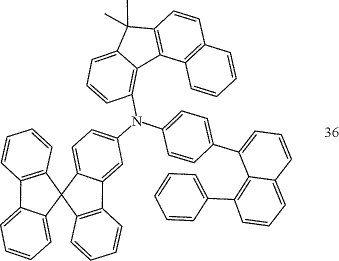 [SMILES: CC1(C)c2cccc(N(c3ccc(-c4cccc5cccc(-c6ccccc6)c45)cc3)c3ccc4c(c3)-c3ccccc3C43c4ccccc4-c4ccccc43)c2-c2c1ccc1ccccc21]

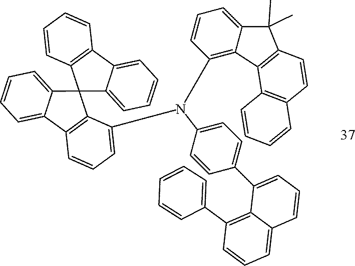 [SMILES: CC1(C)c2cccc(N(c3ccc(-c4cccc5cccc(-c6ccccc6)c45)cc3)c3cccc4c3C3(c5ccccc5-c5ccccc53)c3ccccc3-4)c2-c2c1ccc1ccccc21]

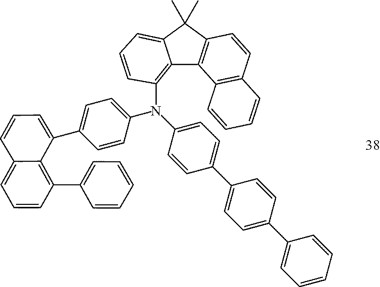 [SMILES: CC1(C)c2cccc(N(c3ccc(-c4ccc(-c5ccccc5)cc4)cc3)c3ccc(-c4cccc5cccc(-c6ccccc6)c45)cc3)c2-c2c1ccc1ccccc21]

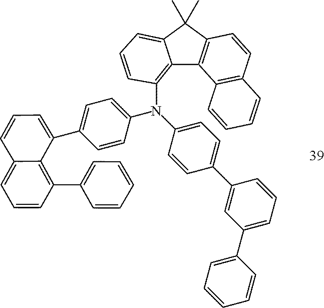 [SMILES: CC1(C)c2cccc(N(c3ccc(-c4cccc(-c5ccccc5)c4)cc3)c3ccc(-c4cccc5cccc(-c6ccccc6)c45)cc3)c2-c2c1ccc1ccccc21]

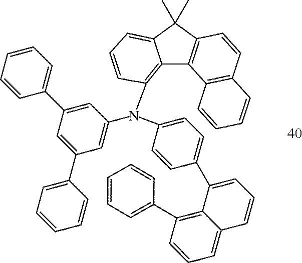 [SMILES: CC1(C)c2cccc(N(c3ccc(-c4cccc5cccc(-c6ccccc6)c45)cc3)c3cc(-c4ccccc4)cc(-c4ccccc4)c3)c2-c2c1ccc1ccccc21]

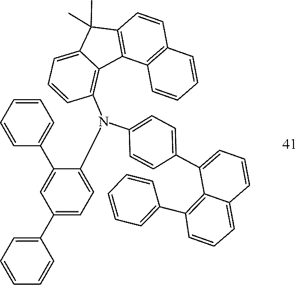 [SMILES: CC1(C)c2cccc(N(c3ccc(-c4cccc5cccc(-c6ccccc6)c45)cc3)c3ccc(-c4ccccc4)cc3-c3ccccc3)c2-c2c1ccc1ccccc21]

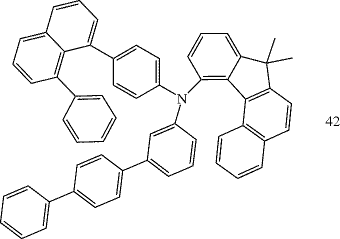 [SMILES: CC1(C)c2cccc(N(c3ccc(-c4cccc5cccc(-c6ccccc6)c45)cc3)c3cccc(-c4ccc(-c5ccccc5)cc4)c3)c2-c2c1ccc1ccccc21]

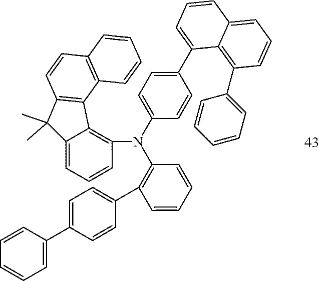 [SMILES: CC1(C)c2cccc(N(c3ccc(-c4cccc5cccc(-c6ccccc6)c45)cc3)c3ccccc3-c3ccc(-c4ccccc4)cc3)c2-c2c1ccc1ccccc21]

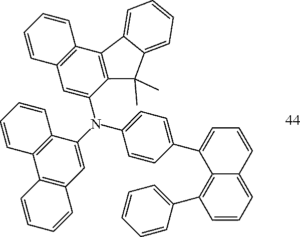 [SMILES: CC1(C)c2ccccc2-c2c1c(N(c1ccc(-c3cccc4cccc(-c5ccccc5)c34)cc1)c1cc3ccccc3c3ccccc13)cc1ccccc21]

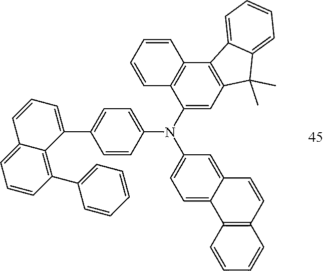 [SMILES: CC1(C)c2ccccc2-c2c1cc(N(c1ccc(-c3cccc4cccc(-c5ccccc5)c34)cc1)c1ccc3c(ccc4ccccc43)c1)c1ccccc21]

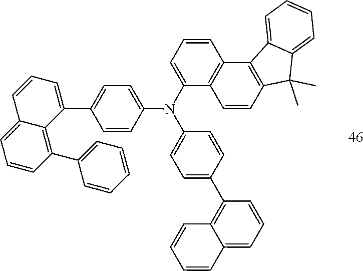 [SMILES: CC1(C)c2ccccc2-c2c1ccc1c(N(c3ccc(-c4cccc5ccccc45)cc3)c3ccc(-c4cccc5cccc(-c6ccccc6)c45)cc3)cccc21]

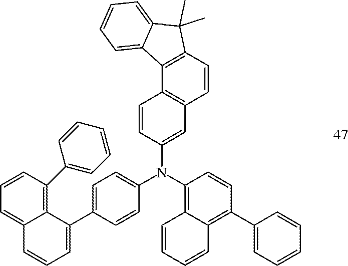 [SMILES: CC1(C)c2ccccc2-c2c1ccc1cc(N(c3ccc(-c4cccc5cccc(-c6ccccc6)c45)cc3)c3ccc(-c4ccccc4)c4ccccc34)ccc21]

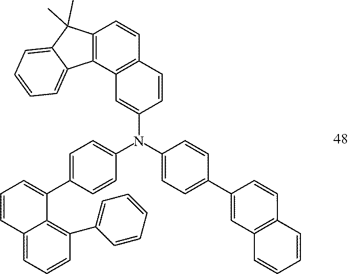 [SMILES: CC1(C)c2ccccc2-c2c1ccc1ccc(N(c3ccc(-c4ccc5ccccc5c4)cc3)c3ccc(-c4cccc5cccc(-c6ccccc6)c45)cc3)cc21]

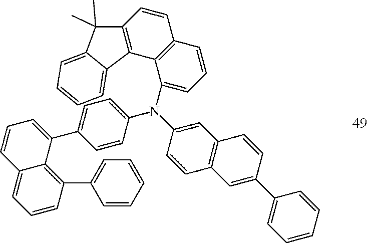 [SMILES: CC1(C)c2ccccc2-c2c1ccc1cccc(N(c3ccc(-c4cccc5cccc(-c6ccccc6)c45)cc3)c3ccc4cc(-c5ccccc5)ccc4c3)c21]

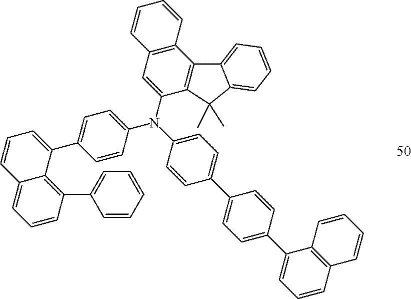 [SMILES: CC1(C)c2ccccc2-c2c1c(N(c1ccc(-c3ccc(-c4cccc5ccccc45)cc3)cc1)c1ccc(-c3cccc4cccc(-c5ccccc5)c34)cc1)cc1ccccc21]

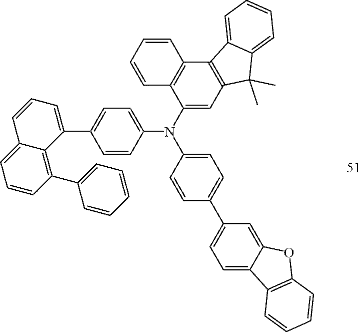 [SMILES: CC1(C)c2ccccc2-c2c1cc(N(c1ccc(-c3ccc4c(c3)oc3ccccc34)cc1)c1ccc(-c3cccc4cccc(-c5ccccc5)c34)cc1)c1ccccc21]

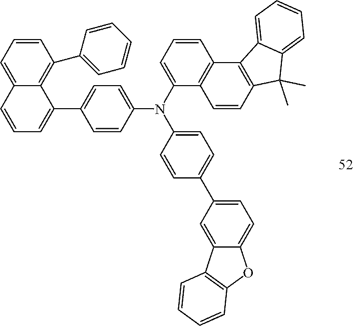 [SMILES: CC1(C)c2ccccc2-c2c1ccc1c(N(c3ccc(-c4ccc5oc6ccccc6c5c4)cc3)c3ccc(-c4cccc5cccc(-c6ccccc6)c45)cc3)cccc21]

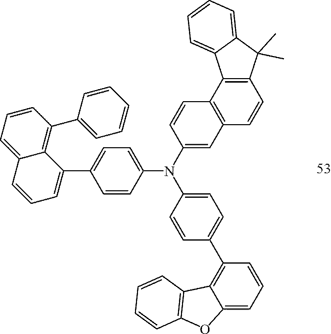 [SMILES: CC1(C)c2ccccc2-c2c1ccc1cc(N(c3ccc(-c4cccc5cccc(-c6ccccc6)c45)cc3)c3ccc(-c4cccc5oc6ccccc6c45)cc3)ccc21]

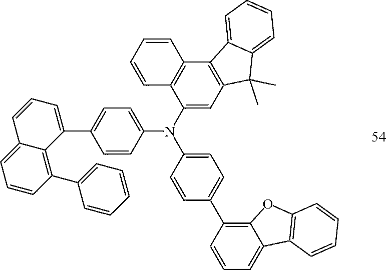 [SMILES: CC1(C)c2ccccc2-c2c1cc(N(c1ccc(-c3cccc4c3oc3ccccc34)cc1)c1ccc(-c3cccc4cccc(-c5ccccc5)c34)cc1)c1ccccc21]

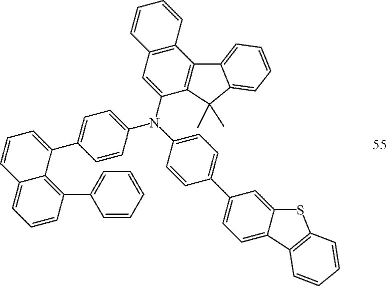 [SMILES: CC1(C)c2ccccc2-c2c1c(N(c1ccc(-c3ccc4c(c3)sc3ccccc34)cc1)c1ccc(-c3cccc4cccc(-c5ccccc5)c34)cc1)cc1ccccc21]

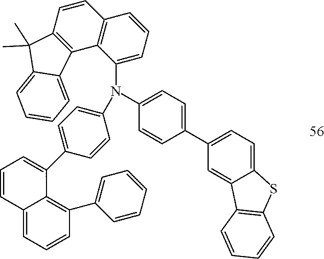 [SMILES: CC1(C)c2ccccc2-c2c1ccc1cccc(N(c3ccc(-c4ccc5sc6ccccc6c5c4)cc3)c3ccc(-c4cccc5cccc(-c6ccccc6)c45)cc3)c21]

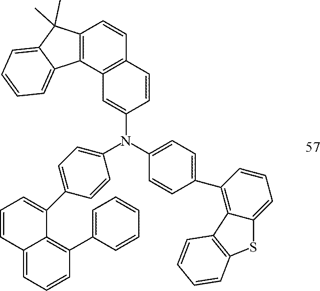 [SMILES: CC1(C)c2ccccc2-c2c1ccc1ccc(N(c3ccc(-c4cccc5cccc(-c6ccccc6)c45)cc3)c3ccc(-c4cccc5sc6ccccc6c45)cc3)cc21]

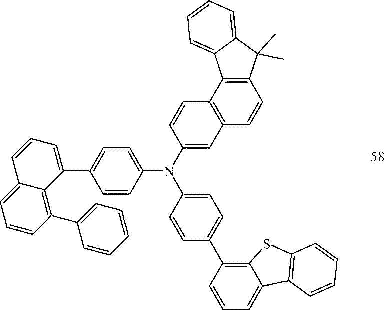 [SMILES: CC1(C)c2ccccc2-c2c1ccc1cc(N(c3ccc(-c4cccc5c4sc4ccccc45)cc3)c3ccc(-c4cccc5cccc(-c6ccccc6)c45)cc3)ccc21]

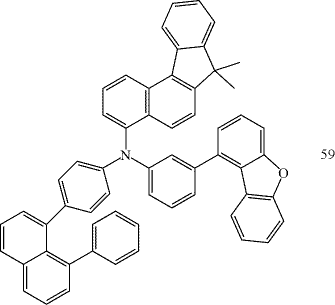 [SMILES: CC1(C)c2ccccc2-c2c1ccc1c(N(c3ccc(-c4cccc5cccc(-c6ccccc6)c45)cc3)c3cccc(-c4cccc5oc6ccccc6c45)c3)cccc21]

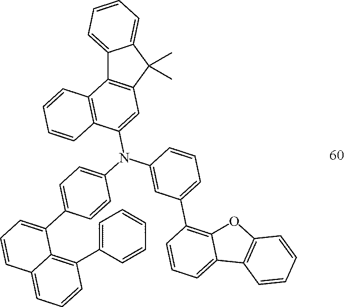 [SMILES: CC1(C)c2ccccc2-c2c1cc(N(c1ccc(-c3cccc4cccc(-c5ccccc5)c34)cc1)c1cccc(-c3cccc4c3oc3ccccc34)c1)c1ccccc21]

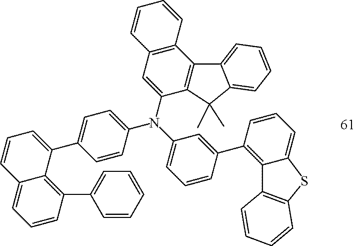 [SMILES: CC1(C)c2ccccc2-c2c1c(N(c1ccc(-c3cccc4cccc(-c5ccccc5)c34)cc1)c1cccc(-c3cccc4sc5ccccc5c34)c1)cc1ccccc21]

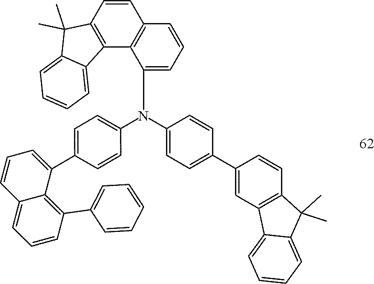 [SMILES: CC1(C)c2ccccc2-c2cc(-c3ccc(N(c4ccc(-c5cccc6cccc(-c7ccccc7)c56)cc4)c4cccc5ccc6c(c45)-c4ccccc4C6(C)C)cc3)ccc21]

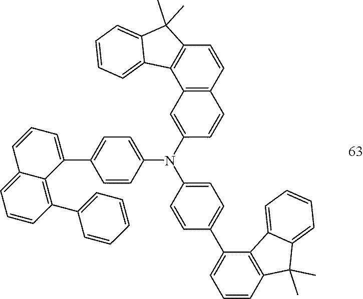 [SMILES: CC1(C)c2ccccc2-c2c(-c3ccc(N(c4ccc(-c5cccc6cccc(-c7ccccc7)c56)cc4)c4ccc5ccc6c(c5c4)-c4ccccc4C6(C)C)cc3)cccc21]

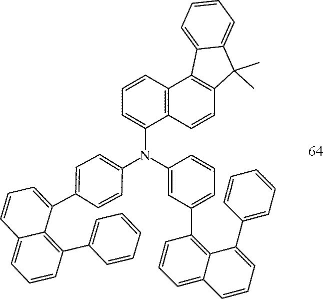 [SMILES: CC1(C)c2ccccc2-c2c1ccc1c(N(c3ccc(-c4cccc5cccc(-c6ccccc6)c45)cc3)c3cccc(-c4cccc5cccc(-c6ccccc6)c45)c3)cccc21]

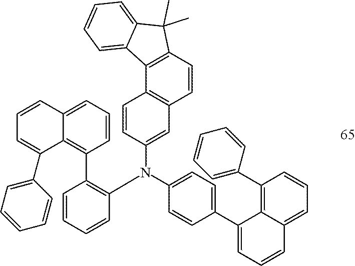 [SMILES: CC1(C)c2ccccc2-c2c1ccc1cc(N(c3ccc(-c4cccc5cccc(-c6ccccc6)c45)cc3)c3ccccc3-c3cccc4cccc(-c5ccccc5)c34)ccc21]

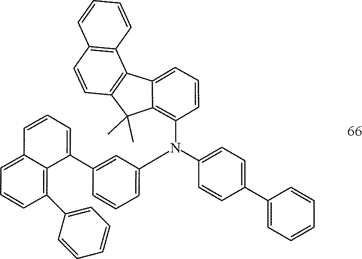 [SMILES: CC1(C)c2ccc3ccccc3c2-c2cccc(N(c3ccc(-c4ccccc4)cc3)c3cccc(-c4cccc5cccc(-c6ccccc6)c45)c3)c21]

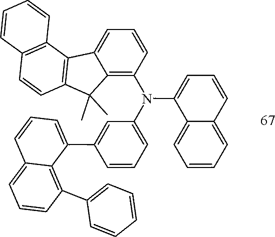 [SMILES: CC1(C)c2ccc3ccccc3c2-c2cccc(N(c3cccc(-c4cccc5cccc(-c6ccccc6)c45)c3)c3cccc4ccccc34)c21]

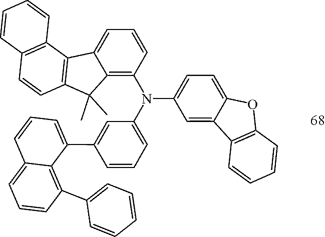 [SMILES: CC1(C)c2ccc3ccccc3c2-c2cccc(N(c3cccc(-c4cccc5cccc(-c6ccccc6)c45)c3)c3ccc4oc5ccccc5c4c3)c21]

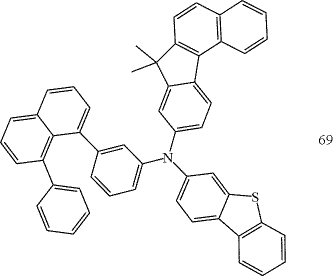 [SMILES: CC1(C)c2cc(N(c3cccc(-c4cccc5cccc(-c6ccccc6)c45)c3)c3ccc4c(c3)sc3ccccc34)ccc2-c2c1ccc1ccccc21]

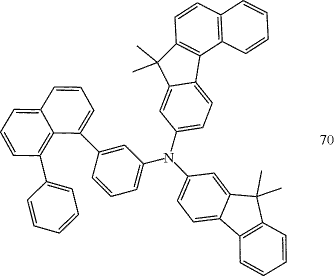 [SMILES: CC1(C)c2ccccc2-c2ccc(N(c3cccc(-c4cccc5cccc(-c6ccccc6)c45)c3)c3ccc4c(c3)C(C)(C)c3ccc5ccccc5c3-4)cc21]

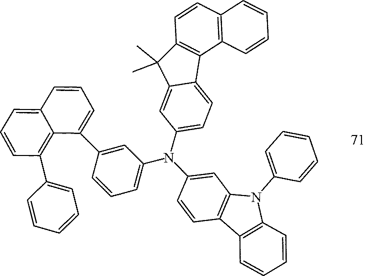 [SMILES: CC1(C)c2cc(N(c3cccc(-c4cccc5cccc(-c6ccccc6)c45)c3)c3ccc4c5ccccc5n(-c5ccccc5)c4c3)ccc2-c2c1ccc1ccccc21]

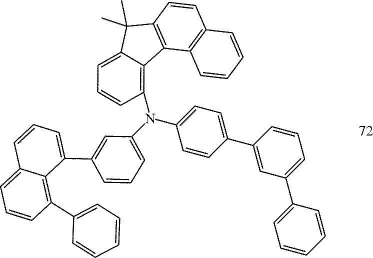 [SMILES: CC1(C)c2cccc(N(c3ccc(-c4cccc(-c5ccccc5)c4)cc3)c3cccc(-c4cccc5cccc(-c6ccccc6)c45)c3)c2-c2c1ccc1ccccc21]

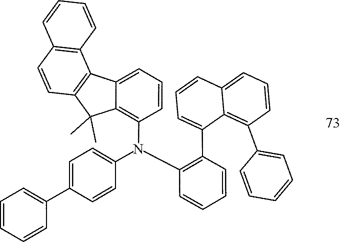 [SMILES: CC1(C)c2ccc3ccccc3c2-c2cccc(N(c3ccc(-c4ccccc4)cc3)c3ccccc3-c3cccc4cccc(-c5ccccc5)c34)c21]

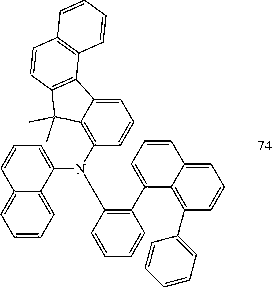 [SMILES: CC1(C)c2ccc3ccccc3c2-c2cccc(N(c3ccccc3-c3cccc4cccc(-c5ccccc5)c34)c3cccc4ccccc34)c21]

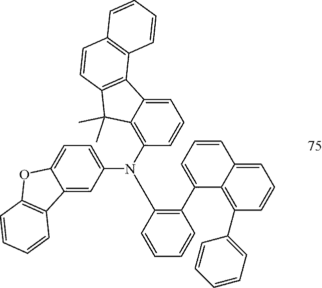 [SMILES: CC1(C)c2ccc3ccccc3c2-c2cccc(N(c3ccc4oc5ccccc5c4c3)c3ccccc3-c3cccc4cccc(-c5ccccc5)c34)c21]

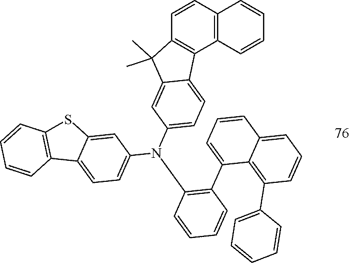 [SMILES: CC1(C)c2cc(N(c3ccc4c(c3)sc3ccccc34)c3ccccc3-c3cccc4cccc(-c5ccccc5)c34)ccc2-c2c1ccc1ccccc21]

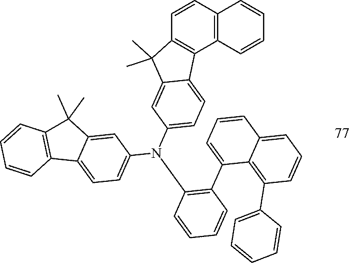 [SMILES: CC1(C)c2ccccc2-c2ccc(N(c3ccc4c(c3)C(C)(C)c3ccc5ccccc5c3-4)c3ccccc3-c3cccc4cccc(-c5ccccc5)c34)cc21]

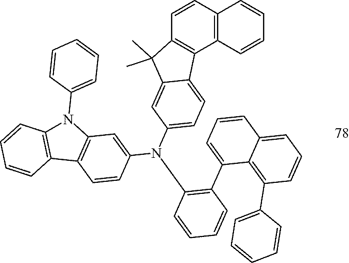 [SMILES: CC1(C)c2cc(N(c3ccc4c5ccccc5n(-c5ccccc5)c4c3)c3ccccc3-c3cccc4cccc(-c5ccccc5)c34)ccc2-c2c1ccc1ccccc21]

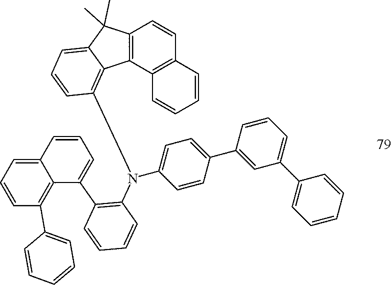 [SMILES: CC1(C)c2cccc(N(c3ccc(-c4cccc(-c5ccccc5)c4)cc3)c3ccccc3-c3cccc4cccc(-c5ccccc5)c34)c2-c2c1ccc1ccccc21]

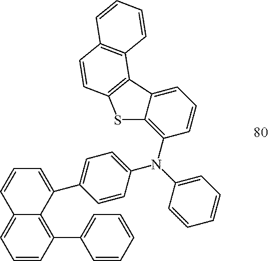 [SMILES: c1ccc(-c2cccc3cccc(-c4ccc(N(c5ccccc5)c5cccc6c5sc5ccc7ccccc7c56)cc4)c23)cc1]

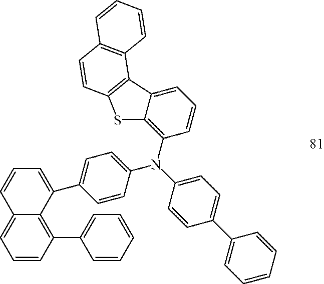 [SMILES: c1ccc(-c2ccc(N(c3ccc(-c4cccc5cccc(-c6ccccc6)c45)cc3)c3cccc4c3sc3ccc5ccccc5c34)cc2)cc1]

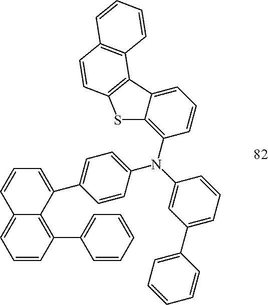 [SMILES: c1ccc(-c2cccc(N(c3ccc(-c4cccc5cccc(-c6ccccc6)c45)cc3)c3cccc4c3sc3ccc5ccccc5c34)c2)cc1]

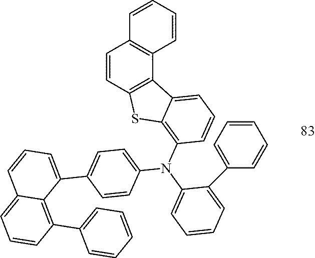 [SMILES: c1ccc(-c2ccccc2N(c2ccc(-c3cccc4cccc(-c5ccccc5)c34)cc2)c2cccc3c2sc2ccc4ccccc4c23)cc1]

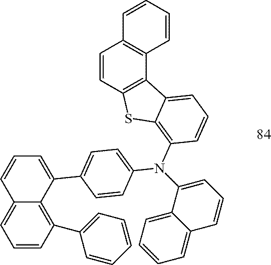 [SMILES: c1ccc(-c2cccc3cccc(-c4ccc(N(c5cccc6ccccc56)c5cccc6c5sc5ccc7ccccc7c56)cc4)c23)cc1]

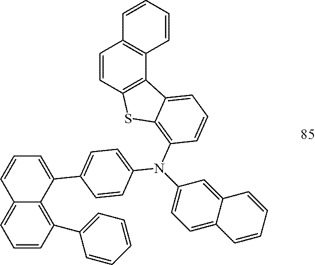 [SMILES: c1ccc(-c2cccc3cccc(-c4ccc(N(c5ccc6ccccc6c5)c5cccc6c5sc5ccc7ccccc7c56)cc4)c23)cc1]

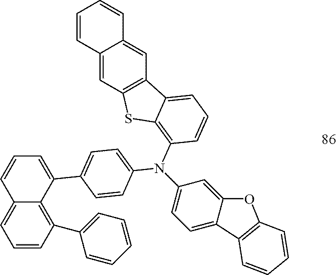 [SMILES: c1ccc(-c2cccc3cccc(-c4ccc(N(c5ccc6c(c5)oc5ccccc56)c5cccc6c5sc5cc7ccccc7cc56)cc4)c23)cc1]

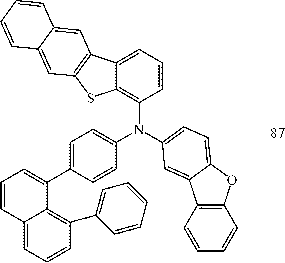 [SMILES: c1ccc(-c2cccc3cccc(-c4ccc(N(c5ccc6oc7ccccc7c6c5)c5cccc6c5sc5cc7ccccc7cc56)cc4)c23)cc1]

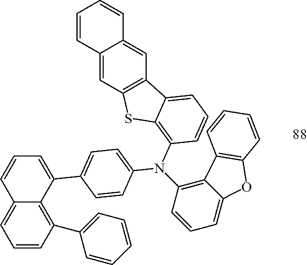 [SMILES: c1ccc(-c2cccc3cccc(-c4ccc(N(c5cccc6c5sc5cc7ccccc7cc56)c5cccc6oc7ccccc7c56)cc4)c23)cc1]

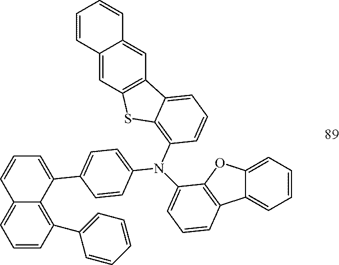 [SMILES: c1ccc(-c2cccc3cccc(-c4ccc(N(c5cccc6c5oc5ccccc56)c5cccc6c5sc5cc7ccccc7cc56)cc4)c23)cc1]

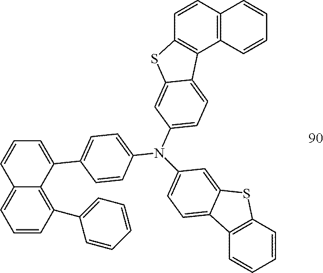 [SMILES: c1ccc(-c2cccc3cccc(-c4ccc(N(c5ccc6c(c5)sc5ccccc56)c5ccc6c(c5)sc5ccc7ccccc7c56)cc4)c23)cc1]

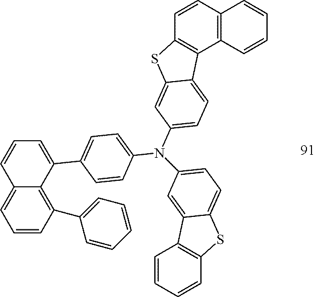 [SMILES: c1ccc(-c2cccc3cccc(-c4ccc(N(c5ccc6c(c5)sc5ccc7ccccc7c56)c5ccc6sc7ccccc7c6c5)cc4)c23)cc1]

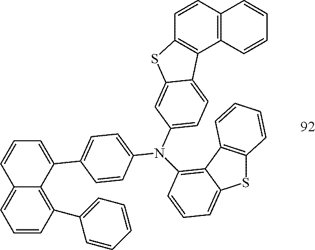 [SMILES: c1ccc(-c2cccc3cccc(-c4ccc(N(c5ccc6c(c5)sc5ccc7ccccc7c56)c5cccc6sc7ccccc7c56)cc4)c23)cc1]

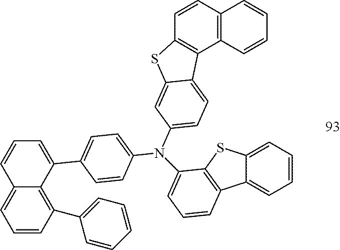 [SMILES: c1ccc(-c2cccc3cccc(-c4ccc(N(c5ccc6c(c5)sc5ccc7ccccc7c56)c5cccc6c5sc5ccccc56)cc4)c23)cc1]

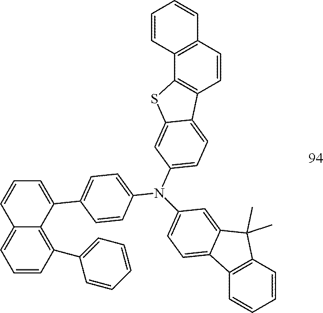 [SMILES: CC1(C)c2ccccc2-c2ccc(N(c3ccc(-c4cccc5cccc(-c6ccccc6)c45)cc3)c3ccc4c(c3)sc3c5ccccc5ccc43)cc21]

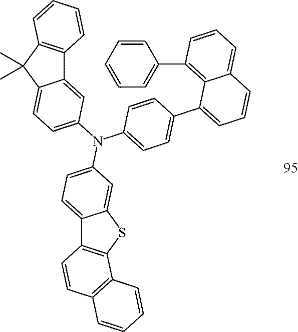 [SMILES: CC1(C)c2ccccc2-c2cc(N(c3ccc(-c4cccc5cccc(-c6ccccc6)c45)cc3)c3ccc4c(c3)sc3c5ccccc5ccc43)ccc21]

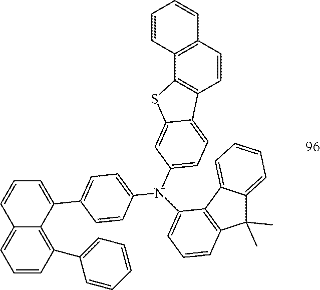 [SMILES: CC1(C)c2ccccc2-c2c(N(c3ccc(-c4cccc5cccc(-c6ccccc6)c45)cc3)c3ccc4c(c3)sc3c5ccccc5ccc43)cccc21]

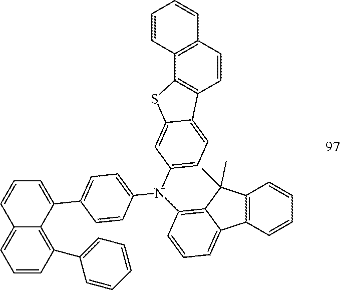 [SMILES: CC1(C)c2ccccc2-c2cccc(N(c3ccc(-c4cccc5cccc(-c6ccccc6)c45)cc3)c3ccc4c(c3)sc3c5ccccc5ccc43)c21]

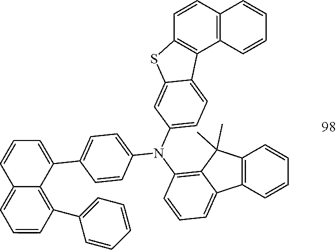 [SMILES: CC1(C)c2ccccc2-c2cccc(N(c3ccc(-c4cccc5cccc(-c6ccccc6)c45)cc3)c3ccc4c(c3)sc3ccc5ccccc5c34)c21]

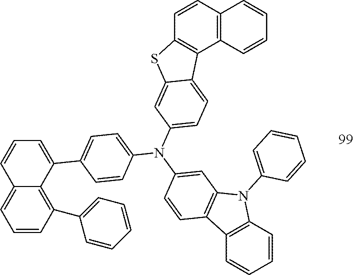 [SMILES: c1ccc(-c2cccc3cccc(-c4ccc(N(c5ccc6c(c5)sc5ccc7ccccc7c56)c5ccc6c7ccccc7n(-c7ccccc7)c6c5)cc4)c23)cc1]

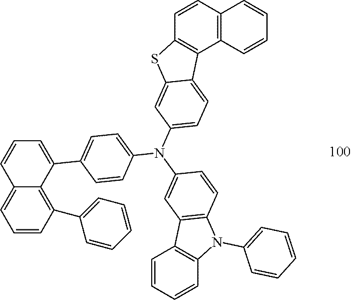 [SMILES: c1ccc(-c2cccc3cccc(-c4ccc(N(c5ccc6c(c5)sc5ccc7ccccc7c56)c5ccc6c(c5)c5ccccc5n6-c5ccccc5)cc4)c23)cc1]

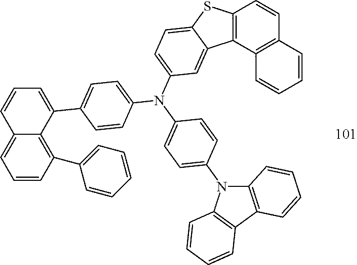 [SMILES: c1ccc(-c2cccc3cccc(-c4ccc(N(c5ccc(-n6c7ccccc7c7ccccc76)cc5)c5ccc6sc7ccc8ccccc8c7c6c5)cc4)c23)cc1]

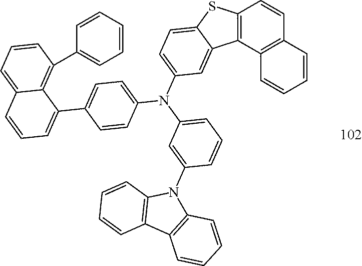 [SMILES: c1ccc(-c2cccc3cccc(-c4ccc(N(c5cccc(-n6c7ccccc7c7ccccc76)c5)c5ccc6sc7ccc8ccccc8c7c6c5)cc4)c23)cc1]

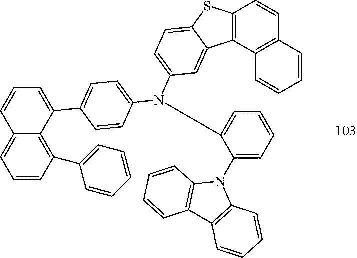 [SMILES: c1ccc(-c2cccc3cccc(-c4ccc(N(c5ccc6sc7ccc8ccccc8c7c6c5)c5ccccc5-n5c6ccccc6c6ccccc65)cc4)c23)cc1]

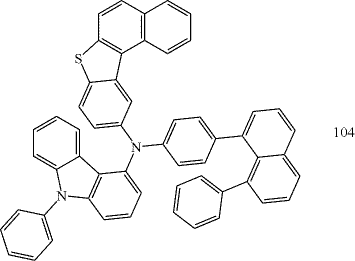 [SMILES: c1ccc(-c2cccc3cccc(-c4ccc(N(c5ccc6sc7ccc8ccccc8c7c6c5)c5cccc6c5c5ccccc5n6-c5ccccc5)cc4)c23)cc1]

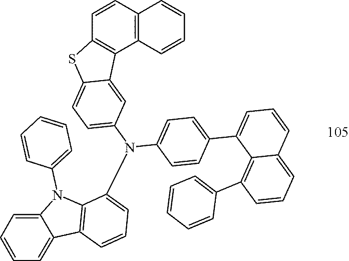 [SMILES: c1ccc(-c2cccc3cccc(-c4ccc(N(c5ccc6sc7ccc8ccccc8c7c6c5)c5cccc6c7ccccc7n(-c7ccccc7)c56)cc4)c23)cc1]

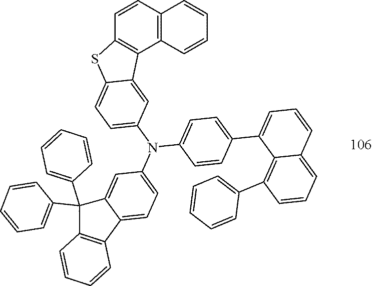 [SMILES: c1ccc(-c2cccc3cccc(-c4ccc(N(c5ccc6c(c5)C(c5ccccc5)(c5ccccc5)c5ccccc5-6)c5ccc6sc7ccc8ccccc8c7c6c5)cc4)c23)cc1]

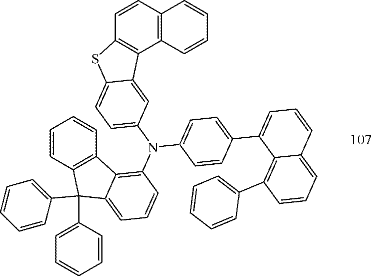 [SMILES: c1ccc(-c2cccc3cccc(-c4ccc(N(c5ccc6sc7ccc8ccccc8c7c6c5)c5cccc6c5-c5ccccc5C6(c5ccccc5)c5ccccc5)cc4)c23)cc1]

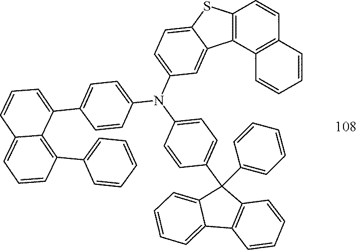 [SMILES: c1ccc(-c2cccc3cccc(-c4ccc(N(c5ccc(C6(c7ccccc7)c7ccccc7-c7ccccc76)cc5)c5ccc6sc7ccc8ccccc8c7c6c5)cc4)c23)cc1]

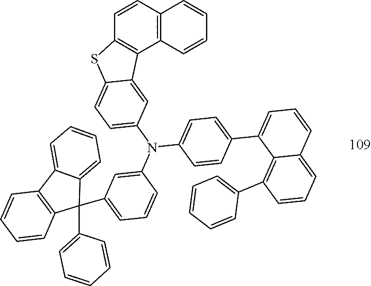 [SMILES: c1ccc(-c2cccc3cccc(-c4ccc(N(c5cccc(C6(c7ccccc7)c7ccccc7-c7ccccc76)c5)c5ccc6sc7ccc8ccccc8c7c6c5)cc4)c23)cc1]

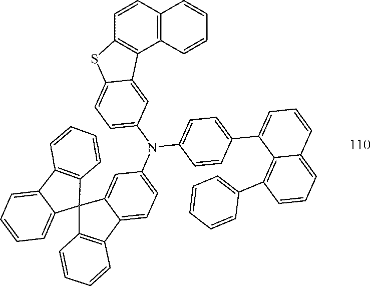 [SMILES: c1ccc(-c2cccc3cccc(-c4ccc(N(c5ccc6c(c5)C5(c7ccccc7-c7ccccc75)c5ccccc5-6)c5ccc6sc7ccc8ccccc8c7c6c5)cc4)c23)cc1]

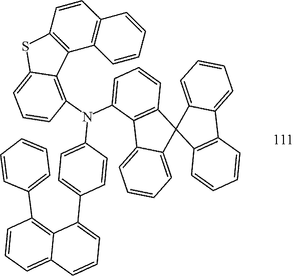 [SMILES: c1ccc(-c2cccc3cccc(-c4ccc(N(c5cccc6c5-c5ccccc5C65c6ccccc6-c6ccccc65)c5cccc6sc7ccc8ccccc8c7c56)cc4)c23)cc1]

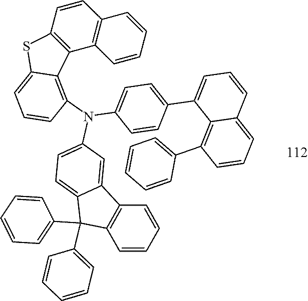 [SMILES: c1ccc(-c2cccc3cccc(-c4ccc(N(c5ccc6c(c5)-c5ccccc5C6(c5ccccc5)c5ccccc5)c5cccc6sc7ccc8ccccc8c7c56)cc4)c23)cc1]

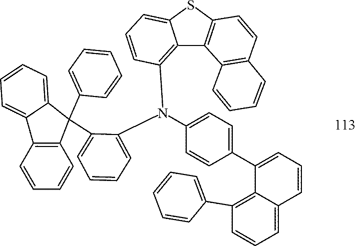 [SMILES: c1ccc(-c2cccc3cccc(-c4ccc(N(c5ccccc5C5(c6ccccc6)c6ccccc6-c6ccccc65)c5cccc6sc7ccc8ccccc8c7c56)cc4)c23)cc1]

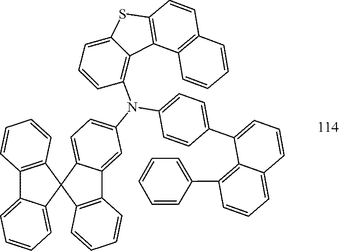 [SMILES: c1ccc(-c2cccc3cccc(-c4ccc(N(c5ccc6c(c5)-c5ccccc5C65c6ccccc6-c6ccccc65)c5cccc6sc7ccc8ccccc8c7c56)cc4)c23)cc1]

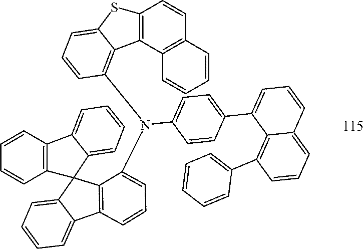 [SMILES: c1ccc(-c2cccc3cccc(-c4ccc(N(c5cccc6c5C5(c7ccccc7-c7ccccc75)c5ccccc5-6)c5cccc6sc7ccc8ccccc8c7c56)cc4)c23)cc1]

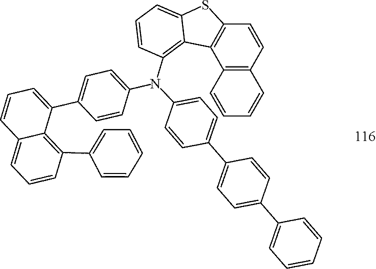 [SMILES: c1ccc(-c2ccc(-c3ccc(N(c4ccc(-c5cccc6cccc(-c7ccccc7)c56)cc4)c4cccc5sc6ccc7ccccc7c6c45)cc3)cc2)cc1]

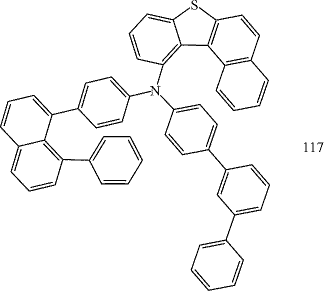 [SMILES: c1ccc(-c2cccc(-c3ccc(N(c4ccc(-c5cccc6cccc(-c7ccccc7)c56)cc4)c4cccc5sc6ccc7ccccc7c6c45)cc3)c2)cc1]

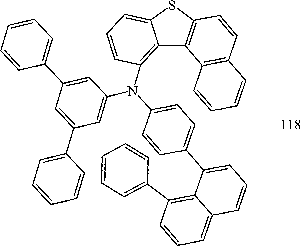 [SMILES: c1ccc(-c2cc(-c3ccccc3)cc(N(c3ccc(-c4cccc5cccc(-c6ccccc6)c45)cc3)c3cccc4sc5ccc6ccccc6c5c34)c2)cc1]

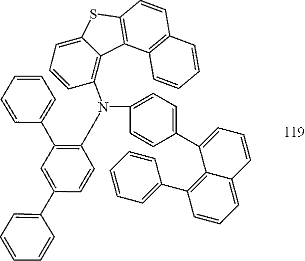 [SMILES: c1ccc(-c2ccc(N(c3ccc(-c4cccc5cccc(-c6ccccc6)c45)cc3)c3cccc4sc5ccc6ccccc6c5c34)c(-c3ccccc3)c2)cc1]

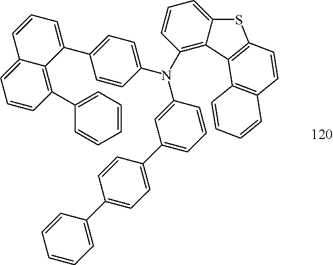 [SMILES: c1ccc(-c2ccc(-c3cccc(N(c4ccc(-c5cccc6cccc(-c7ccccc7)c56)cc4)c4cccc5sc6ccc7ccccc7c6c45)c3)cc2)cc1]

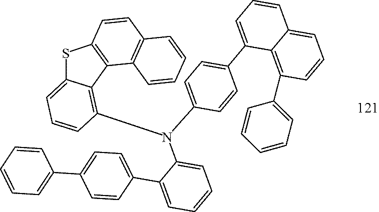 [SMILES: c1ccc(-c2ccc(-c3ccccc3N(c3ccc(-c4cccc5cccc(-c6ccccc6)c45)cc3)c3cccc4sc5ccc6ccccc6c5c34)cc2)cc1]

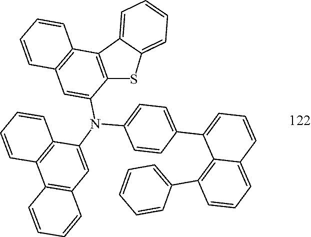 [SMILES: c1ccc(-c2cccc3cccc(-c4ccc(N(c5cc6ccccc6c6ccccc56)c5cc6ccccc6c6c5sc5ccccc56)cc4)c23)cc1]

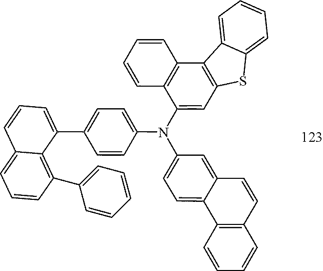 [SMILES: c1ccc(-c2cccc3cccc(-c4ccc(N(c5ccc6c(ccc7ccccc76)c5)c5cc6sc7ccccc7c6c6ccccc56)cc4)c23)cc1]

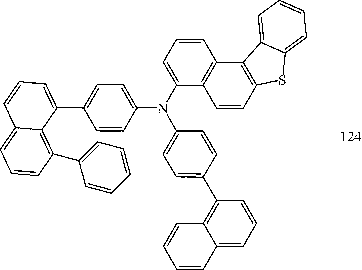 [SMILES: c1ccc(-c2cccc3cccc(-c4ccc(N(c5ccc(-c6cccc7ccccc67)cc5)c5cccc6c5ccc5sc7ccccc7c56)cc4)c23)cc1]

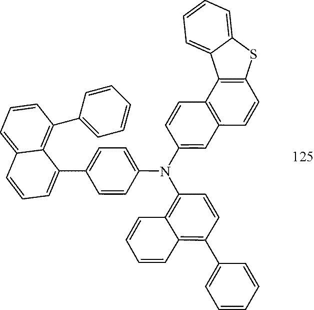 [SMILES: c1ccc(-c2ccc(N(c3ccc(-c4cccc5cccc(-c6ccccc6)c45)cc3)c3ccc4c(ccc5sc6ccccc6c54)c3)c3ccccc23)cc1]

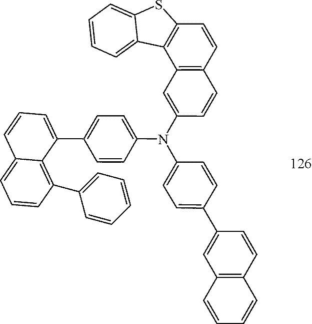 [SMILES: c1ccc(-c2cccc3cccc(-c4ccc(N(c5ccc(-c6ccc7ccccc7c6)cc5)c5ccc6ccc7sc8ccccc8c7c6c5)cc4)c23)cc1]

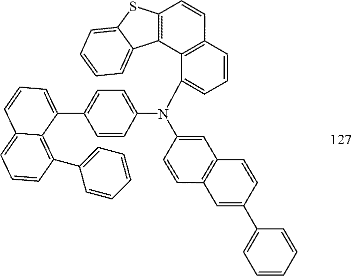 [SMILES: c1ccc(-c2ccc3cc(N(c4ccc(-c5cccc6cccc(-c7ccccc7)c56)cc4)c4cccc5ccc6sc7ccccc7c6c45)ccc3c2)cc1]

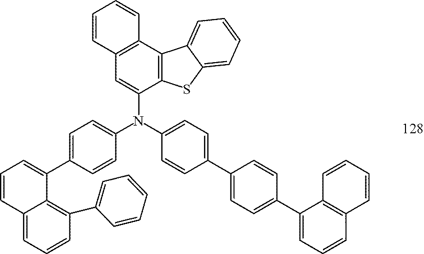 [SMILES: c1ccc(-c2cccc3cccc(-c4ccc(N(c5ccc(-c6ccc(-c7cccc8ccccc78)cc6)cc5)c5cc6ccccc6c6c5sc5ccccc56)cc4)c23)cc1]

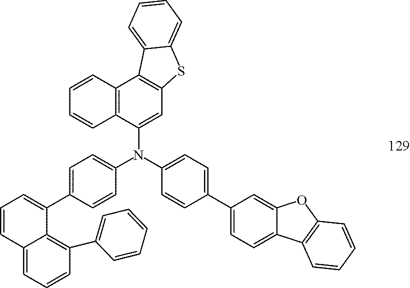 [SMILES: c1ccc(-c2cccc3cccc(-c4ccc(N(c5ccc(-c6ccc7c(c6)oc6ccccc67)cc5)c5cc6sc7ccccc7c6c6ccccc56)cc4)c23)cc1]

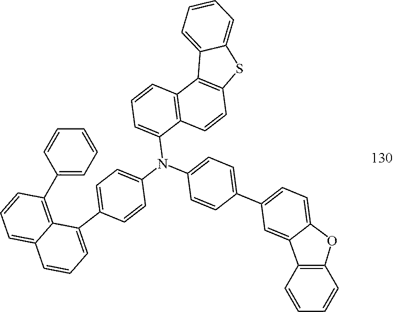

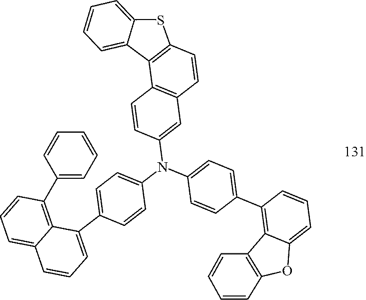

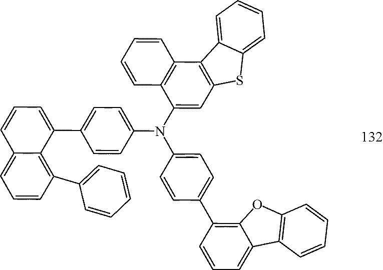 [SMILES: c1ccc(-c2cccc3cccc(-c4ccc(N(c5ccc(-c6cccc7c6oc6ccccc67)cc5)c5cc6sc7ccccc7c6c6ccccc56)cc4)c23)cc1]

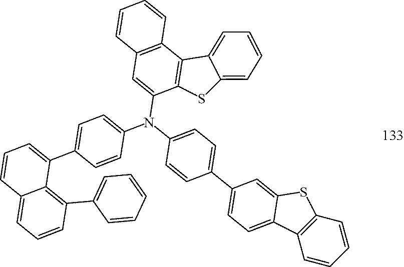 [SMILES: c1ccc(-c2cccc3cccc(-c4ccc(N(c5ccc(-c6ccc7c(c6)sc6ccccc67)cc5)c5cc6ccccc6c6c5sc5ccccc56)cc4)c23)cc1]

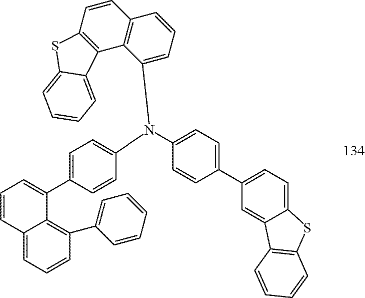 [SMILES: c1ccc(-c2cccc3cccc(-c4ccc(N(c5ccc(-c6ccc7sc8ccccc8c7c6)cc5)c5cccc6ccc7sc8ccccc8c7c56)cc4)c23)cc1]

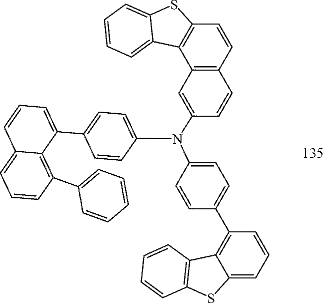 [SMILES: c1ccc(-c2cccc3cccc(-c4ccc(N(c5ccc(-c6cccc7sc8ccccc8c67)cc5)c5ccc6ccc7sc8ccccc8c7c6c5)cc4)c23)cc1]

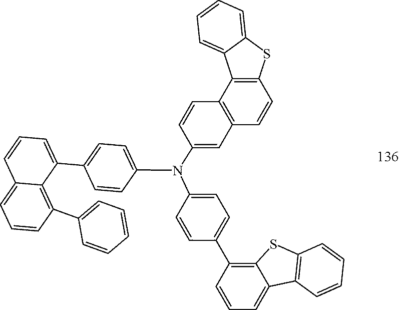 [SMILES: c1ccc(-c2cccc3cccc(-c4ccc(N(c5ccc(-c6cccc7c6sc6ccccc67)cc5)c5ccc6c(ccc7sc8ccccc8c76)c5)cc4)c23)cc1]

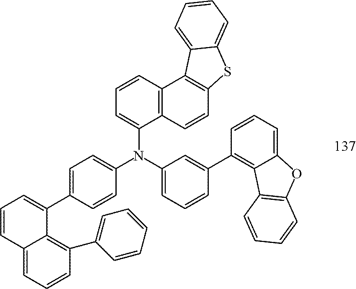 [SMILES: c1ccc(-c2cccc3cccc(-c4ccc(N(c5cccc(-c6cccc7oc8ccccc8c67)c5)c5cccc6c5ccc5sc7ccccc7c56)cc4)c23)cc1]

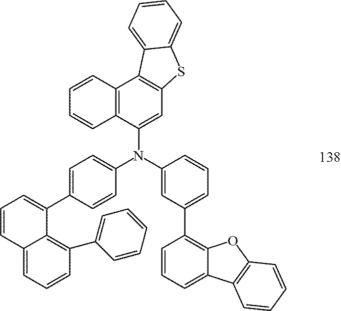 [SMILES: c1ccc(-c2cccc3cccc(-c4ccc(N(c5cccc(-c6cccc7c6oc6ccccc67)c5)c5cc6sc7ccccc7c6c6ccccc56)cc4)c23)cc1]

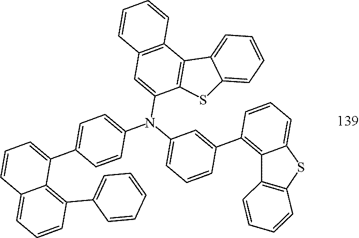 [SMILES: c1ccc(-c2cccc3cccc(-c4ccc(N(c5cccc(-c6cccc7sc8ccccc8c67)c5)c5cc6ccccc6c6c5sc5ccccc56)cc4)c23)cc1]

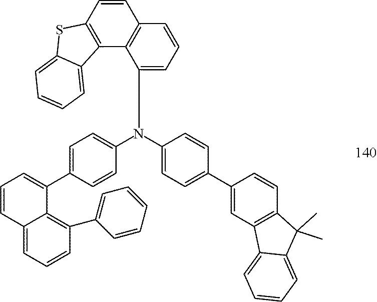 [SMILES: CC1(C)c2ccccc2-c2cc(-c3ccc(N(c4ccc(-c5cccc6cccc(-c7ccccc7)c56)cc4)c4cccc5ccc6sc7ccccc7c6c45)cc3)ccc21]

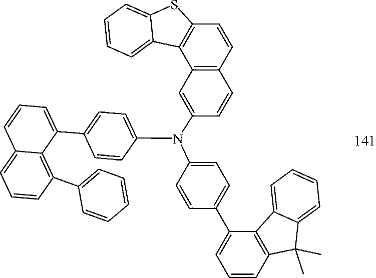 [SMILES: CC1(C)c2ccccc2-c2c(-c3ccc(N(c4ccc(-c5cccc6cccc(-c7ccccc7)c56)cc4)c4ccc5ccc6sc7ccccc7c6c5c4)cc3)cccc21]

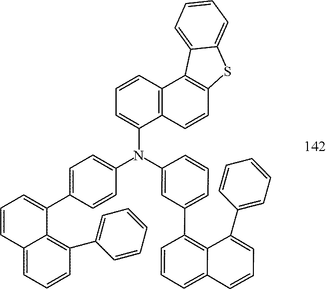 [SMILES: c1ccc(-c2cccc3cccc(-c4ccc(N(c5cccc(-c6cccc7cccc(-c8ccccc8)c67)c5)c5cccc6c5ccc5sc7ccccc7c56)cc4)c23)cc1]

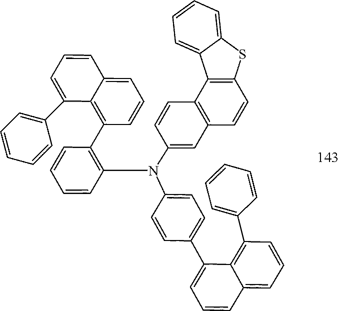 [SMILES: c1ccc(-c2cccc3cccc(-c4ccc(N(c5ccc6c(ccc7sc8ccccc8c76)c5)c5ccccc5-c5cccc6cccc(-c7ccccc7)c56)cc4)c23)cc1]

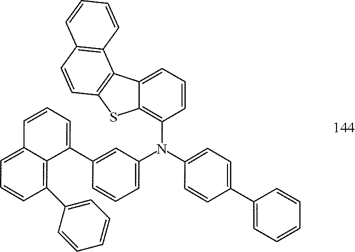 [SMILES: c1ccc(-c2ccc(N(c3cccc(-c4cccc5cccc(-c6ccccc6)c45)c3)c3cccc4c3sc3ccc5ccccc5c34)cc2)cc1]

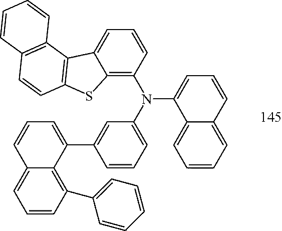 [SMILES: c1ccc(-c2cccc3cccc(-c4cccc(N(c5cccc6ccccc56)c5cccc6c5sc5ccc7ccccc7c56)c4)c23)cc1]

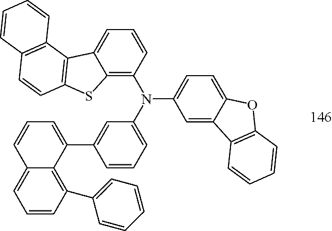 [SMILES: c1ccc(-c2cccc3cccc(-c4cccc(N(c5ccc6oc7ccccc7c6c5)c5cccc6c5sc5ccc7ccccc7c56)c4)c23)cc1]

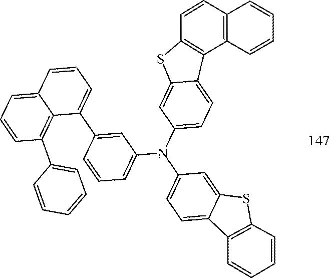 [SMILES: c1ccc(-c2cccc3cccc(-c4cccc(N(c5ccc6c(c5)sc5ccccc56)c5ccc6c(c5)sc5ccc7ccccc7c56)c4)c23)cc1]

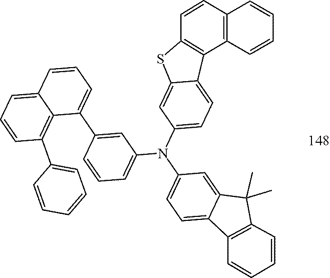 [SMILES: CC1(C)c2ccccc2-c2ccc(N(c3cccc(-c4cccc5cccc(-c6ccccc6)c45)c3)c3ccc4c(c3)sc3ccc5ccccc5c34)cc21]

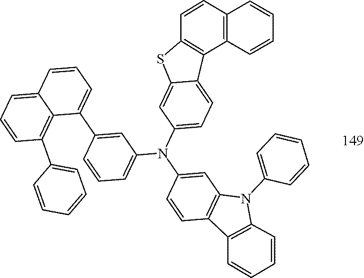 [SMILES: c1ccc(-c2cccc3cccc(-c4cccc(N(c5ccc6c(c5)sc5ccc7ccccc7c56)c5ccc6c7ccccc7n(-c7ccccc7)c6c5)c4)c23)cc1]

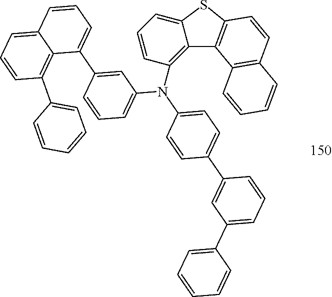 [SMILES: c1ccc(-c2cccc(-c3ccc(N(c4cccc(-c5cccc6cccc(-c7ccccc7)c56)c4)c4cccc5sc6ccc7ccccc7c6c45)cc3)c2)cc1]

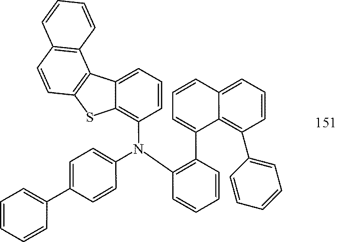 [SMILES: c1ccc(-c2ccc(N(c3ccccc3-c3cccc4cccc(-c5ccccc5)c34)c3cccc4c3sc3ccc5ccccc5c34)cc2)cc1]

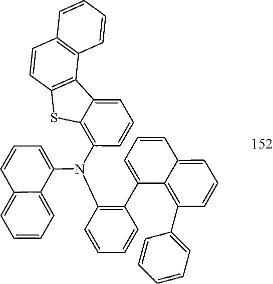 [SMILES: c1ccc(-c2cccc3cccc(-c4ccccc4N(c4cccc5ccccc45)c4cccc5c4sc4ccc6ccccc6c45)c23)cc1]

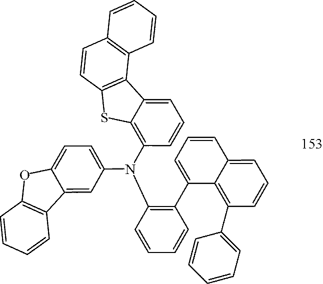 [SMILES: c1ccc(-c2cccc3cccc(-c4ccccc4N(c4ccc5oc6ccccc6c5c4)c4cccc5c4sc4ccc6ccccc6c45)c23)cc1]

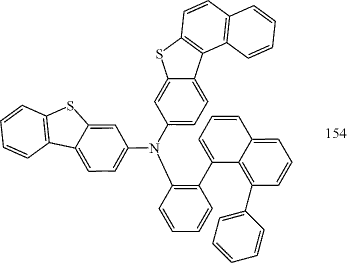 [SMILES: c1ccc(-c2cccc3cccc(-c4ccccc4N(c4ccc5c(c4)sc4ccccc45)c4ccc5c(c4)sc4ccc6ccccc6c45)c23)cc1]

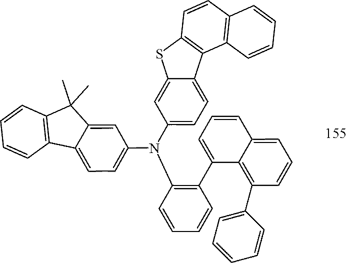 [SMILES: CC1(C)c2ccccc2-c2ccc(N(c3ccc4c(c3)sc3ccc5ccccc5c34)c3ccccc3-c3cccc4cccc(-c5ccccc5)c34)cc21]

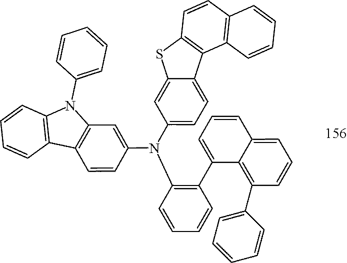 [SMILES: c1ccc(-c2cccc3cccc(-c4ccccc4N(c4ccc5c(c4)sc4ccc6ccccc6c45)c4ccc5c6ccccc6n(-c6ccccc6)c5c4)c23)cc1]

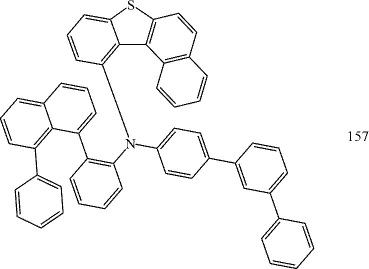 [SMILES: c1ccc(-c2cccc(-c3ccc(N(c4ccccc4-c4cccc5cccc(-c6ccccc6)c45)c4cccc5sc6ccc7ccccc7c6c45)cc3)c2)cc1]

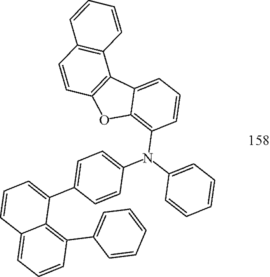 [SMILES: c1ccc(-c2cccc3cccc(-c4ccc(N(c5ccccc5)c5cccc6c5oc5ccc7ccccc7c56)cc4)c23)cc1]

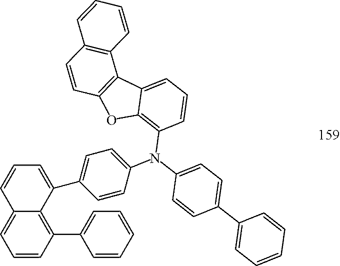 [SMILES: c1ccc(-c2ccc(N(c3ccc(-c4cccc5cccc(-c6ccccc6)c45)cc3)c3cccc4c3oc3ccc5ccccc5c34)cc2)cc1]

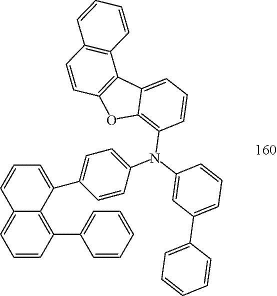 [SMILES: c1ccc(-c2cccc(N(c3ccc(-c4cccc5cccc(-c6ccccc6)c45)cc3)c3cccc4c3oc3ccc5ccccc5c34)c2)cc1]

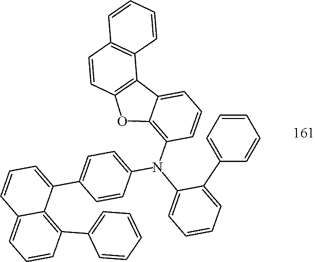 [SMILES: c1ccc(-c2ccccc2N(c2ccc(-c3cccc4cccc(-c5ccccc5)c34)cc2)c2cccc3c2oc2ccc4ccccc4c23)cc1]

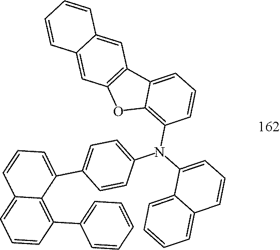 [SMILES: c1ccc(-c2cccc3cccc(-c4ccc(N(c5cccc6ccccc56)c5cccc6c5oc5cc7ccccc7cc56)cc4)c23)cc1]

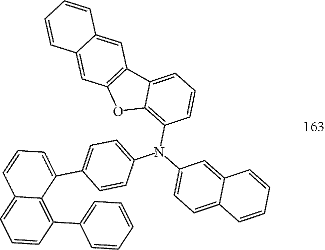 [SMILES: c1ccc(-c2cccc3cccc(-c4ccc(N(c5ccc6ccccc6c5)c5cccc6c5oc5cc7ccccc7cc56)cc4)c23)cc1]

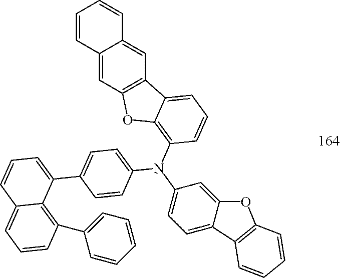 [SMILES: c1ccc(-c2cccc3cccc(-c4ccc(N(c5ccc6c(c5)oc5ccccc56)c5cccc6c5oc5cc7ccccc7cc56)cc4)c23)cc1]

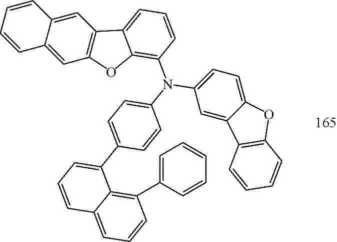 [SMILES: c1ccc(-c2cccc3cccc(-c4ccc(N(c5ccc6oc7ccccc7c6c5)c5cccc6c5oc5cc7ccccc7cc56)cc4)c23)cc1]

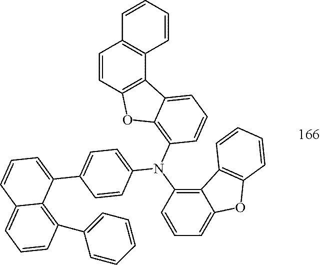 [SMILES: c1ccc(-c2cccc3cccc(-c4ccc(N(c5cccc6c5oc5ccc7ccccc7c56)c5cccc6oc7ccccc7c56)cc4)c23)cc1]

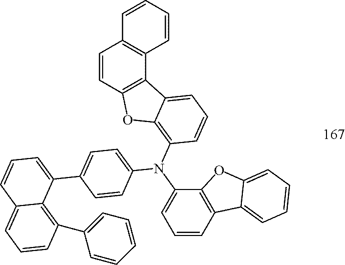 [SMILES: c1ccc(-c2cccc3cccc(-c4ccc(N(c5cccc6c5oc5ccccc56)c5cccc6c5oc5ccc7ccccc7c56)cc4)c23)cc1]

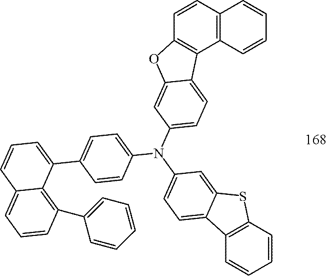 [SMILES: c1ccc(-c2cccc3cccc(-c4ccc(N(c5ccc6c(c5)oc5ccc7ccccc7c56)c5ccc6c(c5)sc5ccccc56)cc4)c23)cc1]

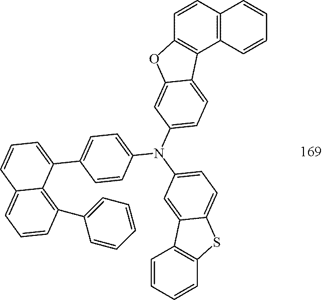 [SMILES: c1ccc(-c2cccc3cccc(-c4ccc(N(c5ccc6c(c5)oc5ccc7ccccc7c56)c5ccc6sc7ccccc7c6c5)cc4)c23)cc1]

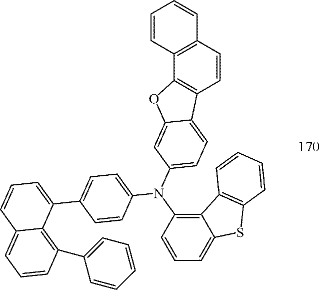 [SMILES: c1ccc(-c2cccc3cccc(-c4ccc(N(c5ccc6c(c5)oc5c7ccccc7ccc65)c5cccc6sc7ccccc7c56)cc4)c23)cc1]

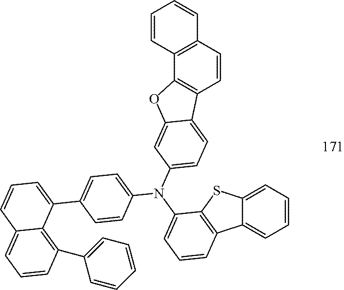 [SMILES: c1ccc(-c2cccc3cccc(-c4ccc(N(c5ccc6c(c5)oc5c7ccccc7ccc65)c5cccc6c5sc5ccccc56)cc4)c23)cc1]

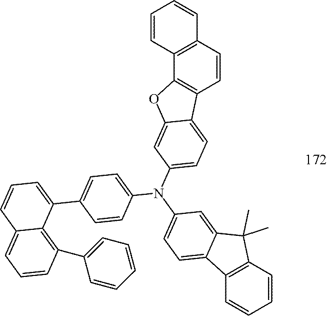 [SMILES: CC1(C)c2ccccc2-c2ccc(N(c3ccc(-c4cccc5cccc(-c6ccccc6)c45)cc3)c3ccc4c(c3)oc3c5ccccc5ccc43)cc21]

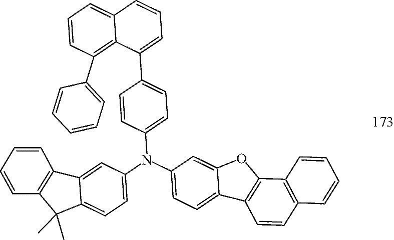 [SMILES: CC1(C)c2ccccc2-c2cc(N(c3ccc(-c4cccc5cccc(-c6ccccc6)c45)cc3)c3ccc4c(c3)oc3c5ccccc5ccc43)ccc21]

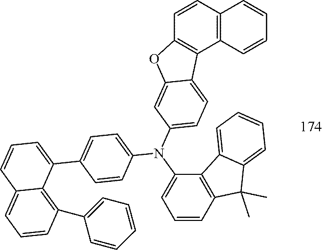 [SMILES: CC1(C)c2ccccc2-c2c(N(c3ccc(-c4cccc5cccc(-c6ccccc6)c45)cc3)c3ccc4c(c3)oc3ccc5ccccc5c34)cccc21]

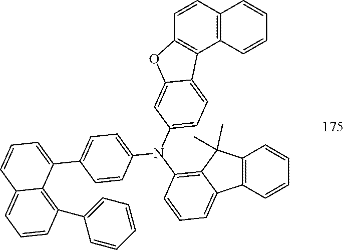 [SMILES: CC1(C)c2ccccc2-c2cccc(N(c3ccc(-c4cccc5cccc(-c6ccccc6)c45)cc3)c3ccc4c(c3)oc3ccc5ccccc5c34)c21]

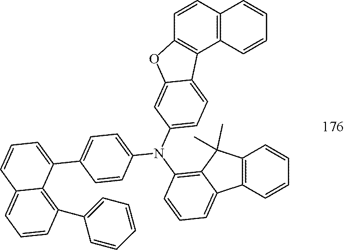 [SMILES: CC1(C)c2ccccc2-c2cccc(N(c3ccc(-c4cccc5cccc(-c6ccccc6)c45)cc3)c3ccc4c(c3)oc3ccc5ccccc5c34)c21]

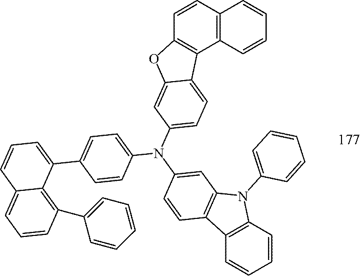 [SMILES: c1ccc(-c2cccc3cccc(-c4ccc(N(c5ccc6c(c5)oc5ccc7ccccc7c56)c5ccc6c7ccccc7n(-c7ccccc7)c6c5)cc4)c23)cc1]

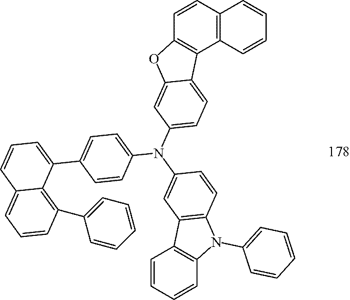 [SMILES: c1ccc(-c2cccc3cccc(-c4ccc(N(c5ccc6c(c5)oc5ccc7ccccc7c56)c5ccc6c(c5)c5ccccc5n6-c5ccccc5)cc4)c23)cc1]

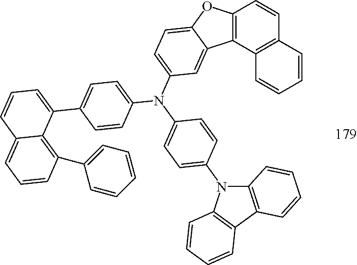 [SMILES: c1ccc(-c2cccc3cccc(-c4ccc(N(c5ccc(-n6c7ccccc7c7ccccc76)cc5)c5ccc6oc7ccc8ccccc8c7c6c5)cc4)c23)cc1]

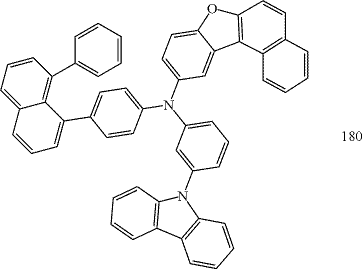 [SMILES: c1ccc(-c2cccc3cccc(-c4ccc(N(c5cccc(-n6c7ccccc7c7ccccc76)c5)c5ccc6oc7ccc8ccccc8c7c6c5)cc4)c23)cc1]

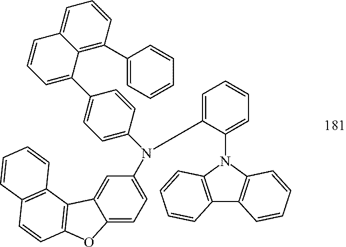 [SMILES: c1ccc(-c2cccc3cccc(-c4ccc(N(c5ccc6oc7ccc8ccccc8c7c6c5)c5ccccc5-n5c6ccccc6c6ccccc65)cc4)c23)cc1]

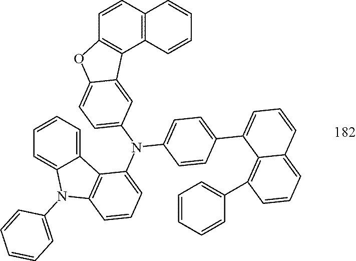 [SMILES: c1ccc(-c2cccc3cccc(-c4ccc(N(c5ccc6oc7ccc8ccccc8c7c6c5)c5cccc6c5c5ccccc5n6-c5ccccc5)cc4)c23)cc1]

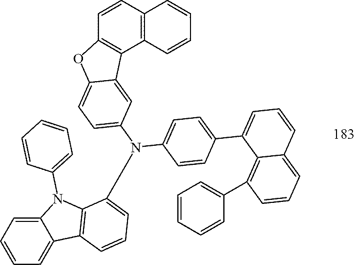 [SMILES: c1ccc(-c2cccc3cccc(-c4ccc(N(c5ccc6oc7ccc8ccccc8c7c6c5)c5cccc6c7ccccc7n(-c7ccccc7)c56)cc4)c23)cc1]

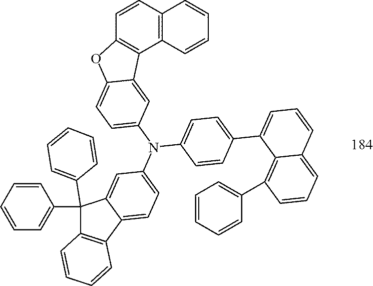 [SMILES: c1ccc(-c2cccc3cccc(-c4ccc(N(c5ccc6c(c5)C(c5ccccc5)(c5ccccc5)c5ccccc5-6)c5ccc6oc7ccc8ccccc8c7c6c5)cc4)c23)cc1]

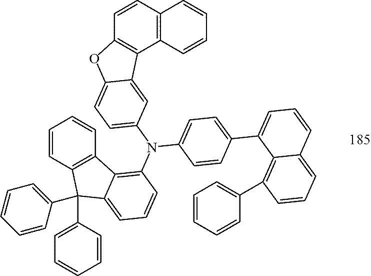 [SMILES: c1ccc(-c2cccc3cccc(-c4ccc(N(c5ccc6oc7ccc8ccccc8c7c6c5)c5cccc6c5-c5ccccc5C6(c5ccccc5)c5ccccc5)cc4)c23)cc1]

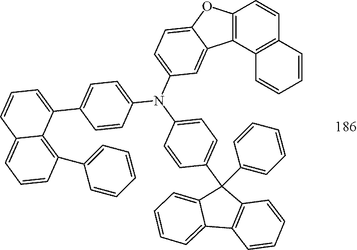 [SMILES: c1ccc(-c2cccc3cccc(-c4ccc(N(c5ccc(C6(c7ccccc7)c7ccccc7-c7ccccc76)cc5)c5ccc6oc7ccc8ccccc8c7c6c5)cc4)c23)cc1]

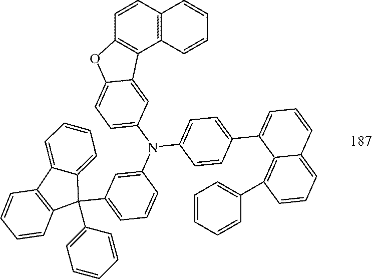 [SMILES: c1ccc(-c2cccc3cccc(-c4ccc(N(c5cccc(C6(c7ccccc7)c7ccccc7-c7ccccc76)c5)c5ccc6oc7ccc8ccccc8c7c6c5)cc4)c23)cc1]

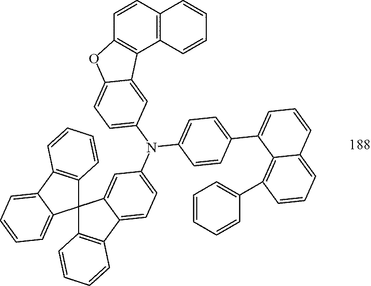 [SMILES: c1ccc(-c2cccc3cccc(-c4ccc(N(c5ccc6c(c5)C5(c7ccccc7-c7ccccc75)c5ccccc5-6)c5ccc6oc7ccc8ccccc8c7c6c5)cc4)c23)cc1]

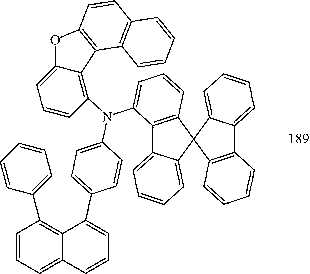 [SMILES: c1ccc(-c2cccc3cccc(-c4ccc(N(c5cccc6c5-c5ccccc5C65c6ccccc6-c6ccccc65)c5cccc6oc7ccc8ccccc8c7c56)cc4)c23)cc1]

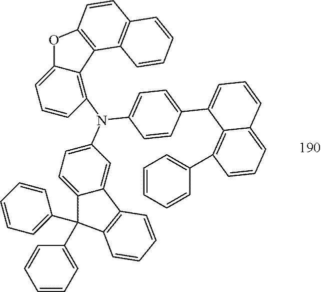 [SMILES: c1ccc(-c2cccc3cccc(-c4ccc(N(c5ccc6c(c5)-c5ccccc5C6(c5ccccc5)c5ccccc5)c5cccc6oc7ccc8ccccc8c7c56)cc4)c23)cc1]

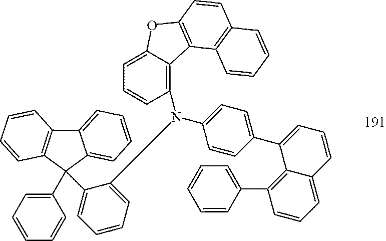 [SMILES: c1ccc(-c2cccc3cccc(-c4ccc(N(c5ccccc5C5(c6ccccc6)c6ccccc6-c6ccccc65)c5cccc6oc7ccc8ccccc8c7c56)cc4)c23)cc1]

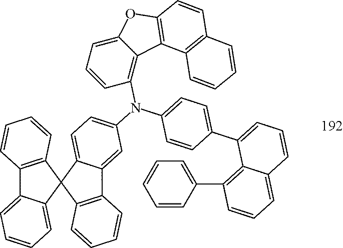 [SMILES: c1ccc(-c2cccc3cccc(-c4ccc(N(c5ccc6c(c5)-c5ccccc5C65c6ccccc6-c6ccccc65)c5cccc6oc7ccc8ccccc8c7c56)cc4)c23)cc1]

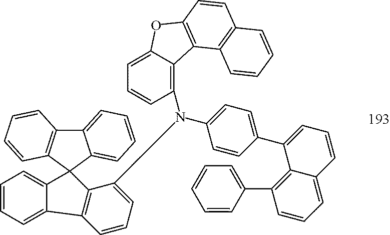 [SMILES: c1ccc(-c2cccc3cccc(-c4ccc(N(c5cccc6c5C5(c7ccccc7-c7ccccc75)c5ccccc5-6)c5cccc6oc7ccc8ccccc8c7c56)cc4)c23)cc1]

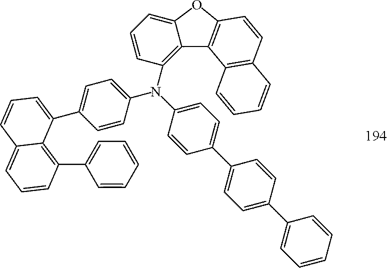 [SMILES: c1ccc(-c2ccc(-c3ccc(N(c4ccc(-c5cccc6cccc(-c7ccccc7)c56)cc4)c4cccc5oc6ccc7ccccc7c6c45)cc3)cc2)cc1]

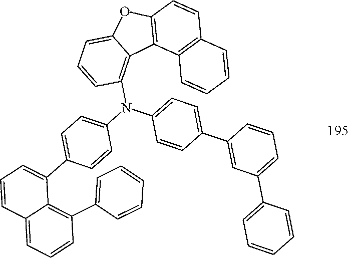 [SMILES: c1ccc(-c2cccc(-c3ccc(N(c4ccc(-c5cccc6cccc(-c7ccccc7)c56)cc4)c4cccc5oc6ccc7ccccc7c6c45)cc3)c2)cc1]

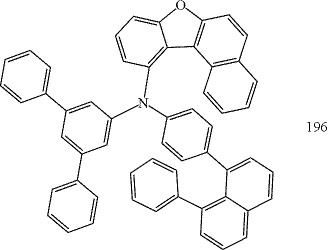 [SMILES: c1ccc(-c2cc(-c3ccccc3)cc(N(c3ccc(-c4cccc5cccc(-c6ccccc6)c45)cc3)c3cccc4oc5ccc6ccccc6c5c34)c2)cc1]

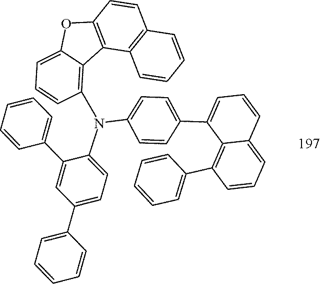 [SMILES: c1ccc(-c2ccc(N(c3ccc(-c4cccc5cccc(-c6ccccc6)c45)cc3)c3cccc4oc5ccc6ccccc6c5c34)c(-c3ccccc3)c2)cc1]

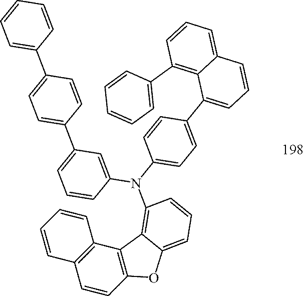 [SMILES: c1ccc(-c2ccc(-c3cccc(N(c4ccc(-c5cccc6cccc(-c7ccccc7)c56)cc4)c4cccc5oc6ccc7ccccc7c6c45)c3)cc2)cc1]

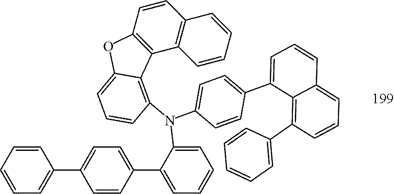 [SMILES: c1ccc(-c2ccc(-c3ccccc3N(c3ccc(-c4cccc5cccc(-c6ccccc6)c45)cc3)c3cccc4oc5ccc6ccccc6c5c34)cc2)cc1]

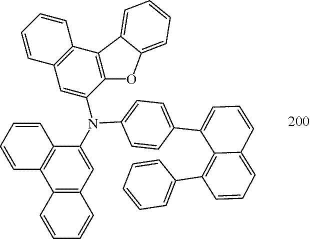 [SMILES: c1ccc(-c2cccc3cccc(-c4ccc(N(c5cc6ccccc6c6ccccc56)c5cc6ccccc6c6c5oc5ccccc56)cc4)c23)cc1]

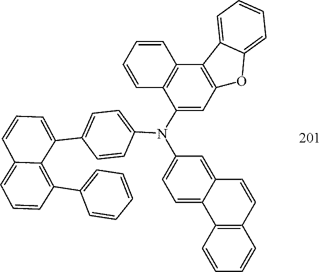 [SMILES: c1ccc(-c2cccc3cccc(-c4ccc(N(c5ccc6c(ccc7ccccc76)c5)c5cc6oc7ccccc7c6c6ccccc56)cc4)c23)cc1]

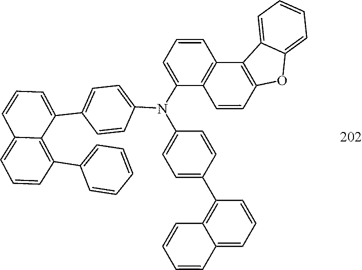 [SMILES: c1ccc(-c2cccc3cccc(-c4ccc(N(c5ccc(-c6cccc7ccccc67)cc5)c5cccc6c5ccc5oc7ccccc7c56)cc4)c23)cc1]

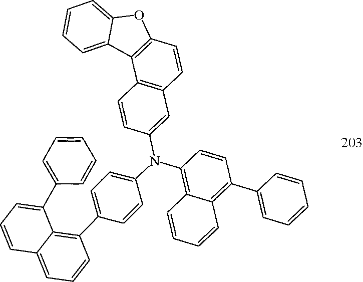 [SMILES: c1ccc(-c2ccc(N(c3ccc(-c4cccc5cccc(-c6ccccc6)c45)cc3)c3ccc4c(ccc5oc6ccccc6c54)c3)c3ccccc23)cc1]

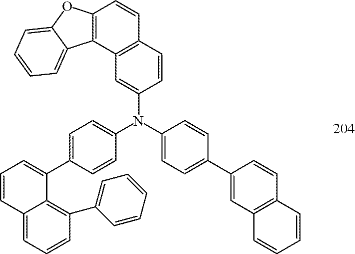 [SMILES: c1ccc(-c2cccc3cccc(-c4ccc(N(c5ccc(-c6ccc7ccccc7c6)cc5)c5ccc6ccc7oc8ccccc8c7c6c5)cc4)c23)cc1]

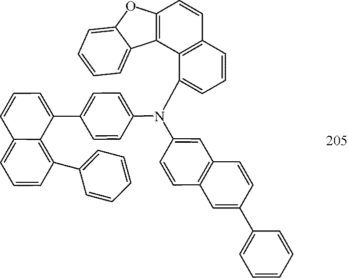 [SMILES: c1ccc(-c2ccc3cc(N(c4ccc(-c5cccc6cccc(-c7ccccc7)c56)cc4)c4cccc5ccc6oc7ccccc7c6c45)ccc3c2)cc1]

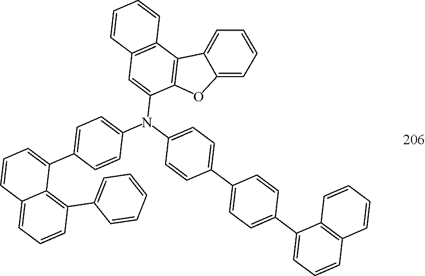 [SMILES: c1ccc(-c2cccc3cccc(-c4ccc(N(c5ccc(-c6ccc(-c7cccc8ccccc78)cc6)cc5)c5cc6ccccc6c6c5oc5ccccc56)cc4)c23)cc1]

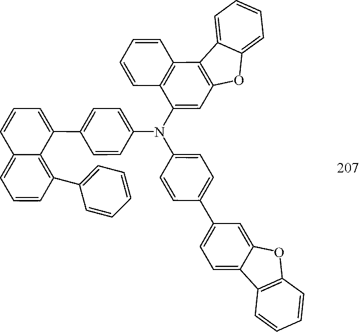 [SMILES: c1ccc(-c2cccc3cccc(-c4ccc(N(c5ccc(-c6ccc7c(c6)oc6ccccc67)cc5)c5cc6oc7ccccc7c6c6ccccc56)cc4)c23)cc1]

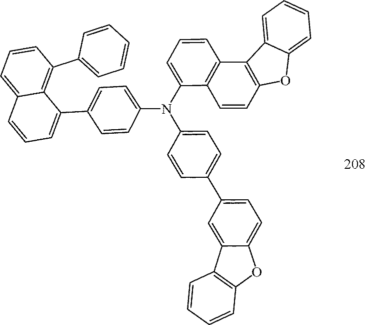 [SMILES: c1ccc(-c2cccc3cccc(-c4ccc(N(c5ccc(-c6ccc7oc8ccccc8c7c6)cc5)c5cccc6c5ccc5oc7ccccc7c56)cc4)c23)cc1]

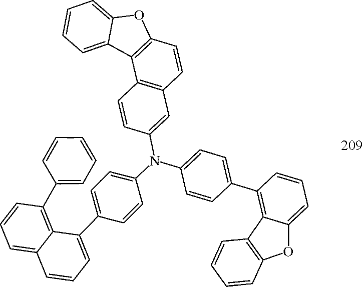 [SMILES: c1ccc(-c2cccc3cccc(-c4ccc(N(c5ccc(-c6cccc7oc8ccccc8c67)cc5)c5ccc6c(ccc7oc8ccccc8c76)c5)cc4)c23)cc1]

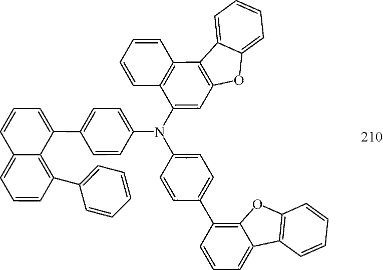 [SMILES: c1ccc(-c2cccc3cccc(-c4ccc(N(c5ccc(-c6cccc7c6oc6ccccc67)cc5)c5cc6oc7ccccc7c6c6ccccc56)cc4)c23)cc1]

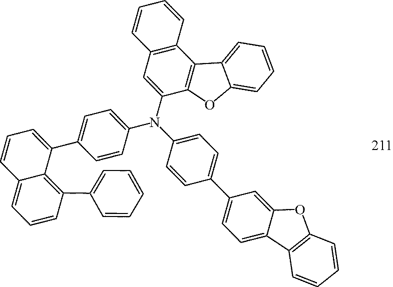 [SMILES: c1ccc(-c2cccc3cccc(-c4ccc(N(c5ccc(-c6ccc7c(c6)oc6ccccc67)cc5)c5cc6ccccc6c6c5oc5ccccc56)cc4)c23)cc1]

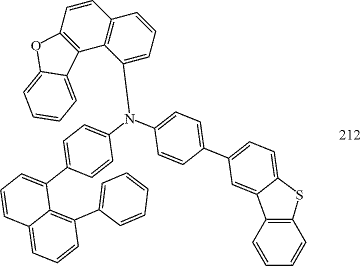 [SMILES: c1ccc(-c2cccc3cccc(-c4ccc(N(c5ccc(-c6ccc7sc8ccccc8c7c6)cc5)c5cccc6ccc7oc8ccccc8c7c56)cc4)c23)cc1]

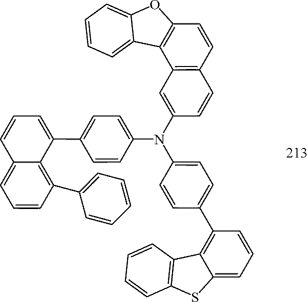 [SMILES: c1ccc(-c2cccc3cccc(-c4ccc(N(c5ccc(-c6cccc7sc8ccccc8c67)cc5)c5ccc6ccc7oc8ccccc8c7c6c5)cc4)c23)cc1]

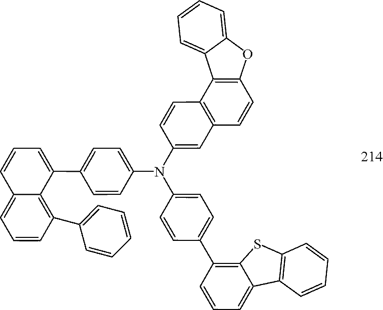 [SMILES: c1ccc(-c2cccc3cccc(-c4ccc(N(c5ccc(-c6cccc7c6sc6ccccc67)cc5)c5ccc6c(ccc7oc8ccccc8c76)c5)cc4)c23)cc1]

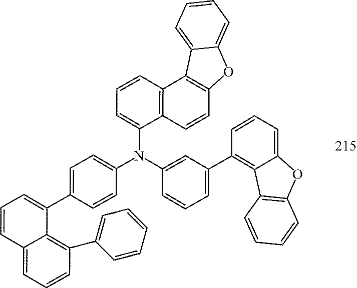 [SMILES: c1ccc(-c2cccc3cccc(-c4ccc(N(c5cccc(-c6cccc7oc8ccccc8c67)c5)c5cccc6c5ccc5oc7ccccc7c56)cc4)c23)cc1]

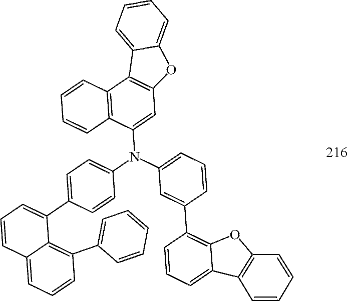 [SMILES: c1ccc(-c2cccc3cccc(-c4ccc(N(c5cccc(-c6cccc7c6oc6ccccc67)c5)c5cc6oc7ccccc7c6c6ccccc56)cc4)c23)cc1]

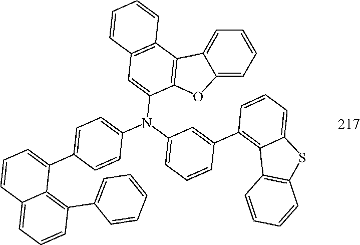 [SMILES: c1ccc(-c2cccc3cccc(-c4ccc(N(c5cccc(-c6cccc7sc8ccccc8c67)c5)c5cc6ccccc6c6c5oc5ccccc56)cc4)c23)cc1]

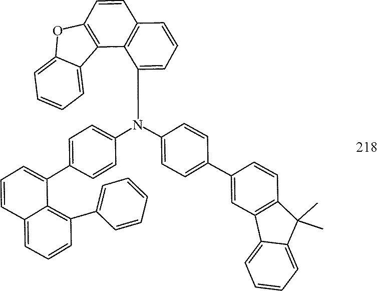 [SMILES: CC1(C)c2ccccc2-c2cc(-c3ccc(N(c4ccc(-c5cccc6cccc(-c7ccccc7)c56)cc4)c4cccc5ccc6oc7ccccc7c6c45)cc3)ccc21]

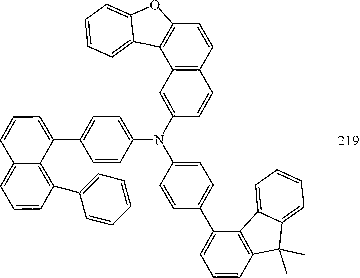 [SMILES: CC1(C)c2ccccc2-c2c(-c3ccc(N(c4ccc(-c5cccc6cccc(-c7ccccc7)c56)cc4)c4ccc5ccc6oc7ccccc7c6c5c4)cc3)cccc21]

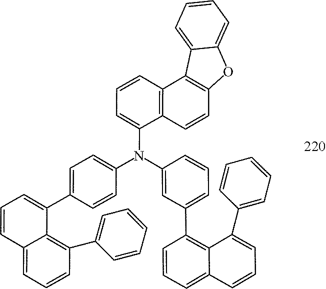 [SMILES: c1ccc(-c2cccc3cccc(-c4ccc(N(c5cccc(-c6cccc7cccc(-c8ccccc8)c67)c5)c5cccc6c5ccc5oc7ccccc7c56)cc4)c23)cc1]

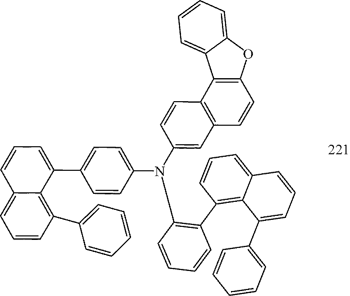 [SMILES: c1ccc(-c2cccc3cccc(-c4ccc(N(c5ccc6c(ccc7oc8ccccc8c76)c5)c5ccccc5-c5cccc6cccc(-c7ccccc7)c56)cc4)c23)cc1]

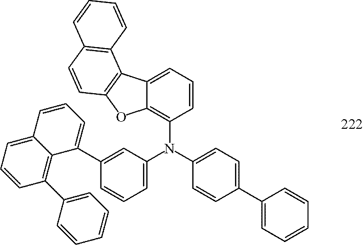 [SMILES: c1ccc(-c2ccc(N(c3cccc(-c4cccc5cccc(-c6ccccc6)c45)c3)c3cccc4c3oc3ccc5ccccc5c34)cc2)cc1]

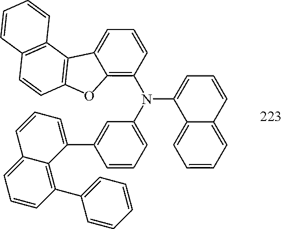 [SMILES: c1ccc(-c2cccc3cccc(-c4cccc(N(c5cccc6ccccc56)c5cccc6c5oc5ccc7ccccc7c56)c4)c23)cc1]

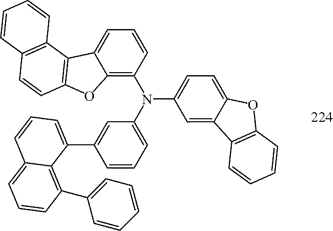 [SMILES: c1ccc(-c2cccc3cccc(-c4cccc(N(c5ccc6oc7ccccc7c6c5)c5cccc6c5oc5ccc7ccccc7c56)c4)c23)cc1]

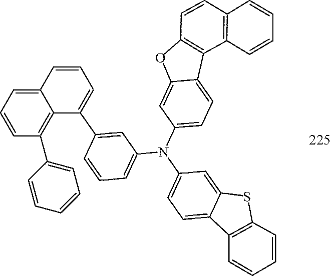 [SMILES: c1ccc(-c2cccc3cccc(-c4cccc(N(c5ccc6c(c5)oc5ccc7ccccc7c56)c5ccc6c(c5)sc5ccccc56)c4)c23)cc1]

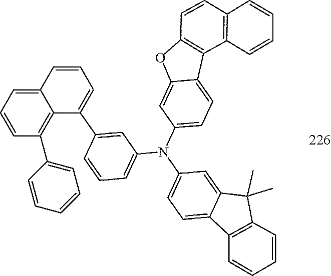 [SMILES: CC1(C)c2ccccc2-c2ccc(N(c3cccc(-c4cccc5cccc(-c6ccccc6)c45)c3)c3ccc4c(c3)oc3ccc5ccccc5c34)cc21]

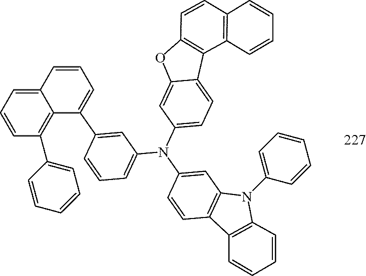 [SMILES: c1ccc(-c2cccc3cccc(-c4cccc(N(c5ccc6c(c5)oc5ccc7ccccc7c56)c5ccc6c7ccccc7n(-c7ccccc7)c6c5)c4)c23)cc1]

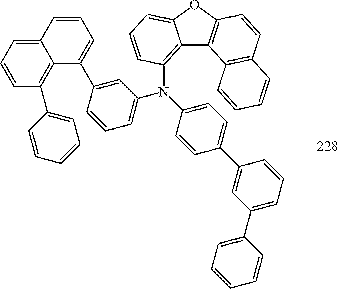 [SMILES: c1ccc(-c2cccc(-c3ccc(N(c4cccc(-c5cccc6cccc(-c7ccccc7)c56)c4)c4cccc5oc6ccc7ccccc7c6c45)cc3)c2)cc1]

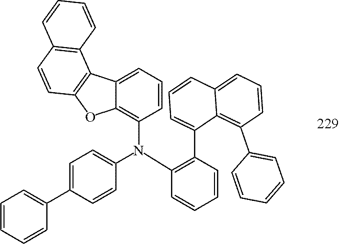 [SMILES: c1ccc(-c2ccc(N(c3ccccc3-c3cccc4cccc(-c5ccccc5)c34)c3cccc4c3oc3ccc5ccccc5c34)cc2)cc1]

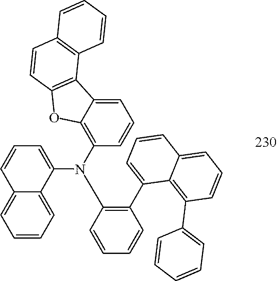 [SMILES: c1ccc(-c2cccc3cccc(-c4ccccc4N(c4cccc5ccccc45)c4cccc5c4oc4ccc6ccccc6c45)c23)cc1]

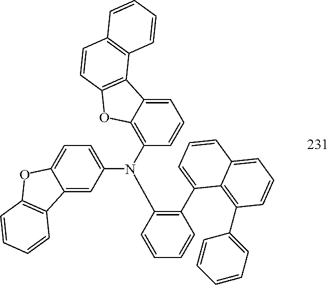 [SMILES: c1ccc(-c2cccc3cccc(-c4ccccc4N(c4ccc5oc6ccccc6c5c4)c4cccc5c4oc4ccc6ccccc6c45)c23)cc1]

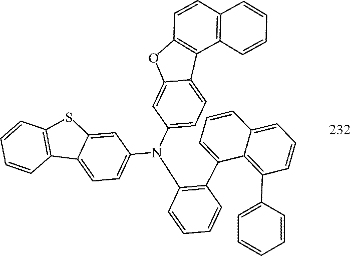 [SMILES: c1ccc(-c2cccc3cccc(-c4ccccc4N(c4ccc5c(c4)oc4ccc6ccccc6c45)c4ccc5c(c4)sc4ccccc45)c23)cc1]

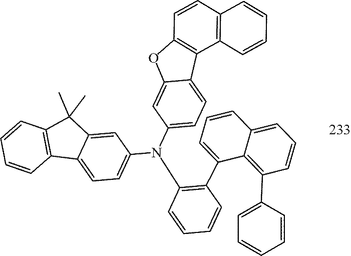 [SMILES: CC1(C)c2ccccc2-c2ccc(N(c3ccc4c(c3)oc3ccc5ccccc5c34)c3ccccc3-c3cccc4cccc(-c5ccccc5)c34)cc21]

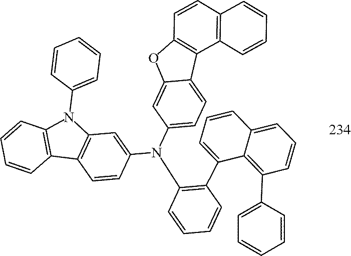 [SMILES: c1ccc(-c2cccc3cccc(-c4ccccc4N(c4ccc5c(c4)oc4ccc6ccccc6c45)c4ccc5c6ccccc6n(-c6ccccc6)c5c4)c23)cc1]

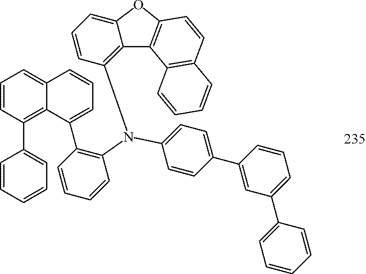 [SMILES: c1ccc(-c2cccc(-c3ccc(N(c4ccccc4-c4cccc5cccc(-c6ccccc6)c45)c4cccc5oc6ccc7ccccc7c6c45)cc3)c2)cc1]

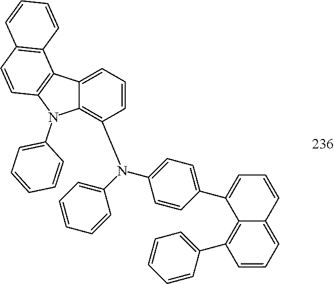 [SMILES: c1ccc(-c2cccc3cccc(-c4ccc(N(c5ccccc5)c5cccc6c7c8ccccc8ccc7n(-c7ccccc7)c56)cc4)c23)cc1]

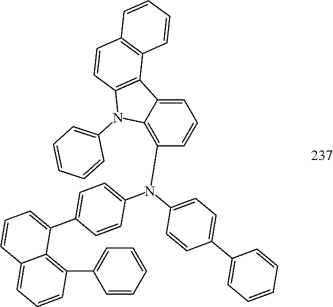 [SMILES: c1ccc(-c2ccc(N(c3ccc(-c4cccc5cccc(-c6ccccc6)c45)cc3)c3cccc4c5c6ccccc6ccc5n(-c5ccccc5)c34)cc2)cc1]

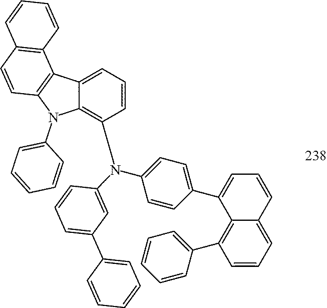 [SMILES: c1ccc(-c2cccc(N(c3ccc(-c4cccc5cccc(-c6ccccc6)c45)cc3)c3cccc4c5c6ccccc6ccc5n(-c5ccccc5)c34)c2)cc1]

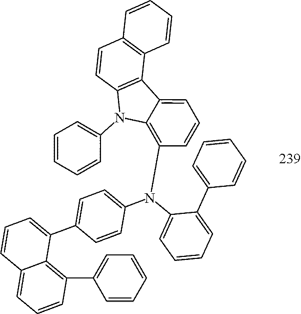 [SMILES: c1ccc(-c2ccccc2N(c2ccc(-c3cccc4cccc(-c5ccccc5)c34)cc2)c2cccc3c4c5ccccc5ccc4n(-c4ccccc4)c23)cc1]

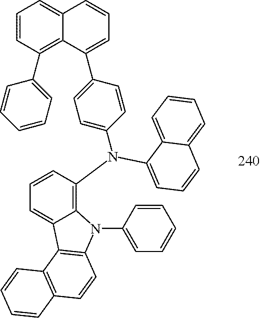 [SMILES: c1ccc(-c2cccc3cccc(-c4ccc(N(c5cccc6ccccc56)c5cccc6c7c8ccccc8ccc7n(-c7ccccc7)c56)cc4)c23)cc1]

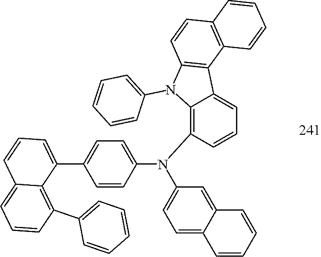 [SMILES: c1ccc(-c2cccc3cccc(-c4ccc(N(c5ccc6ccccc6c5)c5cccc6c7c8ccccc8ccc7n(-c7ccccc7)c56)cc4)c23)cc1]

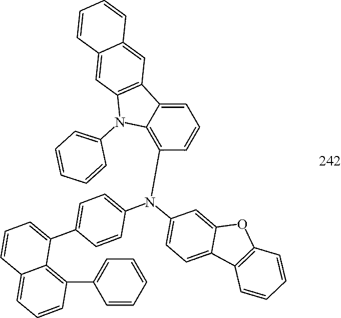 [SMILES: c1ccc(-c2cccc3cccc(-c4ccc(N(c5ccc6c(c5)oc5ccccc56)c5cccc6c7cc8ccccc8cc7n(-c7ccccc7)c56)cc4)c23)cc1]

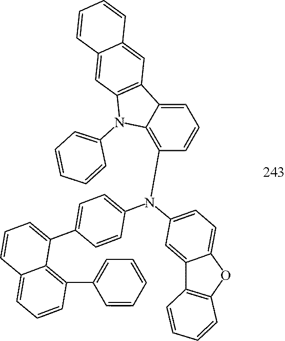 [SMILES: c1ccc(-c2cccc3cccc(-c4ccc(N(c5ccc6oc7ccccc7c6c5)c5cccc6c7cc8ccccc8cc7n(-c7ccccc7)c56)cc4)c23)cc1]

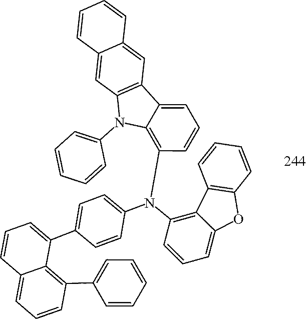 [SMILES: c1ccc(-c2cccc3cccc(-c4ccc(N(c5cccc6oc7ccccc7c56)c5cccc6c7cc8ccccc8cc7n(-c7ccccc7)c56)cc4)c23)cc1]

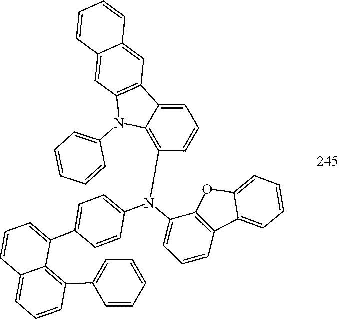 [SMILES: c1ccc(-c2cccc3cccc(-c4ccc(N(c5cccc6c5oc5ccccc56)c5cccc6c7cc8ccccc8cc7n(-c7ccccc7)c56)cc4)c23)cc1]

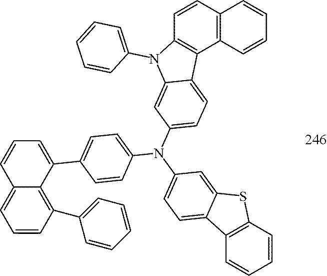 [SMILES: c1ccc(-c2cccc3cccc(-c4ccc(N(c5ccc6c(c5)sc5ccccc56)c5ccc6c7c8ccccc8ccc7n(-c7ccccc7)c6c5)cc4)c23)cc1]

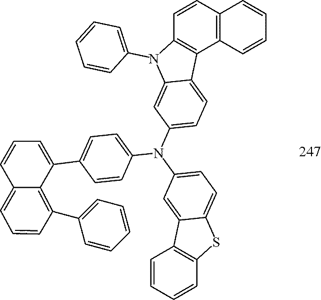 [SMILES: c1ccc(-c2cccc3cccc(-c4ccc(N(c5ccc6sc7ccccc7c6c5)c5ccc6c7c8ccccc8ccc7n(-c7ccccc7)c6c5)cc4)c23)cc1]

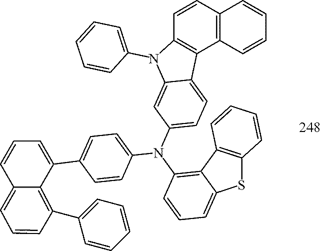 [SMILES: c1ccc(-c2cccc3cccc(-c4ccc(N(c5ccc6c7c8ccccc8ccc7n(-c7ccccc7)c6c5)c5cccc6sc7ccccc7c56)cc4)c23)cc1]

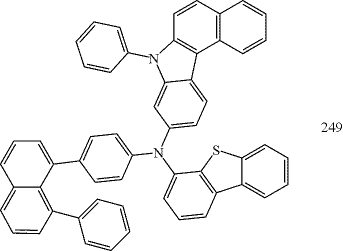 [SMILES: c1ccc(-c2cccc3cccc(-c4ccc(N(c5ccc6c7c8ccccc8ccc7n(-c7ccccc7)c6c5)c5cccc6c5sc5ccccc56)cc4)c23)cc1]

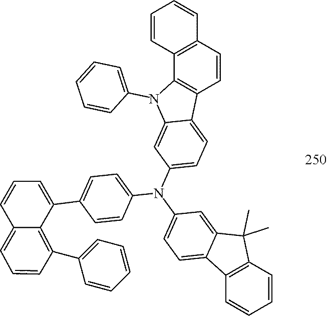 [SMILES: CC1(C)c2ccccc2-c2ccc(N(c3ccc(-c4cccc5cccc(-c6ccccc6)c45)cc3)c3ccc4c5ccc6ccccc6c5n(-c5ccccc5)c4c3)cc21]

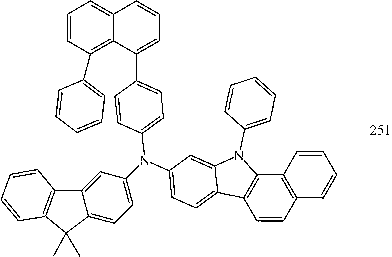 [SMILES: CC1(C)c2ccccc2-c2cc(N(c3ccc(-c4cccc5cccc(-c6ccccc6)c45)cc3)c3ccc4c5ccc6ccccc6c5n(-c5ccccc5)c4c3)ccc21]

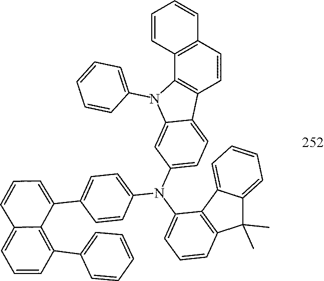 [SMILES: CC1(C)c2ccccc2-c2c(N(c3ccc(-c4cccc5cccc(-c6ccccc6)c45)cc3)c3ccc4c5ccc6ccccc6c5n(-c5ccccc5)c4c3)cccc21]

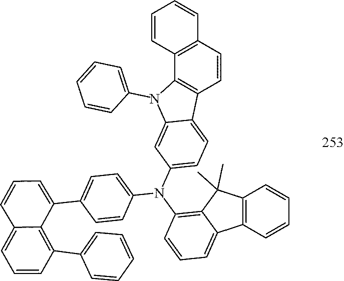 [SMILES: CC1(C)c2ccccc2-c2cccc(N(c3ccc(-c4cccc5cccc(-c6ccccc6)c45)cc3)c3ccc4c5ccc6ccccc6c5n(-c5ccccc5)c4c3)c21]

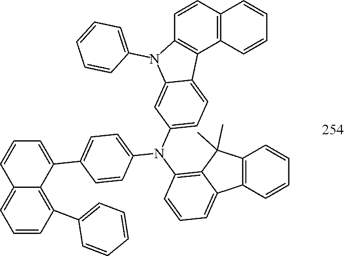 [SMILES: CC1(C)c2ccccc2-c2cccc(N(c3ccc(-c4cccc5cccc(-c6ccccc6)c45)cc3)c3ccc4c5c6ccccc6ccc5n(-c5ccccc5)c4c3)c21]

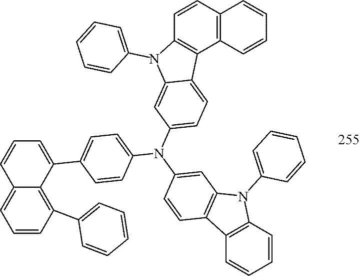 [SMILES: c1ccc(-c2cccc3cccc(-c4ccc(N(c5ccc6c7ccccc7n(-c7ccccc7)c6c5)c5ccc6c7c8ccccc8ccc7n(-c7ccccc7)c6c5)cc4)c23)cc1]

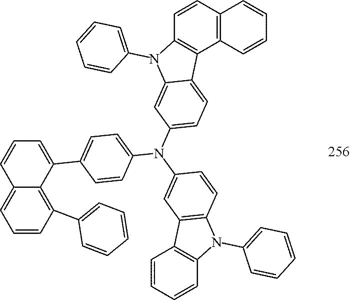 [SMILES: c1ccc(-c2cccc3cccc(-c4ccc(N(c5ccc6c(c5)c5ccccc5n6-c5ccccc5)c5ccc6c7c8ccccc8ccc7n(-c7ccccc7)c6c5)cc4)c23)cc1]

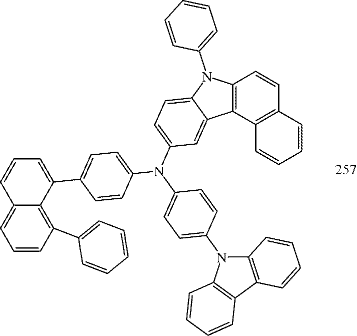 [SMILES: c1ccc(-c2cccc3cccc(-c4ccc(N(c5ccc(-n6c7ccccc7c7ccccc76)cc5)c5ccc6c(c5)c5c7ccccc7ccc5n6-c5ccccc5)cc4)c23)cc1]

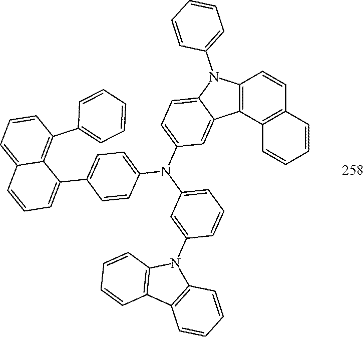 [SMILES: c1ccc(-c2cccc3cccc(-c4ccc(N(c5cccc(-n6c7ccccc7c7ccccc76)c5)c5ccc6c(c5)c5c7ccccc7ccc5n6-c5ccccc5)cc4)c23)cc1]

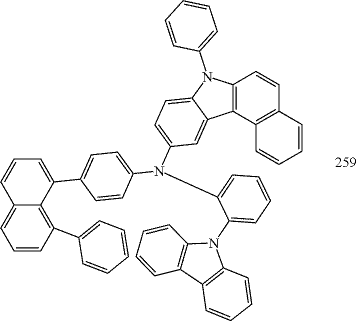 [SMILES: c1ccc(-c2cccc3cccc(-c4ccc(N(c5ccc6c(c5)c5c7ccccc7ccc5n6-c5ccccc5)c5ccccc5-n5c6ccccc6c6ccccc65)cc4)c23)cc1]

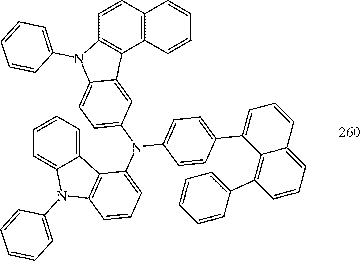 [SMILES: c1ccc(-c2cccc3cccc(-c4ccc(N(c5ccc6c(c5)c5c7ccccc7ccc5n6-c5ccccc5)c5cccc6c5c5ccccc5n6-c5ccccc5)cc4)c23)cc1]

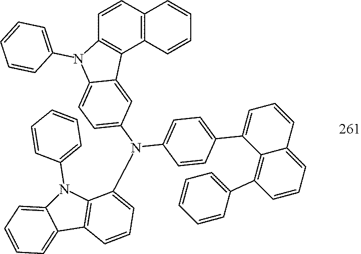 [SMILES: c1ccc(-c2cccc3cccc(-c4ccc(N(c5ccc6c(c5)c5c7ccccc7ccc5n6-c5ccccc5)c5cccc6c7ccccc7n(-c7ccccc7)c56)cc4)c23)cc1]

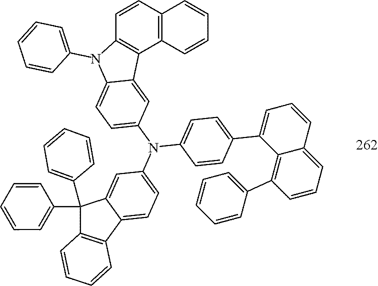 [SMILES: c1ccc(-c2cccc3cccc(-c4ccc(N(c5ccc6c(c5)C(c5ccccc5)(c5ccccc5)c5ccccc5-6)c5ccc6c(c5)c5c7ccccc7ccc5n6-c5ccccc5)cc4)c23)cc1]

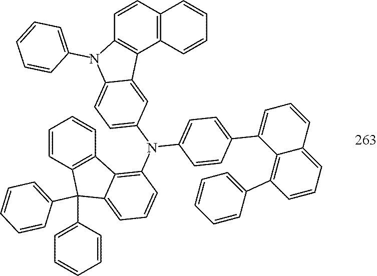 [SMILES: c1ccc(-c2cccc3cccc(-c4ccc(N(c5ccc6c(c5)c5c7ccccc7ccc5n6-c5ccccc5)c5cccc6c5-c5ccccc5C6(c5ccccc5)c5ccccc5)cc4)c23)cc1]

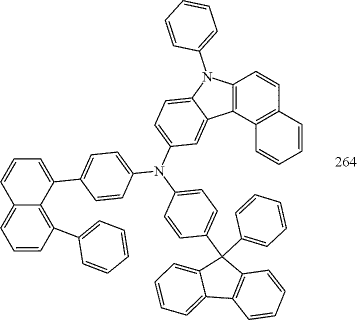 [SMILES: c1ccc(-c2cccc3cccc(-c4ccc(N(c5ccc(C6(c7ccccc7)c7ccccc7-c7ccccc76)cc5)c5ccc6c(c5)c5c7ccccc7ccc5n6-c5ccccc5)cc4)c23)cc1]

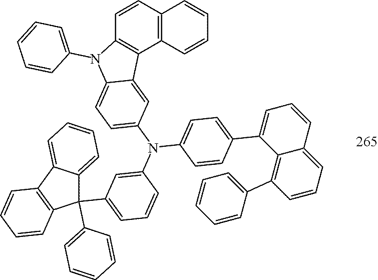 [SMILES: c1ccc(-c2cccc3cccc(-c4ccc(N(c5cccc(C6(c7ccccc7)c7ccccc7-c7ccccc76)c5)c5ccc6c(c5)c5c7ccccc7ccc5n6-c5ccccc5)cc4)c23)cc1]

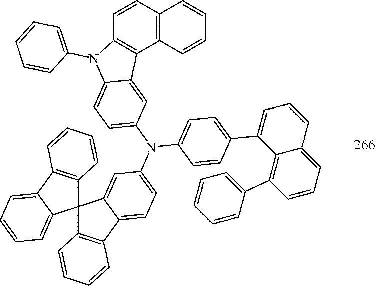 [SMILES: c1ccc(-c2cccc3cccc(-c4ccc(N(c5ccc6c(c5)C5(c7ccccc7-c7ccccc75)c5ccccc5-6)c5ccc6c(c5)c5c7ccccc7ccc5n6-c5ccccc5)cc4)c23)cc1]

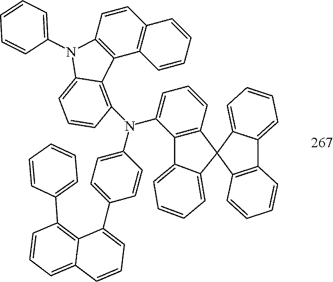 [SMILES: c1ccc(-c2cccc3cccc(-c4ccc(N(c5cccc6c5-c5ccccc5C65c6ccccc6-c6ccccc65)c5cccc6c5c5c7ccccc7ccc5n6-c5ccccc5)cc4)c23)cc1]

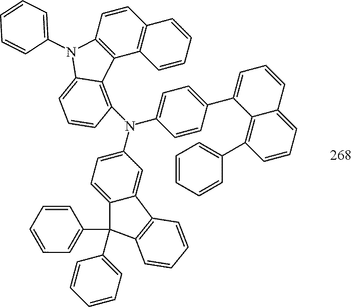 [SMILES: c1ccc(-c2cccc3cccc(-c4ccc(N(c5ccc6c(c5)-c5ccccc5C6(c5ccccc5)c5ccccc5)c5cccc6c5c5c7ccccc7ccc5n6-c5ccccc5)cc4)c23)cc1]

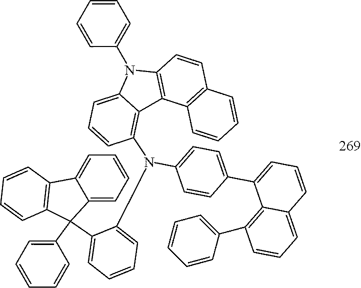 [SMILES: c1ccc(-c2cccc3cccc(-c4ccc(N(c5ccccc5C5(c6ccccc6)c6ccccc6-c6ccccc65)c5cccc6c5c5c7ccccc7ccc5n6-c5ccccc5)cc4)c23)cc1]

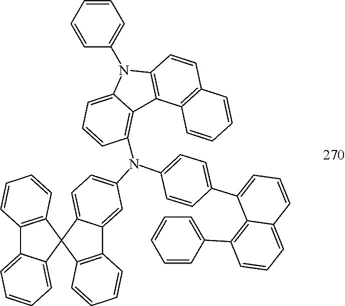 [SMILES: c1ccc(-c2cccc3cccc(-c4ccc(N(c5ccc6c(c5)-c5ccccc5C65c6ccccc6-c6ccccc65)c5cccc6c5c5c7ccccc7ccc5n6-c5ccccc5)cc4)c23)cc1]

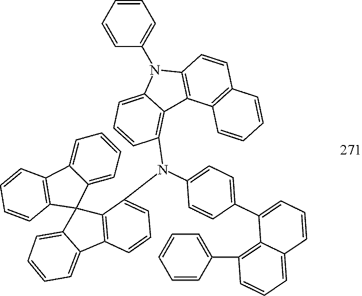 [SMILES: c1ccc(-c2cccc3cccc(-c4ccc(N(c5cccc6c5C5(c7ccccc7-c7ccccc75)c5ccccc5-6)c5cccc6c5c5c7ccccc7ccc5n6-c5ccccc5)cc4)c23)cc1]

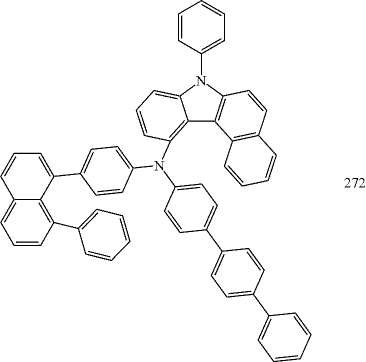 [SMILES: c1ccc(-c2ccc(-c3ccc(N(c4ccc(-c5cccc6cccc(-c7ccccc7)c56)cc4)c4cccc5c4c4c6ccccc6ccc4n5-c4ccccc4)cc3)cc2)cc1]

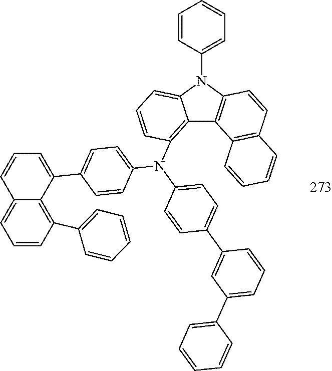 [SMILES: c1ccc(-c2cccc(-c3ccc(N(c4ccc(-c5cccc6cccc(-c7ccccc7)c56)cc4)c4cccc5c4c4c6ccccc6ccc4n5-c4ccccc4)cc3)c2)cc1]

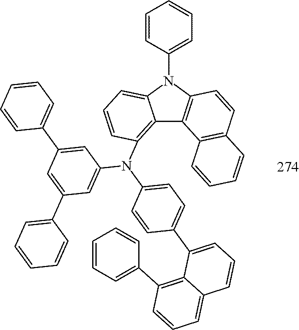 [SMILES: c1ccc(-c2cc(-c3ccccc3)cc(N(c3ccc(-c4cccc5cccc(-c6ccccc6)c45)cc3)c3cccc4c3c3c5ccccc5ccc3n4-c3ccccc3)c2)cc1]

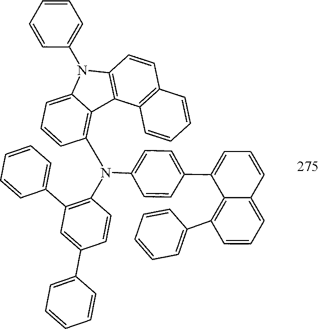 [SMILES: c1ccc(-c2ccc(N(c3ccc(-c4cccc5cccc(-c6ccccc6)c45)cc3)c3cccc4c3c3c5ccccc5ccc3n4-c3ccccc3)c(-c3ccccc3)c2)cc1]

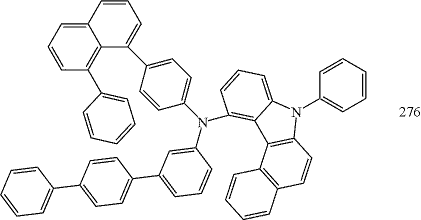 [SMILES: c1ccc(-c2ccc(-c3cccc(N(c4ccc(-c5cccc6cccc(-c7ccccc7)c56)cc4)c4cccc5c4c4c6ccccc6ccc4n5-c4ccccc4)c3)cc2)cc1]

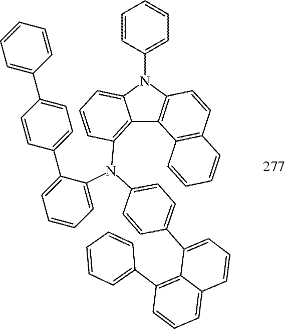 [SMILES: c1ccc(-c2ccc(-c3ccccc3N(c3ccc(-c4cccc5cccc(-c6ccccc6)c45)cc3)c3cccc4c3c3c5ccccc5ccc3n4-c3ccccc3)cc2)cc1]

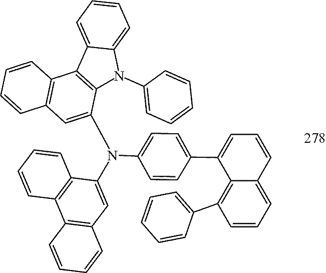 [SMILES: c1ccc(-c2cccc3cccc(-c4ccc(N(c5cc6ccccc6c6ccccc56)c5cc6ccccc6c6c7ccccc7n(-c7ccccc7)c56)cc4)c23)cc1]

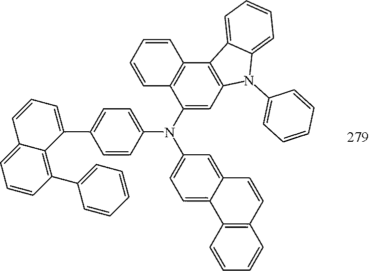 [SMILES: c1ccc(-c2cccc3cccc(-c4ccc(N(c5ccc6c(ccc7ccccc76)c5)c5cc6c(c7ccccc57)c5ccccc5n6-c5ccccc5)cc4)c23)cc1]

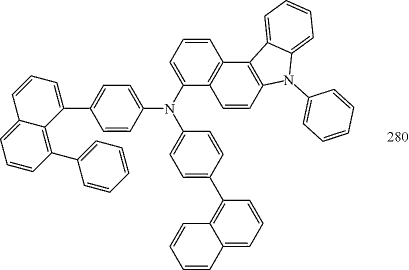 [SMILES: c1ccc(-c2cccc3cccc(-c4ccc(N(c5ccc(-c6cccc7ccccc67)cc5)c5cccc6c5ccc5c6c6ccccc6n5-c5ccccc5)cc4)c23)cc1]

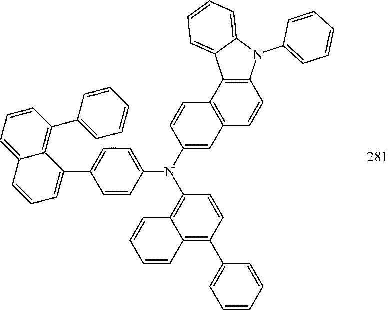 [SMILES: c1ccc(-c2ccc(N(c3ccc(-c4cccc5cccc(-c6ccccc6)c45)cc3)c3ccc4c(ccc5c4c4ccccc4n5-c4ccccc4)c3)c3ccccc23)cc1]

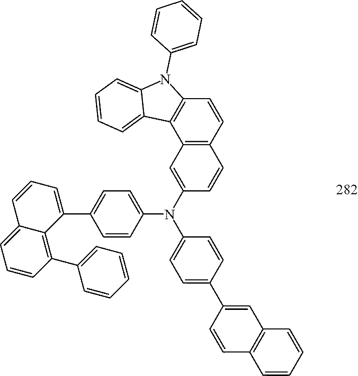 [SMILES: c1ccc(-c2cccc3cccc(-c4ccc(N(c5ccc(-c6ccc7ccccc7c6)cc5)c5ccc6ccc7c(c6c5)c5ccccc5n7-c5ccccc5)cc4)c23)cc1]

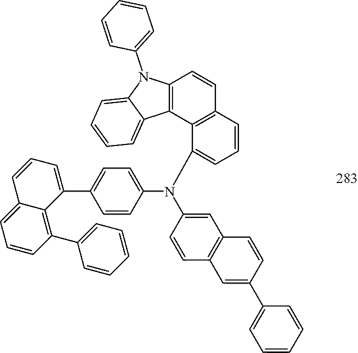 [SMILES: c1ccc(-c2ccc3cc(N(c4ccc(-c5cccc6cccc(-c7ccccc7)c56)cc4)c4cccc5ccc6c(c7ccccc7n6-c6ccccc6)c45)ccc3c2)cc1]

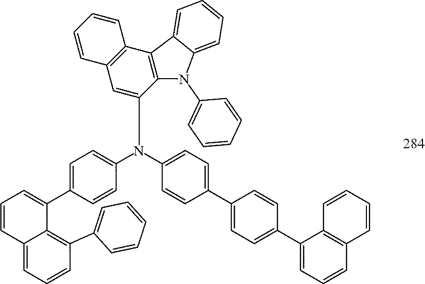 [SMILES: c1ccc(-c2cccc3cccc(-c4ccc(N(c5ccc(-c6ccc(-c7cccc8ccccc78)cc6)cc5)c5cc6ccccc6c6c7ccccc7n(-c7ccccc7)c56)cc4)c23)cc1]

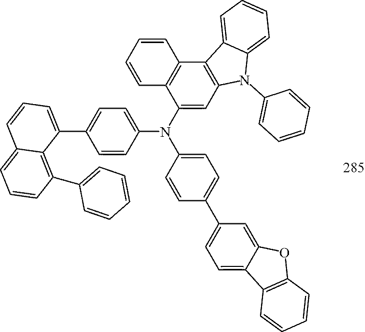 [SMILES: c1ccc(-c2cccc3cccc(-c4ccc(N(c5ccc(-c6ccc7c(c6)oc6ccccc67)cc5)c5cc6c(c7ccccc57)c5ccccc5n6-c5ccccc5)cc4)c23)cc1]

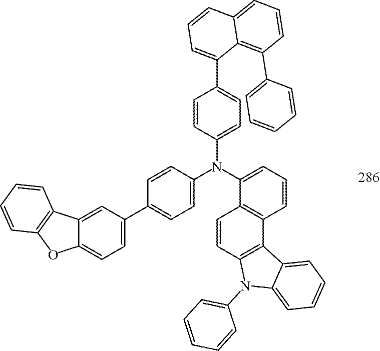 [SMILES: c1ccc(-c2cccc3cccc(-c4ccc(N(c5ccc(-c6ccc7oc8ccccc8c7c6)cc5)c5cccc6c5ccc5c6c6ccccc6n5-c5ccccc5)cc4)c23)cc1]

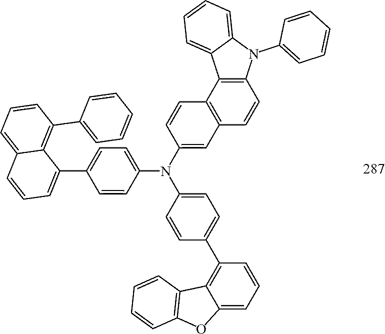 [SMILES: c1ccc(-c2cccc3cccc(-c4ccc(N(c5ccc(-c6cccc7oc8ccccc8c67)cc5)c5ccc6c(ccc7c6c6ccccc6n7-c6ccccc6)c5)cc4)c23)cc1]

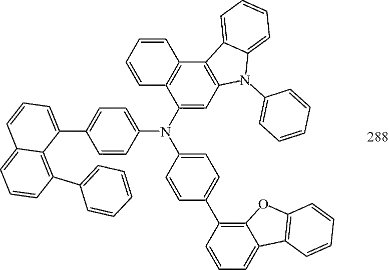 [SMILES: c1ccc(-c2cccc3cccc(-c4ccc(N(c5ccc(-c6cccc7c6oc6ccccc67)cc5)c5cc6c(c7ccccc57)c5ccccc5n6-c5ccccc5)cc4)c23)cc1]

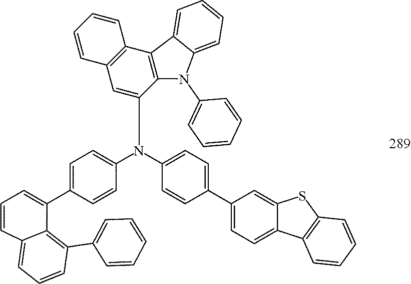 [SMILES: c1ccc(-c2cccc3cccc(-c4ccc(N(c5ccc(-c6ccc7c(c6)sc6ccccc67)cc5)c5cc6ccccc6c6c7ccccc7n(-c7ccccc7)c56)cc4)c23)cc1]

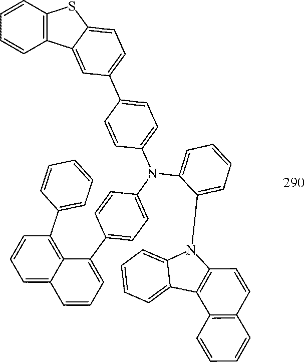 [SMILES: c1ccc(-c2cccc3cccc(-c4ccc(N(c5ccc(-c6ccc7sc8ccccc8c7c6)cc5)c5ccccc5-n5c6ccccc6c6c7ccccc7ccc65)cc4)c23)cc1]

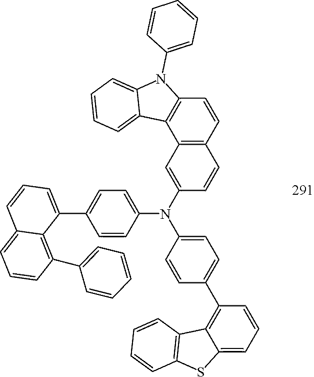 [SMILES: c1ccc(-c2cccc3cccc(-c4ccc(N(c5ccc(-c6cccc7sc8ccccc8c67)cc5)c5ccc6ccc7c(c6c5)c5ccccc5n7-c5ccccc5)cc4)c23)cc1]

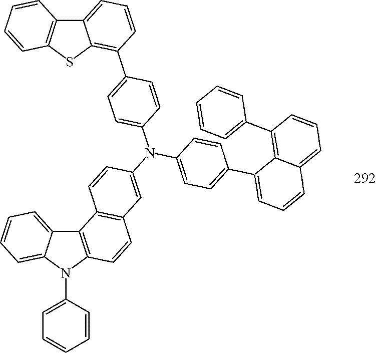 [SMILES: c1ccc(-c2cccc3cccc(-c4ccc(N(c5ccc(-c6cccc7c6sc6ccccc67)cc5)c5ccc6c(ccc7c6c6ccccc6n7-c6ccccc6)c5)cc4)c23)cc1]

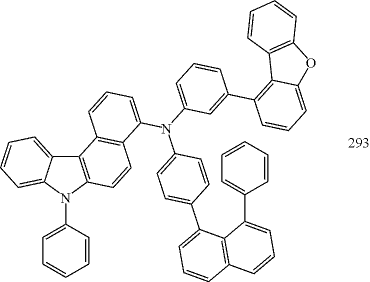 [SMILES: c1ccc(-c2cccc3cccc(-c4ccc(N(c5cccc(-c6cccc7oc8ccccc8c67)c5)c5cccc6c5ccc5c6c6ccccc6n5-c5ccccc5)cc4)c23)cc1]

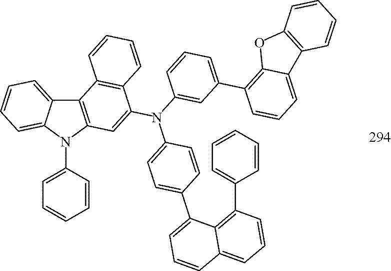 [SMILES: c1ccc(-c2cccc3cccc(-c4ccc(N(c5cccc(-c6cccc7c6oc6ccccc67)c5)c5cc6c(c7ccccc57)c5ccccc5n6-c5ccccc5)cc4)c23)cc1]

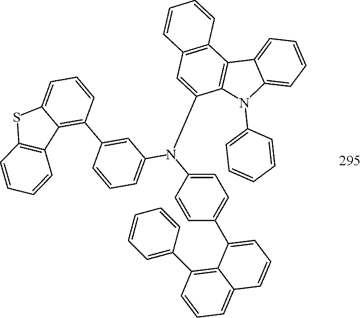 [SMILES: c1ccc(-c2cccc3cccc(-c4ccc(N(c5cccc(-c6cccc7sc8ccccc8c67)c5)c5cc6ccccc6c6c7ccccc7n(-c7ccccc7)c56)cc4)c23)cc1]

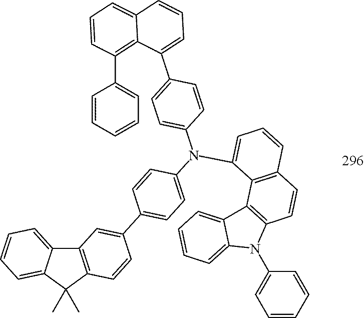 [SMILES: CC1(C)c2ccccc2-c2cc(-c3ccc(N(c4ccc(-c5cccc6cccc(-c7ccccc7)c56)cc4)c4cccc5ccc6c(c7ccccc7n6-c6ccccc6)c45)cc3)ccc21]

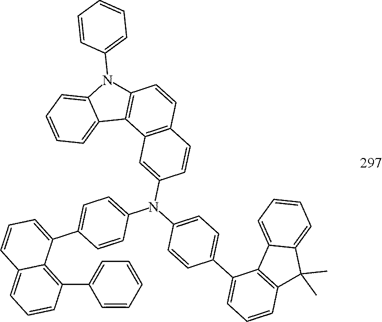 [SMILES: CC1(C)c2ccccc2-c2c(-c3ccc(N(c4ccc(-c5cccc6cccc(-c7ccccc7)c56)cc4)c4ccc5ccc6c(c5c4)c4ccccc4n6-c4ccccc4)cc3)cccc21]

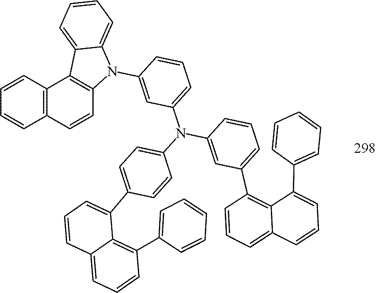 [SMILES: c1ccc(-c2cccc3cccc(-c4ccc(N(c5cccc(-c6cccc7cccc(-c8ccccc8)c67)c5)c5cccc(-n6c7ccccc7c7c8ccccc8ccc76)c5)cc4)c23)cc1]

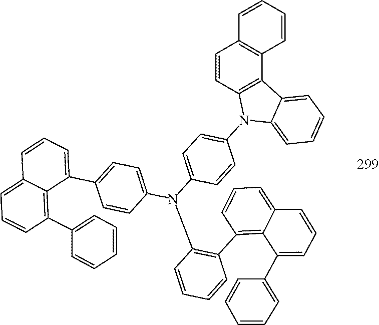 [SMILES: c1ccc(-c2cccc3cccc(-c4ccc(N(c5ccc(-n6c7ccccc7c7c8ccccc8ccc76)cc5)c5ccccc5-c5cccc6cccc(-c7ccccc7)c56)cc4)c23)cc1]

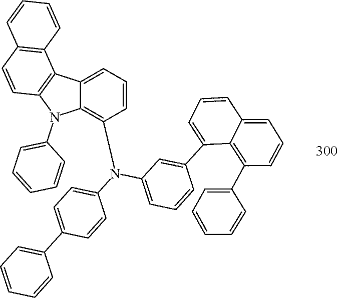 [SMILES: c1ccc(-c2ccc(N(c3cccc(-c4cccc5cccc(-c6ccccc6)c45)c3)c3cccc4c5c6ccccc6ccc5n(-c5ccccc5)c34)cc2)cc1]

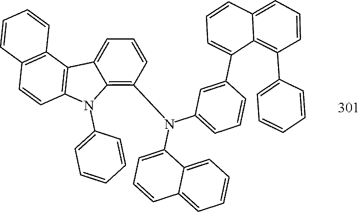 [SMILES: c1ccc(-c2cccc3cccc(-c4cccc(N(c5cccc6ccccc56)c5cccc6c7c8ccccc8ccc7n(-c7ccccc7)c56)c4)c23)cc1]

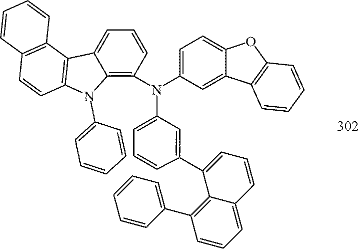 [SMILES: c1ccc(-c2cccc3cccc(-c4cccc(N(c5ccc6oc7ccccc7c6c5)c5cccc6c7c8ccccc8ccc7n(-c7ccccc7)c56)c4)c23)cc1]

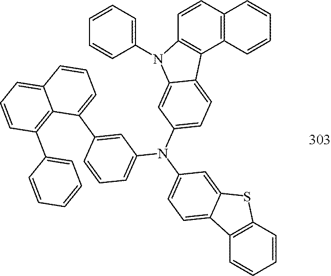 [SMILES: c1ccc(-c2cccc3cccc(-c4cccc(N(c5ccc6c(c5)sc5ccccc56)c5ccc6c7c8ccccc8ccc7n(-c7ccccc7)c6c5)c4)c23)cc1]

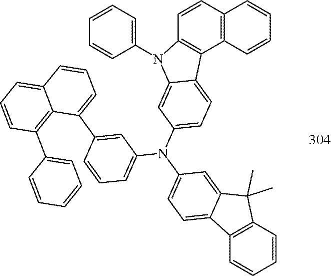 [SMILES: CC1(C)c2ccccc2-c2ccc(N(c3cccc(-c4cccc5cccc(-c6ccccc6)c45)c3)c3ccc4c5c6ccccc6ccc5n(-c5ccccc5)c4c3)cc21]

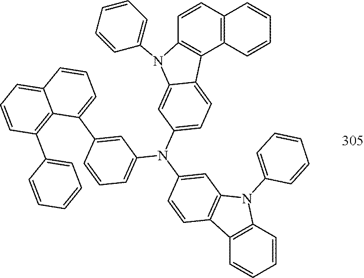 [SMILES: c1ccc(-c2cccc3cccc(-c4cccc(N(c5ccc6c7ccccc7n(-c7ccccc7)c6c5)c5ccc6c7c8ccccc8ccc7n(-c7ccccc7)c6c5)c4)c23)cc1]

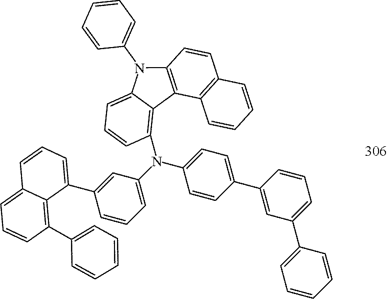 [SMILES: c1ccc(-c2cccc(-c3ccc(N(c4cccc(-c5cccc6cccc(-c7ccccc7)c56)c4)c4cccc5c4c4c6ccccc6ccc4n5-c4ccccc4)cc3)c2)cc1]

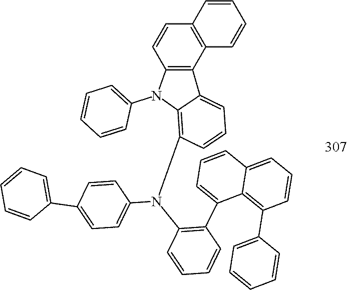 [SMILES: c1ccc(-c2ccc(N(c3ccccc3-c3cccc4cccc(-c5ccccc5)c34)c3cccc4c5c6ccccc6ccc5n(-c5ccccc5)c34)cc2)cc1]

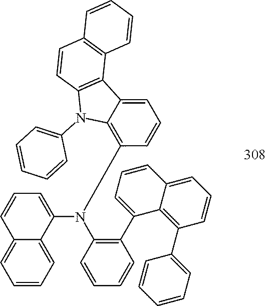 [SMILES: c1ccc(-c2cccc3cccc(-c4ccccc4N(c4cccc5ccccc45)c4cccc5c6c7ccccc7ccc6n(-c6ccccc6)c45)c23)cc1]

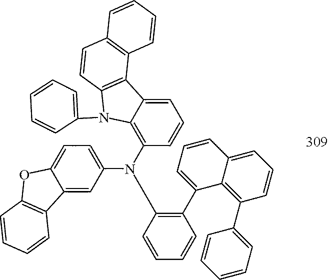 [SMILES: c1ccc(-c2cccc3cccc(-c4ccccc4N(c4ccc5oc6ccccc6c5c4)c4cccc5c6c7ccccc7ccc6n(-c6ccccc6)c45)c23)cc1]

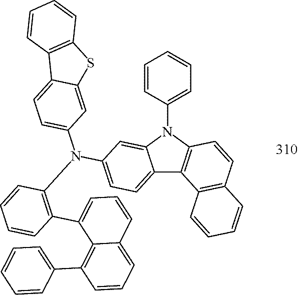 [SMILES: c1ccc(-c2cccc3cccc(-c4ccccc4N(c4ccc5c(c4)sc4ccccc45)c4ccc5c6c7ccccc7ccc6n(-c6ccccc6)c5c4)c23)cc1]

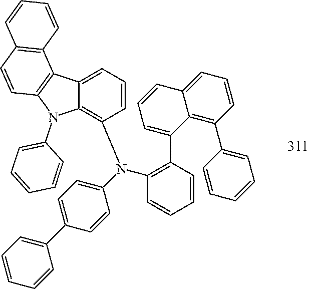 [SMILES: c1ccc(-c2ccc(N(c3ccccc3-c3cccc4cccc(-c5ccccc5)c34)c3cccc4c5c6ccccc6ccc5n(-c5ccccc5)c34)cc2)cc1]

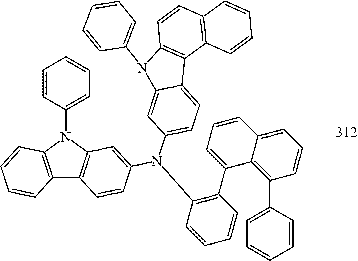 [SMILES: c1ccc(-c2cccc3cccc(-c4ccccc4N(c4ccc5c6ccccc6n(-c6ccccc6)c5c4)c4ccc5c6c7ccccc7ccc6n(-c6ccccc6)c5c4)c23)cc1]

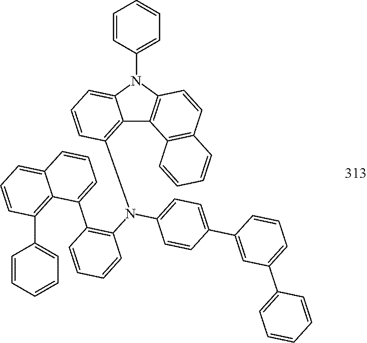 [SMILES: c1ccc(-c2cccc(-c3ccc(N(c4ccccc4-c4cccc5cccc(-c6ccccc6)c45)c4cccc5c4c4c6ccccc6ccc4n5-c4ccccc4)cc3)c2)cc1]

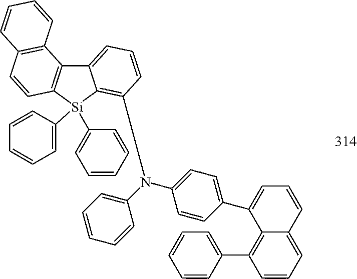 [SMILES: c1ccc(-c2cccc3cccc(-c4ccc(N(c5ccccc5)c5cccc6c5[Si](c5ccccc5)(c5ccccc5)c5ccc7ccccc7c5-6)cc4)c23)cc1]

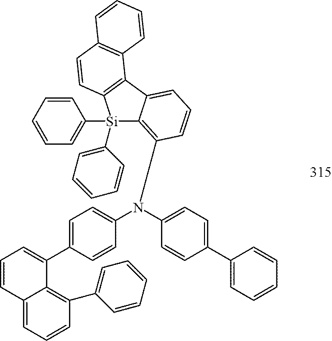 [SMILES: c1ccc(-c2ccc(N(c3ccc(-c4cccc5cccc(-c6ccccc6)c45)cc3)c3cccc4c3[Si](c3ccccc3)(c3ccccc3)c3ccc5ccccc5c3-4)cc2)cc1]

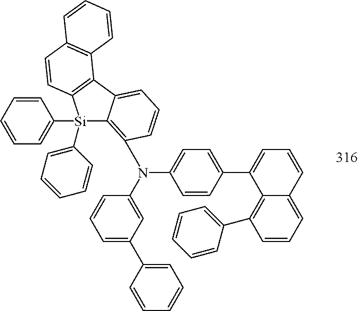 [SMILES: c1ccc(-c2cccc(N(c3ccc(-c4cccc5cccc(-c6ccccc6)c45)cc3)c3cccc4c3[Si](c3ccccc3)(c3ccccc3)c3ccc5ccccc5c3-4)c2)cc1]

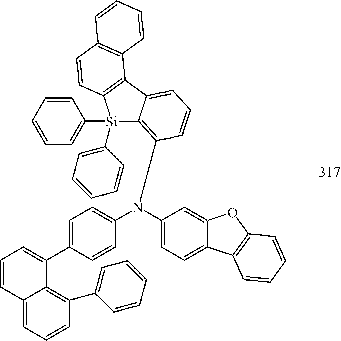 [SMILES: c1ccc(-c2cccc3cccc(-c4ccc(N(c5ccc6c(c5)oc5ccccc56)c5cccc6c5[Si](c5ccccc5)(c5ccccc5)c5ccc7ccccc7c5-6)cc4)c23)cc1]

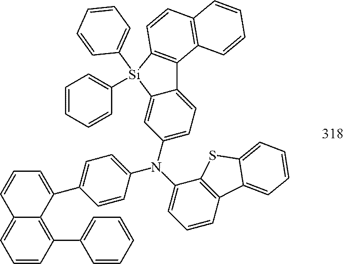 [SMILES: c1ccc(-c2cccc3cccc(-c4ccc(N(c5ccc6c(c5)[Si](c5ccccc5)(c5ccccc5)c5ccc7ccccc7c5-6)c5cccc6c5sc5ccccc56)cc4)c23)cc1]

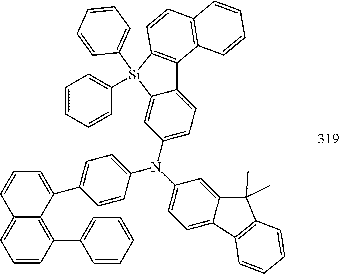 [SMILES: CC1(C)c2ccccc2-c2ccc(N(c3ccc(-c4cccc5cccc(-c6ccccc6)c45)cc3)c3ccc4c(c3)[Si](c3ccccc3)(c3ccccc3)c3ccc5ccccc5c3-4)cc21]

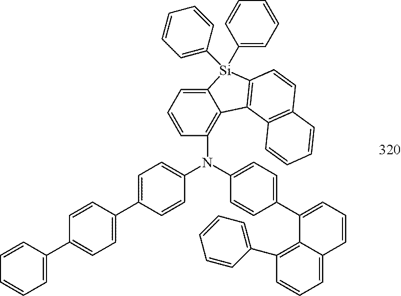 [SMILES: c1ccc(-c2ccc(-c3ccc(N(c4ccc(-c5cccc6cccc(-c7ccccc7)c56)cc4)c4cccc5c4-c4c(ccc6ccccc46)[Si]5(c4ccccc4)c4ccccc4)cc3)cc2)cc1]

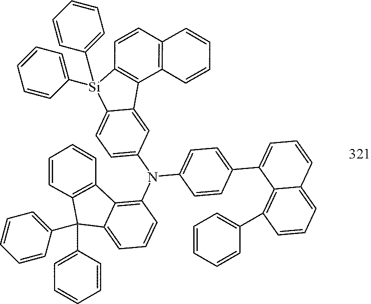 [SMILES: c1ccc(-c2cccc3cccc(-c4ccc(N(c5ccc6c(c5)-c5c(ccc7ccccc57)[Si]6(c5ccccc5)c5ccccc5)c5cccc6c5-c5ccccc5C6(c5ccccc5)c5ccccc5)cc4)c23)cc1]

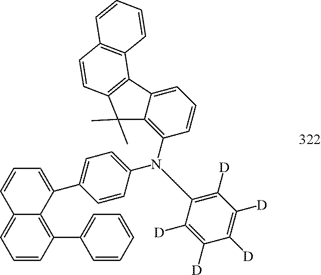 [SMILES: [2H]c1c([2H])c([2H])c(N(c2ccc(-c3cccc4cccc(-c5ccccc5)c34)cc2)c2cccc3c2C(C)(C)c2ccc4ccccc4c2-3)c([2H])c1[2H]]

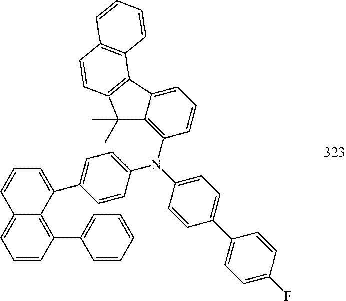 [SMILES: CC1(C)c2ccc3ccccc3c2-c2cccc(N(c3ccc(-c4ccc(F)cc4)cc3)c3ccc(-c4cccc5cccc(-c6ccccc6)c45)cc3)c21]

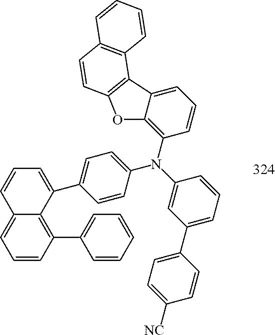

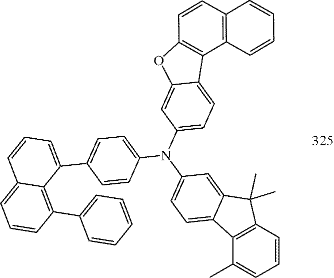 [SMILES: Cc1cccc2c1-c1ccc(N(c3ccc(-c4cccc5cccc(-c6ccccc6)c45)cc3)c3ccc4c(c3)oc3ccc5ccccc5c34)cc1C2(C)C]

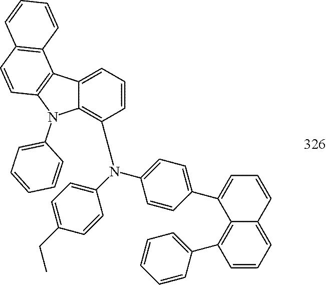 [SMILES: CCc1ccc(N(c2ccc(-c3cccc4cccc(-c5ccccc5)c34)cc2)c2cccc3c4c5ccccc5ccc4n(-c4ccccc4)c23)cc1]

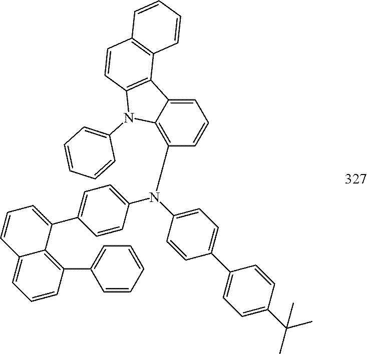 [SMILES: CC(C)(C)c1ccc(-c2ccc(N(c3ccc(-c4cccc5cccc(-c6ccccc6)c45)cc3)c3cccc4c5c6ccccc6ccc5n(-c5ccccc5)c34)cc2)cc1]

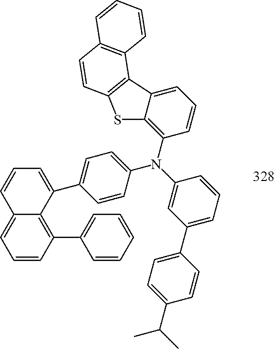 [SMILES: CC(C)c1ccc(-c2cccc(N(c3ccc(-c4cccc5cccc(-c6ccccc6)c45)cc3)c3cccc4c3sc3ccc5ccccc5c34)c2)cc1]

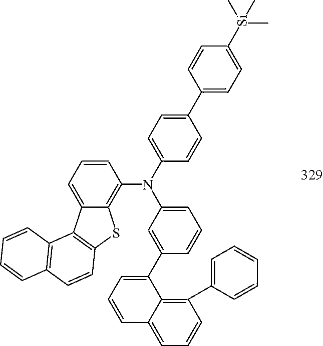 [SMILES: C[Si](C)(C)c1ccc(-c2ccc(N(c3cccc(-c4cccc5cccc(-c6ccccc6)c45)c3)c3cccc4c3sc3ccc5ccccc5c34)cc2)cc1]

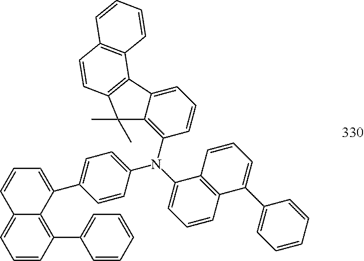 [SMILES: CC1(C)c2ccc3ccccc3c2-c2cccc(N(c3ccc(-c4cccc5cccc(-c6ccccc6)c45)cc3)c3cccc4c(-c5ccccc5)cccc34)c21]

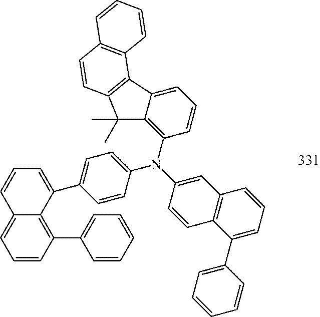 [SMILES: CC1(C)c2ccc3ccccc3c2-c2cccc(N(c3ccc(-c4cccc5cccc(-c6ccccc6)c45)cc3)c3ccc4c(-c5ccccc5)cccc4c3)c21]

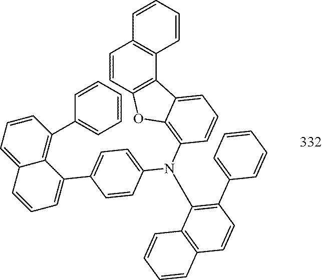 [SMILES: c1ccc(-c2ccc3ccccc3c2N(c2ccc(-c3cccc4cccc(-c5ccccc5)c34)cc2)c2cccc3c2oc2ccc4ccccc4c23)cc1]

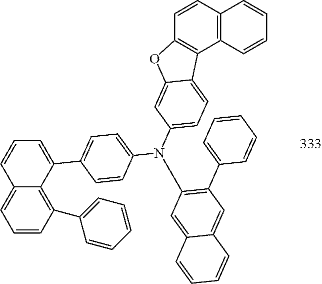 [SMILES: c1ccc(-c2cc3ccccc3cc2N(c2ccc(-c3cccc4cccc(-c5ccccc5)c34)cc2)c2ccc3c(c2)oc2ccc4ccccc4c23)cc1]

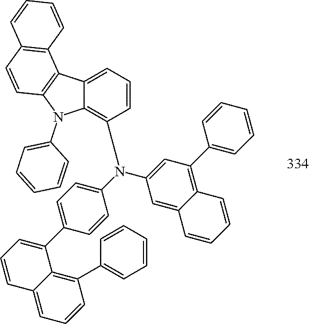 [SMILES: c1ccc(-c2cc(N(c3ccc(-c4cccc5cccc(-c6ccccc6)c45)cc3)c3cccc4c5c6ccccc6ccc5n(-c5ccccc5)c34)cc3ccccc23)cc1]

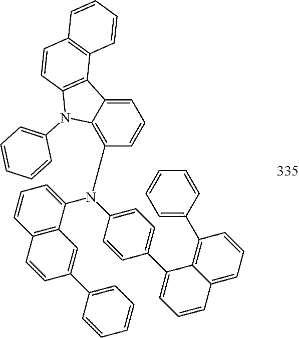 [SMILES: c1ccc(-c2ccc3cccc(N(c4ccc(-c5cccc6cccc(-c7ccccc7)c56)cc4)c4cccc5c6c7ccccc7ccc6n(-c6ccccc6)c45)c3c2)cc1]

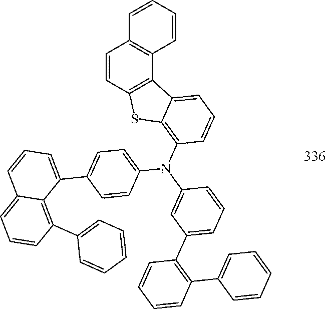 [SMILES: c1ccc(-c2ccccc2-c2cccc(N(c3ccc(-c4cccc5cccc(-c6ccccc6)c45)cc3)c3cccc4c3sc3ccc5ccccc5c34)c2)cc1]

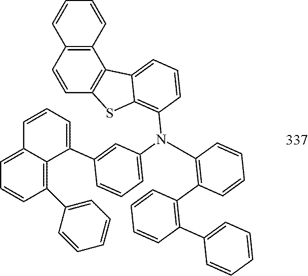 [SMILES: c1ccc(-c2ccccc2-c2ccccc2N(c2cccc(-c3cccc4cccc(-c5ccccc5)c34)c2)c2cccc3c2sc2ccc4ccccc4c23)cc1]

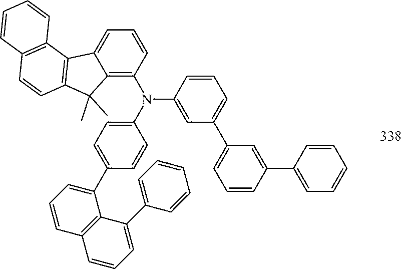 [SMILES: CC1(C)c2ccc3ccccc3c2-c2cccc(N(c3ccc(-c4cccc5cccc(-c6ccccc6)c45)cc3)c3cccc(-c4cccc(-c5ccccc5)c4)c3)c21]

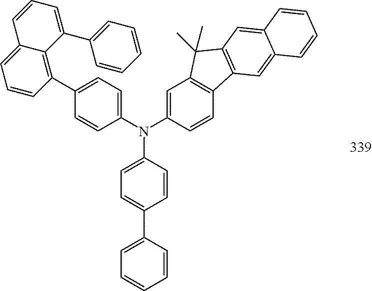 [SMILES: CC1(C)c2cc(N(c3ccc(-c4ccccc4)cc3)c3ccc(-c4cccc5cccc(-c6ccccc6)c45)cc3)ccc2-c2cc3ccccc3cc21]

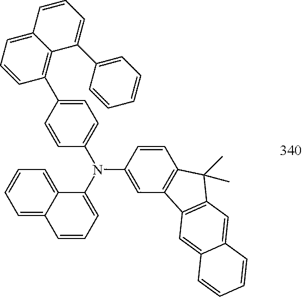 [SMILES: CC1(C)c2ccc(N(c3ccc(-c4cccc5cccc(-c6ccccc6)c45)cc3)c3cccc4ccccc34)cc2-c2cc3ccccc3cc21]

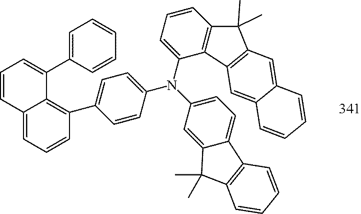 [SMILES: CC1(C)c2ccccc2-c2ccc(N(c3ccc(-c4cccc5cccc(-c6ccccc6)c45)cc3)c3cccc4c3-c3cc5ccccc5cc3C4(C)C)cc21]

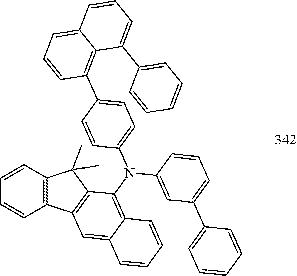 [SMILES: CC1(C)c2ccccc2-c2cc3ccccc3c(N(c3ccc(-c4cccc5cccc(-c6ccccc6)c45)cc3)c3cccc(-c4ccccc4)c3)c21]

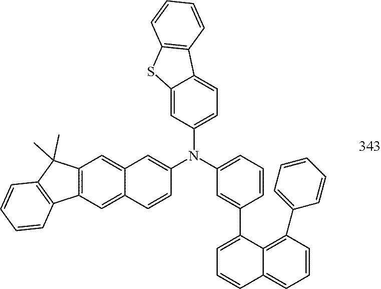 [SMILES: CC1(C)c2ccccc2-c2cc3ccc(N(c4cccc(-c5cccc6cccc(-c7ccccc7)c56)c4)c4ccc5c(c4)sc4ccccc45)cc3cc21]

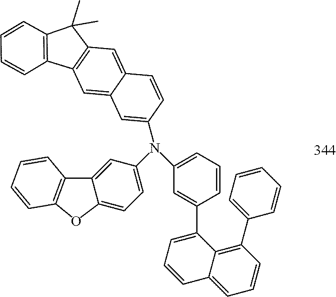 [SMILES: CC1(C)c2ccccc2-c2cc3cc(N(c4cccc(-c5cccc6cccc(-c7ccccc7)c56)c4)c4ccc5oc6ccccc6c5c4)ccc3cc21]

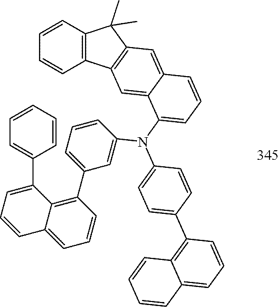 [SMILES: CC1(C)c2ccccc2-c2cc3c(N(c4ccc(-c5cccc6ccccc56)cc4)c4cccc(-c5cccc6cccc(-c7ccccc7)c56)c4)cccc3cc21]

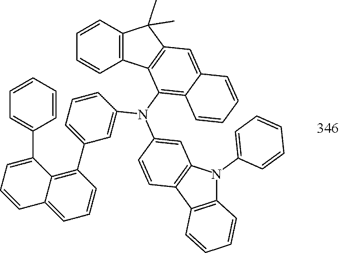 [SMILES: CC1(C)c2ccccc2-c2c1cc1ccccc1c2N(c1cccc(-c2cccc3cccc(-c4ccccc4)c23)c1)c1ccc2c3ccccc3n(-c3ccccc3)c2c1]

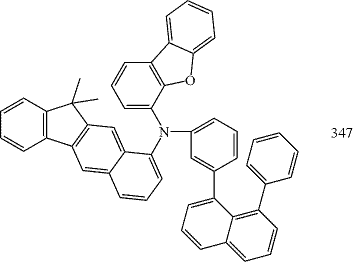 [SMILES: CC1(C)c2ccccc2-c2cc3cccc(N(c4cccc(-c5cccc6cccc(-c7ccccc7)c56)c4)c4cccc5c4oc4ccccc45)c3cc21]

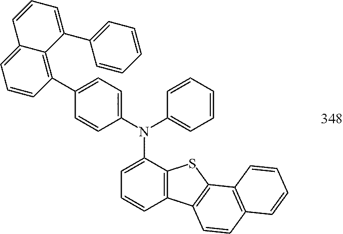 [SMILES: c1ccc(-c2cccc3cccc(-c4ccc(N(c5ccccc5)c5cccc6c5sc5c7ccccc7ccc65)cc4)c23)cc1]

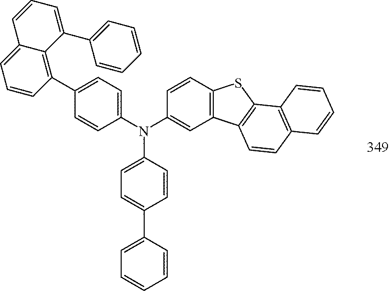 [SMILES: c1ccc(-c2ccc(N(c3ccc(-c4cccc5cccc(-c6ccccc6)c45)cc3)c3ccc4sc5c6ccccc6ccc5c4c3)cc2)cc1]

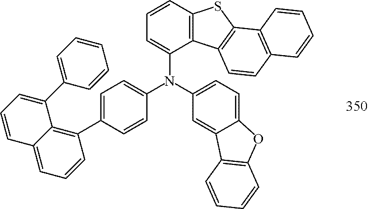 [SMILES: c1ccc(-c2cccc3cccc(-c4ccc(N(c5ccc6oc7ccccc7c6c5)c5cccc6sc7c8ccccc8ccc7c56)cc4)c23)cc1]

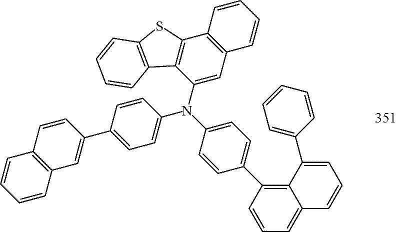 [SMILES: c1ccc(-c2cccc3cccc(-c4ccc(N(c5ccc(-c6ccc7ccccc7c6)cc5)c5cc6ccccc6c6sc7ccccc7c56)cc4)c23)cc1]

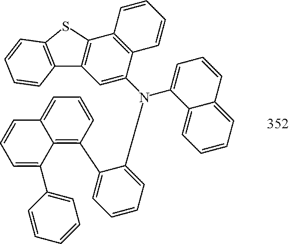 [SMILES: c1ccc(-c2cccc3cccc(-c4ccccc4N(c4cccc5ccccc45)c4cc5c6ccccc6sc5c5ccccc45)c23)cc1]

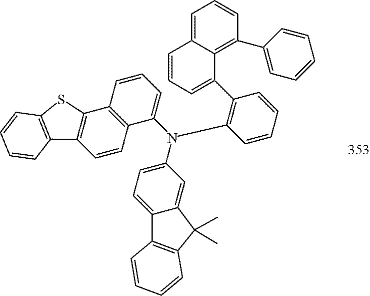 [SMILES: CC1(C)c2ccccc2-c2ccc(N(c3ccccc3-c3cccc4cccc(-c5ccccc5)c34)c3cccc4c3ccc3c5ccccc5sc43)cc21]

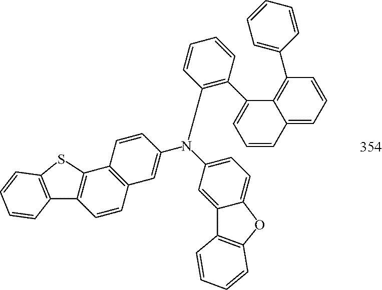 [SMILES: c1ccc(-c2cccc3cccc(-c4ccccc4N(c4ccc5c(ccc6c7ccccc7sc56)c4)c4ccc5oc6ccccc6c5c4)c23)cc1]

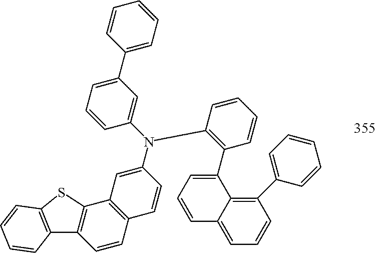 [SMILES: c1ccc(-c2cccc(N(c3ccc4ccc5c6ccccc6sc5c4c3)c3ccccc3-c3cccc4cccc(-c5ccccc5)c34)c2)cc1]

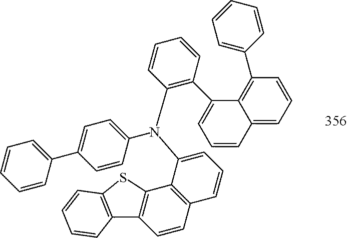 [SMILES: c1ccc(-c2ccc(N(c3ccccc3-c3cccc4cccc(-c5ccccc5)c34)c3cccc4ccc5c6ccccc6sc5c34)cc2)cc1]

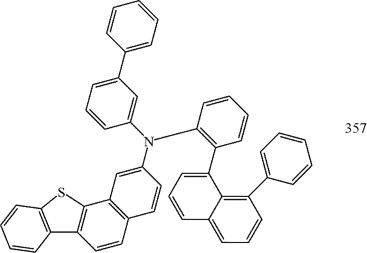 [SMILES: c1ccc(-c2cccc(N(c3ccc4ccc5c6ccccc6sc5c4c3)c3ccccc3-c3cccc4cccc(-c5ccccc5)c34)c2)cc1]

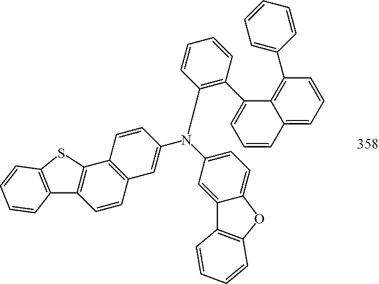 [SMILES: c1ccc(-c2cccc3cccc(-c4ccccc4N(c4ccc5c(ccc6c7ccccc7sc56)c4)c4ccc5oc6ccccc6c5c4)c23)cc1]

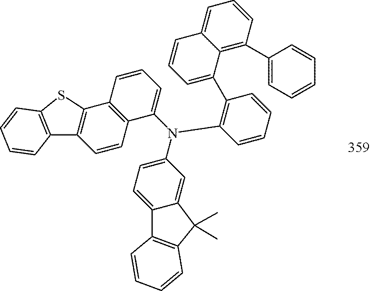 [SMILES: CC1(C)c2ccccc2-c2ccc(N(c3ccccc3-c3cccc4cccc(-c5ccccc5)c34)c3cccc4c3ccc3c5ccccc5sc43)cc21]

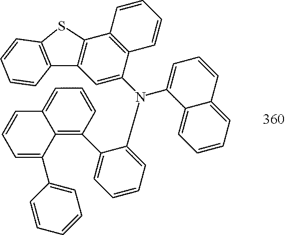 [SMILES: c1ccc(-c2cccc3cccc(-c4ccccc4N(c4cccc5ccccc45)c4cc5c6ccccc6sc5c5ccccc45)c23)cc1]

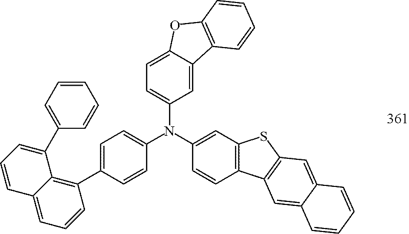 [SMILES: c1ccc(-c2cccc3cccc(-c4ccc(N(c5ccc6c(c5)sc5cc7ccccc7cc56)c5ccc6oc7ccccc7c6c5)cc4)c23)cc1]

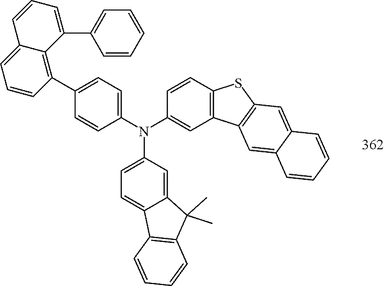 [SMILES: CC1(C)c2ccccc2-c2ccc(N(c3ccc(-c4cccc5cccc(-c6ccccc6)c45)cc3)c3ccc4sc5cc6ccccc6cc5c4c3)cc21]

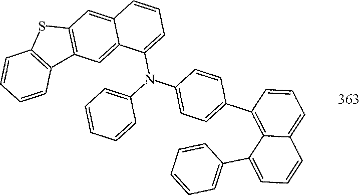 [SMILES: c1ccc(-c2cccc3cccc(-c4ccc(N(c5ccccc5)c5cccc6cc7sc8ccccc8c7cc56)cc4)c23)cc1]

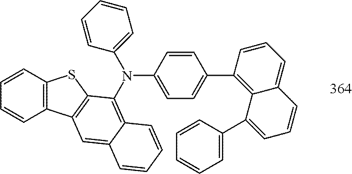 [SMILES: c1ccc(-c2cccc3cccc(-c4ccc(N(c5ccccc5)c5c6ccccc6cc6c5sc5ccccc56)cc4)c23)cc1]

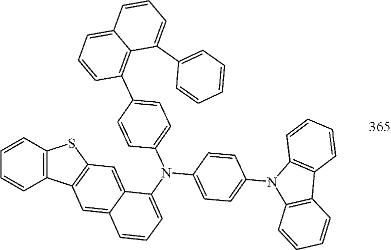 [SMILES: c1ccc(-c2cccc3cccc(-c4ccc(N(c5ccc(-n6c7ccccc7c7ccccc76)cc5)c5cccc6cc7c(cc56)sc5ccccc57)cc4)c23)cc1]

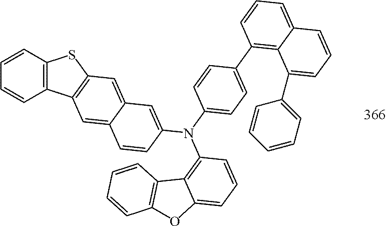 [SMILES: c1ccc(-c2cccc3cccc(-c4ccc(N(c5ccc6cc7c(cc6c5)sc5ccccc57)c5cccc6oc7ccccc7c56)cc4)c23)cc1]

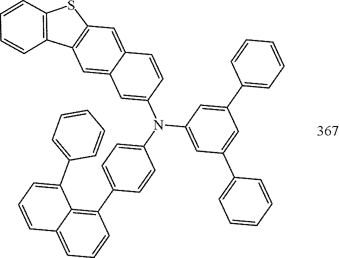 [SMILES: c1ccc(-c2cc(-c3ccccc3)cc(N(c3ccc(-c4cccc5cccc(-c6ccccc6)c45)cc3)c3ccc4cc5sc6ccccc6c5cc4c3)c2)cc1]

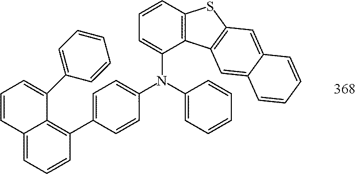 [SMILES: c1ccc(-c2cccc3cccc(-c4ccc(N(c5ccccc5)c5cccc6sc7cc8ccccc8cc7c56)cc4)c23)cc1]

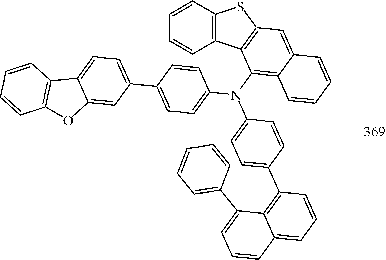 [SMILES: c1ccc(-c2cccc3cccc(-c4ccc(N(c5ccc(-c6ccc7c(c6)oc6ccccc67)cc5)c5c6ccccc6cc6sc7ccccc7c56)cc4)c23)cc1]

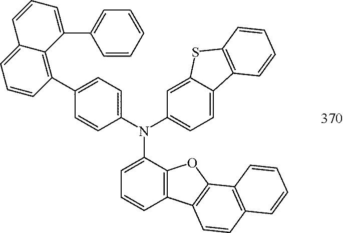 [SMILES: c1ccc(-c2cccc3cccc(-c4ccc(N(c5ccc6c(c5)sc5ccccc56)c5cccc6c5oc5c7ccccc7ccc65)cc4)c23)cc1]

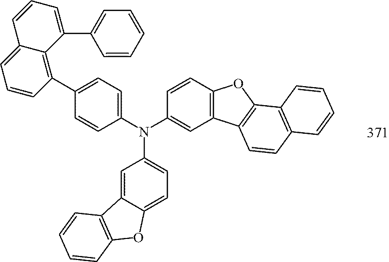 [SMILES: c1ccc(-c2cccc3cccc(-c4ccc(N(c5ccc6oc7ccccc7c6c5)c5ccc6oc7c8ccccc8ccc7c6c5)cc4)c23)cc1]

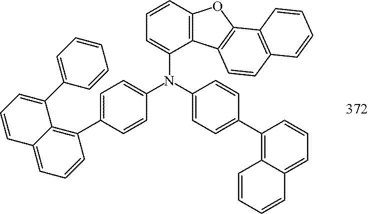 [SMILES: c1ccc(-c2cccc3cccc(-c4ccc(N(c5ccc(-c6cccc7ccccc67)cc5)c5cccc6oc7c8ccccc8ccc7c56)cc4)c23)cc1]

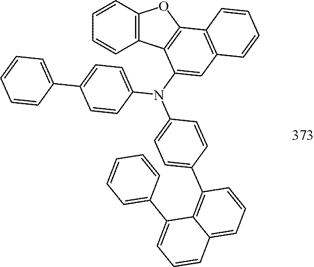 [SMILES: c1ccc(-c2ccc(N(c3ccc(-c4cccc5cccc(-c6ccccc6)c45)cc3)c3cc4ccccc4c4oc5ccccc5c34)cc2)cc1]

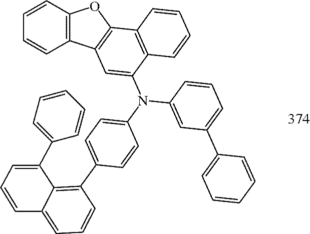 [SMILES: c1ccc(-c2cccc(N(c3ccc(-c4cccc5cccc(-c6ccccc6)c45)cc3)c3cc4c5ccccc5oc4c4ccccc34)c2)cc1]

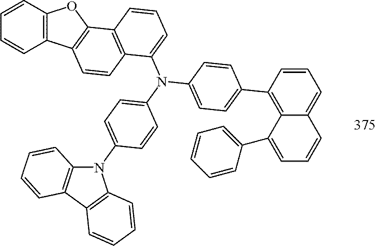 [SMILES: c1ccc(-c2cccc3cccc(-c4ccc(N(c5ccc(-n6c7ccccc7c7ccccc76)cc5)c5cccc6c5ccc5c7ccccc7oc65)cc4)c23)cc1]

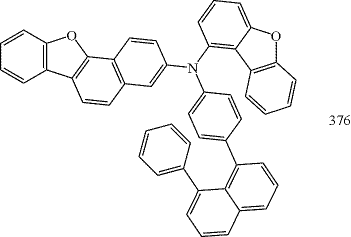 [SMILES: c1ccc(-c2cccc3cccc(-c4ccc(N(c5ccc6c(ccc7c8ccccc8oc67)c5)c5cccc6oc7ccccc7c56)cc4)c23)cc1]

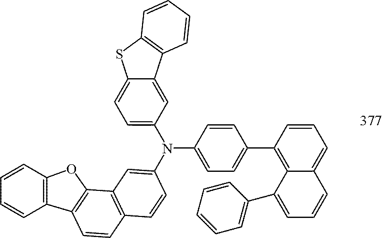 [SMILES: c1ccc(-c2cccc3cccc(-c4ccc(N(c5ccc6sc7ccccc7c6c5)c5ccc6ccc7c8ccccc8oc7c6c5)cc4)c23)cc1]

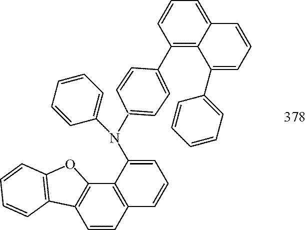

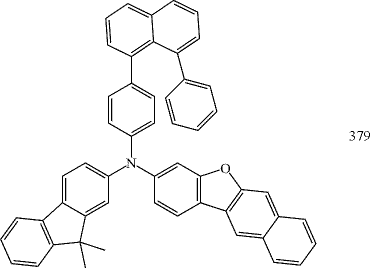 [SMILES: CC1(C)c2ccccc2-c2ccc(N(c3ccc(-c4cccc5cccc(-c6ccccc6)c45)cc3)c3ccc4c(c3)oc3cc5ccccc5cc34)cc21]

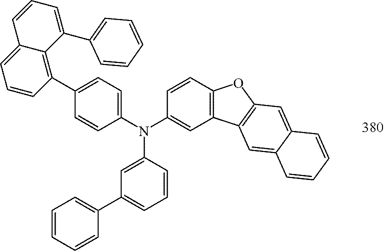 [SMILES: c1ccc(-c2cccc(N(c3ccc(-c4cccc5cccc(-c6ccccc6)c45)cc3)c3ccc4oc5cc6ccccc6cc5c4c3)c2)cc1]

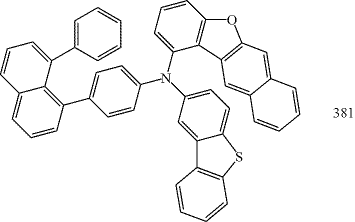 [SMILES: c1ccc(-c2cccc3cccc(-c4ccc(N(c5ccc6sc7ccccc7c6c5)c5cccc6oc7cc8ccccc8cc7c56)cc4)c23)cc1]

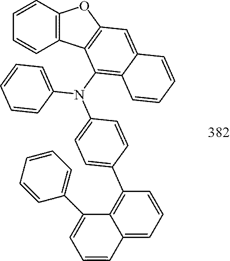 [SMILES: c1ccc(-c2cccc3cccc(-c4ccc(N(c5ccccc5)c5c6ccccc6cc6oc7ccccc7c56)cc4)c23)cc1]

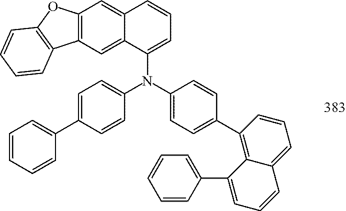 [SMILES: c1ccc(-c2ccc(N(c3ccc(-c4cccc5cccc(-c6ccccc6)c45)cc3)c3cccc4cc5oc6ccccc6c5cc34)cc2)cc1]

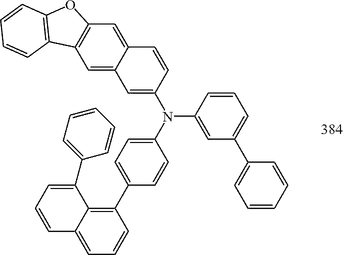 [SMILES: c1ccc(-c2cccc(N(c3ccc(-c4cccc5cccc(-c6ccccc6)c45)cc3)c3ccc4cc5oc6ccccc6c5cc4c3)c2)cc1]

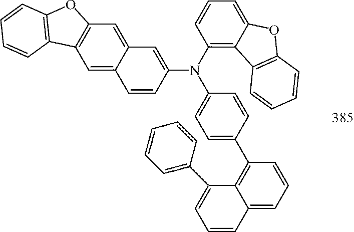 [SMILES: c1ccc(-c2cccc3cccc(-c4ccc(N(c5ccc6cc7c(cc6c5)oc5ccccc57)c5cccc6oc7ccccc7c56)cc4)c23)cc1]

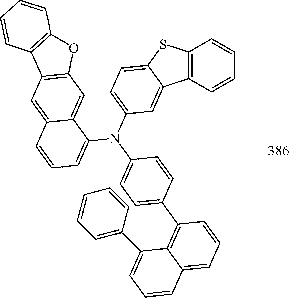 [SMILES: c1ccc(-c2cccc3cccc(-c4ccc(N(c5ccc6sc7ccccc7c6c5)c5cccc6cc7c(cc56)oc5ccccc57)cc4)c23)cc1]

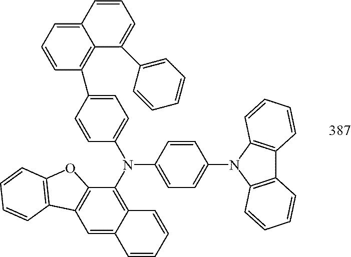 [SMILES: c1ccc(-c2cccc3cccc(-c4ccc(N(c5ccc(-n6c7ccccc7c7ccccc76)cc5)c5c6ccccc6cc6c5oc5ccccc56)cc4)c23)cc1]

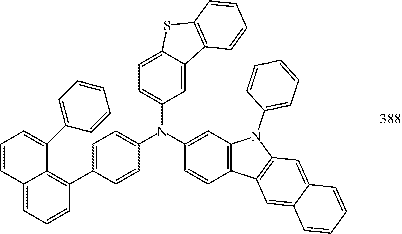 [SMILES: c1ccc(-c2cccc3cccc(-c4ccc(N(c5ccc6sc7ccccc7c6c5)c5ccc6c7cc8ccccc8cc7n(-c7ccccc7)c6c5)cc4)c23)cc1]

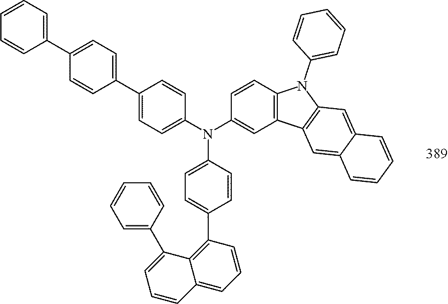 [SMILES: c1ccc(-c2ccc(-c3ccc(N(c4ccc(-c5cccc6cccc(-c7ccccc7)c56)cc4)c4ccc5c(c4)c4cc6ccccc6cc4n5-c4ccccc4)cc3)cc2)cc1]

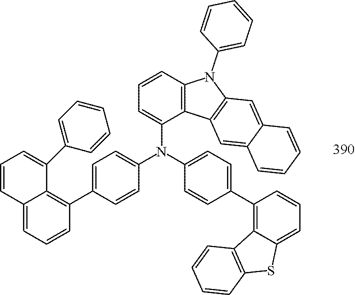 [SMILES: c1ccc(-c2cccc3cccc(-c4ccc(N(c5ccc(-c6cccc7sc8ccccc8c67)cc5)c5cccc6c5c5cc7ccccc7cc5n6-c5ccccc5)cc4)c23)cc1]

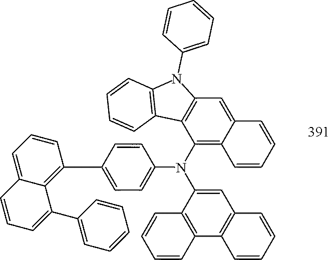 [SMILES: c1ccc(-c2cccc3cccc(-c4ccc(N(c5cc6ccccc6c6ccccc56)c5c6ccccc6cc6c5c5ccccc5n6-c5ccccc5)cc4)c23)cc1]

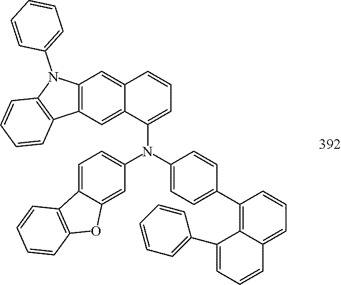 [SMILES: c1ccc(-c2cccc3cccc(-c4ccc(N(c5ccc6c(c5)oc5ccccc56)c5cccc6cc7c(cc56)c5ccccc5n7-c5ccccc5)cc4)c23)cc1]

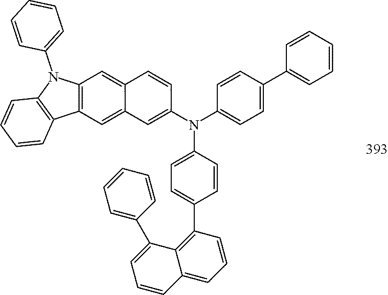 [SMILES: c1ccc(-c2ccc(N(c3ccc(-c4cccc5cccc(-c6ccccc6)c45)cc3)c3ccc4cc5c(cc4c3)c3ccccc3n5-c3ccccc3)cc2)cc1]

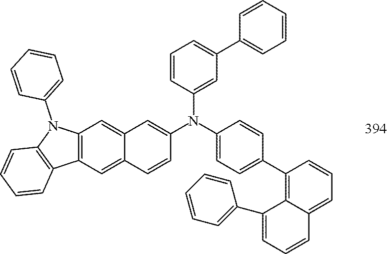 [SMILES: c1ccc(-c2cccc(N(c3ccc(-c4cccc5cccc(-c6ccccc6)c45)cc3)c3ccc4cc5c6ccccc6n(-c6ccccc6)c5cc4c3)c2)cc1]

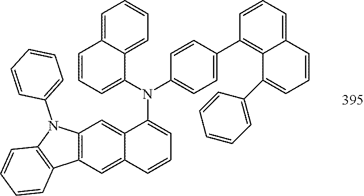 [SMILES: c1ccc(-c2cccc3cccc(-c4ccc(N(c5cccc6ccccc56)c5cccc6cc7c8ccccc8n(-c8ccccc8)c7cc56)cc4)c23)cc1]

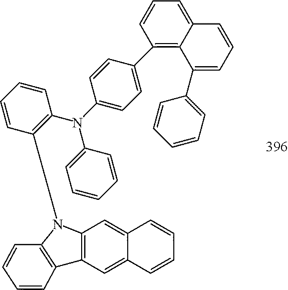 [SMILES: c1ccc(-c2cccc3cccc(-c4ccc(N(c5ccccc5)c5ccccc5-n5c6ccccc6c6cc7ccccc7cc65)cc4)c23)cc1]

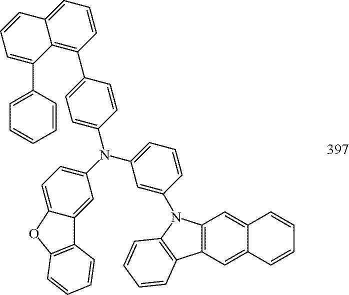 [SMILES: c1ccc(-c2cccc3cccc(-c4ccc(N(c5cccc(-n6c7ccccc7c7cc8ccccc8cc76)c5)c5ccc6oc7ccccc7c6c5)cc4)c23)cc1]

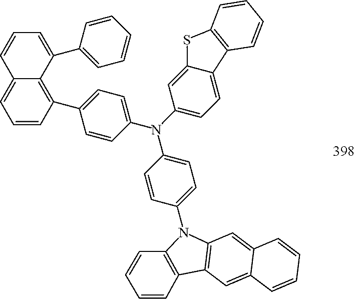 [SMILES: c1ccc(-c2cccc3cccc(-c4ccc(N(c5ccc(-n6c7ccccc7c7cc8ccccc8cc76)cc5)c5ccc6c(c5)sc5ccccc56)cc4)c23)cc1]

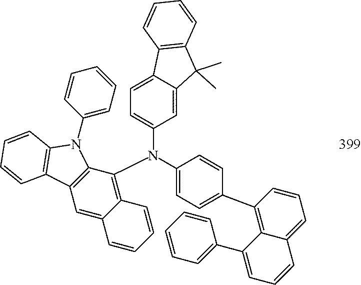 [SMILES: CC1(C)c2ccccc2-c2ccc(N(c3ccc(-c4cccc5cccc(-c6ccccc6)c45)cc3)c3c4ccccc4cc4c5ccccc5n(-c5ccccc5)c34)cc21]

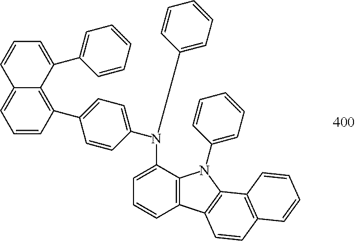 [SMILES: c1ccc(-c2cccc3cccc(-c4ccc(N(c5ccccc5)c5cccc6c7ccc8ccccc8c7n(-c7ccccc7)c56)cc4)c23)cc1]

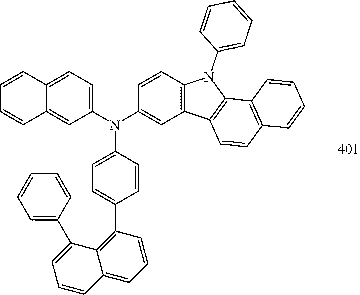 [SMILES: c1ccc(-c2cccc3cccc(-c4ccc(N(c5ccc6ccccc6c5)c5ccc6c(c5)c5ccc7ccccc7c5n6-c5ccccc5)cc4)c23)cc1]

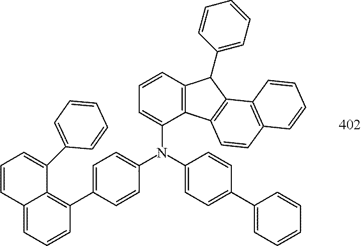 [SMILES: c1ccc(-c2ccc(N(c3ccc(-c4cccc5cccc(-c6ccccc6)c45)cc3)c3cccc4c3-c3ccc5ccccc5c3C4c3ccccc3)cc2)cc1]

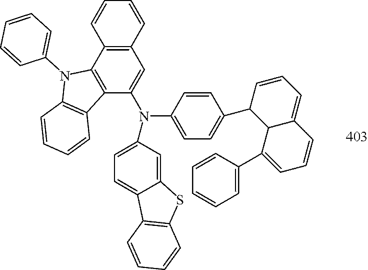 [SMILES: C1=CC2=CC=CC(c3ccc(N(c4ccc5c(c4)sc4ccccc45)c4cc5ccccc5c5c4c4ccccc4n5-c4ccccc4)cc3)C2C(c2ccccc2)=C1]

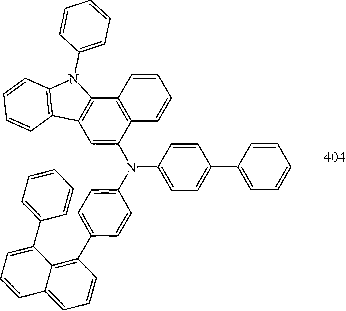 [SMILES: c1ccc(-c2ccc(N(c3ccc(-c4cccc5cccc(-c6ccccc6)c45)cc3)c3cc4c5ccccc5n(-c5ccccc5)c4c4ccccc34)cc2)cc1]

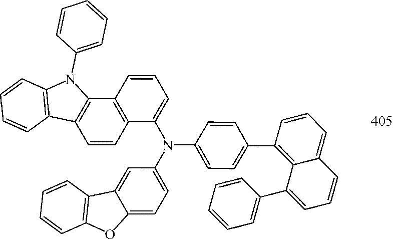 [SMILES: c1ccc(-c2cccc3cccc(-c4ccc(N(c5ccc6oc7ccccc7c6c5)c5cccc6c5ccc5c7ccccc7n(-c7ccccc7)c65)cc4)c23)cc1]

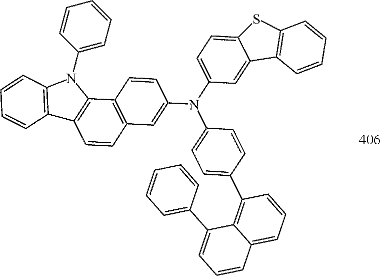 [SMILES: c1ccc(-c2cccc3cccc(-c4ccc(N(c5ccc6c(ccc7c8ccccc8n(-c8ccccc8)c67)c5)c5ccc6sc7ccccc7c6c5)cc4)c23)cc1]

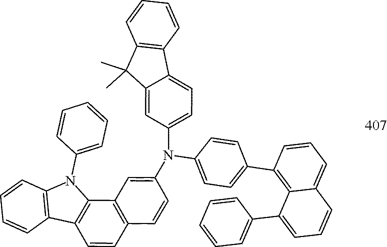 [SMILES: CC1(C)c2ccccc2-c2ccc(N(c3ccc(-c4cccc5cccc(-c6ccccc6)c45)cc3)c3ccc4ccc5c6ccccc6n(-c6ccccc6)c5c4c3)cc21]

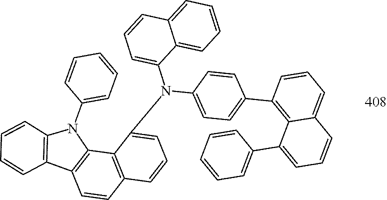 [SMILES: c1ccc(-c2cccc3cccc(-c4ccc(N(c5cccc6ccccc56)c5cccc6ccc7c8ccccc8n(-c8ccccc8)c7c56)cc4)c23)cc1]

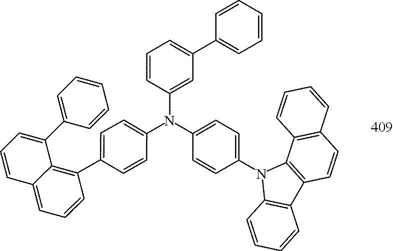 [SMILES: c1ccc(-c2cccc(N(c3ccc(-c4cccc5cccc(-c6ccccc6)c45)cc3)c3ccc(-n4c5ccccc5c5ccc6ccccc6c54)cc3)c2)cc1]

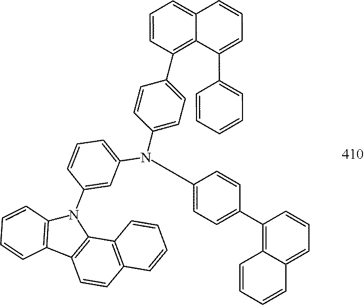 [SMILES: c1ccc(-c2cccc3cccc(-c4ccc(N(c5ccc(-c6cccc7ccccc67)cc5)c5cccc(-n6c7ccccc7c7ccc8ccccc8c76)c5)cc4)c23)cc1]

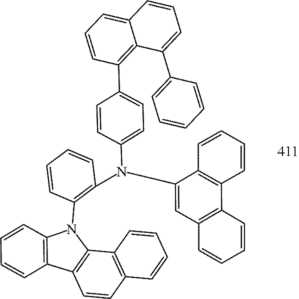 [SMILES: c1ccc(-c2cccc3cccc(-c4ccc(N(c5ccccc5-n5c6ccccc6c6ccc7ccccc7c65)c5cc6ccccc6c6ccccc56)cc4)c23)cc1]

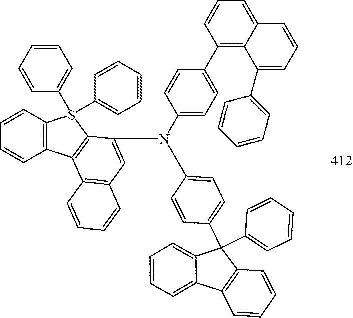 [SMILES: c1ccc(-c2cccc3cccc(-c4ccc(N(c5ccc(C6(c7ccccc7)c7ccccc7-c7ccccc76)cc5)c5cc6ccccc6c6c5S(c5ccccc5)(c5ccccc5)c5ccccc5-6)cc4)c23)cc1]

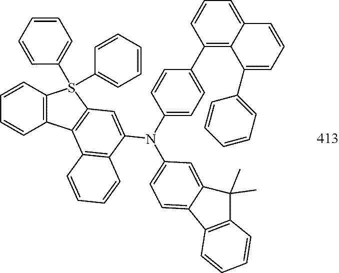 [SMILES: CC1(C)c2ccccc2-c2ccc(N(c3ccc(-c4cccc5cccc(-c6ccccc6)c45)cc3)c3cc4c(c5ccccc35)-c3ccccc3S4(c3ccccc3)c3ccccc3)cc21]

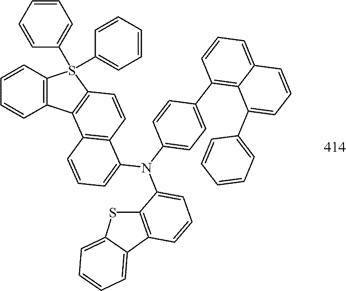 [SMILES: c1ccc(-c2cccc3cccc(-c4ccc(N(c5cccc6c7c(ccc56)S(c5ccccc5)(c5ccccc5)c5ccccc5-7)c5cccc6c5sc5ccccc56)cc4)c23)cc1]

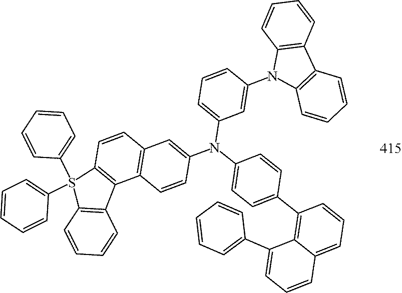 [SMILES: c1ccc(-c2cccc3cccc(-c4ccc(N(c5cccc(-n6c7ccccc7c7ccccc76)c5)c5ccc6c7c(ccc6c5)S(c5ccccc5)(c5ccccc5)c5ccccc5-7)cc4)c23)cc1]

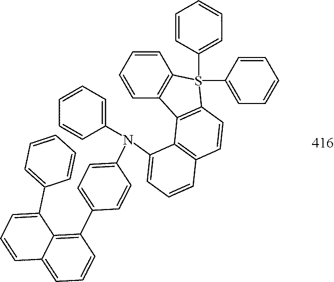 [SMILES: c1ccc(-c2cccc3cccc(-c4ccc(N(c5ccccc5)c5cccc6ccc7c(c56)-c5ccccc5S7(c5ccccc5)c5ccccc5)cc4)c23)cc1]

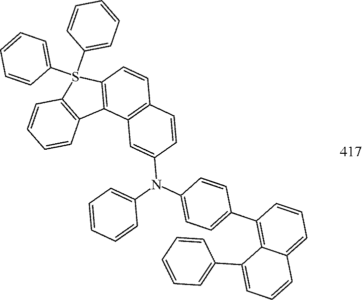 [SMILES: c1ccc(-c2cccc3cccc(-c4ccc(N(c5ccccc5)c5ccc6ccc7c(c6c5)-c5ccccc5S7(c5ccccc5)c5ccccc5)cc4)c23)cc1]

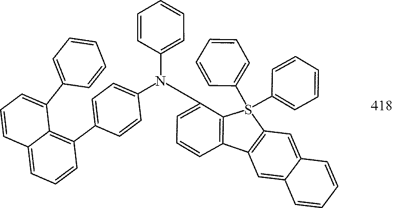 [SMILES: c1ccc(-c2cccc3cccc(-c4ccc(N(c5ccccc5)c5cccc6c5S(c5ccccc5)(c5ccccc5)c5cc7ccccc7cc5-6)cc4)c23)cc1]

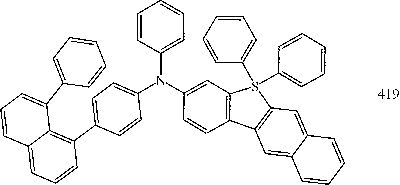 [SMILES: c1ccc(-c2cccc3cccc(-c4ccc(N(c5ccccc5)c5ccc6c(c5)S(c5ccccc5)(c5ccccc5)c5cc7ccccc7cc5-6)cc4)c23)cc1]

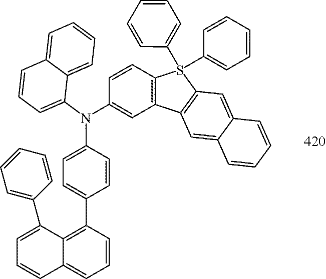 [SMILES: c1ccc(-c2cccc3cccc(-c4ccc(N(c5ccc6c(c5)-c5cc7ccccc7cc5S6(c5ccccc5)c5ccccc5)c5cccc6ccccc56)cc4)c23)cc1]

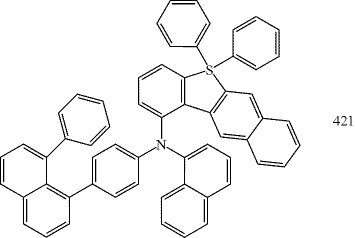 [SMILES: c1ccc(-c2cccc3cccc(-c4ccc(N(c5cccc6c5-c5cc7ccccc7cc5S6(c5ccccc5)c5ccccc5)c5cccc6ccccc56)cc4)c23)cc1]

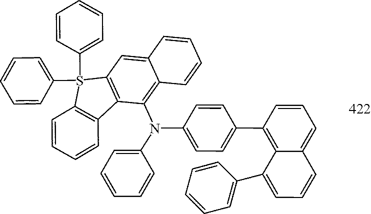 [SMILES: c1ccc(-c2cccc3cccc(-c4ccc(N(c5ccccc5)c5c6c(cc7ccccc57)S(c5ccccc5)(c5ccccc5)c5ccccc5-6)cc4)c23)cc1]

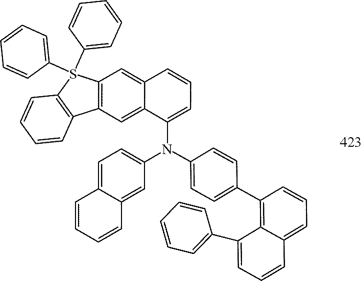 [SMILES: c1ccc(-c2cccc3cccc(-c4ccc(N(c5ccc6ccccc6c5)c5cccc6cc7c(cc56)-c5ccccc5S7(c5ccccc5)c5ccccc5)cc4)c23)cc1]

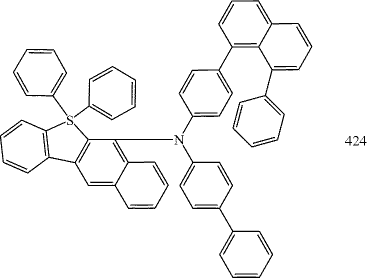 [SMILES: c1ccc(-c2ccc(N(c3ccc(-c4cccc5cccc(-c6ccccc6)c45)cc3)c3c4c(cc5ccccc35)-c3ccccc3S4(c3ccccc3)c3ccccc3)cc2)cc1]

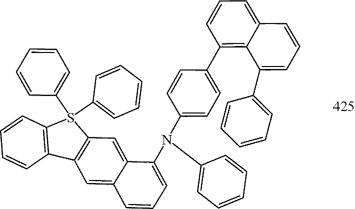 [SMILES: c1ccc(-c2cccc3cccc(-c4ccc(N(c5ccccc5)c5cccc6cc7c(cc56)S(c5ccccc5)(c5ccccc5)c5ccccc5-7)cc4)c23)cc1]

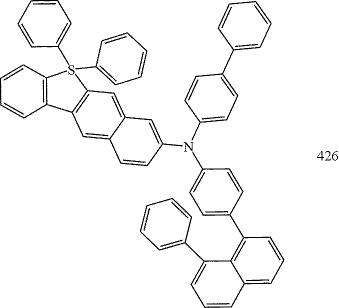 [SMILES: c1ccc(-c2ccc(N(c3ccc(-c4cccc5cccc(-c6ccccc6)c45)cc3)c3ccc4cc5c(cc4c3)S(c3ccccc3)(c3ccccc3)c3ccccc3-5)cc2)cc1]

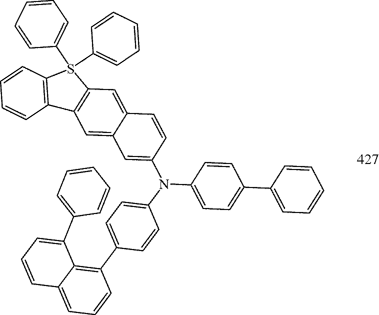 [SMILES: c1ccc(-c2ccc(N(c3ccc(-c4cccc5cccc(-c6ccccc6)c45)cc3)c3ccc4cc5c(cc4c3)-c3ccccc3S5(c3ccccc3)c3ccccc3)cc2)cc1]

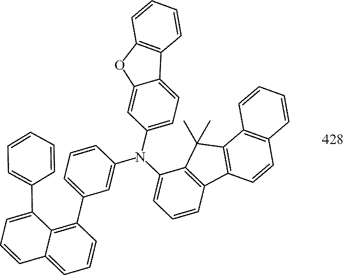 [SMILES: CC1(C)c2c(cccc2N(c2cccc(-c3cccc4cccc(-c5ccccc5)c34)c2)c2ccc3c(c2)oc2ccccc23)-c2ccc3ccccc3c21]

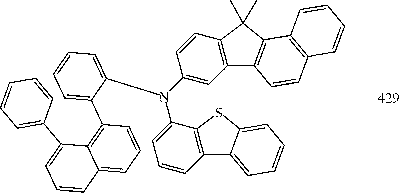 [SMILES: CC1(C)c2ccc(N(c3ccccc3-c3cccc4cccc(-c5ccccc5)c34)c3cccc4c3sc3ccccc34)cc2-c2ccc3ccccc3c21]

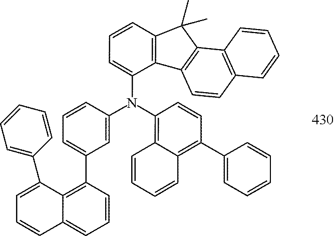 [SMILES: CC1(C)c2cccc(N(c3cccc(-c4cccc5cccc(-c6ccccc6)c45)c3)c3ccc(-c4ccccc4)c4ccccc34)c2-c2ccc3ccccc3c21]

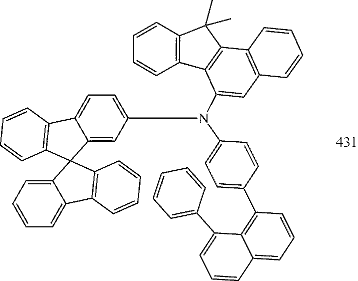 [SMILES: CC1(C)c2ccccc2-c2c(N(c3ccc(-c4cccc5cccc(-c6ccccc6)c45)cc3)c3ccc4c(c3)C3(c5ccccc5-c5ccccc53)c3ccccc3-4)cc3ccccc3c21]

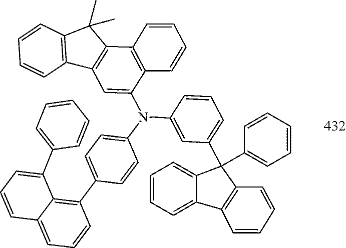 [SMILES: CC1(C)c2ccccc2-c2cc(N(c3ccc(-c4cccc5cccc(-c6ccccc6)c45)cc3)c3cccc(C4(c5ccccc5)c5ccccc5-c5ccccc54)c3)c3ccccc3c21]

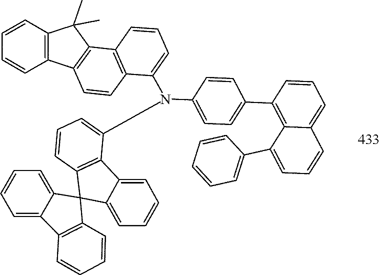 [SMILES: CC1(C)c2ccccc2-c2ccc3c(N(c4ccc(-c5cccc6cccc(-c7ccccc7)c56)cc4)c4cccc5c4-c4ccccc4C54c5ccccc5-c5ccccc54)cccc3c21]

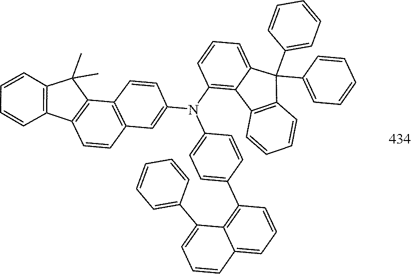 [SMILES: CC1(C)c2ccccc2-c2ccc3cc(N(c4ccc(-c5cccc6cccc(-c7ccccc7)c56)cc4)c4cccc5c4-c4ccccc4C5(c4ccccc4)c4ccccc4)ccc3c21]

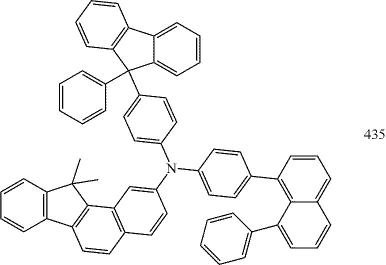 [SMILES: CC1(C)c2ccccc2-c2ccc3ccc(N(c4ccc(-c5cccc6cccc(-c7ccccc7)c56)cc4)c4ccc(C5(c6ccccc6)c6ccccc6-c6ccccc65)cc4)cc3c21]

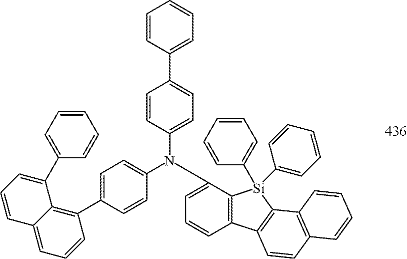 [SMILES: c1ccc(-c2ccc(N(c3ccc(-c4cccc5cccc(-c6ccccc6)c45)cc3)c3cccc4c3[Si](c3ccccc3)(c3ccccc3)c3c-4ccc4ccccc34)cc2)cc1]

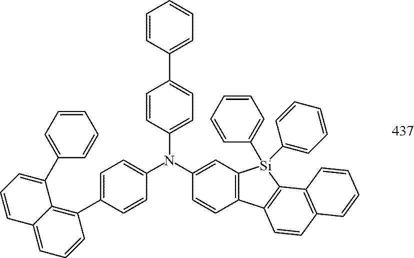 [SMILES: c1ccc(-c2ccc(N(c3ccc(-c4cccc5cccc(-c6ccccc6)c45)cc3)c3ccc4c(c3)[Si](c3ccccc3)(c3ccccc3)c3c-4ccc4ccccc34)cc2)cc1]

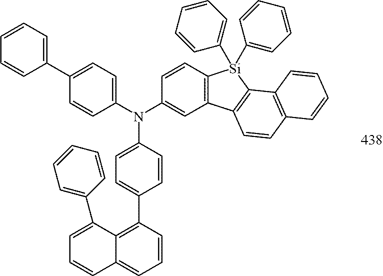 [SMILES: c1ccc(-c2ccc(N(c3ccc(-c4cccc5cccc(-c6ccccc6)c45)cc3)c3ccc4c(c3)-c3ccc5ccccc5c3[Si]4(c3ccccc3)c3ccccc3)cc2)cc1]

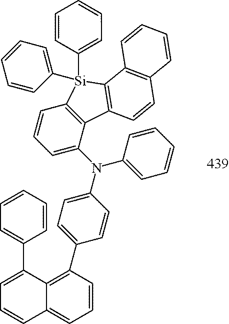 [SMILES: c1ccc(-c2cccc3cccc(-c4ccc(N(c5ccccc5)c5cccc6c5-c5ccc7ccccc7c5[Si]6(c5ccccc5)c5ccccc5)cc4)c23)cc1]

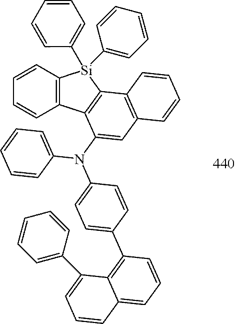 [SMILES: c1ccc(-c2cccc3cccc(-c4ccc(N(c5ccccc5)c5cc6ccccc6c6c5-c5ccccc5[Si]6(c5ccccc5)c5ccccc5)cc4)c23)cc1]

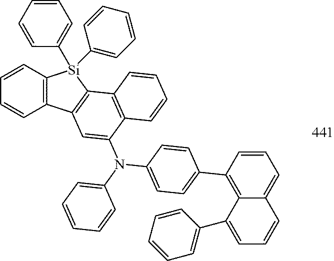 [SMILES: c1ccc(-c2cccc3cccc(-c4ccc(N(c5ccccc5)c5cc6c(c7ccccc57)[Si](c5ccccc5)(c5ccccc5)c5ccccc5-6)cc4)c23)cc1]

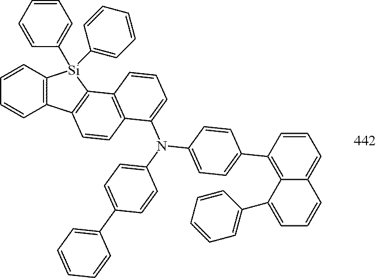 [SMILES: c1ccc(-c2ccc(N(c3ccc(-c4cccc5cccc(-c6ccccc6)c45)cc3)c3cccc4c5c(ccc34)-c3ccccc3[Si]5(c3ccccc3)c3ccccc3)cc2)cc1]

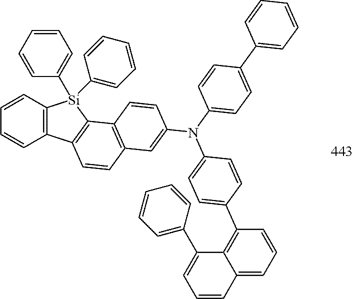 [SMILES: c1ccc(-c2ccc(N(c3ccc(-c4cccc5cccc(-c6ccccc6)c45)cc3)c3ccc4c5c(ccc4c3)-c3ccccc3[Si]5(c3ccccc3)c3ccccc3)cc2)cc1]

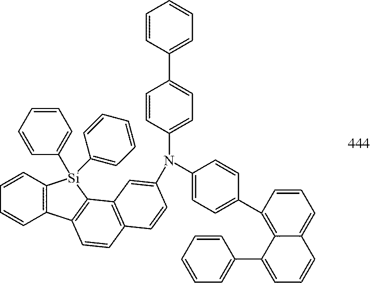 [SMILES: c1ccc(-c2ccc(N(c3ccc(-c4cccc5cccc(-c6ccccc6)c45)cc3)c3ccc4ccc5c(c4c3)[Si](c3ccccc3)(c3ccccc3)c3ccccc3-5)cc2)cc1]

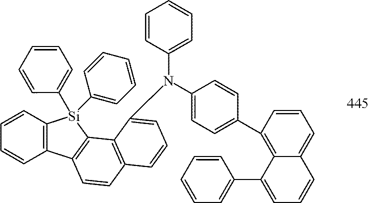 [SMILES: c1ccc(-c2cccc3cccc(-c4ccc(N(c5ccccc5)c5cccc6ccc7c(c56)[Si](c5ccccc5)(c5ccccc5)c5ccccc5-7)cc4)c23)cc1]

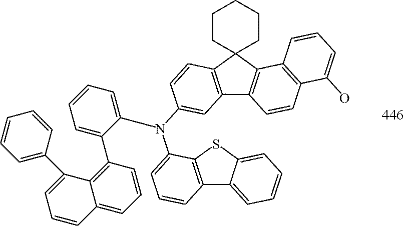 [SMILES: Oc1cccc2c3c(ccc12)-c1cc(N(c2ccccc2-c2cccc4cccc(-c5ccccc5)c24)c2cccc4c2sc2ccccc24)ccc1C31CCCCC1]

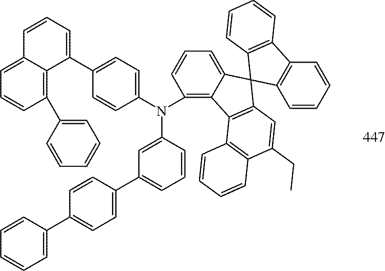 [SMILES: CCc1cc2c(c3ccccc13)-c1c(N(c3ccc(-c4cccc5cccc(-c6ccccc6)c45)cc3)c3cccc(-c4ccc(-c5ccccc5)cc4)c3)cccc1C21c2ccccc2-c2ccccc21]

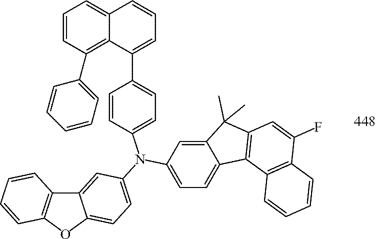 [SMILES: CC1(C)c2cc(N(c3ccc(-c4cccc5cccc(-c6ccccc6)c45)cc3)c3ccc4oc5ccccc5c4c3)ccc2-c2c1cc(F)c1ccccc21]

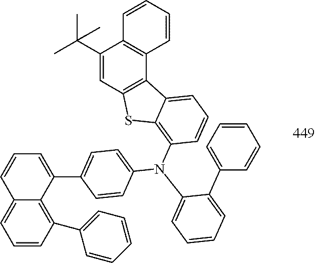 [SMILES: CC(C)(C)c1cc2sc3c(N(c4ccc(-c5cccc6cccc(-c7ccccc7)c56)cc4)c4ccccc4-c4ccccc4)cccc3c2c2ccccc12]

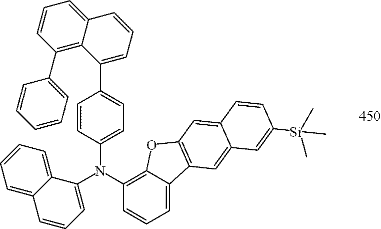 [SMILES: C[Si](C)(C)c1ccc2cc3oc4c(N(c5ccc(-c6cccc7cccc(-c8ccccc8)c67)cc5)c5cccc6ccccc56)cccc4c3cc2c1]

In a second aspect, the present application provides an electronic component, including: an anode and a cathode that are arranged oppositely, and a functional layer arranged between the anode and the cathode, where the functional layer includes the organic compound of the present application.

The functional layer includes an EBL, and the EBL includes the organic compound.

Optionally, the electronic component is an OLED or a photoelectric conversion device.

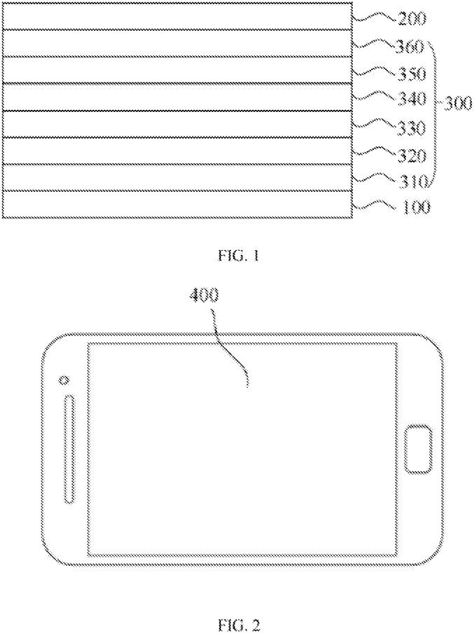

In an embodiment of the present application, the electronic component is an OLED. As shown in FIG. 1, the OLED of the present application includes an anode 100, a cathode 200, and at least one functional layer 300 between the anode and the cathode; the functional layer 300 includes an HTL 320, an EBL 330, an organic electroluminescent layer 340, and an ETL 350; the HTL 320, the EBL 330, the organic electroluminescent layer 340, the ETL 350, and the EIL 360 are sequentially formed on the anode 100; and the EBL 330 includes the organic compound described in the first aspect of the present application, and preferably includes at least one of Compounds 1 to 450.

Optionally, the anode 100 is preferably made of a material with a large work function that facilitates the injection of holes into the functional layer. Specific examples of the anode material include, but are not limited to: metals such as nickel, platinum, vanadium, chromium, copper, zinc, and gold or alloys thereof; metal oxides such as zinc oxide, indium oxide, indium tin oxide (ITO), and indium zinc oxide (IZO); a recombination of a metal and an oxide such as ZnO : Al or SnO₂ : Sb; or conductive polymers such as poly(3-methylthiophene), poly[3,4-(ethylene-1,2-dioxy)thiophene] (PEDT), polypyrrole (PPy), and polyaniline (PANI). Preferably, a transparent electrode containing ITO is adopted as the anode.

Optionally, the HTL 320 includes one or more hole transport materials, and the hole transport materials are selected from carbazole polymers, carbazole-linked triarylamine compounds, or other compounds, which is not particularly limited in the present application. In an embodiment of the present application, the HTL 320 includes N.N′-di- 1 -naphthalenyl-N,N′-diphenyl-[1, 1 -biphenyl]-4,4′-diamine (NPB).

Optionally, the EBL 330 is arranged to block electrons transmitted from the organic light-emitting layer 340, thereby ensuring that an electron and a hole can be efficiently recombined in the organic light-emitting layer 340; the EBL 330 can also block excitons diffused from the organic light-emitting layer 340 to reduce the triplet-state quenching of the excitons, thereby ensuring the light-emitting efficiency of the OLED; and a compound of the EBL 340 has a relatively-high LUMO value, which can effectively block the transmission and diffusion of electrons and excitons from the organic light-emitting layer 340 to an anode 110. Preferably, the EBL 340 includes the organic compound of the present application.

A material of the organic electroluminescent layer 340 may be a metal chelate compound, a bisstyryl derivative, an aromatic amine derivative, a dibenzofuran derivative, or the like, which is not particularly limited in the present application. In an embodiment of the present application, the organic electroluminescent layer 340 consists of BH-01 and BD-01.

The ETL 350 has a single-layer structure or a multi-layer structure, which may include one or more electron transport materials. The electron transport materials are selected from benzimidazole derivatives, oxadiazole derivatives, quinoxaline derivatives, or other electron transport materials, which are not particularly limited in the present application. For example, the ETL 350 consists of ET-06 and LiQ.

Optionally, the cathode 200 is made of a material with a small work function that facilitates the injection of electrons into the functional layer. Specific examples of the cathode material include, but are not limited to: metals such as magnesium, calcium, sodium, potassium, titanium, indium, yttrium, lithium, gadolinium, aluminum, silver, tin, and lead or alloys thereof; or multi-layer materials such as LiF/Al, Liq/Al, LiO_{2/}Al, LiF/Ca, LiF/Al, and BaF_{2/}Ca. Preferably, a metal electrode including silver and magnesium is adopted as the cathode.

Optionally, an HlL. 310 is further arranged between the anode 100 and the HTL 320 to enhance the ability to inject holes into the HTL 320. The HIL 310 is made of a benzidine derivative, a starburst arylamine compound, a phthalocyanine derivative, or another material, which is not particularly limited in the present application. For example, the HlL 310 includes F4~TCNQ.

Optionally, an ElL 360 is further arranged between the cathode 200 and the ETL 350 to enhance the ability to inject electrons into the ETL 350. The ElL 360 includes an inorganic material such as an alkali metal sulfide and an alkali metal halide, or includes a complex of an alkali metal and an organic substance. In an embodiment of the present application, the ElL 360 includes Yb.

In a third aspect, the present application provides an electronic device, which includes the electronic component provided in the second aspect of the present application.

As shown in FIG. 2, the present application provides an electronic device 400, and the electronic device 400 includes the OLED. The electronic device 400 may be a display device, a lighting device, an optical communication device, or another electronic device, including but not limited to computer screen, mobile phone screen, television set, electronic paper, emergency light, and optical module.

The present application will be described in detail below with reference to synthesis examples, but the following description is provided to explain the present application rather than limit the scope of the present application in any way.

### Synthesis Examples

Those skilled in the art will recognize that the chemical reactions described in the present application can be used to appropriately prepare many other compounds of the present application, and other methods for preparing the compounds of the present application are considered to be within the scope of the present application. For example, the synthesis of non-illustrative compounds according to the present application can be successfully completed by those skilled in the art through modified methods, such as appropriately protecting interfering groups, using other known reagents in addition to those described in the present application, or conventionally modifying reaction conditions. In addition, reactions disclosed by the present application or known reaction conditions are also recognized to be applicable to the preparation of other compounds of the present application.

### Synthesis of a Compound 2

### 1. Synthesis of lM A-1

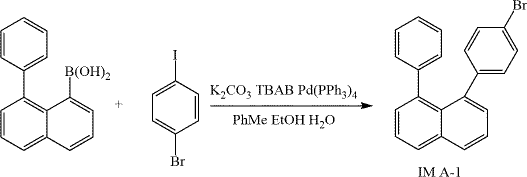

Nitrogen was introduced into a three-necked flask equipped with a mechanical stirrer, a thermometer, and a spherical condenser at 0.100 L/min to allow nitrogen replacement for 15 min, then 8-phenyl-l-naphthaleneboronic acid (100 g, 0.403 mol), p-bromoiodobenzene (103.7 g, 0.366 mol), K₂CO₃ (101.3 g. 0.733 mol), tetrabutylammonium bromide (TBAB) (2.3 g, 0.007 mol), Pd(PPh₃)₄ (4.2 g, 0.004 mol), toluene (600 mL), ethanol (200 mL), and water (100 mL) were added, and a resulting mixture was stirred and heated to allow a reaction under reflux for 12 h; after the reaction was completed, a resulting reaction system was cooled to room temperature, toluene was added for extraction, and a resulting organic phase was separated, dried with anhydrous magnesium sulfate, and filtered, and a filtrate was subjected to vacuum distillation for concentration to obtain a crude product, and the crude product was purified through silica gel chromatography with a dichloromethane (DCM)/n-heptane system to obtain a white solid IM A-l (117.9 g, yield: 90%).

IM A-X listed in Table 1 was synthesized with reference to the synthesis method of the lM A-l, except that a raw material 1 was used instead of the p-bromoiodobenzene. The main raw materials used, the synthesized intermediates and yields thereof were shown in Table 1.

TABLE 1

| Raw material 1 | IM A-X | Yield/% |
|---|---|---|
| 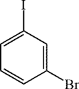 | 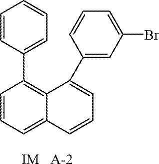 | 89 |
| 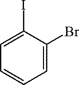 | 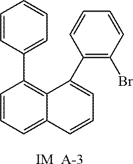 | 86 |

### 2. Synthesis of lM B-1

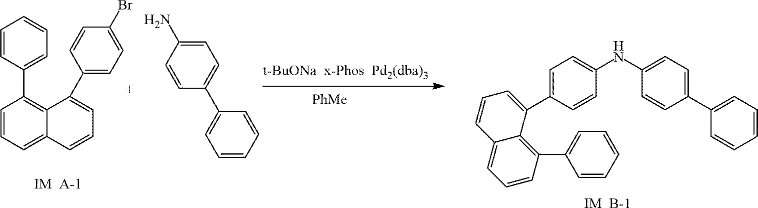

Nitrogen was introduced into a three-necked flask equipped with a mechanical stirrer, a thermometer, and a spherical condenser at 0. 100 L/min to allow nitrogen replacement for 15 min, then the IM A-l (20 g, 0.056 mol), 4-aminobiphenyl (9.5 g, 0.056 mol), and toluene (160 mL) were added, and a resulting mixture was stirred under reflux for 30 min and then cooled to 70° C. to 80° C.; then sodium terl-butoxide (10.76 g, 0.112 mol), 2-dieyclohexylphosphino-2.4,6-triisopropylbiphenyl (X-phos) (0.53 g, 0.001 mol), and tris(dibenzylideneacetone)dipalladium (0.4576 g, 0.0005 mol) were added; after a temperature of a resulting system was stable, a reaction was conducted under reflux for 4 h and then stopped; a resulting reaction solution was cooled to room temperature, toluene was added for extraction, and a resulting organic phase was separated, washed with water until neutral, dried with anhydrous magnesium sulfate, and filtered; and a filtrate was subjected to vacuum distillation for concentration to obtain a crude product, and the crude product was purified through silica gel chromatography with a DCM/n-heptane system to obtain a white solid lM B-1 (21.3 g. yield: 85%).

IM B-X listed in Table 2 was synthesized with reference to the synthesis method of the lM B-1, except that IM A-X was used instead of the IM A-l and a raw material 2 was used instead of the 4-aminobiphenyl. The main raw materials used, the synthesized intermediates and yields thereof were shown in Table 2.

TABLE 2

| IM A-X | Raw material 2 | IM B-X | Yield /% |
|---|---|---|---|
| 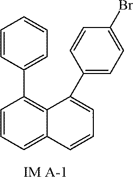 | 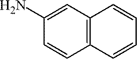 [SMILES: Nc1ccc2ccccc2c1] | 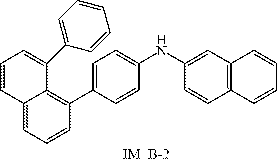 | 81 |
| | 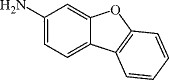 [SMILES: Nc1ccc2c(c1)oc1ccccc12] | 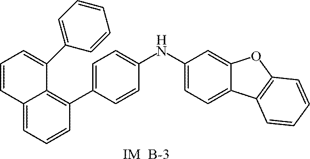 | 82 |
| | 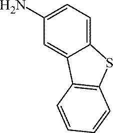 [SMILES: Nc1ccc2sc3ccccc3c2c1] | 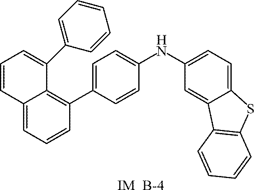 | 79 |
| | 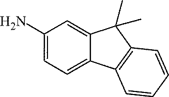 [SMILES: CC1(C)c2ccccc2-c2ccc(N)cc21] | 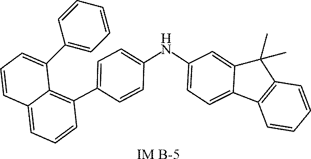 | 82 |
| | 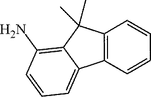 [SMILES: CC1(C)c2ccccc2-c2cccc(N)c21] | I 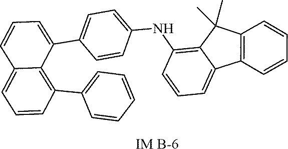 I | 75 |
| | 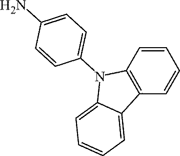 [SMILES: Nc1ccc(-n2c3ccccc3c3ccccc32)cc1] | 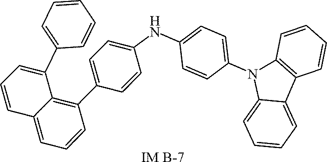 | 81 |
| | 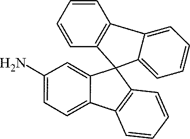 [SMILES: Nc1ccc2c(c1)C1(c3ccccc3-c3ccccc31)c1ccccc1-2] | 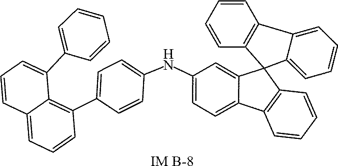 | 82 |
| | 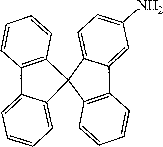 [SMILES: Nc1ccc2c(c1)-c1ccccc1C21c2ccccc2-c2ccccc21] | 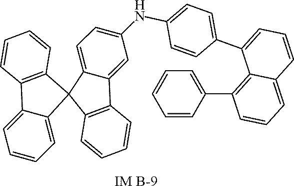 | 72 |
| | 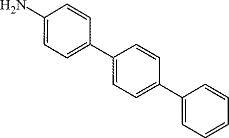 [SMILES: Nc1ccc(-c2ccc(-c3ccccc3)cc2)cc1] | 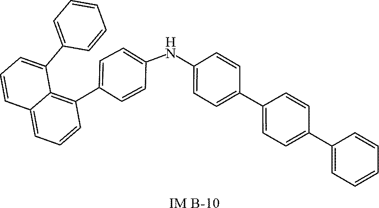 | 84 |
| | 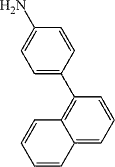 [SMILES: Nc1ccc(-c2cccc3ccccc23)cc1] | 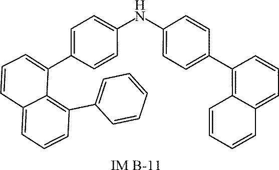 | 79 |
| | 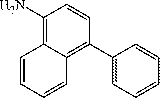 [SMILES: Nc1ccc(-c2ccccc2)c2ccccc12] | 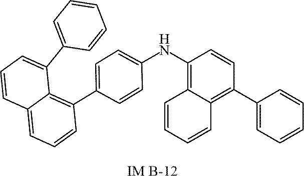 | 76 |
| | 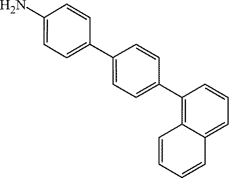 [SMILES: Nc1ccc(-c2ccc(-c3cccc4ccccc34)cc2)cc1] | 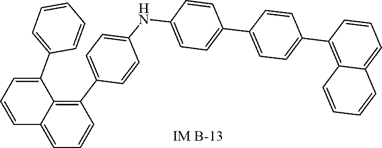 | 81 |
| | 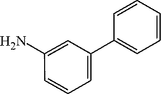 [SMILES: Nc1cccc(-c2ccccc2)c1] | 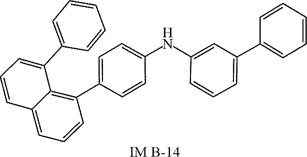 | 81 |
| | 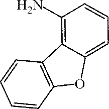 [SMILES: Nc1cccc2oc3ccccc3c12] | 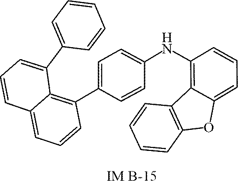 | 73 |
| | 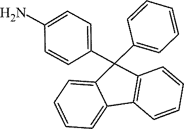 [SMILES: Nc1ccc(C2(c3ccccc3)c3ccccc3-c3ccccc32)cc1] | 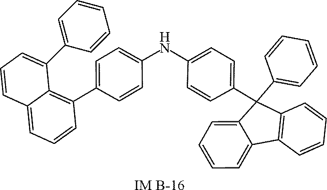 | 75 |
| | 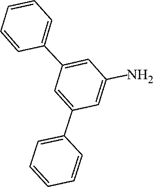 [SMILES: Nc1cc(-c2ccccc2)cc(-c2ccccc2)c1] | 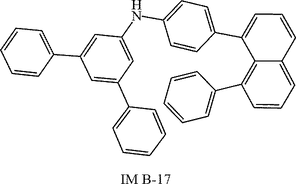 | 74 |
| | 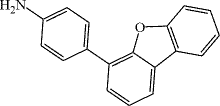 [SMILES: Nc1ccc(-c2cccc3c2oc2ccccc23)cc1] | 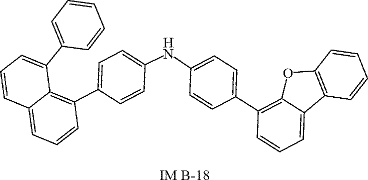 | 79 |
| | 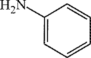 | 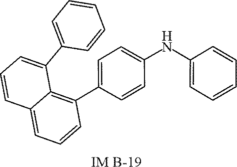 | 85 |
| | 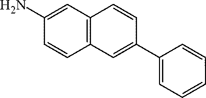 [SMILES: Nc1ccc2cc(-c3ccccc3)ccc2c1] | 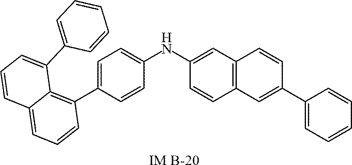 | 84 |
| | 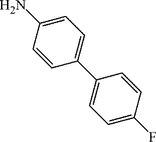 [SMILES: Nc1ccc(-c2ccc(F)cc2)cc1] | 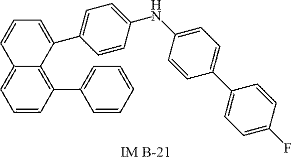 | 82 |
| | 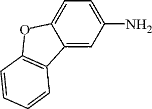 [SMILES: Nc1ccc2oc3ccccc3c2c1] | 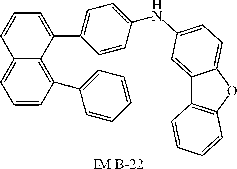 | 85 |
| 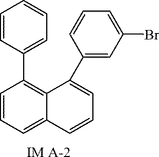 | 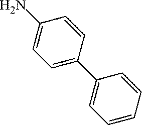 [SMILES: Nc1ccc(-c2ccccc2)cc1] | 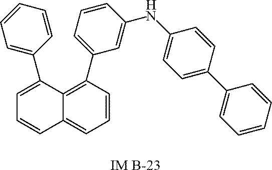 | 76 |
| | 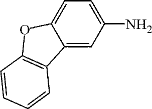 [SMILES: Nc1ccc2oc3ccccc3c2c1] | 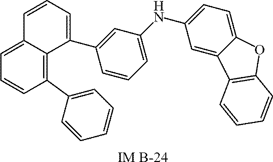 | 78 |
| | 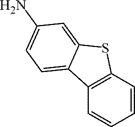 [SMILES: Nc1ccc2c(c1)sc1ccccc12] | 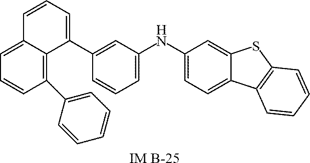 | 76 |
| | 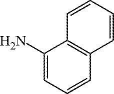 [SMILES: Nc1cccc2ccccc12] | 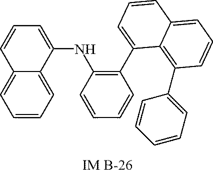 | 69 |
| 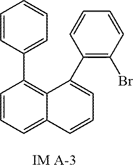 | 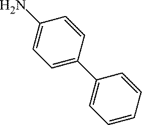 [SMILES: Nc1ccc(-c2ccccc2)cc1] | 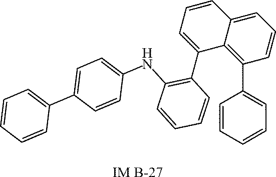 | 81 |
| | 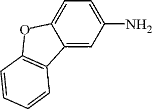 [SMILES: Nc1ccc2oc3ccccc3c2c1] | 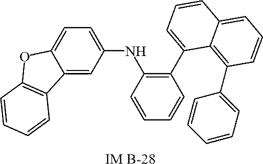 | 69 |

### 3. Synthesis of a Compound 2

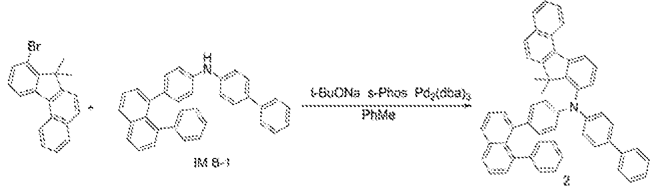 [SMILES: CC1(C)c2cc3ccccc3cc2-c2cccc(Br)c21] Nitrogen was introduced into a three-necked flask equipped with a mechanical stirrer, a thermometer, and a spherical condenser at 0. 100 L/min to allow nitrogen replacement for 15 min, then the IM B-1 (13.6 g, 30.4 mmol), 8-bromo-7,7-dimethyl-7H-benzofluorene (9.8 g, 30.4 mmol), and toluene (100 mL) were added, a resulting mixture was stirred under reflux until a clear solution was obtained, and then the solution was cooled to 70° C. to 80° C.; then sodium tert-butoxide (4.4 g, 45.7 mmol), 2-dicyclohexylphosphino-2’,6′-dimelhoxybiphenyl (s-Phos) (0.25 g, 0.61 mmol), and tris(dibenzylideneacelone)dipalladium (0.28 g, 0.330 mmol) were added; after a temperature of a resulting system was stable, a reaction was conducted under reflux for 6 h and then stopped; a resulting reaction solution was cooled to room temperature, toluene was added for extraction, and a resulting organic phase was separated, washed with water until neutral, dried with anhydrous magnesium sulfate, and filtered; and a filtrate was subjected to vacuum distillation for concentration to obtain a crude product, and the crude product was purified through silica gel chromatography with an ethyl acetate/n-heptane system, and subjected to concentration and then to recrystallization with a toluene/n-heptane system to obtain a white solid compound 2 (17 g, yield: 81.3%, MS: (m/z) = 670.31 [M+H]⁺).

The compounds listed in Table 3 were each synthesized with reference to the synthesis method of the compound 2, except that a raw material 3 was used instead of the S-bromo~7,7-dimethyl~7H~benzofluorene and lM B-X was used instead of B-1. The main raw materials used, the synthesized compounds, yield and MS data thereof were shown in Table 3.

TABLE 3

| Raw material 3 | IM B-X | Compound | Yield /% | MS (m/z) /[M+H]⁺ |
|---|---|---|---|---|
| 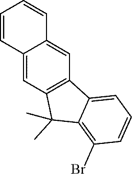 | 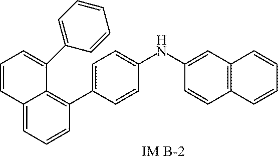 | 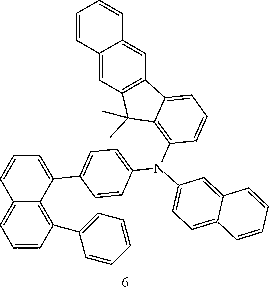 | 82 | 664.29 |
| | 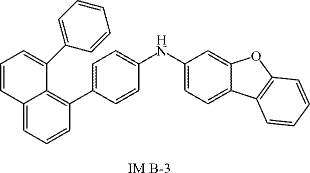 | 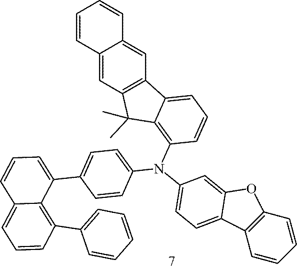 [SMILES: CC1(C)c2cc(Br)ccc2-c2ccc3ccccc3c21] | 81 | 704.29 |
| 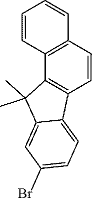 | 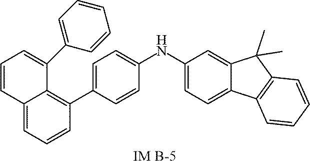 | 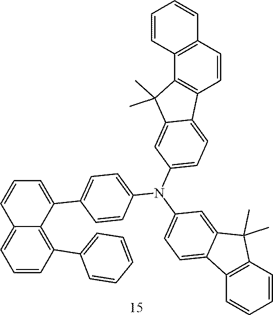 [SMILES: CC1(C)c2cc(Br)ccc2-c2c1ccc1ccccc21] | 76 | 730.34 |
| 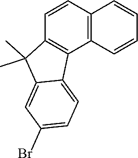 | 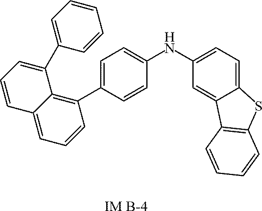 | 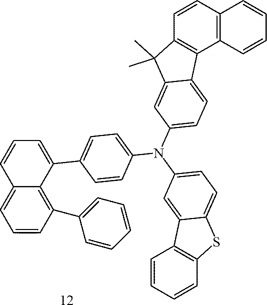 | 75 | 720.29 |
| | 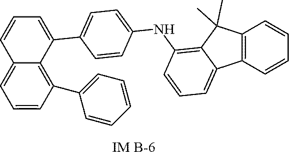 | 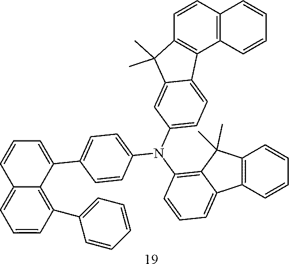 [SMILES: CC1(C)c2ccc(Br)cc2-c2c1ccc1ccccc21] | 71 | 730.34 |
| 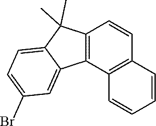 | 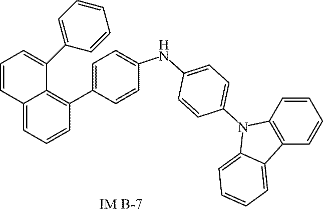 | 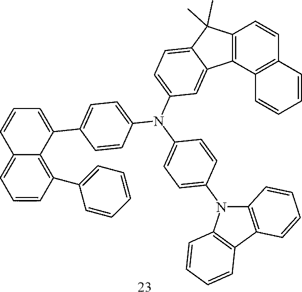 | 75 | 779.33 |
| | 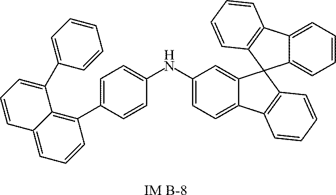 | 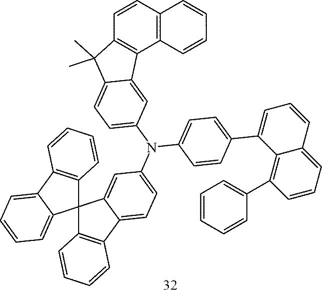 [SMILES: CC1(C)c2cccc(Br)c2-c2c1ccc1ccccc21] | 73 | 852.36 |
| 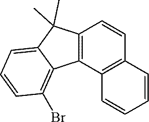 | 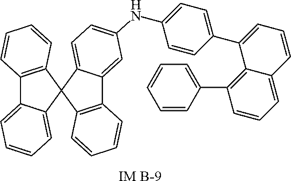 | 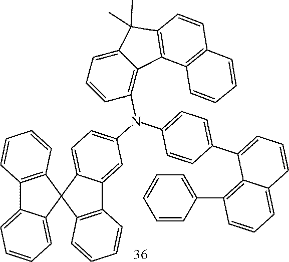 | 69 | 852.36 |
| | 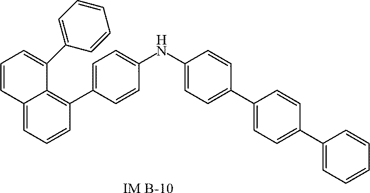 | 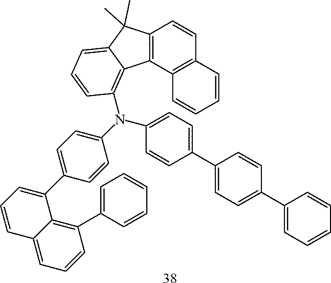 [SMILES: CC1(C)c2ccccc2-c2c1ccc1c(Br)cccc21] | 78 | 766.34 |
| 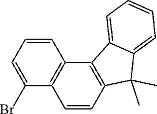 | 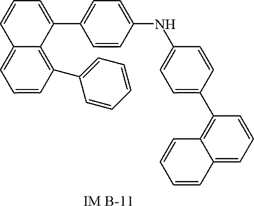 | 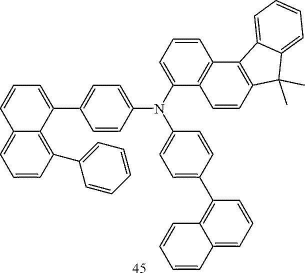 [SMILES: CC1(C)c2ccccc2-c2c1ccc1cc(Br)ccc21] | 79 | 740.32 |
| 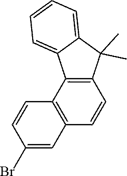 | 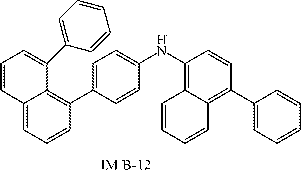 | 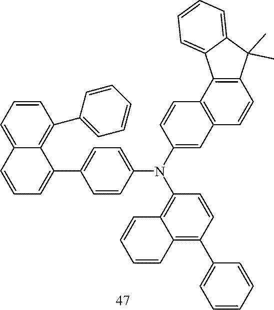 [SMILES: CC1(C)c2ccccc2-c2c1c(Br)cc1ccccc21] | 76 | 740.32 |
| 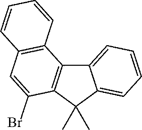 | 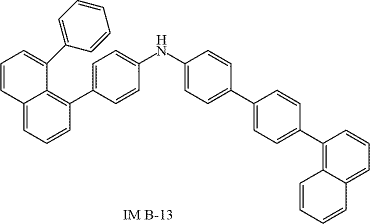 | 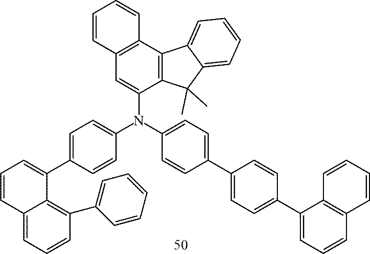 [SMILES: CC1(C)c2ccc3ccccc3c2-c2cccc(Br)c21] | 79 | 816.36 |
| 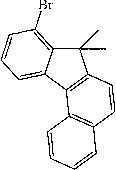 | 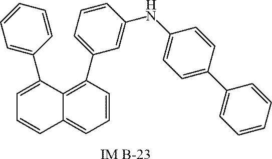 | 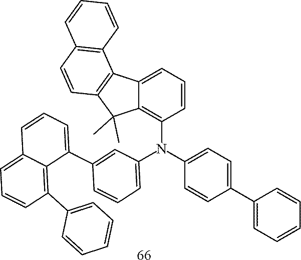 | 81 | 690.31 |
| | 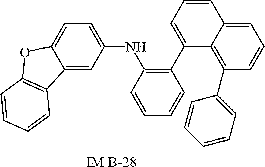 | 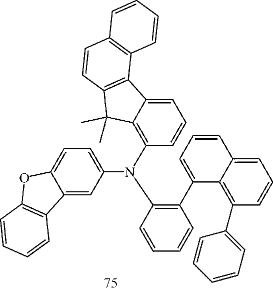 | 69 | 704.29 |
| | 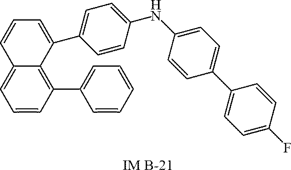 | 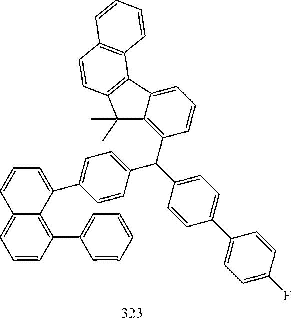 [SMILES: CC1(C)c2cc(Cl)ccc2-c2c1ccc1ccccc21] | 71 | 708.30 |
| 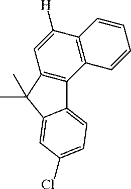 | 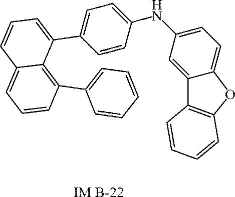 | 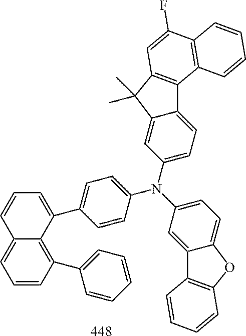 [SMILES: Brc1cccc2c1sc1ccc3ccccc3c12] | 54 | 722.28 |
| 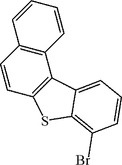 | 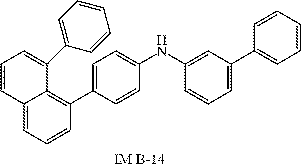 | 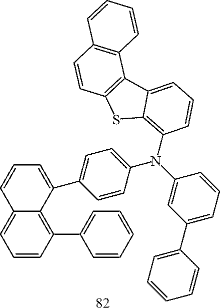 [SMILES: Brc1cccc2c1sc1cc3ccccc3cc12] | 74 | 680.23 |
| 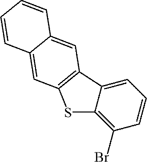 | 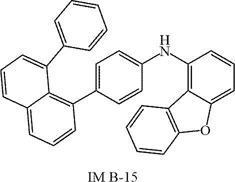 | 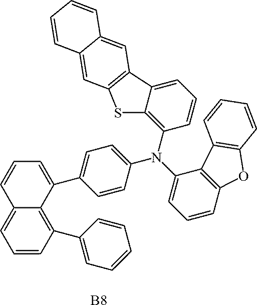 [SMILES: Brc1ccc2c(c1)sc1c3ccccc3ccc21] | 72 | 694.21 |
| 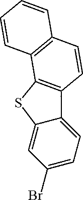 | 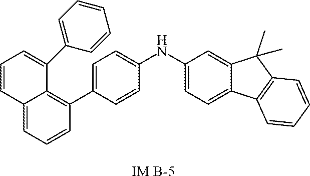 | 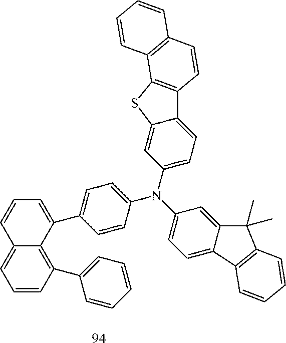 [SMILES: Brc1ccc2c(c1)sc1ccc3ccccc3c12] | 79 | 720.26 |
| 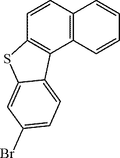 | 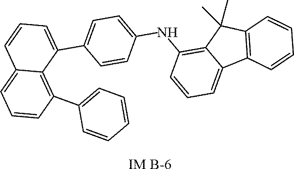 | 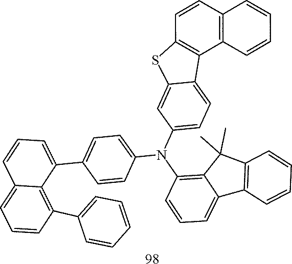 [SMILES: Brc1ccc2sc3ccc4ccccc4c3c2c1] | 71 | 720.26 |
| 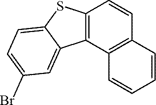 | 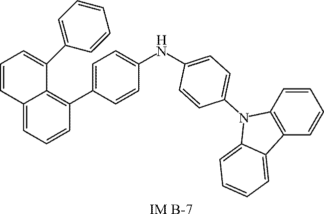 | 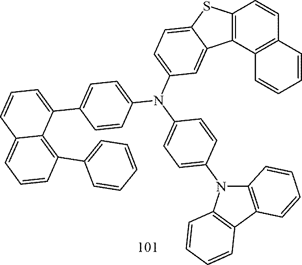 | 73 | 769.26 |
| | 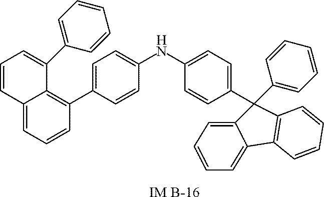 | 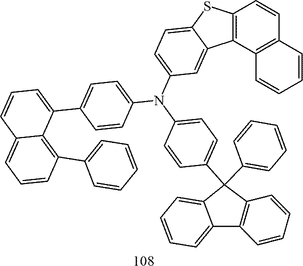 [SMILES: Brc1cccc2sc3ccc4ccccc4c3c12] | 68 | 844.30 |
| 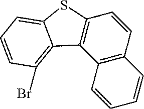 | 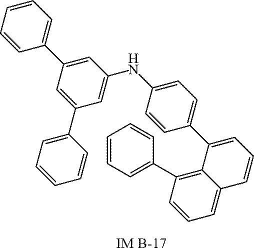 | 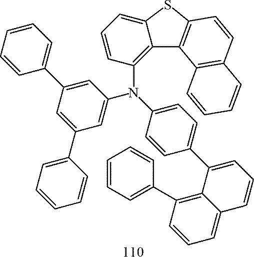 [SMILES: Brc1cc2sc3ccccc3c2c2ccccc12] | 69 | 756.26 |
| 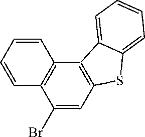 | 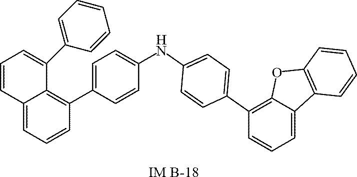 | 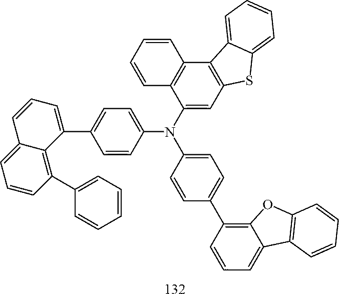 [SMILES: Brc1cc2c3ccccc3sc2c2ccccc12] | 67 | 770.24 |
| 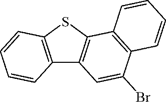 | 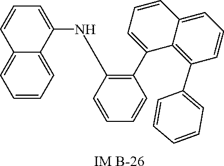 | 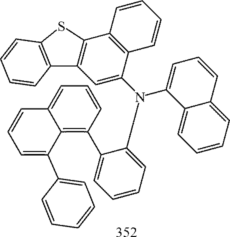 [SMILES: Brc1cccc2c1oc1ccc3ccccc3c12] | 59 | 654.22 |
| 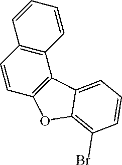 | 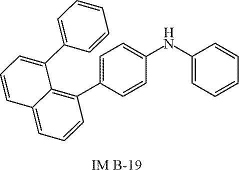 | 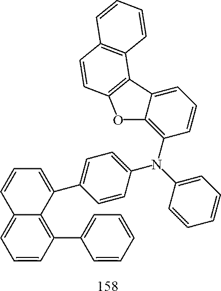 | 78 | 588.22 |
| | 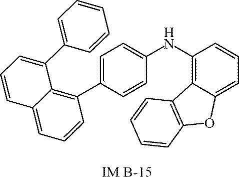 | 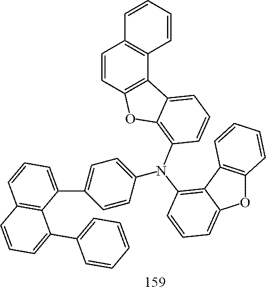 | 64 | 678.24 |
| | 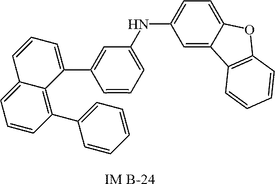 | 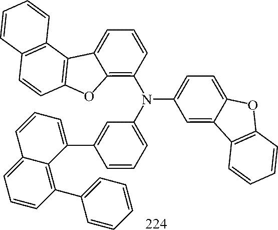 | 59 | 678.24 |
| | 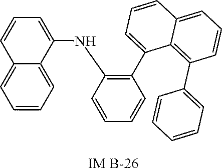 | 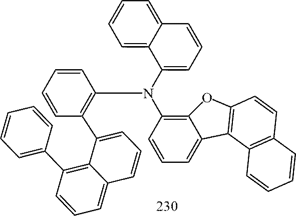 [SMILES: Brc1cccc2c1oc1cc3ccccc3cc12] | 60 | 638.24 |
| 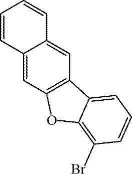 | 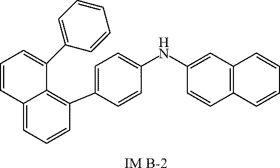 | 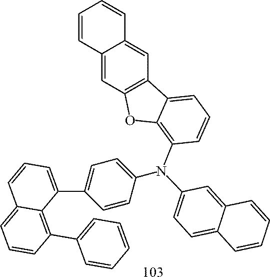 [SMILES: Brc1cccc2oc3c4ccccc4ccc3c12 ; Brc1ccc2oc3ccc4ccccc4c3c2c1] | 75 | 638.24 |
| 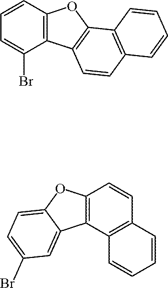 | 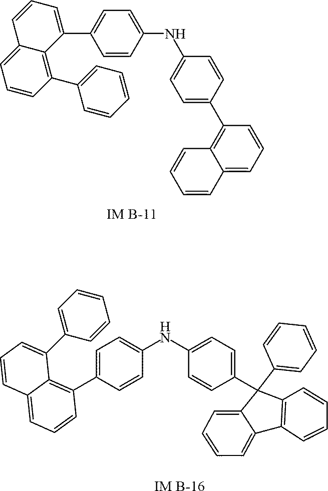 | 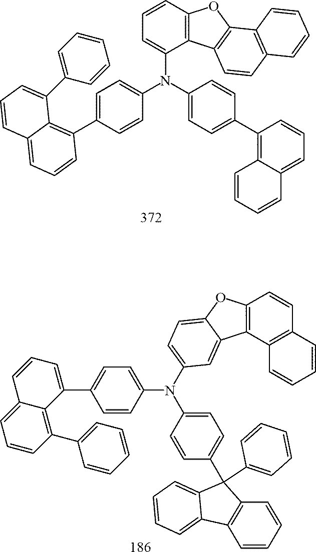 [SMILES: Brc1cccc2ccc3oc4ccccc4c3c12 ; Brc1cccc2c3c4ccccc4ccc3n(-c3ccccc3)c12] | 58 | 714.27 |
| | | | 68 | 828.32 |
| 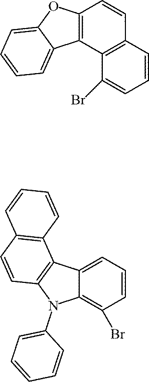 | 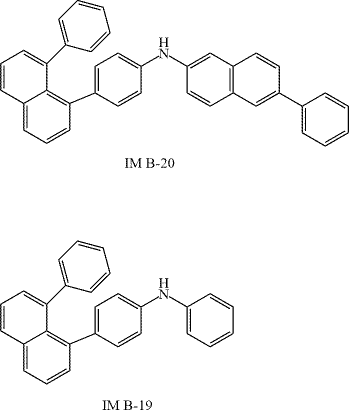 | 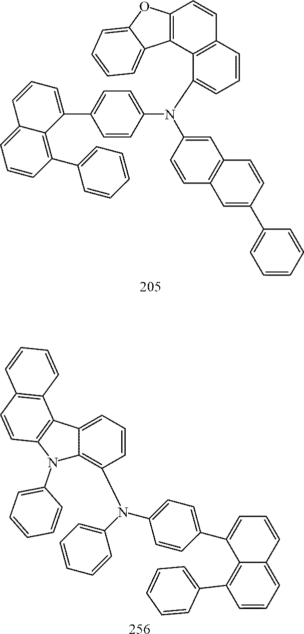 | 71 | 714.27 |
| | | | 74 | 663.27 |
| | 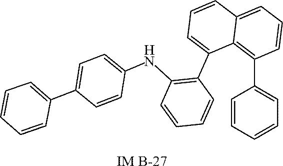 | 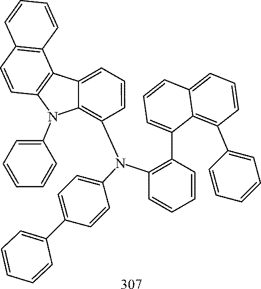 [SMILES: Brc1ccc2c3c4ccccc4ccc3n(-c3ccccc3)c2c1] | 69 | 739.30 |
| 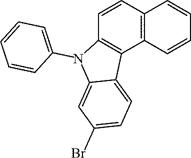 | 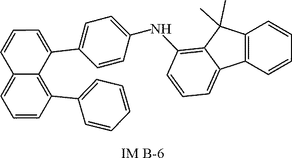 | 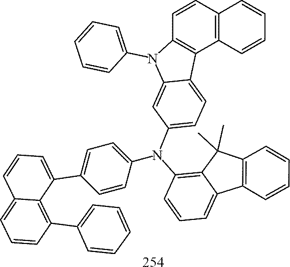 | 59 | 779.23 |
| | 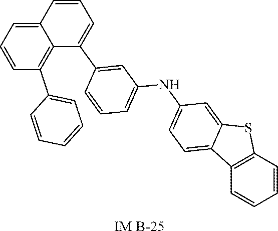 | 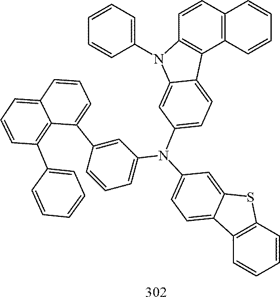 [SMILES: Brc1ccc2cc3c(cc2c1)c1ccccc1n3-c1ccccc1] | 68 | 769.26 |
| 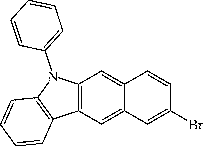 | 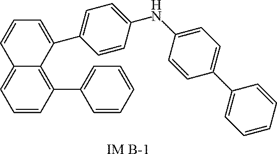 | 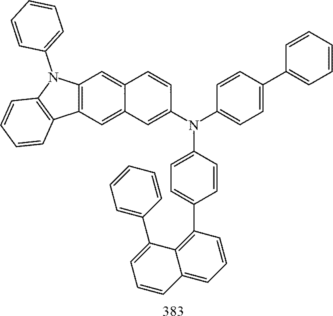 [SMILES: Brc1cc2c(c3ccccc13)-c1ccccc1[Si]2(c1ccccc1)c1ccccc1] | 64 | 739.30 |
| 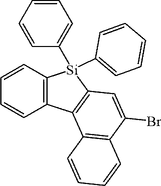 | 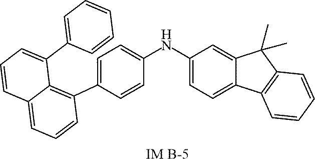 | 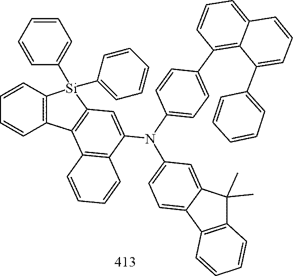 | 53 | 870.35 |

### Nuclear Magnetic Resonance (NMR) Data of Some Compounds Were Shown in Table 4 Below

TABLE 4

| | |
|---|---|
| Compound | NMR data |
| Compound 2 | ¹H-NMR(400 MHz, CD₂Cl₂): δ(ppm) 8.51-8.11(m, 2H), 8.02(t, 5H), 7.89-7.71(m, 10H), 7.65-7.43(m, 5H), 7.39(d, 6H), 7.31(d, 2H), 7.25(d, H), 7.13(d, 2H), 1.93(s, 6H). |
| Compound 164 | ¹H-NMR(400 MHz. CD₂Cl₂): δ(ppm) 8.97(s, 1H), 8.53(s, 1H), 8.35-8.13(t, 1H), 8.09-7.98(m. 3H), 7.82-7.45(m, 20H), 7.37(s, 1H), 7.25(d. 1H), 7.16(d, 1H), 7.05(d, 2H). |

### Fabrication and Performance Evaluation of OLEDs

### Example 1

### Blue Light-Emitting OLED

The OEI.D was fabricated through the following process:

An ITO anode substrate with a thickness of 1.500 Å was cut into a size of 40 mm (length) × 40 mm (width) × 0.7 mm (thickness), then the substrate was processed through photolithography into an experimental substrate with cathode, anode, and insulating layer patterns, the experimental substrate was subjected to a surface treatment with ultraviolet (UV)-ozone and O₂ : N₂ plasma to increase a work function of the anode (experimental substrate), and surfaces of the ITO substrate were cleaned with an organic solvent to remove scums and oil stains on the surface of the ITO substrate.

The compound F4-TCNQ was vacuum-deposited on the experimental substrate (anode) to form an HlL with a thickness of 100 Å. The compound NPB was vacuum-deposited on the HlL to form an HTL with a thickness of 1 200 Å.

The compound 2 was vacuum-deposited on the HTL to form an EBL with a thickness of 100 Å.

BH-01 and BD-01 were deposited on the EBL in a ratio of 98%:2% to form an organic electroluminescent layer (EML) with a thickness of 220 Å.

ET-06 and LiQ were deposited on the organic electroluminescent layer (EML) in a weight ratio of 1:1 to form an ETL with a thickness of 300 Å.

Yb was deposited on the ETL to form an EIL with a thickness of 15 Å, and then magnesium (Mg) and silver (Ag) were deposited on the EIL in a deposition rate ratio of 1:9 to form a cathode with a thickness of 120 Å.

The compound CP-05 was deposited on the cathode to form an organic capping layer (CPL) with a thickness of 650 Å, thereby completing the fabrication of the OLED.

### Examples 2 to 40

Blue light-emitting OLEDs were each fabricated by the same method as in Example 1, except that the remaining compounds listed in “Table 6 were each used instead of the compound 2 in the formation of the EBL.

### Comparative Examples 1 to 4

Blue light-emitting OLEDs in Comparative Examples 1 to 4 were each fabricated by the same method as in Example 1, except that compounds A, B, C, and D were each used instead of the compound 2 to form the EBL.

The structures of the main materials used in the above examples and comparative examples were shown in Table 5:

TABLE 5

| | | | |
|---|---|---|---|
| 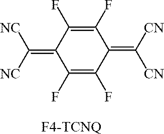 | 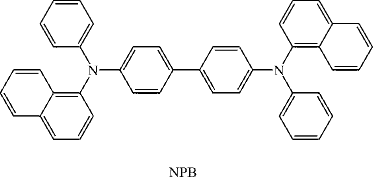 | 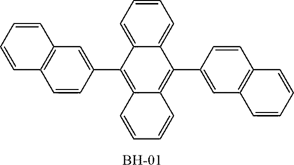 | 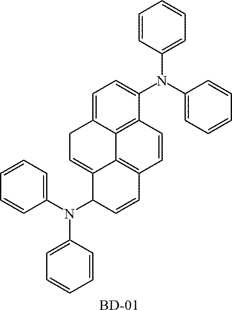 |
| 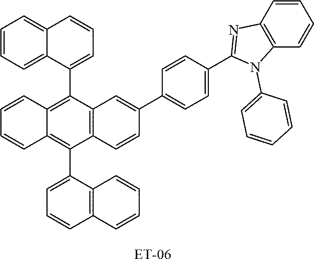 | 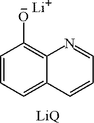 | 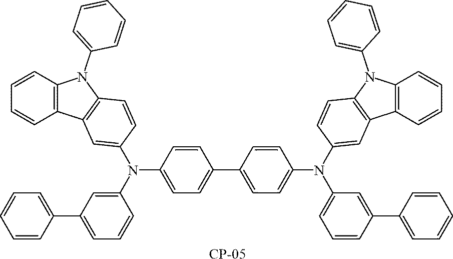 | 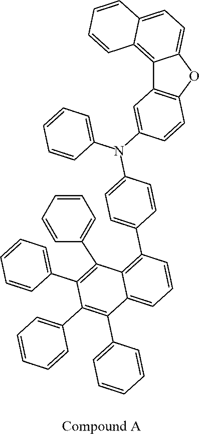 |
| 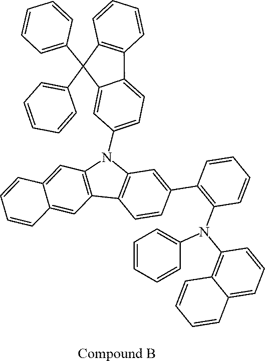 | 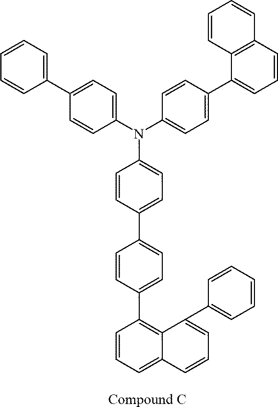 | 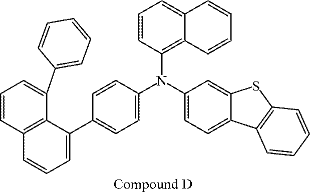 | |

The performance of each of the OLEDs fabricated in the examples and comparative examples was shown in Table 6, where the IVL (voltage, efficiency, and chromaticity coordinates) data and the T95 life span were tested at a current density of 15 mA/cm².

TABLE 6

| Example No. | Compound | Working voltage Volt (V) | Light-emitting efficiency (Cd/A) | Current power (lm/W) | Chromaticity coordinate CIE-x, CIE-y | External quantum efficiency (EQE, %) | T95 life span (h) |
|---|---|---|---|---|---|---|---|
| Example 1 | Compound 2 | 3.93 | 6.51 | 5.20 | 0.14, 0.05 | 13.39 | 242 |
| Example 2 | Compound 6 | 3.90 | 6.61 | 5.32 | 0.14, 0.05 | 13.60 | 246 |
| Example 3 | Compound 7 | 3.91 | 6.46 | 5.19 | 0.14, 0.05 | 13.29 | 275 |
| Example 4 | Compound 12 | 3.91 | 6.49 | 5.21 | 0.14, 0.05 | 13.35 | 230 |
| Example 5 | Compound 15 | 3.95 | 6.52 | 5.19 | 0.14, 0.05 | 13.41 | 280 |
| Example 6 | Compound 19 | 3.89 | 6.61 | 5.34 | 0.14, 0.05 | 13.60 | 250 |
| Example 7 | Compound 23 | 3.91 | 6.67 | 5.36 | 0.14, 0.05 | 13.72 | 246 |
| Example 8 | Compound 32 | 3.94 | 6.45 | 5.14 | 0.14, 0.05 | 13.27 | 282 |
| Example 9 | Compound 36 | 3.94 | 6.47 | 5.16 | 0.14, 0.05 | 13.31 | 251 |
| Example 10 | Compound 38 | 3.92 | 6.50 | 5.21 | 0.14, 0.05 | 13.37 | 276 |
| Example 11 | Compound 46 | 3.90 | 6.65 | 5.36 | 0.14, 0.05 | 13.68 | 253 |
| Example 12 | Compound 47 | 3.94 | 6.67 | 5.32 | 0.14, 0.05 | 13.72 | 243 |
| Example 13 | Compound 50 | 3.97 | 6.48 | 5.13 | 0.14, 0.05 | 13.33 | 283 |
| Example 14 | Compound 66 | 3.92 | 6.49 | 5.20 | 0.14, 0.05 | 13.35 | 265 |
| Example 15 | Compound 75 | 3.92 | 6.46 | 5.18 | 0.14, 0.05 | 13.29 | 248 |
| Example 16 | Compound 82 | 3.93 | 6.45 | 5.16 | 0.14, 0.05 | 13.27 | 240 |
| Example 17 | Compound 88 | 3.96 | 6.60 | 5.24 | 0.14, 0.05 | 13.58 | 265 |
| Example 18 | Compound 94 | 3.90 | 6.54 | 5.27 | 0.14, 0.05 | 13.45 | 283 |
| Example 19 | Compound 98 | 3.91 | 6.67 | 5.36 | 0.14, 0.05 | 13.72 | 276 |
| Example 20 | Compound 101 | 3.96 | 6.65 | 5.28 | 0.14, 0.05 | 13.68 | 277 |
| Example 21 | Compound 108 | 3.97 | 6.46 | 5.11 | 0.14, 0.05 | 13.29 | 265 |
| Example 22 | Compound 118 | 3.95 | 6.65 | 5.29 | 0.14, 0.05 | 13.68 | 269 |
| Example 23 | Compound 132 | 3.93 | 6.55 | 5.24 | 0.14, 0.05 | 13.47 | 270 |
| Example 24 | Compound 158 | 3.89 | 6.68 | 5.39 | 0.14, 0.05 | 13.74 | 281 |
| Example 25 | Compound 163 | 3.93 | 6.69 | 5.35 | 0.14, 0.05 | 13.76 | 261 |
| Example 26 | Compound 166 | 3.95 | 6.58 | 5.23 | 0.14, 0.05 | 13.54 | 254 |
| Example 27 | Compound 186 | 3.93 | 6.51 | 5.20 | 0.14, 0.05 | 13.39 | 273 |
| Examine 28 | Compound 205 | 3.91 | 6.48 | 5.21 | 0.14, 0.05 | 13.33 | 232 |
| Example 29 | Compound 224 | 3.92 | 6.56 | 5.26 | 0.14, 0.05 | 13.49 | 263 |
| Example 30 | Compound 230 | 3.92 | 6.60 | 5.29 | 0.14, 0.05 | 13.58 | 248 |
| Example 31 | Compound 236 | 3.94 | 6.58 | 5.25 | 0.14, 0.05 | 13.54 | 271 |
| Example 32 | Compound 254 | 3.91 | 6.56 | 5.27 | 0.14, 0.05 | 13.49 | 282 |
| Example 33 | Compound 303 | 3.93 | 6.69 | 5.35 | 0.14, 0.05 | 13.76 | 233 |
| Example 34 | Compound 307 | 3.94 | 6.60 | 5.26 | 0.14, 0.05 | 13.58 | 278 |
| Example 35 | Compound 323 | 3.92 | 6.56 | 5.26 | 0.14, 0.05 | 13.49 | 263 |
| Example 36 | Compound 352 | 3.90 | 6.54 | 5.27 | 0.14, 0.05 | 13.45 | 283 |
| Example 37 | Compound 372 | 3.91 | 6.48 | 5.21 | 0.14, 0.05 | 13.33 | 232 |
| Example 38 | Compound 393 | 3.90 | 6.51 | 5.24 | 0.14, 0.05 | 13.29 | 240 |
| Example 39 | Compound 413 | 3.89 | 6.45 | 5.20 | 0.14, 0.05 | 13.11 | 231 |
| Example 40 | Compound 448 | 3.90 | 6.53 | 5.26 | 0.14, 0.05 | 13.34 | 235 |
| Comparative Example 1 | Compound A | 4.09 | 5.52 | 4.24 | 0.14, 0.05 | 11.35 | 182 |
| Comparative Example 2 | Compound B | 4.17 | 5.69 | 4.28 | 0.14, 0.05 | 11.70 | 179 |
| Comparative Example 3 | Compound C | 4.11 | 5.54 | 4.23 | 0.14, 0.05 | 11.40 | 176 |
| Comparative Example 4 | Compound D | 4.14 | 5.60 | 4.26 | 0.14, 0.05 | 11.55 | 180 |

It can be seen from the results in Table 6 that, compared with the OLEDs corresponding to well-known compounds fabricated in Comparative Examples 1 to 4, the OLEDs with the organic compound of the present application as an EBL fabricated in Examples 1 to 40 have a driving voltage reduced by at least 0.12 V, a light-emitting efficiency (Cd/A) increased by at least 13.4%, an EQE increased by at least 13.4%, and a life span increased by at least 26.4%. Therefore, the organic compound of the present application can improve both the light-emitting efficiency and the life span. It can be seen from the above data that, when the organic compound of the present application is used as an EBL of an electronic component, the light-emitting efficiency (Cd/A), EQE, and life span (T95) of the electronic component are all significantly improved. Therefore, the organic compound of the present application can be used in an EBL to fabricate an OLED with high light-emitting efficiency and long life span.

The above embodiments are only used for describing the technical solutions of the present application, and are not intended to limit the present application. Although the present application is described in detail with reference to the above examples, those of ordinary skill in the art should understand that they can still modify the technical solutions described in the above examples, or make equivalent substitutions for some technical features therein. These modifications or substitutions do not make the essence of the corresponding technical solutions deviate from the spirit and scope of the technical solutions of the examples of the present application.

1. An organic compound with a structure shown in formula 1: 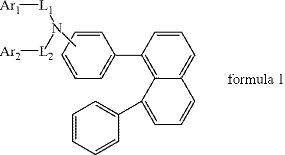
wherein Ar₁ is selected from the group consisting of formula I, formula II, and formula III: 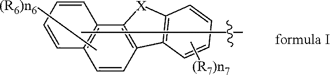 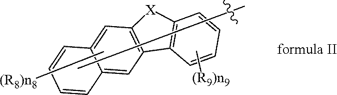 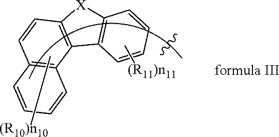
wherein 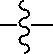
represents a chemical bond;
X is selected from the group consisting of C(R₁R₂), O, S, N(R₃), and Si(R₄R₅);
R₁, R₂, R₃, R₄, and R₅ are the same or different, and are each independently selected from the group consisting of alkyl with 1 to 5 carbon atoms, aryl with 6 to 12 carbon atoms, and heteroaryl with 3 to 12 carbon atoms; or R₁ and R₂ form a saturated or unsaturated 5-13 membered ring; or R₄ and R₅ form a saturated or unsaturated 5-13 membered ring;
R₆, R₇, R₈, R₉, R₁₀, and R₁₁ are the same or different, and are each independently selected from the group consisting of deuterium, halogen, cyano, alkyl with 1 to 5 carbon atoms, trialkylsilyl with 3 to 12 carbon atoms, aryl with 6 to 12 carbon atoms, and heteroaryl with 3 to 12 carbon atoms;
R₆, R₇, R₈, R₉, R₁₀, and R₁₁ are collectively represented by Rᵢ; n₆, n₇, n₈, n₉, n₁₀, and n₁₁ are collectively represented by ni; ni indicates the number of Rᵢ, and i is a variable of 6, 7, 8, 9, 10, or 11; when i is 6, 8, or 10, ni is selected from the group consisting of 0, 1, 2, 3, 4, 5, and 6; when i is 7, 9, or 11, nᵢ is selected from the group consisting of 0, 1, 2, 3, and 4; and when nᵢ is greater than 1, any two Rᵢ are the same or different;
Ar₂ is selected from the group consisting of substituted or unsubstituted aryl with 6 to 40 carbon atoms and substituted or unsubstituted heteroaryl with 3 to 30 carbon atoms;
L₁ and L₂ are the same or different, and are each independently selected from the group consisting of a single bond, substituted or unsubstituted arylene with 6 to 20 carbon atoms, and substituted or unsubstituted heteroarylene with 3 to 20 carbon atoms;
substituents in Li, L₂, and Ar₂ are the same or different, and are each independently selected from the group consisting of deuterium, halogen, cyano, alkyl with 1 to 10 carbon atoms, trialkylsilyl with 3 to 12 carbon atoms, aryl with 6 to 20 carbon atoms, heteroaryl with 3 to 20 carbon atoms, haloalkyl with 1 to 10 carbon atoms, and alkoxy with 1 to 10 carbon atoms; and optionally, any two adjacent substituents in Ar₂ form a saturated or unsaturated 3-15 membered ring.

2. The organic compound according to claim 1, wherein L₁ and L₂ are each independently selected from the group consisting of a single bond and substituted or unsubstituted arylene with 6 to 12 carbon atoms.

3. The organic compound according to claim 1, wherein L₁ and L₂ are each independently selected from the group consisting of a single bond, substituted or unsubstituted phenylene, substituted or unsubstituted naphthylene, and substituted or unsubstituted biphenylene; and
preferably, substituents in L₁ and L₂ are each independently selected from the group consisting of deuterium, fluorine, cyano, methyl, ethyl, isopropyl, tert-butyl, phenyl, naphthyl, and pyridyl.

4. The organic compound according to claim 1, wherein L₁ and L₂ are each independently selected from the group consisting of a single bond and a substituted or unsubstituted group V; an unsubstituted group V is selected from the group consisting of the following groups: 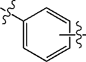 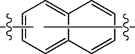 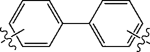
a substituted group V has one or more substituents, and the one or more substituents are each independently selected from the group consisting of deuterium, fluorine, cyano, methyl, ethyl, isopropyl, tert-butyl, phenyl, naphthyl, and pyridyl; and when the substituted group V has two or more substituents, the two or more substituents are the same or different.

5. The organic compound according to claim 1, wherein L₁ and L₂ are each independently selected from the group consisting of a single bond and the following groups: 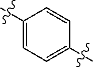 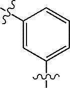 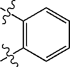 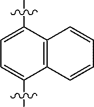 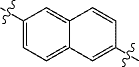 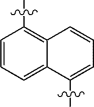 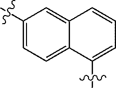 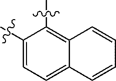 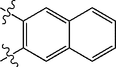 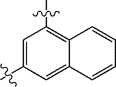 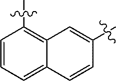 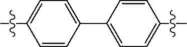 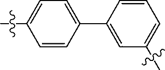 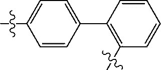 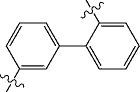 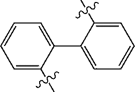 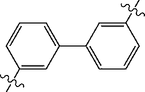 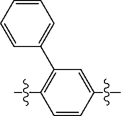 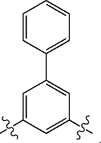
.

6. The organic compound according to claim 1, wherein Ar₂ is selected from the group consisting of substituted or unsubstituted aryl with 6 to 25 carbon atoms and substituted or unsubstituted heteroaryl with 3 to 20 carbon atoms;
preferably, a substituent in Ar₂ is independently selected from the group consisting of deuterium, fluorine, cyano, trimethylsilyl, alkyl with 1 to 5 carbon atoms, aryl with 6 to 12 carbon atoms, and heteroaryl with 3 to 12 carbon atoms; and optionally, any two adjacent substituents in Ar₂ form a saturated or unsaturated 5-13 membered ring.

7. The organic compound according to claim 1, wherein Ar₂ is selected from the group consisting of substituted or unsubstituted phenyl, substituted or unsubstituted biphenyl, substituted or unsubstituted naphthyl, substituted or unsubstituted dibenzofuranyl, substituted or unsubstituted dibenzothiophenyl, substituted or unsubstituted fluorenyl, substituted or unsubstituted phenanthryl, and substituted or unsubstituted carbazolyl;
preferably, a substituent in Ar₂ is independently selected from the group consisting of deuterium, cyano, fluorine, trimethylsilyl, methyl, ethyl, isopropyl, tert-butyl, phenyl, naphthyl, biphenyl, and pyridyl; and optionally, any two adjacent substituents in Ar₂ form a fluorene ring.

8. The organic compound according to claim 1, wherein Ar₂ is a substituted or unsubstituted group W; an unsubstituted group W is selected from the group consisting of the following groups: 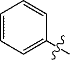 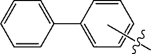 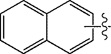 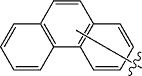 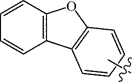 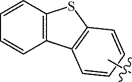 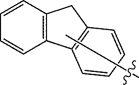 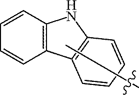 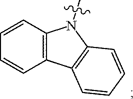
a substituted group W has one or more substituents, and the one or more substituents are each independently selected from the group consisting of deuterium, cyano, fluorine, trimethylsilyl, methyl, ethyl, isopropyl, tert-butyl, phenyl, naphthyl, biphenyl, and pyridyl; and when the substituted group W has two or more substituents, the two or more substituents are the same or different.

9. The organic compound according to claim 1, wherein Ar₂ is selected from the group consisting of the following groups: 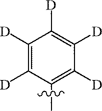 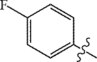 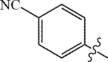 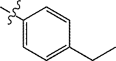 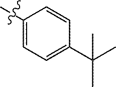 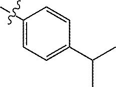 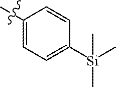 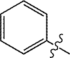 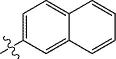 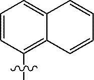 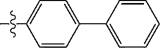 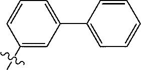 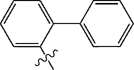 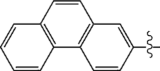 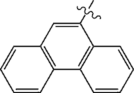 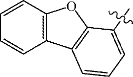 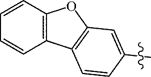 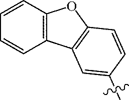 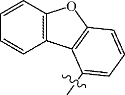 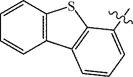 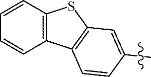 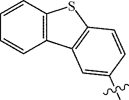 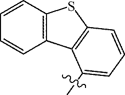 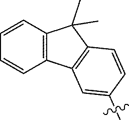 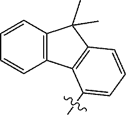 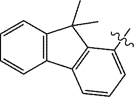 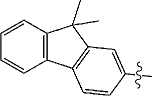 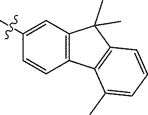 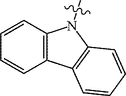 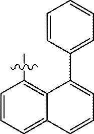 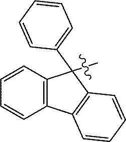 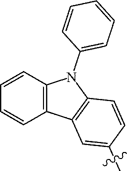 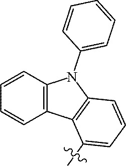 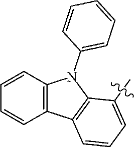 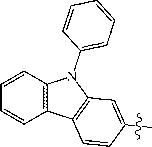 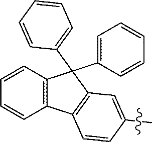 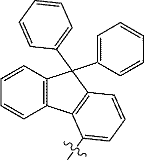 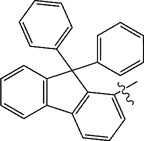 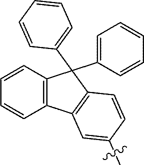 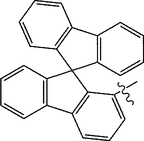 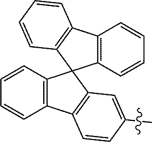 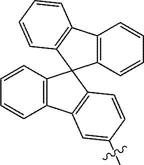 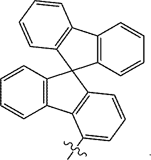
.

10. The organic compound according to claim 1, wherein R₁, R₂, R₃, R₄, and R₅ are each independently selected from the group consisting of methyl, ethyl, isopropyl, tert-butyl, phenyl, naphthyl, biphenyl, pyridyl, pyrimidinyl, quinolinyl, isoquinolinyl, carbazolyl, dibenzofuranyl, and dibenzothiophenyl; or, R₁ and R₂ form cyclopentane, cyclohexane, or a fluorene ring; or, R₄ and R₅ form cyclopentane, cyclohexane, or a fluorene ring.

11. The organic compound according to claim 1, wherein R₆, R₇, R₈, R₉, R₁₀, and R₁₁ are each independently selected from the group consisting of deuterium, fluorine, cyano, methyl, ethyl, isopropyl, tert-butyl, trimethylsilyl, phenyl, naphthyl, biphenyl, pyridyl, pyrimidinyl, quinolinyl, isoquinolinyl, carbazolyl, dibenzofuranyl, and dibenzothiophenyl.

12. The organic compound according to claim 1, wherein Ar₁ is a substituted or unsubstituted group Q; an unsubstituted group Q is selected from the group consisting of the following groups: 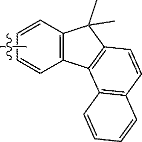 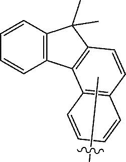 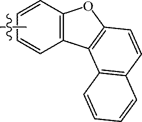 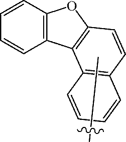 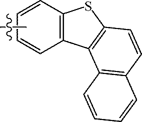 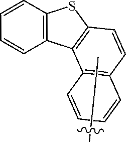 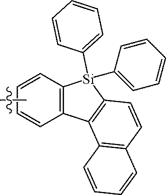 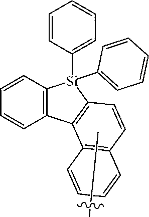 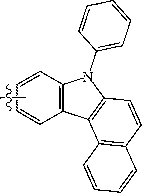 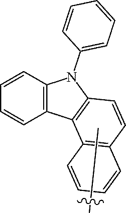 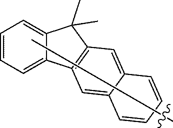 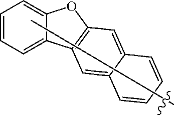 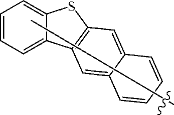 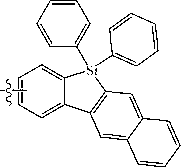 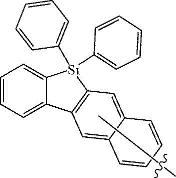 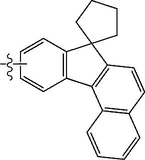 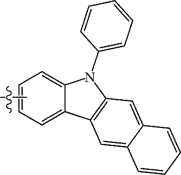 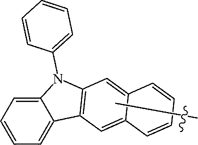 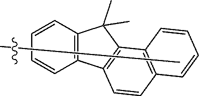 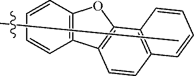 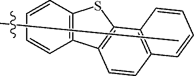 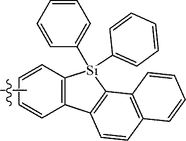 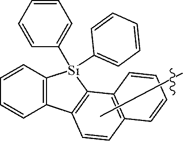 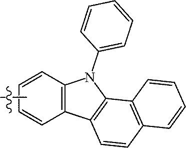 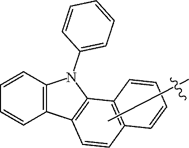 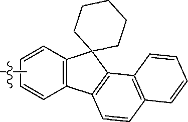 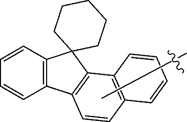 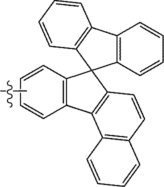 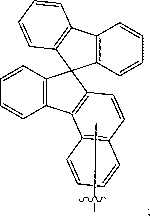
a substituted group Q has one or more substituents, and the one or more substituents are each independently selected from the group consisting of deuterium, cyano, fluorine, methyl, ethyl, isopropyl, tert-butyl, phenyl, naphthyl, biphenyl, pyridyl, cyclohexyl, carbazolyl, and trimethylsilyl; and when the substituted group Q has two or more substituents, the two or more substituents are the same or different.

13. The organic compound according to claim 1, wherein Ar₁ is selected from the group consisting of the following groups: 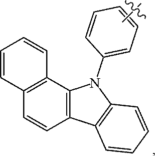 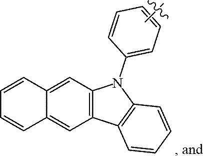 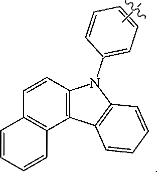 [SMILES: CC1(C)c2ccc3ccccc3c2-c2cccc(N(c3ccccc3)c3ccc(-c4cccc5cccc(-c6ccccc6)c45)cc3)c21]
.

14. The organic compound according to claim 1, wherein the organic compound is selected from the group consisting of the following compounds:

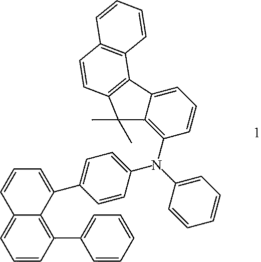 [SMILES: CC1(C)c2ccc3ccccc3c2-c2cccc(N(c3ccc(-c4ccccc4)cc3)c3ccc(-c4cccc5cccc(-c6ccccc6)c45)cc3)c21]

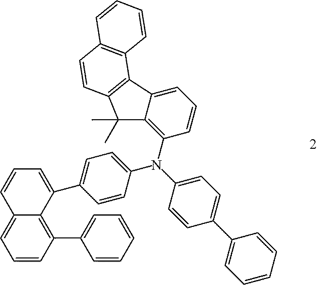 [SMILES: CC1(C)c2ccc3ccccc3c2-c2cccc(N(c3ccc(-c4cccc5cccc(-c6ccccc6)c45)cc3)c3cccc(-c4ccccc4)c3)c21]

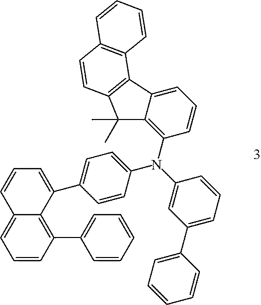 [SMILES: CC1(C)c2ccc3ccccc3c2-c2cccc(N(c3ccc(-c4cccc5cccc(-c6ccccc6)c45)cc3)c3ccccc3-c3ccccc3)c21]

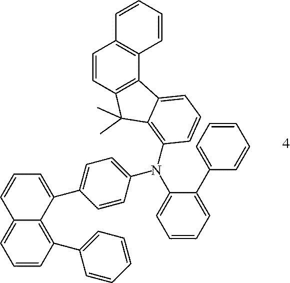 [SMILES: CC1(C)c2cc3ccccc3cc2-c2cccc(N(c3ccc(-c4cccc5cccc(-c6ccccc6)c45)cc3)c3cccc4ccccc34)c21]

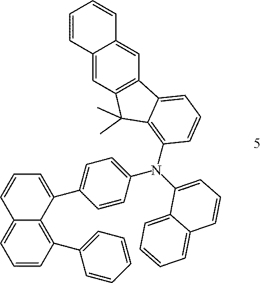 [SMILES: CC1(C)c2cc3ccccc3cc2-c2cccc(N(c3ccc(-c4cccc5cccc(-c6ccccc6)c45)cc3)c3ccc4ccccc4c3)c21]

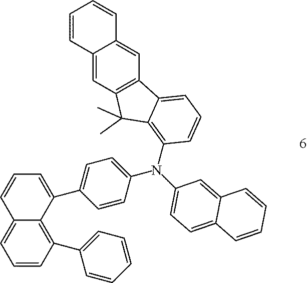 [SMILES: CC1(C)c2cc3ccccc3cc2-c2cccc(N(c3ccc(-c4cccc5cccc(-c6ccccc6)c45)cc3)c3ccc4c(c3)oc3ccccc34)c21]

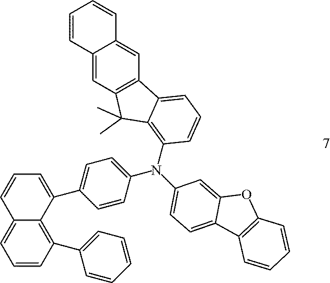 [SMILES: CC1(C)c2cc3ccccc3cc2-c2cccc(N(c3ccc(-c4cccc5cccc(-c6ccccc6)c45)cc3)c3ccc4oc5ccccc5c4c3)c21]

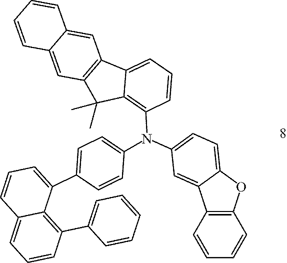 [SMILES: CC1(C)c2ccc3ccccc3c2-c2cccc(N(c3ccc(-c4cccc5cccc(-c6ccccc6)c45)cc3)c3cccc4oc5ccccc5c34)c21]

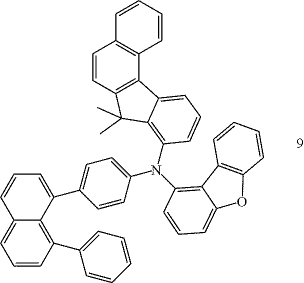 [SMILES: CC1(C)c2ccc3ccccc3c2-c2cccc(N(c3ccc(-c4cccc5cccc(-c6ccccc6)c45)cc3)c3cccc4c3oc3ccccc34)c21]

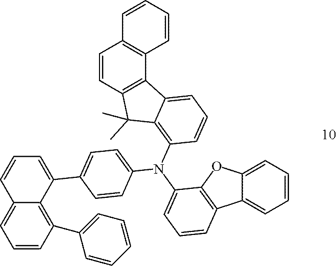 [SMILES: CC1(C)c2cc(N(c3ccc(-c4cccc5cccc(-c6ccccc6)c45)cc3)c3ccc4c(c3)sc3ccccc34)ccc2-c2c1ccc1ccccc21]

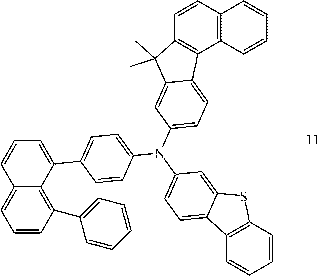 [SMILES: CC1(C)c2cc(N(c3ccc(-c4cccc5cccc(-c6ccccc6)c45)cc3)c3ccc4sc5ccccc5c4c3)ccc2-c2c1ccc1ccccc21]

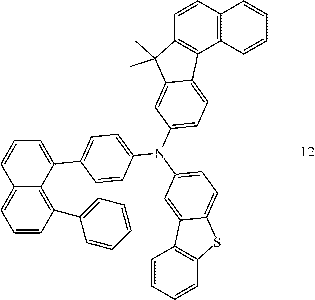 [SMILES: CC1(C)c2cc(N(c3ccc(-c4cccc5cccc(-c6ccccc6)c45)cc3)c3cccc4sc5ccccc5c34)ccc2-c2ccc3ccccc3c21]

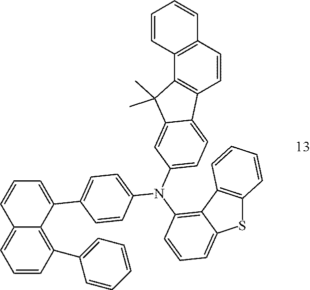 [SMILES: CC1(C)c2cc(N(c3ccc(-c4cccc5cccc(-c6ccccc6)c45)cc3)c3cccc4c3sc3ccccc34)ccc2-c2ccc3ccccc3c21]

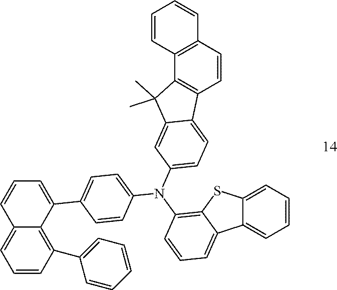 [SMILES: CC1(C)c2ccccc2-c2ccc(N(c3ccc(-c4cccc5cccc(-c6ccccc6)c45)cc3)c3ccc4c(c3)C(C)(C)c3c-4ccc4ccccc34)cc21]

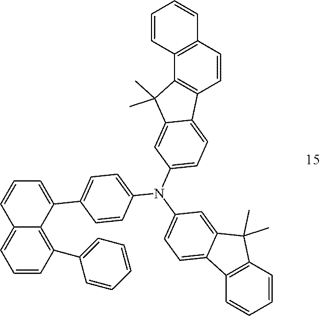 [SMILES: CC1(C)c2ccccc2-c2cc(N(c3ccc(-c4cccc5cccc(-c6ccccc6)c45)cc3)c3ccc4c(c3)C(C)(C)c3c-4ccc4ccccc34)ccc21]

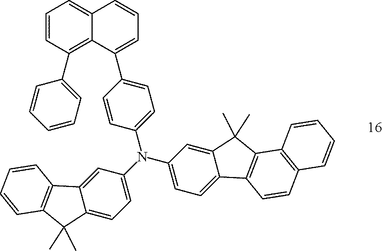 [SMILES: CC1(C)c2cc(N(c3ccc(-c4cccc5cccc(-c6ccccc6)c45)cc3)c3ccc4sc5ccccc5c4c3)ccc2-c2c1ccc1ccccc21]

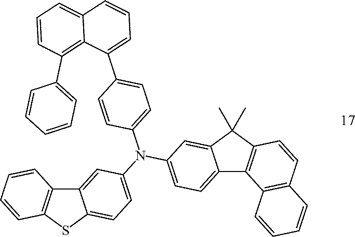 [SMILES: CC1(C)c2ccccc2-c2c(N(c3ccc(-c4cccc5cccc(-c6ccccc6)c45)cc3)c3ccc4c(c3)C(C)(C)c3ccc5ccccc5c3-4)cccc21]

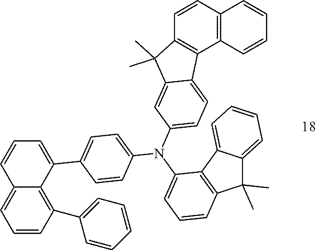 [SMILES: CC1(C)c2cc(N(c3ccc(-c4cccc5cccc(-c6ccccc6)c45)cc3)c3cccc4c3C(C)(C)c3ccccc3-4)ccc2-c2c1ccc1ccccc21]

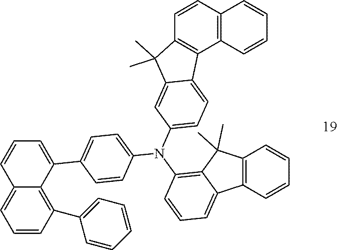 [SMILES: CC1(C)c2ccc(N(c3ccc(-c4cccc5cccc(-c6ccccc6)c45)cc3)c3cccc4c3C(C)(C)c3ccccc3-4)cc2-c2c1ccc1ccccc21]

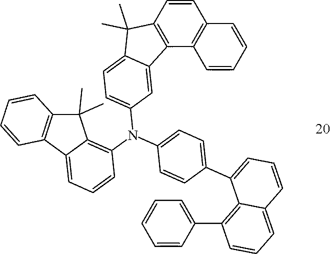 [SMILES: CC1(C)c2cc(N(c3ccc(-c4cccc5cccc(-c6ccccc6)c45)cc3)c3ccc4c5ccccc5n(-c5ccccc5)c4c3)ccc2-c2c1ccc1ccccc21]

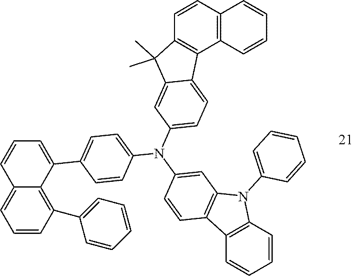 [SMILES: CC1(C)c2cc(N(c3ccc(-c4cccc5cccc(-c6ccccc6)c45)cc3)c3ccc4c(c3)c3ccccc3n4-c3ccccc3)ccc2-c2c1ccc1ccccc21]

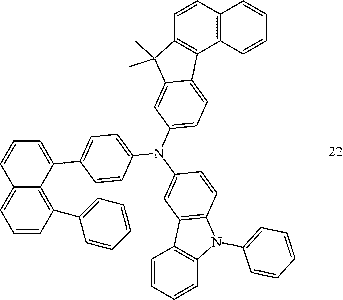 [SMILES: CC1(C)c2ccc(N(c3ccc(-c4cccc5cccc(-c6ccccc6)c45)cc3)c3ccc(-n4c5ccccc5c5ccccc54)cc3)cc2-c2c1ccc1ccccc21]

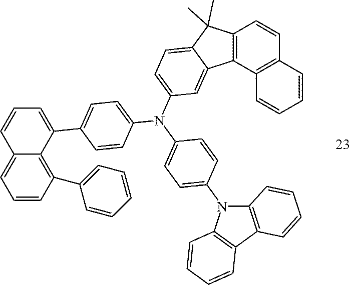 [SMILES: CC1(C)c2ccc(N(c3ccc(-c4cccc5cccc(-c6ccccc6)c45)cc3)c3cccc(-n4c5ccccc5c5ccccc54)c3)cc2-c2c1ccc1ccccc21]

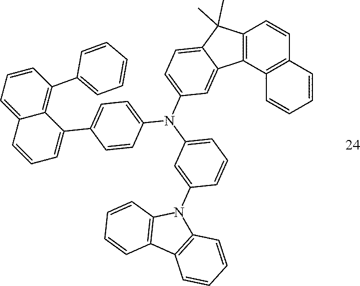 [SMILES: CC1(C)c2ccc(N(c3ccc(-c4cccc5cccc(-c6ccccc6)c45)cc3)c3ccccc3-n3c4ccccc4c4ccccc43)cc2-c2c1ccc1ccccc21]

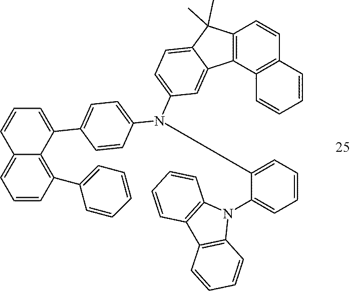 [SMILES: CC1(C)c2ccc(N(c3ccc(-c4cccc5cccc(-c6ccccc6)c45)cc3)c3cccc4c3c3ccccc3n4-c3ccccc3)cc2-c2c1ccc1ccccc21]

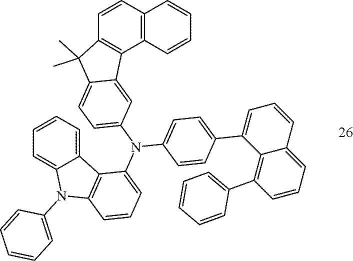 [SMILES: CC1(C)c2ccc(N(c3ccc(-c4cccc5cccc(-c6ccccc6)c45)cc3)c3cccc4c5ccccc5n(-c5ccccc5)c34)cc2-c2c1ccc1ccccc21]

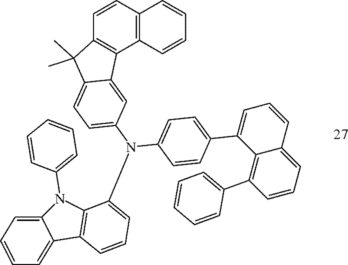 [SMILES: CC1(C)c2ccc(N(c3ccc(-c4cccc5cccc(-c6ccccc6)c45)cc3)c3ccc4c(c3)C(c3ccccc3)(c3ccccc3)c3ccccc3-4)cc2-c2c1ccc1ccccc21]

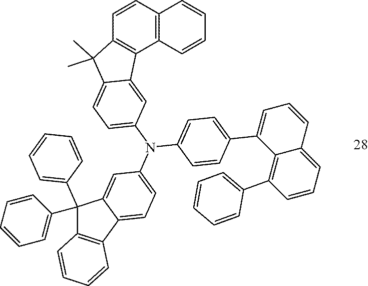 [SMILES: CC1(C)c2ccc(N(c3ccc(-c4cccc5cccc(-c6ccccc6)c45)cc3)c3cccc4c3-c3ccccc3C4(c3ccccc3)c3ccccc3)cc2-c2c1ccc1ccccc21]

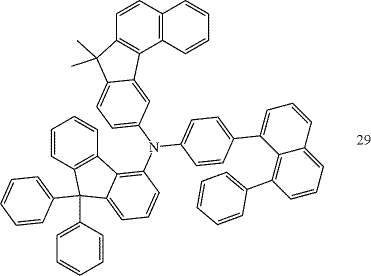 [SMILES: CC1(C)c2ccc(N(c3ccc(-c4cccc5cccc(-c6ccccc6)c45)cc3)c3ccc(C4(c5ccccc5)c5ccccc5-c5ccccc54)cc3)cc2-c2c1ccc1ccccc21]

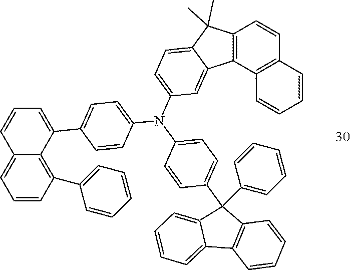 [SMILES: CC1(C)c2ccc(N(c3ccc(-c4cccc5cccc(-c6ccccc6)c45)cc3)c3cccc(C4(c5ccccc5)c5ccccc5-c5ccccc54)c3)cc2-c2c1ccc1ccccc21]

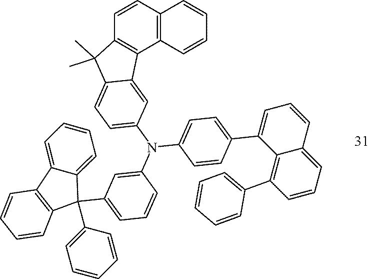 [SMILES: CC1(C)c2ccc(N(c3ccc(-c4cccc5cccc(-c6ccccc6)c45)cc3)c3ccc4c(c3)C3(c5ccccc5-c5ccccc53)c3ccccc3-4)cc2-c2c1ccc1ccccc21]

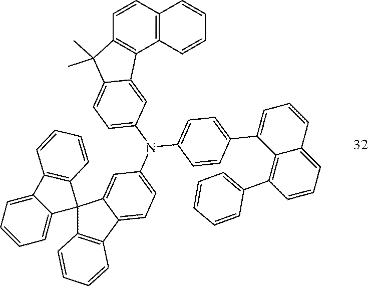 [SMILES: CC1(C)c2cccc(N(c3ccc(-c4cccc5cccc(-c6ccccc6)c45)cc3)c3cccc4c3-c3ccccc3C43c4ccccc4-c4ccccc43)c2-c2c1ccc1ccccc21]

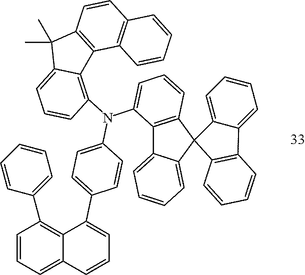 [SMILES: CC1(C)c2cccc(N(c3ccc(-c4cccc5cccc(-c6ccccc6)c45)cc3)c3ccc4c(c3)-c3ccccc3C4(c3ccccc3)c3ccccc3)c2-c2c1ccc1ccccc21]

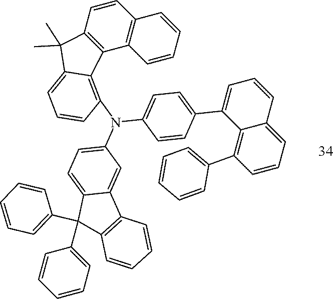 [SMILES: CC1(C)c2cccc(N(c3ccc(-c4cccc5cccc(-c6ccccc6)c45)cc3)c3ccccc3C3(c4ccccc4)c4ccccc4-c4ccccc43)c2-c2c1ccc1ccccc21]

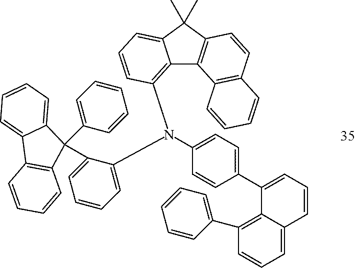 [SMILES: CC1(C)c2cccc(N(c3ccc(-c4cccc5cccc(-c6ccccc6)c45)cc3)c3ccc4c(c3)-c3ccccc3C43c4ccccc4-c4ccccc43)c2-c2c1ccc1ccccc21]

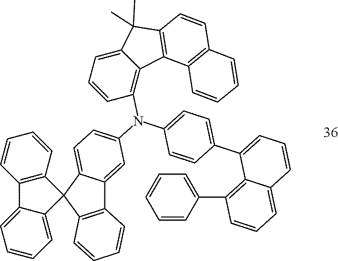 [SMILES: CC1(C)c2cccc(N(c3ccc(-c4cccc5cccc(-c6ccccc6)c45)cc3)c3cccc4c3C3(c5ccccc5-c5ccccc53)c3ccccc3-4)c2-c2c1ccc1ccccc21]

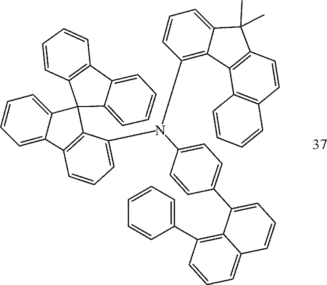 [SMILES: CC1(C)c2cccc(N(c3ccc(-c4ccc(-c5ccccc5)cc4)cc3)c3ccc(-c4cccc5cccc(-c6ccccc6)c45)cc3)c2-c2c1ccc1ccccc21]

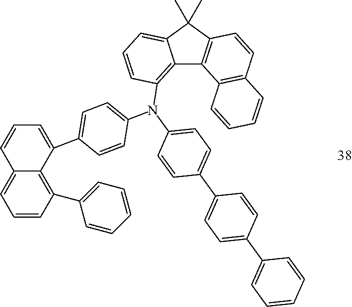 [SMILES: CC1(C)c2cccc(N(c3ccc(-c4cccc(-c5ccccc5)c4)cc3)c3ccc(-c4cccc5cccc(-c6ccccc6)c45)cc3)c2-c2c1ccc1ccccc21]

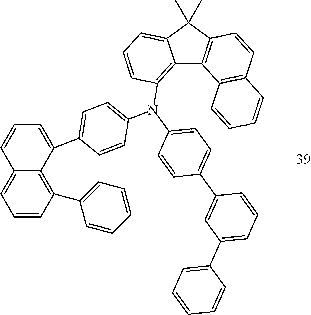 [SMILES: CC1(C)c2cccc(N(c3ccc(-c4cccc5cccc(-c6ccccc6)c45)cc3)c3cc(-c4ccccc4)cc(-c4ccccc4)c3)c2-c2c1ccc1ccccc21]

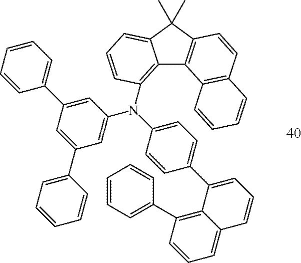 [SMILES: CC1(C)c2cccc(N(c3ccc(-c4cccc5cccc(-c6ccccc6)c45)cc3)c3ccc(-c4ccccc4)cc3-c3ccccc3)c2-c2c1ccc1ccccc21]

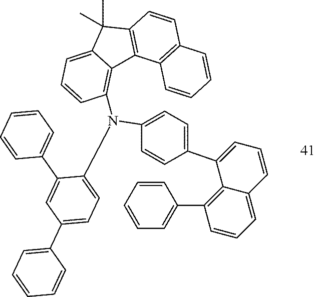 [SMILES: CC1(C)c2cccc(N(c3ccc(-c4cccc5cccc(-c6ccccc6)c45)cc3)c3cccc(-c4ccc(-c5ccccc5)cc4)c3)c2-c2c1ccc1ccccc21]

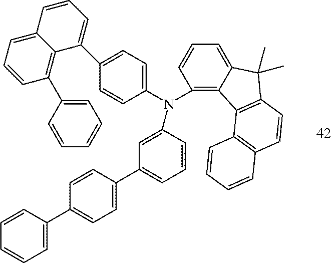 [SMILES: CC1(C)c2cccc(N(c3ccc(-c4cccc5cccc(-c6ccccc6)c45)cc3)c3ccccc3-c3ccc(-c4ccccc4)cc3)c2-c2c1ccc1ccccc21]

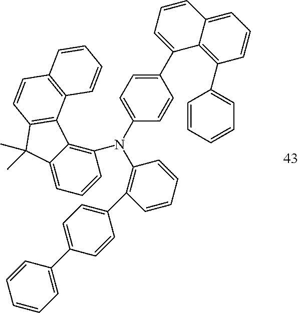 [SMILES: CC1(C)c2ccccc2-c2c1c(N(c1ccc(-c3cccc4cccc(-c5ccccc5)c34)cc1)c1cc3ccccc3c3ccccc13)cc1ccccc21]

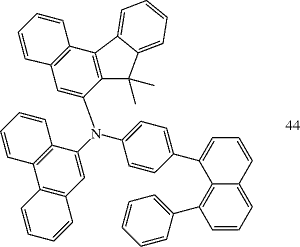 [SMILES: CC1(C)c2ccccc2-c2c1cc(N(c1ccc(-c3cccc4cccc(-c5ccccc5)c34)cc1)c1ccc3c(ccc4ccccc43)c1)c1ccccc21]

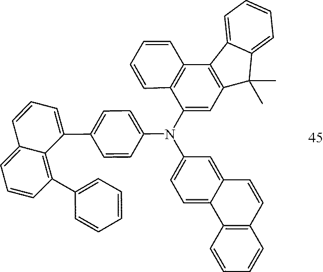 [SMILES: CC1(C)c2ccccc2-c2c1cc(N(c1ccc(-c3cccc4ccccc34)cc1)c1ccc(-c3cccc4cccc(-c5ccccc5)c34)cc1)c1ccccc21]

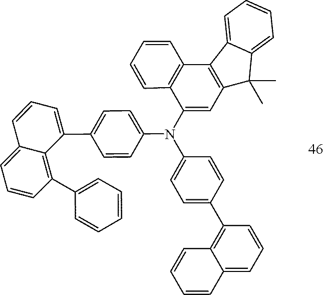 [SMILES: CC1(C)c2ccccc2-c2c1ccc1cc(N(c3ccc(-c4cccc5cccc(-c6ccccc6)c45)cc3)c3ccc(-c4ccccc4)c4ccccc34)ccc21]

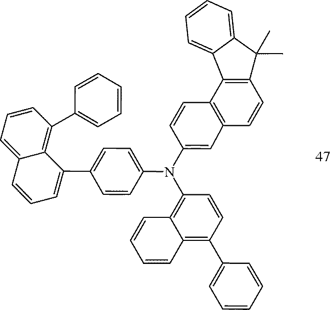 [SMILES: CC1(C)c2ccccc2-c2c1ccc1ccc(N(c3ccc(-c4ccc5ccccc5c4)cc3)c3ccc(-c4cccc5cccc(-c6ccccc6)c45)cc3)cc21]

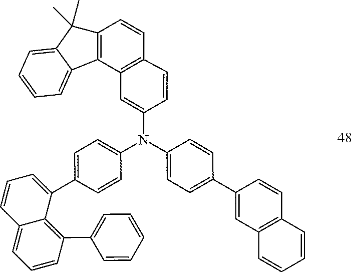 [SMILES: CC1(C)c2ccccc2-c2c1ccc1cccc(N(c3ccc(-c4cccc5cccc(-c6ccccc6)c45)cc3)c3ccc4cc(-c5ccccc5)ccc4c3)c21]

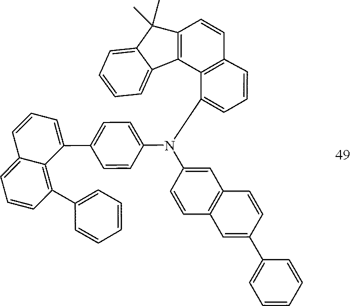 [SMILES: CC1(C)c2ccccc2-c2c1c(N(c1ccc(-c3ccc(-c4cccc5ccccc45)cc3)cc1)c1ccc(-c3cccc4cccc(-c5ccccc5)c34)cc1)cc1ccccc21]

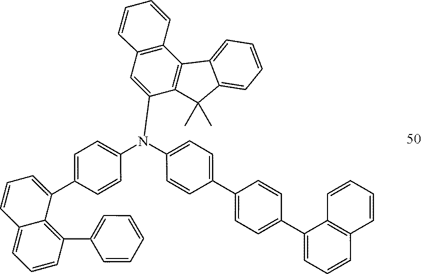 [SMILES: CC1(C)c2ccccc2-c2c1cc(N(c1ccc(-c3ccc4c(c3)oc3ccccc34)cc1)c1ccc(-c3cccc4cccc(-c5ccccc5)c34)cc1)c1ccccc21]

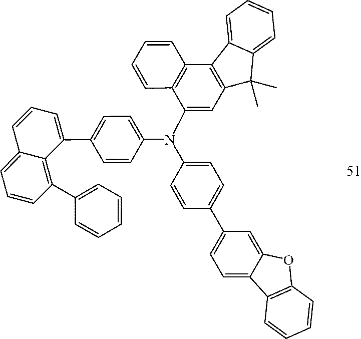 [SMILES: CC1(C)c2ccccc2-c2c1ccc1c(N(c3ccc(-c4ccc5oc6ccccc6c5c4)cc3)c3ccc(-c4cccc5cccc(-c6ccccc6)c45)cc3)cccc21]

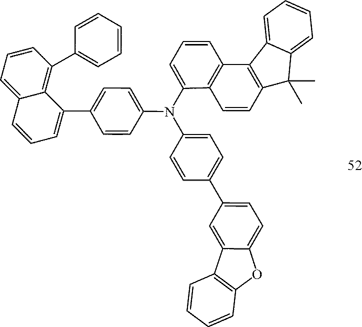 [SMILES: CC1(C)c2ccccc2-c2c1ccc1cc(N(c3ccc(-c4cccc5cccc(-c6ccccc6)c45)cc3)c3ccc(-c4cccc5oc6ccccc6c45)cc3)ccc21]

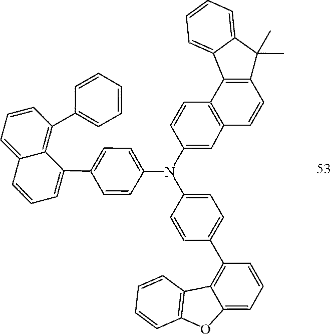 [SMILES: CC1(C)c2ccccc2-c2c1cc(N(c1ccc(-c3cccc4c3oc3ccccc34)cc1)c1ccc(-c3cccc4cccc(-c5ccccc5)c34)cc1)c1ccccc21]

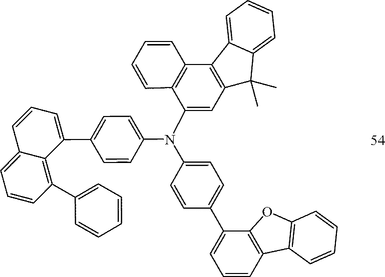 [SMILES: CC1(C)c2ccccc2-c2c1c(N(c1ccc(-c3ccc4c(c3)sc3ccccc34)cc1)c1ccc(-c3cccc4cccc(-c5ccccc5)c34)cc1)cc1ccccc21]

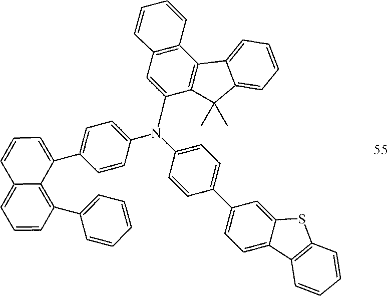 [SMILES: CC1(C)c2ccccc2-c2c1ccc1cccc(N(c3ccc(-c4ccc5sc6ccccc6c5c4)cc3)c3ccc(-c4cccc5cccc(-c6ccccc6)c45)cc3)c21]

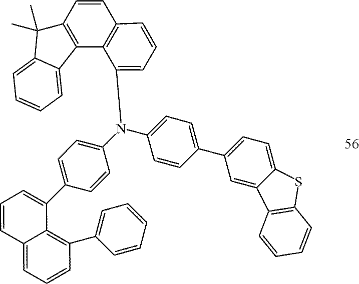 [SMILES: CC1(C)c2ccccc2-c2c1ccc1ccc(N(c3ccc(-c4cccc5cccc(-c6ccccc6)c45)cc3)c3ccc(-c4cccc5sc6ccccc6c45)cc3)cc21]

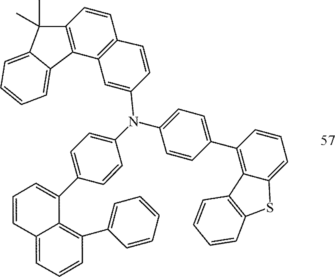 [SMILES: CC1(C)c2ccccc2-c2c1ccc1cc(N(c3ccc(-c4cccc5c4sc4ccccc45)cc3)c3ccc(-c4cccc5cccc(-c6ccccc6)c45)cc3)ccc21]

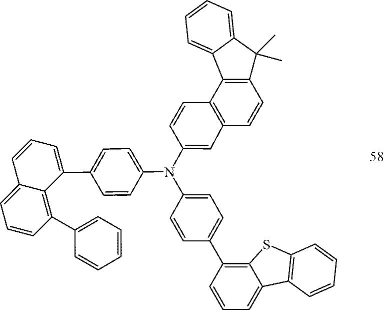 [SMILES: CC1(C)c2ccccc2-c2c1ccc1c(N(c3ccc(-c4cccc5cccc(-c6ccccc6)c45)cc3)c3cccc(-c4cccc5oc6ccccc6c45)c3)cccc21]

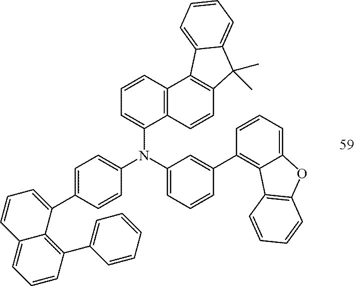 [SMILES: CC1(C)c2ccccc2-c2c1cc(N(c1ccc(-c3cccc4cccc(-c5ccccc5)c34)cc1)c1cccc(-c3cccc4c3oc3ccccc34)c1)c1ccccc21]

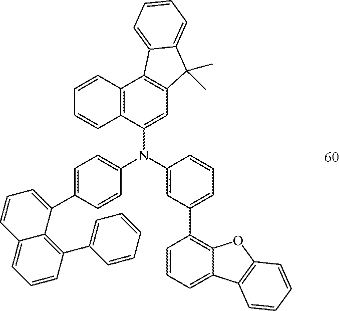 [SMILES: CC1(C)c2ccccc2-c2c1c(N(c1ccc(-c3cccc4cccc(-c5ccccc5)c34)cc1)c1cccc(-c3cccc4sc5ccccc5c34)c1)cc1ccccc21]

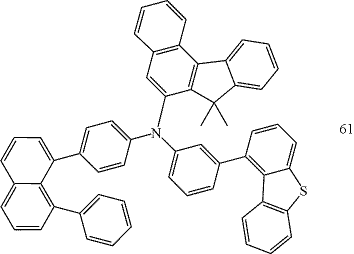 [SMILES: CC1(C)c2ccccc2-c2cc(-c3ccc(N(c4ccc(-c5cccc6cccc(-c7ccccc7)c56)cc4)c4cccc5ccc6c(c45)-c4ccccc4C6(C)C)cc3)ccc21]

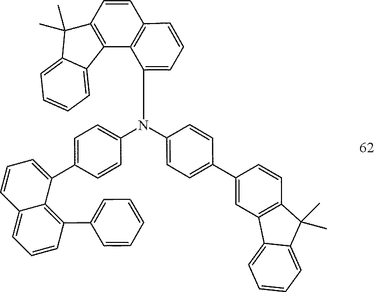 [SMILES: CC1(C)c2ccccc2-c2c(-c3ccc(N(c4ccc(-c5cccc6cccc(-c7ccccc7)c56)cc4)c4ccc5ccc6c(c5c4)-c4ccccc4C6(C)C)cc3)cccc21]

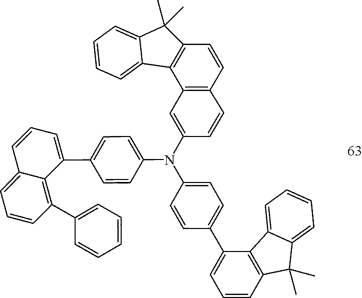 [SMILES: CC1(C)c2ccccc2-c2c1ccc1c(N(c3ccc(-c4cccc5cccc(-c6ccccc6)c45)cc3)c3cccc(-c4cccc5cccc(-c6ccccc6)c45)c3)cccc21]

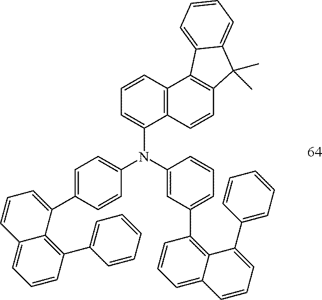 [SMILES: CC1(C)c2ccccc2-c2c1ccc1cc(N(c3ccc(-c4cccc5cccc(-c6ccccc6)c45)cc3)c3ccccc3-c3cccc4cccc(-c5ccccc5)c34)ccc21]

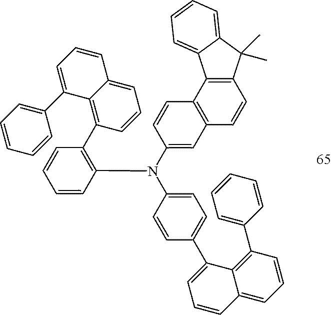 [SMILES: CC1(C)c2ccc3ccccc3c2-c2cccc(N(c3ccc(-c4ccccc4)cc3)c3cccc(-c4cccc5cccc(-c6ccccc6)c45)c3)c21]

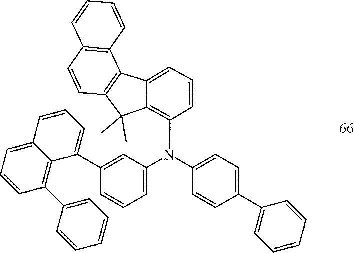 [SMILES: CC1(C)c2ccc3ccccc3c2-c2cccc(N(c3cccc(-c4cccc5cccc(-c6ccccc6)c45)c3)c3cccc4ccccc34)c21]

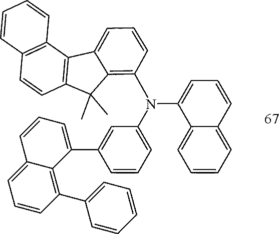 [SMILES: CC1(C)c2ccc3ccccc3c2-c2cccc(N(c3cccc(-c4cccc5cccc(-c6ccccc6)c45)c3)c3ccc4oc5ccccc5c4c3)c21]

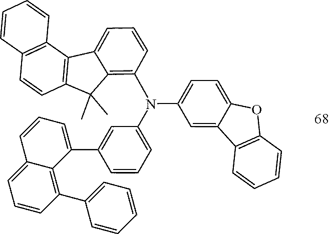 [SMILES: CC1(C)c2cc(N(c3cccc(-c4cccc5cccc(-c6ccccc6)c45)c3)c3ccc4c(c3)sc3ccccc34)ccc2-c2c1ccc1ccccc21]

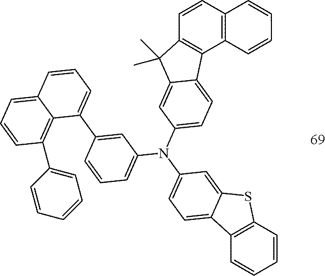 [SMILES: CC1(C)c2ccccc2-c2ccc(N(c3cccc(-c4cccc5cccc(-c6ccccc6)c45)c3)c3ccc4c(c3)C(C)(C)c3ccc5ccccc5c3-4)cc21]

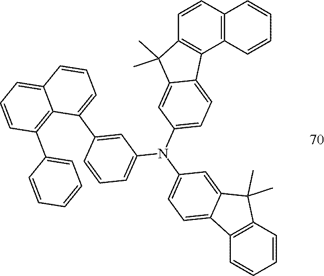 [SMILES: CC1(C)c2cc(N(c3cccc(-c4cccc5cccc(-c6ccccc6)c45)c3)c3ccc4c5ccccc5n(-c5ccccc5)c4c3)ccc2-c2c1ccc1ccccc21]

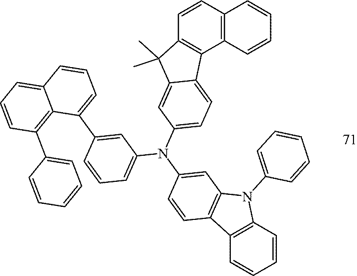 [SMILES: CC1(C)c2cccc(N(c3ccc(-c4cccc(-c5ccccc5)c4)cc3)c3cccc(-c4cccc5cccc(-c6ccccc6)c45)c3)c2-c2c1ccc1ccccc21]

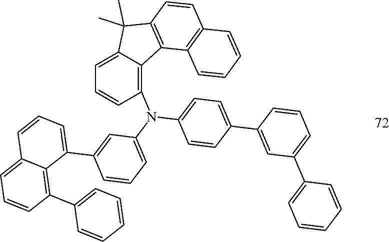 [SMILES: CC1(C)c2ccc3ccccc3c2-c2cccc(N(c3ccc(-c4ccccc4)cc3)c3ccccc3-c3cccc4cccc(-c5ccccc5)c34)c21]

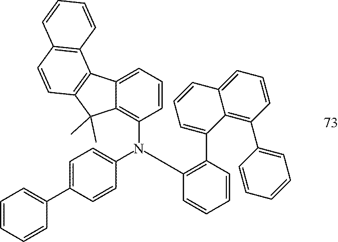 [SMILES: CC1(C)c2ccc3ccccc3c2-c2cccc(N(c3ccccc3-c3cccc4cccc(-c5ccccc5)c34)c3cccc4ccccc34)c21]

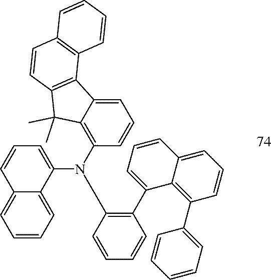 [SMILES: CC1(C)c2ccc3ccccc3c2-c2cccc(N(c3ccc4oc5ccccc5c4c3)c3ccccc3-c3cccc4cccc(-c5ccccc5)c34)c21]

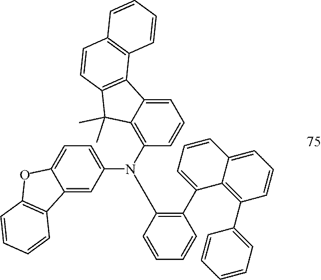 [SMILES: CC1(C)c2cc(N(c3ccc4c(c3)sc3ccccc34)c3ccccc3-c3cccc4cccc(-c5ccccc5)c34)ccc2-c2c1ccc1ccccc21]

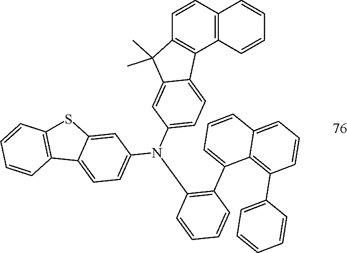 [SMILES: CC1(C)c2ccccc2-c2ccc(N(c3ccc4c(c3)C(C)(C)c3ccc5ccccc5c3-4)c3ccccc3-c3cccc4cccc(-c5ccccc5)c34)cc21]

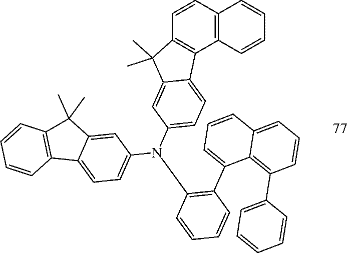 [SMILES: CC1(C)c2cc(N(c3ccc4c5ccccc5n(-c5ccccc5)c4c3)c3ccccc3-c3cccc4cccc(-c5ccccc5)c34)ccc2-c2c1ccc1ccccc21]

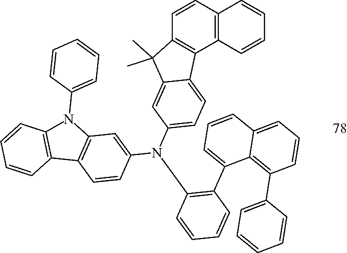 [SMILES: CC1(C)c2cccc(N(c3ccc(-c4cccc(-c5ccccc5)c4)cc3)c3ccccc3-c3cccc4cccc(-c5ccccc5)c34)c2-c2c1ccc1ccccc21]

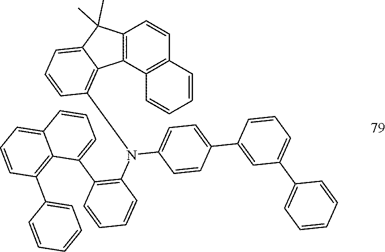 [SMILES: c1ccc(-c2cccc3cccc(-c4ccc(N(c5ccccc5)c5cccc6c5sc5ccc7ccccc7c56)cc4)c23)cc1]

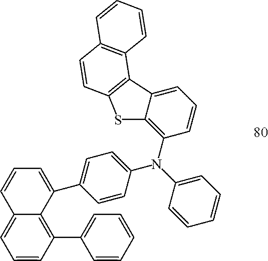 [SMILES: c1ccc(-c2ccc(N(c3ccc(-c4cccc5cccc(-c6ccccc6)c45)cc3)c3cccc4c3sc3ccc5ccccc5c34)cc2)cc1]

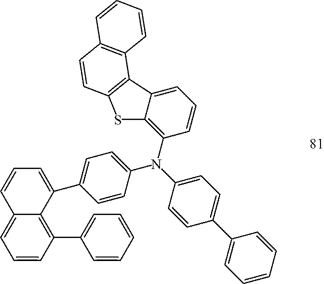 [SMILES: c1ccc(-c2cccc(N(c3ccc(-c4cccc5cccc(-c6ccccc6)c45)cc3)c3cccc4c3sc3ccc5ccccc5c34)c2)cc1]

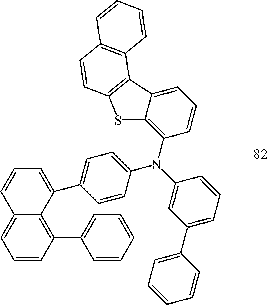 [SMILES: c1ccc(-c2ccccc2N(c2ccc(-c3cccc4cccc(-c5ccccc5)c34)cc2)c2cccc3c2sc2ccc4ccccc4c23)cc1]

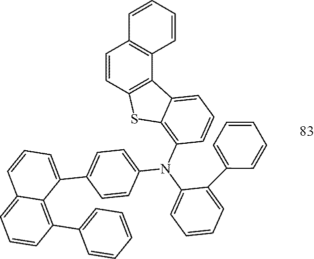 [SMILES: c1ccc(-c2cccc3cccc(-c4ccc(N(c5cccc6ccccc56)c5cccc6c5sc5ccc7ccccc7c56)cc4)c23)cc1]

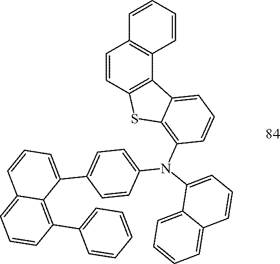 [SMILES: c1ccc(-c2cccc3cccc(-c4ccc(N(c5ccc6ccccc6c5)c5cccc6c5sc5ccc7ccccc7c56)cc4)c23)cc1]

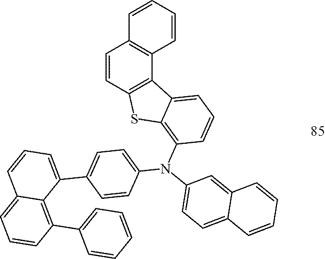 [SMILES: c1ccc(-c2cccc3cccc(-c4ccc(N(c5ccc6c(c5)oc5ccccc56)c5cccc6c5sc5cc7ccccc7cc56)cc4)c23)cc1]

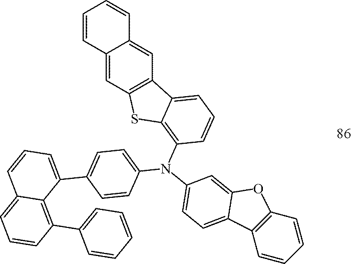 [SMILES: c1ccc(-c2cccc3cccc(-c4ccc(N(c5ccc6oc7ccccc7c6c5)c5cccc6c5sc5cc7ccccc7cc56)cc4)c23)cc1]

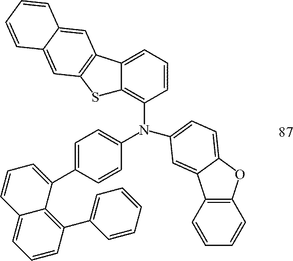 [SMILES: c1ccc(-c2cccc3cccc(-c4ccc(N(c5cccc6c5sc5cc7ccccc7cc56)c5cccc6oc7ccccc7c56)cc4)c23)cc1]

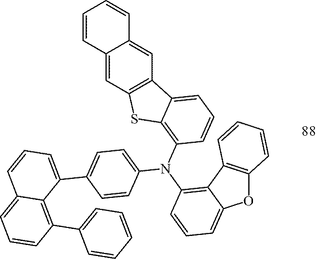 [SMILES: c1ccc(-c2cccc3cccc(-c4ccc(N(c5cccc6c5oc5ccccc56)c5cccc6c5sc5cc7ccccc7cc56)cc4)c23)cc1]

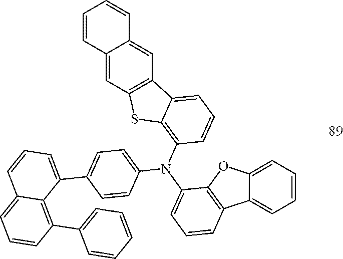 [SMILES: c1ccc(-c2cccc3cccc(-c4ccc(N(c5ccc6c(c5)sc5ccccc56)c5ccc6c(c5)sc5ccc7ccccc7c56)cc4)c23)cc1]

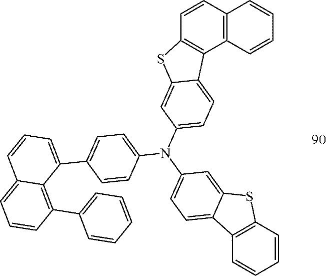 [SMILES: c1ccc(-c2cccc3cccc(-c4ccc(N(c5ccc6c(c5)sc5ccc7ccccc7c56)c5ccc6sc7ccccc7c6c5)cc4)c23)cc1]

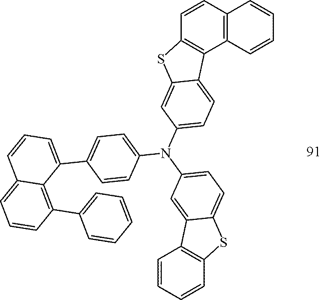 [SMILES: c1ccc(-c2cccc3cccc(-c4ccc(N(c5ccc6c(c5)sc5ccc7ccccc7c56)c5cccc6sc7ccccc7c56)cc4)c23)cc1]

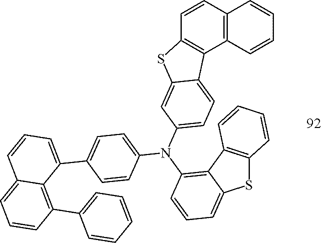 [SMILES: c1ccc(-c2cccc3cccc(-c4ccc(N(c5ccc6c(c5)sc5ccc7ccccc7c56)c5cccc6c5sc5ccccc56)cc4)c23)cc1]

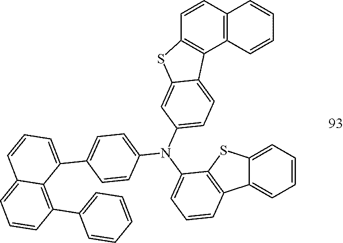 [SMILES: CC1(C)c2ccccc2-c2ccc(N(c3ccc(-c4cccc5cccc(-c6ccccc6)c45)cc3)c3ccc4c(c3)sc3c5ccccc5ccc43)cc21]

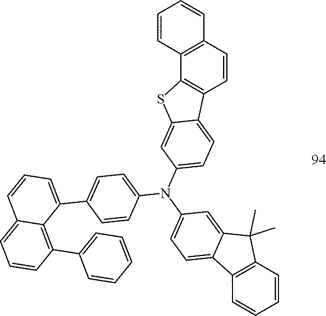 [SMILES: CC1(C)c2ccccc2-c2cc(N(c3ccc(-c4cccc5cccc(-c6ccccc6)c45)cc3)c3ccc4c(c3)sc3c5ccccc5ccc43)ccc21]

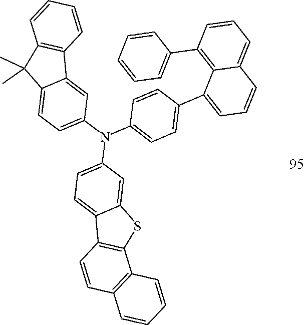 [SMILES: CC1(C)c2ccccc2-c2c(N(c3ccc(-c4cccc5cccc(-c6ccccc6)c45)cc3)c3ccc4c(c3)sc3c5ccccc5ccc43)cccc21]

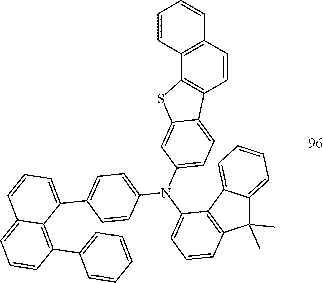 [SMILES: CC1(C)c2ccccc2-c2cccc(N(c3ccc(-c4cccc5cccc(-c6ccccc6)c45)cc3)c3ccc4c(c3)sc3c5ccccc5ccc43)c21]

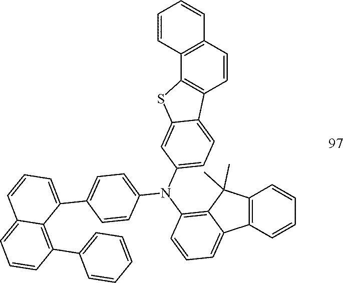 [SMILES: CC1(C)c2ccccc2-c2cccc(N(c3ccc(-c4cccc5cccc(-c6ccccc6)c45)cc3)c3ccc4c(c3)sc3ccc5ccccc5c34)c21]

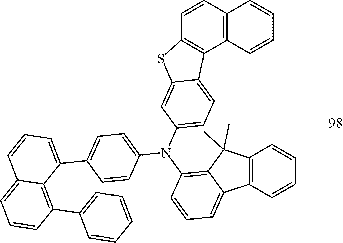 [SMILES: c1ccc(-c2cccc3cccc(-c4ccc(N(c5ccc6c(c5)sc5ccc7ccccc7c56)c5ccc6c7ccccc7n(-c7ccccc7)c6c5)cc4)c23)cc1]

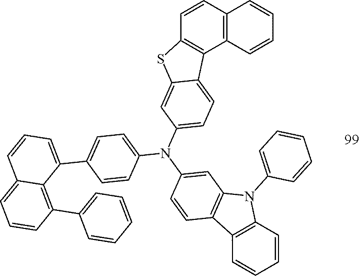 [SMILES: c1ccc(-c2cccc3cccc(-c4ccc(N(c5ccc6c(c5)sc5ccc7ccccc7c56)c5ccc6c(c5)c5ccccc5n6-c5ccccc5)cc4)c23)cc1]

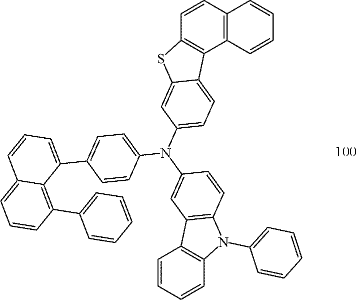 [SMILES: c1ccc(-c2cccc3cccc(-c4ccc(N(c5ccc(-n6c7ccccc7c7ccccc76)cc5)c5ccc6sc7ccc8ccccc8c7c6c5)cc4)c23)cc1]

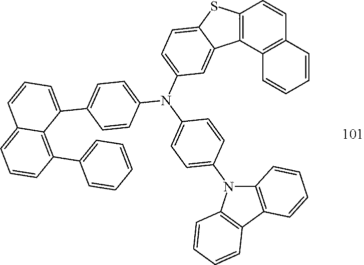 [SMILES: c1ccc(-c2cccc3cccc(-c4ccc(N(c5cccc(-n6c7ccccc7c7ccccc76)c5)c5ccc6sc7ccc8ccccc8c7c6c5)cc4)c23)cc1]

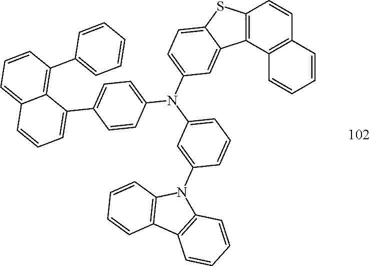 [SMILES: c1ccc(-c2cccc3cccc(-c4ccc(N(c5ccc6sc7ccc8ccccc8c7c6c5)c5ccccc5-n5c6ccccc6c6ccccc65)cc4)c23)cc1]

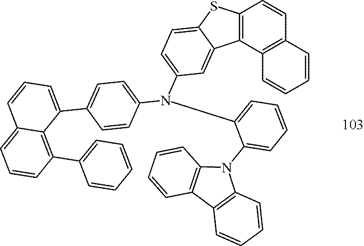 [SMILES: c1ccc(-c2cccc3cccc(-c4ccc(N(c5ccc6sc7ccc8ccccc8c7c6c5)c5cccc6c5c5ccccc5n6-c5ccccc5)cc4)c23)cc1]

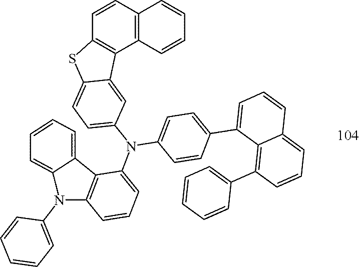 [SMILES: c1ccc(-c2cccc3cccc(-c4ccc(N(c5ccc6sc7ccc8ccccc8c7c6c5)c5cccc6c7ccccc7n(-c7ccccc7)c56)cc4)c23)cc1]

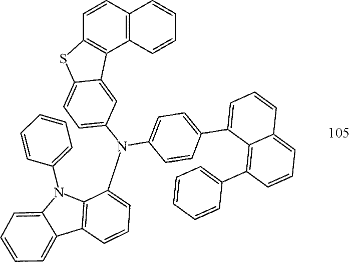 [SMILES: c1ccc(-c2cccc3cccc(-c4ccc(N(c5ccc6c(c5)C(c5ccccc5)(c5ccccc5)c5ccccc5-6)c5ccc6sc7ccc8ccccc8c7c6c5)cc4)c23)cc1]

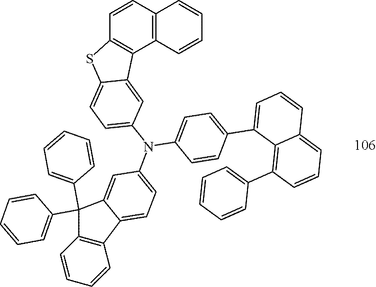 [SMILES: c1ccc(-c2cccc3cccc(-c4ccc(N(c5ccc6sc7ccc8ccccc8c7c6c5)c5cccc6c5-c5ccccc5C6(c5ccccc5)c5ccccc5)cc4)c23)cc1]

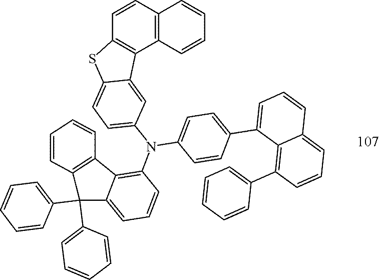 [SMILES: c1ccc(-c2cccc3cccc(-c4ccc(N(c5ccc(C6(c7ccccc7)c7ccccc7-c7ccccc76)cc5)c5ccc6sc7ccc8ccccc8c7c6c5)cc4)c23)cc1]

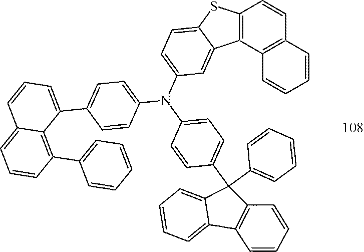 [SMILES: c1ccc(-c2cccc3cccc(-c4ccc(N(c5cccc(C6(c7ccccc7)c7ccccc7-c7ccccc76)c5)c5ccc6sc7ccc8ccccc8c7c6c5)cc4)c23)cc1]

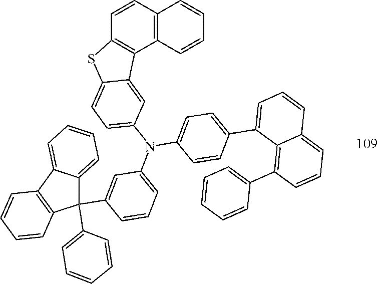 [SMILES: c1ccc(-c2cccc3cccc(-c4ccc(N(c5ccc6c(c5)C5(c7ccccc7-c7ccccc75)c5ccccc5-6)c5ccc6sc7ccc8ccccc8c7c6c5)cc4)c23)cc1]

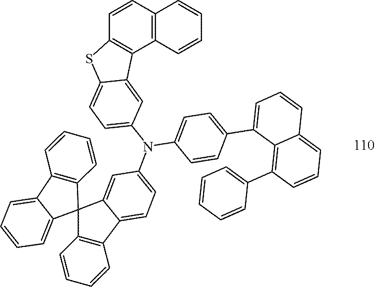 [SMILES: c1ccc(-c2cccc3cccc(-c4ccc(N(c5cccc6c5-c5ccccc5C65c6ccccc6-c6ccccc65)c5cccc6sc7ccc8ccccc8c7c56)cc4)c23)cc1]

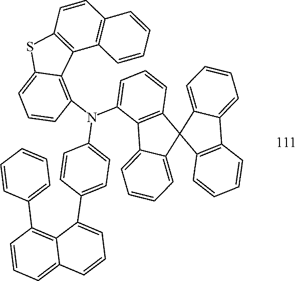 [SMILES: c1ccc(-c2cccc3cccc(-c4ccc(N(c5ccc6c(c5)-c5ccccc5C6(c5ccccc5)c5ccccc5)c5cccc6sc7ccc8ccccc8c7c56)cc4)c23)cc1]

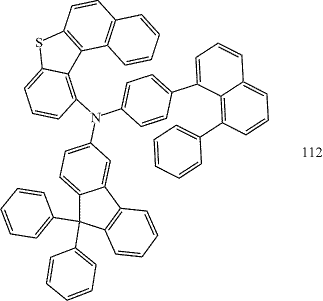 [SMILES: c1ccc(-c2cccc3cccc(-c4ccc(N(c5ccccc5C5(c6ccccc6)c6ccccc6-c6ccccc65)c5cccc6sc7ccc8ccccc8c7c56)cc4)c23)cc1]

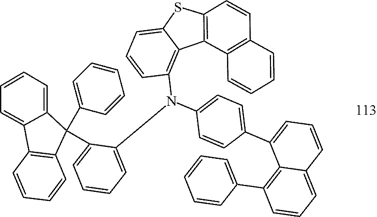 [SMILES: c1ccc(-c2cccc3cccc(-c4ccc(N(c5ccc6c(c5)-c5ccccc5C65c6ccccc6-c6ccccc65)c5cccc6sc7ccc8ccccc8c7c56)cc4)c23)cc1]

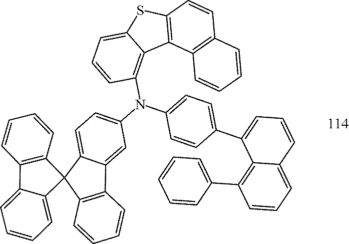 [SMILES: c1ccc(-c2cccc3cccc(-c4ccc(N(c5cccc6c5C5(c7ccccc7-c7ccccc75)c5ccccc5-6)c5cccc6sc7ccc8ccccc8c7c56)cc4)c23)cc1]

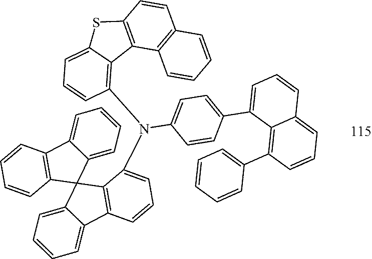 [SMILES: c1ccc(-c2ccc(-c3ccc(N(c4ccc(-c5cccc6cccc(-c7ccccc7)c56)cc4)c4cccc5sc6ccc7ccccc7c6c45)cc3)cc2)cc1]

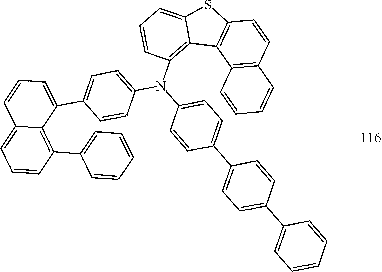 [SMILES: c1ccc(-c2cccc(-c3ccc(N(c4ccc(-c5cccc6cccc(-c7ccccc7)c56)cc4)c4cccc5sc6ccc7ccccc7c6c45)cc3)c2)cc1]

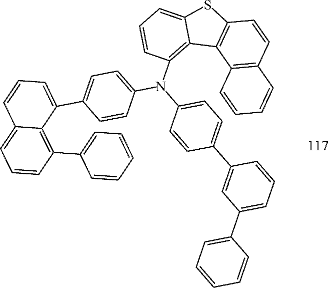 [SMILES: c1ccc(-c2cc(-c3ccccc3)cc(N(c3ccc(-c4cccc5cccc(-c6ccccc6)c45)cc3)c3cccc4sc5ccc6ccccc6c5c34)c2)cc1]

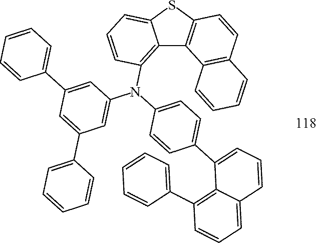 [SMILES: c1ccc(-c2ccc(N(c3ccc(-c4cccc5cccc(-c6ccccc6)c45)cc3)c3cccc4sc5ccc6ccccc6c5c34)c(-c3ccccc3)c2)cc1]

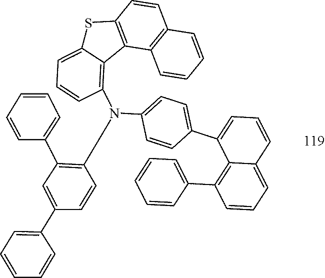 [SMILES: c1ccc(-c2ccc(-c3cccc(N(c4ccc(-c5cccc6cccc(-c7ccccc7)c56)cc4)c4cccc5sc6ccc7ccccc7c6c45)c3)cc2)cc1]

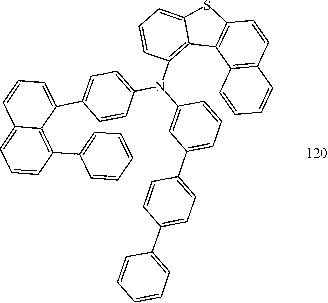 [SMILES: c1ccc(-c2ccc(-c3ccccc3N(c3ccc(-c4cccc5cccc(-c6ccccc6)c45)cc3)c3cccc4sc5ccc6ccccc6c5c34)cc2)cc1]

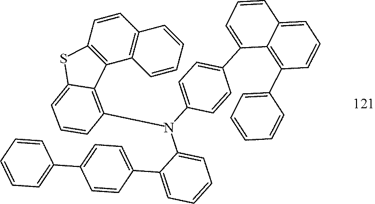 [SMILES: c1ccc(-c2cccc3cccc(-c4ccc(N(c5cc6ccccc6c6ccccc56)c5cc6ccccc6c6c5sc5ccccc56)cc4)c23)cc1]

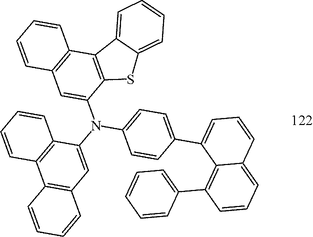 [SMILES: c1ccc(-c2cccc3cccc(-c4ccc(N(c5ccc6c(ccc7ccccc76)c5)c5cc6sc7ccccc7c6c6ccccc56)cc4)c23)cc1]

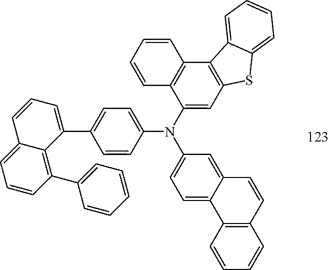 [SMILES: c1ccc(-c2cccc3cccc(-c4ccc(N(c5ccc(-c6cccc7ccccc67)cc5)c5cccc6c5ccc5sc7ccccc7c56)cc4)c23)cc1]

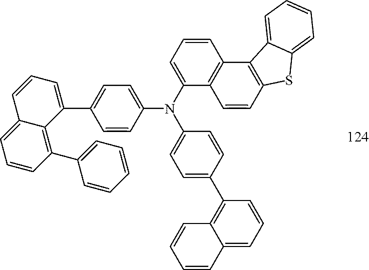 [SMILES: c1ccc(-c2ccc(N(c3ccc(-c4cccc5cccc(-c6ccccc6)c45)cc3)c3ccc4c(ccc5sc6ccccc6c54)c3)c3ccccc23)cc1]

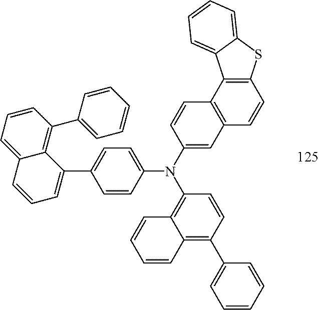 [SMILES: c1ccc(-c2cccc3cccc(-c4ccc(N(c5ccc(-c6ccc7ccccc7c6)cc5)c5ccc6ccc7sc8ccccc8c7c6c5)cc4)c23)cc1]

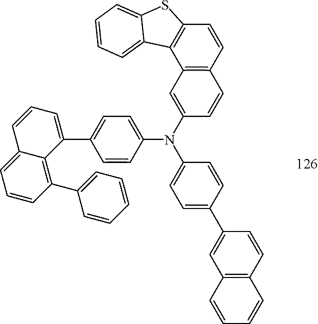 [SMILES: c1ccc(-c2ccc3cc(N(c4ccc(-c5cccc6cccc(-c7ccccc7)c56)cc4)c4cccc5ccc6sc7ccccc7c6c45)ccc3c2)cc1]

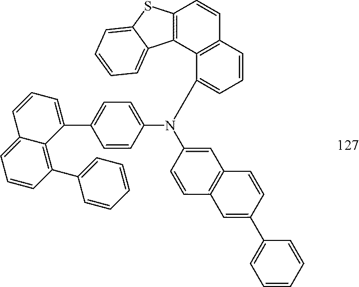 [SMILES: c1ccc(-c2cccc3cccc(-c4ccc(N(c5ccc(-c6ccc(-c7cccc8ccccc78)cc6)cc5)c5cc6ccccc6c6c5sc5ccccc56)cc4)c23)cc1]

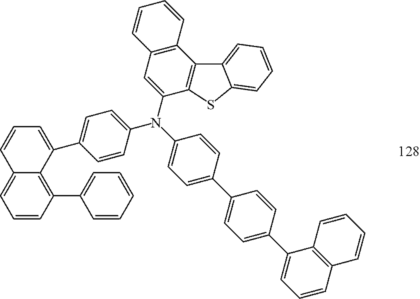 [SMILES: c1ccc(-c2cccc3cccc(-c4ccc(N(c5ccc(-c6ccc7c(c6)oc6ccccc67)cc5)c5cc6sc7ccccc7c6c6ccccc56)cc4)c23)cc1]

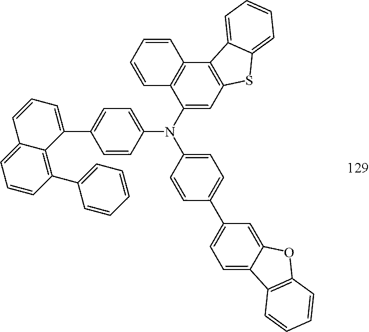 [SMILES: c1ccc(-c2cccc3cccc(-c4ccc(N(c5ccc(-c6ccc7oc8ccccc8c7c6)cc5)c5cccc6c5ccc5sc7ccccc7c56)cc4)c23)cc1]

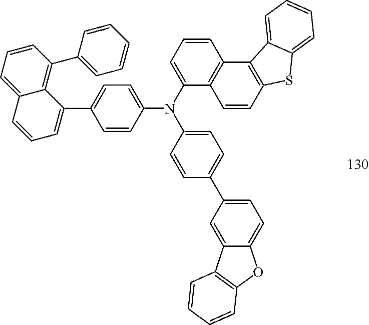 [SMILES: c1ccc(-c2cccc3cccc(-c4ccc(N(c5ccc(-c6cccc7oc8ccccc8c67)cc5)c5ccc6c(ccc7sc8ccccc8c76)c5)cc4)c23)cc1]

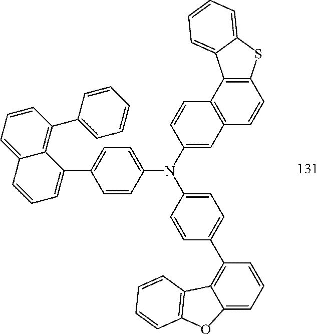 [SMILES: c1ccc(-c2cccc3cccc(-c4ccc(N(c5ccc(-c6cccc7c6oc6ccccc67)cc5)c5cc6sc7ccccc7c6c6ccccc56)cc4)c23)cc1]

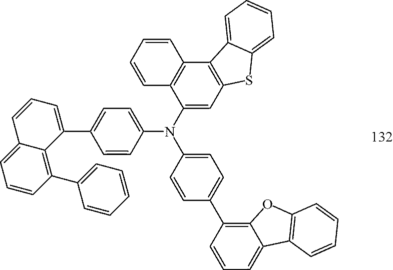 [SMILES: c1ccc(-c2cccc3cccc(-c4ccc(N(c5ccc(-c6ccc7c(c6)sc6ccccc67)cc5)c5cc6ccccc6c6c5sc5ccccc56)cc4)c23)cc1]

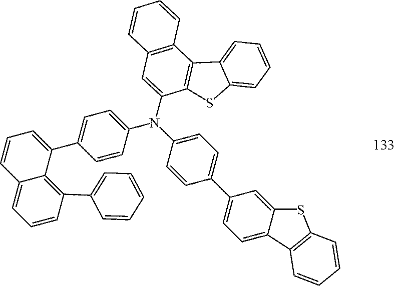 [SMILES: c1ccc(-c2cccc3cccc(-c4ccc(N(c5ccc(-c6ccc7sc8ccccc8c7c6)cc5)c5cccc6ccc7sc8ccccc8c7c56)cc4)c23)cc1]

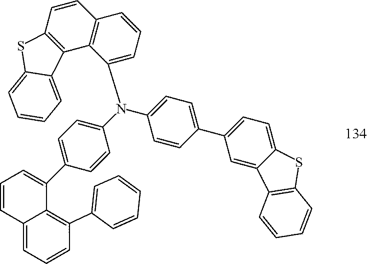 [SMILES: c1ccc(-c2cccc3cccc(-c4ccc(N(c5ccc(-c6cccc7sc8ccccc8c67)cc5)c5ccc6ccc7sc8ccccc8c7c6c5)cc4)c23)cc1]

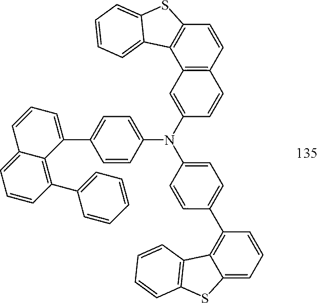 [SMILES: c1ccc(-c2cccc3cccc(-c4ccc(N(c5ccc(-c6cccc7c6sc6ccccc67)cc5)c5ccc6c(ccc7sc8ccccc8c76)c5)cc4)c23)cc1]

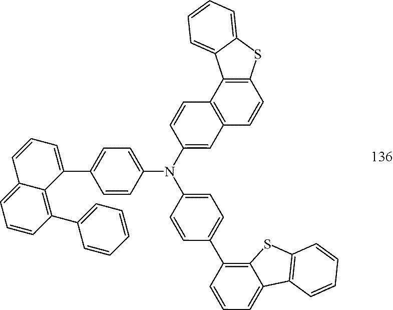 [SMILES: c1ccc(-c2cccc3cccc(-c4ccc(N(c5cccc(-c6cccc7oc8ccccc8c67)c5)c5cccc6c5ccc5sc7ccccc7c56)cc4)c23)cc1]

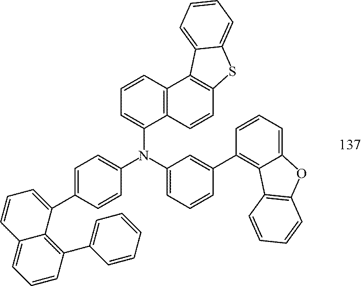 [SMILES: c1ccc(-c2cccc3cccc(-c4ccc(N(c5cccc(-c6cccc7c6oc6ccccc67)c5)c5cc6sc7ccccc7c6c6ccccc56)cc4)c23)cc1]

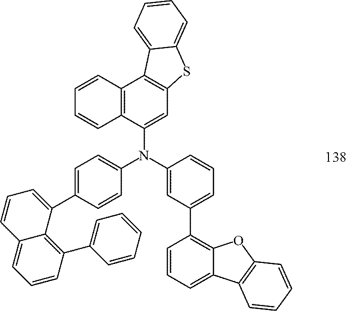 [SMILES: c1ccc(-c2cccc3cccc(-c4ccc(N(c5cccc(-c6cccc7sc8ccccc8c67)c5)c5cc6ccccc6c6c5sc5ccccc56)cc4)c23)cc1]

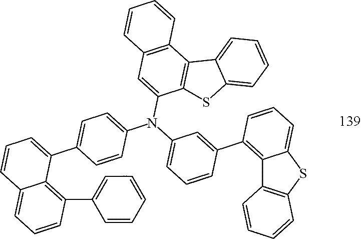 [SMILES: CC1(C)c2ccccc2-c2cc(-c3ccc(N(c4ccc(-c5cccc6cccc(-c7ccccc7)c56)cc4)c4cccc5ccc6sc7ccccc7c6c45)cc3)ccc21]

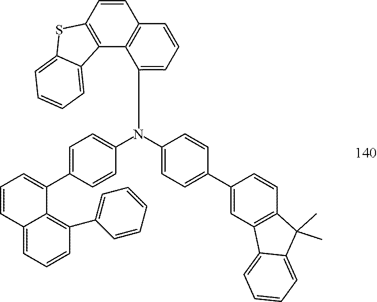 [SMILES: CC1(C)c2ccccc2-c2c(-c3ccc(N(c4ccc(-c5cccc6cccc(-c7ccccc7)c56)cc4)c4ccc5ccc6sc7ccccc7c6c5c4)cc3)cccc21]

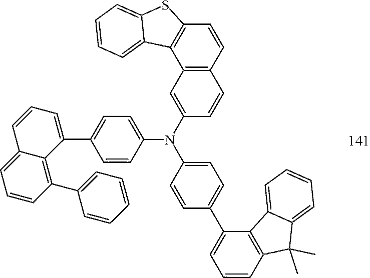 [SMILES: c1ccc(-c2cccc3cccc(-c4ccc(N(c5cccc(-c6cccc7cccc(-c8ccccc8)c67)c5)c5cccc6c5ccc5sc7ccccc7c56)cc4)c23)cc1]

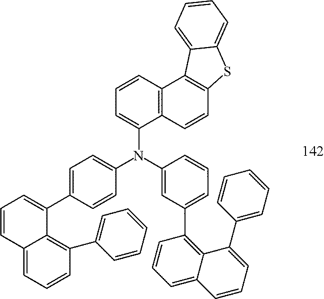 [SMILES: c1ccc(-c2cccc3cccc(-c4ccc(N(c5ccc6c(ccc7sc8ccccc8c76)c5)c5ccccc5-c5cccc6cccc(-c7ccccc7)c56)cc4)c23)cc1]

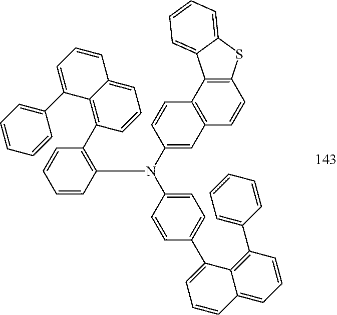 [SMILES: c1ccc(-c2ccc(N(c3cccc(-c4cccc5cccc(-c6ccccc6)c45)c3)c3cccc4c3sc3ccc5ccccc5c34)cc2)cc1]

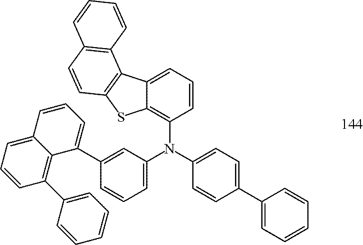 [SMILES: c1ccc(-c2cccc3cccc(-c4cccc(N(c5cccc6ccccc56)c5cccc6c5sc5ccc7ccccc7c56)c4)c23)cc1]

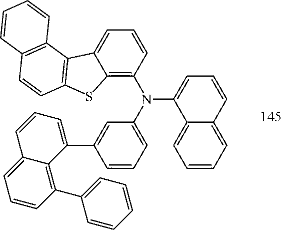 [SMILES: c1ccc(-c2cccc3cccc(-c4cccc(N(c5ccc6oc7ccccc7c6c5)c5cccc6c5sc5ccc7ccccc7c56)c4)c23)cc1]

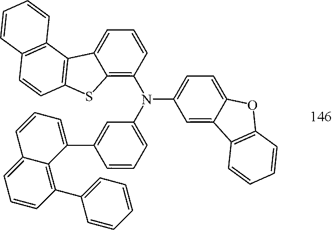 [SMILES: c1ccc(-c2cccc3cccc(-c4cccc(N(c5ccc6c(c5)sc5ccccc56)c5ccc6c(c5)sc5ccc7ccccc7c56)c4)c23)cc1]

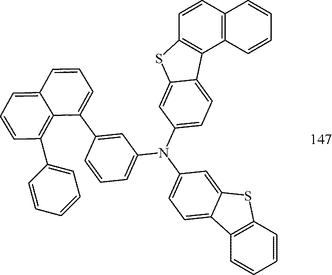 [SMILES: CC1(C)c2ccccc2-c2ccc(N(c3cccc(-c4cccc5cccc(-c6ccccc6)c45)c3)c3ccc4c(c3)sc3ccc5ccccc5c34)cc21]

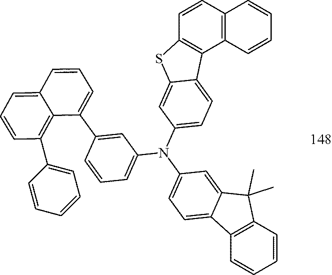 [SMILES: c1ccc(-c2cccc3cccc(-c4cccc(N(c5ccc6c(c5)sc5ccc7ccccc7c56)c5ccc6c7ccccc7n(-c7ccccc7)c6c5)c4)c23)cc1]

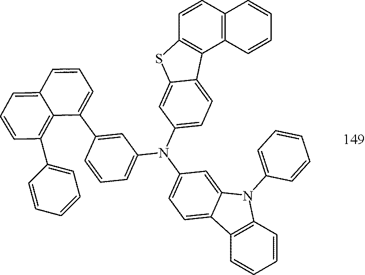 [SMILES: c1ccc(-c2cccc(-c3ccc(N(c4cccc(-c5cccc6cccc(-c7ccccc7)c56)c4)c4cccc5sc6ccc7ccccc7c6c45)cc3)c2)cc1]

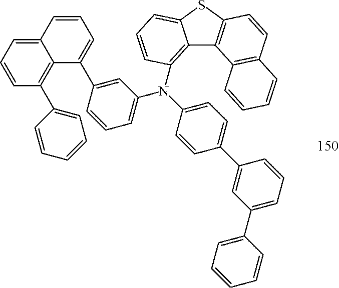 [SMILES: c1ccc(-c2ccc(N(c3ccccc3-c3cccc4cccc(-c5ccccc5)c34)c3cccc4c3sc3ccc5ccccc5c34)cc2)cc1]

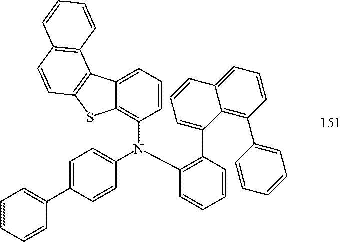 [SMILES: c1ccc(-c2cccc3cccc(-c4ccccc4N(c4cccc5ccccc45)c4cccc5c4sc4ccc6ccccc6c45)c23)cc1]

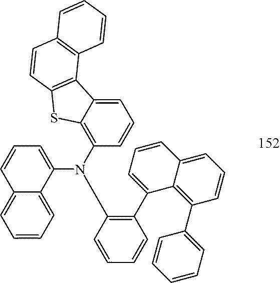 [SMILES: c1ccc(-c2cccc3cccc(-c4ccccc4N(c4ccc5oc6ccccc6c5c4)c4cccc5c4sc4ccc6ccccc6c45)c23)cc1]

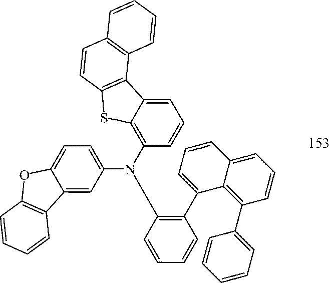 [SMILES: c1ccc(-c2cccc3cccc(-c4ccccc4N(c4ccc5c(c4)sc4ccccc45)c4ccc5c(c4)sc4ccc6ccccc6c45)c23)cc1]

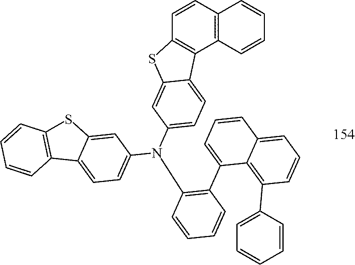 [SMILES: CC1(C)c2ccccc2-c2ccc(N(c3ccc4c(c3)sc3ccc5ccccc5c34)c3ccccc3-c3cccc4cccc(-c5ccccc5)c34)cc21]

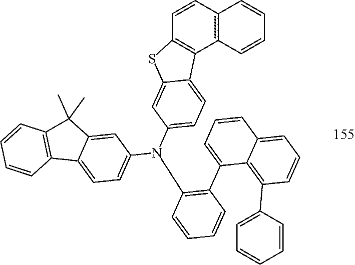 [SMILES: c1ccc(-c2cccc3cccc(-c4ccccc4N(c4ccc5c(c4)sc4ccc6ccccc6c45)c4ccc5c6ccccc6n(-c6ccccc6)c5c4)c23)cc1]

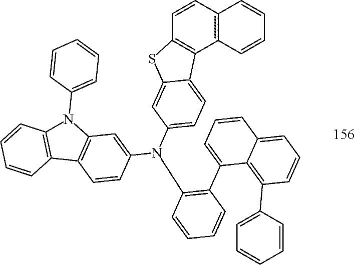 [SMILES: c1ccc(-c2cccc(-c3ccc(N(c4ccccc4-c4cccc5cccc(-c6ccccc6)c45)c4cccc5sc6ccc7ccccc7c6c45)cc3)c2)cc1]

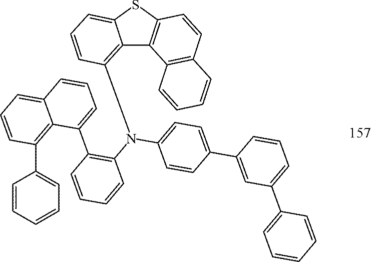 [SMILES: c1ccc(-c2cccc3cccc(-c4ccc(N(c5ccccc5)c5cccc6c5oc5ccc7ccccc7c56)cc4)c23)cc1]

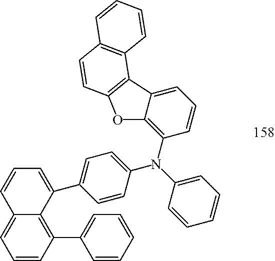 [SMILES: c1ccc(-c2ccc(N(c3ccc(-c4cccc5cccc(-c6ccccc6)c45)cc3)c3cccc4c3oc3ccc5ccccc5c34)cc2)cc1]

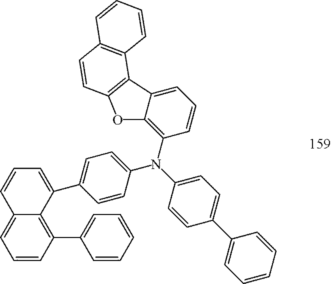 [SMILES: c1ccc(-c2cccc(N(c3ccc(-c4cccc5cccc(-c6ccccc6)c45)cc3)c3cccc4c3oc3ccc5ccccc5c34)c2)cc1]

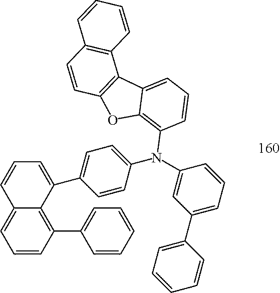 [SMILES: c1ccc(-c2ccccc2N(c2ccc(-c3cccc4cccc(-c5ccccc5)c34)cc2)c2cccc3c2oc2ccc4ccccc4c23)cc1]

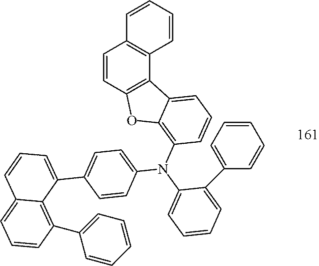 [SMILES: c1ccc(-c2cccc3cccc(-c4ccc(N(c5cccc6ccccc56)c5cccc6c5oc5cc7ccccc7cc56)cc4)c23)cc1]

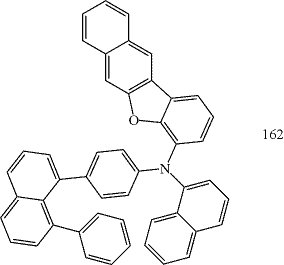 [SMILES: c1ccc(-c2cccc3cccc(-c4ccc(N(c5ccc6ccccc6c5)c5cccc6c5oc5cc7ccccc7cc56)cc4)c23)cc1]

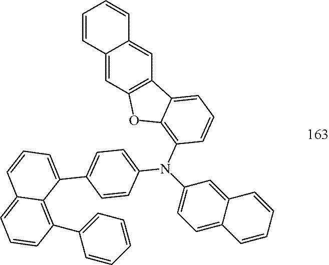 [SMILES: c1ccc(-c2cccc3cccc(-c4ccc(N(c5ccc6c(c5)oc5ccccc56)c5cccc6c5oc5cc7ccccc7cc56)cc4)c23)cc1]

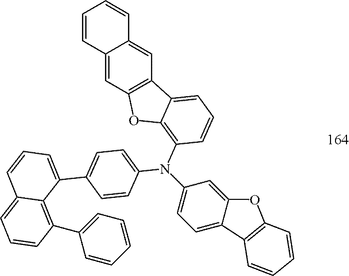 [SMILES: c1ccc(-c2cccc3cccc(-c4ccc(N(c5ccc6oc7ccccc7c6c5)c5cccc6c5oc5cc7ccccc7cc56)cc4)c23)cc1]

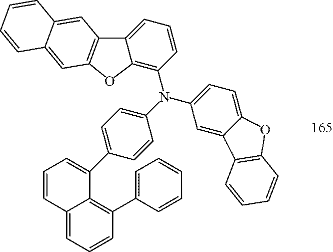 [SMILES: c1ccc(-c2cccc3cccc(-c4ccc(N(c5cccc6c5oc5ccc7ccccc7c56)c5cccc6oc7ccccc7c56)cc4)c23)cc1]

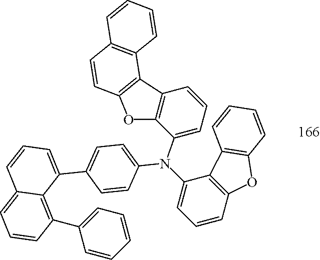 [SMILES: c1ccc(-c2cccc3cccc(-c4ccc(N(c5cccc6c5oc5ccccc56)c5cccc6c5oc5ccc7ccccc7c56)cc4)c23)cc1]

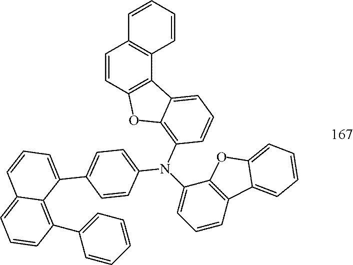 [SMILES: c1ccc(-c2cccc3cccc(-c4ccc(N(c5ccc6c(c5)oc5ccc7ccccc7c56)c5ccc6c(c5)sc5ccccc56)cc4)c23)cc1]

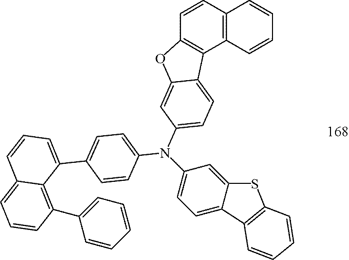 [SMILES: c1ccc(-c2cccc3cccc(-c4ccc(N(c5ccc6c(c5)oc5ccc7ccccc7c56)c5ccc6sc7ccccc7c6c5)cc4)c23)cc1]

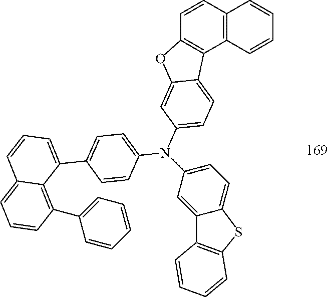 [SMILES: c1ccc(-c2cccc3cccc(-c4ccc(N(c5ccc6c(c5)oc5c7ccccc7ccc65)c5cccc6sc7ccccc7c56)cc4)c23)cc1]

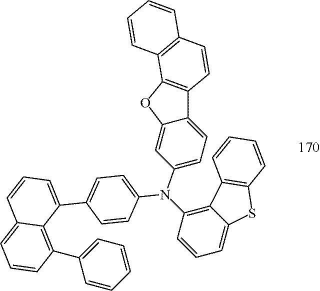 [SMILES: c1ccc(-c2cccc3cccc(-c4ccc(N(c5ccc6c(c5)oc5c7ccccc7ccc65)c5cccc6c5sc5ccccc56)cc4)c23)cc1]

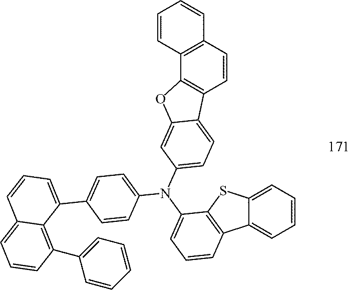 [SMILES: CC1(C)c2ccccc2-c2ccc(N(c3ccc(-c4cccc5cccc(-c6ccccc6)c45)cc3)c3ccc4c(c3)oc3c5ccccc5ccc43)cc21]

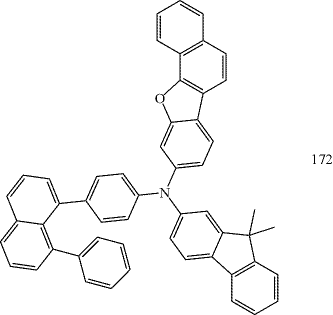 [SMILES: CC1(C)c2ccccc2-c2cc(N(c3ccc(-c4cccc5cccc(-c6ccccc6)c45)cc3)c3ccc4c(c3)oc3c5ccccc5ccc43)ccc21]

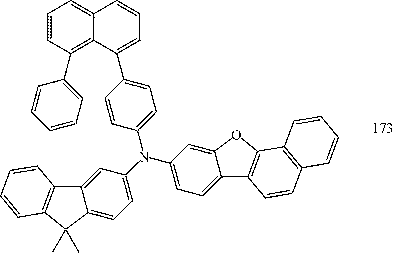

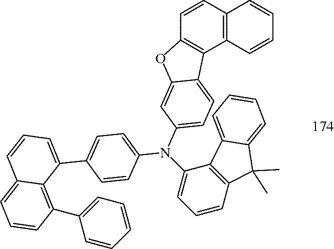 [SMILES: CC1(C)c2ccccc2-c2cccc(N(c3ccc(-c4cccc5cccc(-c6ccccc6)c45)cc3)c3ccc4c(c3)oc3ccc5ccccc5c34)c21]

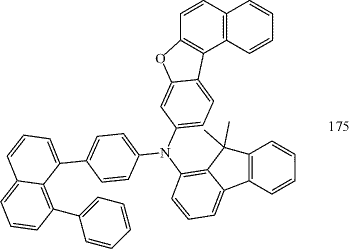 [SMILES: CC1(C)c2ccccc2-c2cccc(N(c3ccc(-c4cccc5cccc(-c6ccccc6)c45)cc3)c3ccc4c(c3)oc3ccc5ccccc5c34)c21]

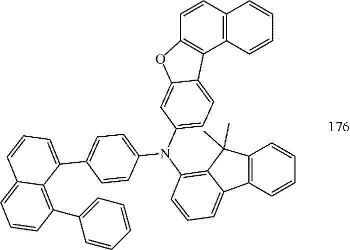 [SMILES: c1ccc(-c2cccc3cccc(-c4ccc(N(c5ccc6c(c5)oc5ccc7ccccc7c56)c5ccc6c7ccccc7n(-c7ccccc7)c6c5)cc4)c23)cc1]

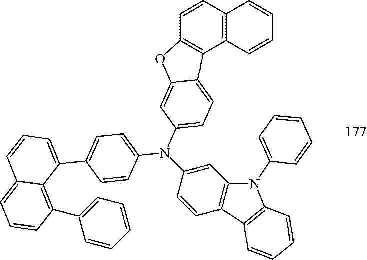 [SMILES: c1ccc(-c2cccc3cccc(-c4ccc(N(c5ccc6c(c5)oc5ccc7ccccc7c56)c5ccc6c(c5)c5ccccc5n6-c5ccccc5)cc4)c23)cc1]

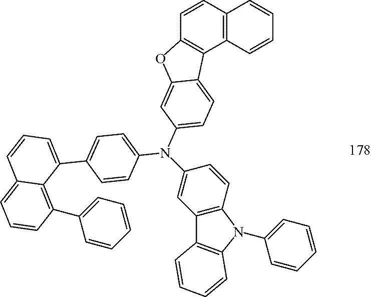 [SMILES: c1ccc(-c2cccc3cccc(-c4ccc(N(c5ccc(-n6c7ccccc7c7ccccc76)cc5)c5ccc6oc7ccc8ccccc8c7c6c5)cc4)c23)cc1]

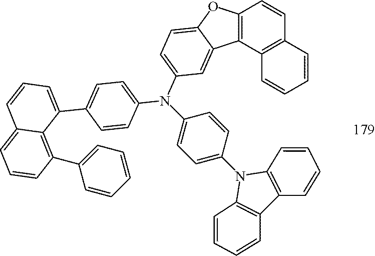 [SMILES: c1ccc(-c2cccc3cccc(-c4ccc(N(c5cccc(-n6c7ccccc7c7ccccc76)c5)c5ccc6oc7ccc8ccccc8c7c6c5)cc4)c23)cc1]

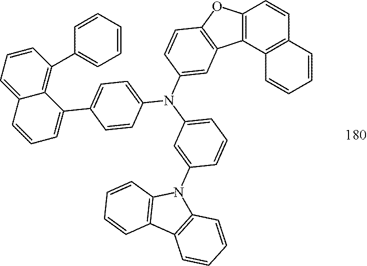 [SMILES: c1ccc(-c2cccc3cccc(-c4ccc(N(c5ccc6oc7ccc8ccccc8c7c6c5)c5ccccc5-n5c6ccccc6c6ccccc65)cc4)c23)cc1]

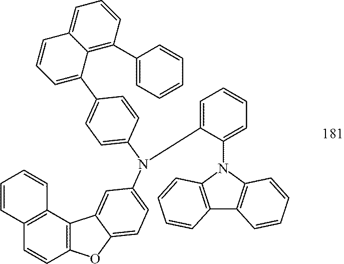 [SMILES: c1ccc(-c2cccc3cccc(-c4ccc(N(c5ccc6oc7ccc8ccccc8c7c6c5)c5cccc6c5c5ccccc5n6-c5ccccc5)cc4)c23)cc1]

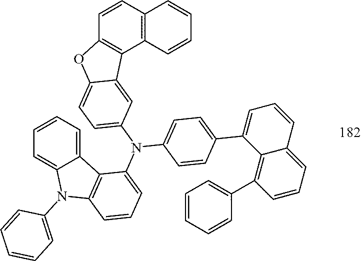 [SMILES: c1ccc(-c2cccc3cccc(-c4ccc(N(c5ccc6oc7ccc8ccccc8c7c6c5)c5cccc6c7ccccc7n(-c7ccccc7)c56)cc4)c23)cc1]

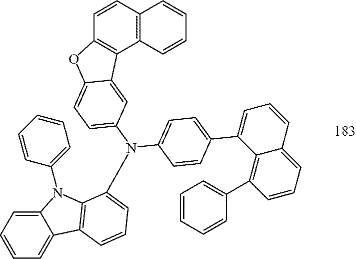 [SMILES: c1ccc(-c2cccc3cccc(-c4ccc(N(c5ccc6c(c5)C(c5ccccc5)(c5ccccc5)c5ccccc5-6)c5ccc6oc7ccc8ccccc8c7c6c5)cc4)c23)cc1]

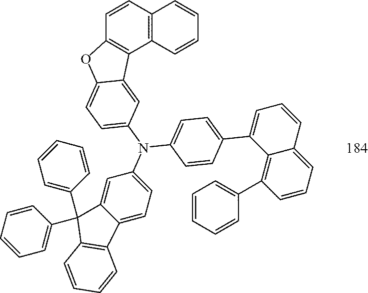 [SMILES: c1ccc(-c2cccc3cccc(-c4ccc(N(c5ccc6oc7ccc8ccccc8c7c6c5)c5cccc6c5-c5ccccc5C6(c5ccccc5)c5ccccc5)cc4)c23)cc1]

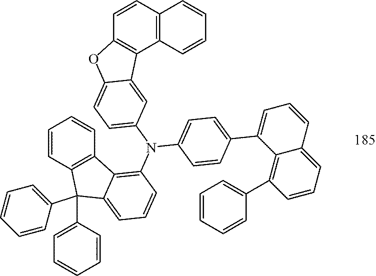 [SMILES: c1ccc(-c2cccc3cccc(-c4ccc(N(c5ccc(C6(c7ccccc7)c7ccccc7-c7ccccc76)cc5)c5ccc6oc7ccc8ccccc8c7c6c5)cc4)c23)cc1]

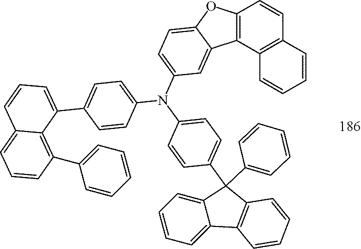 [SMILES: c1ccc(-c2cccc3cccc(-c4ccc(N(c5cccc(C6(c7ccccc7)c7ccccc7-c7ccccc76)c5)c5ccc6oc7ccc8ccccc8c7c6c5)cc4)c23)cc1]

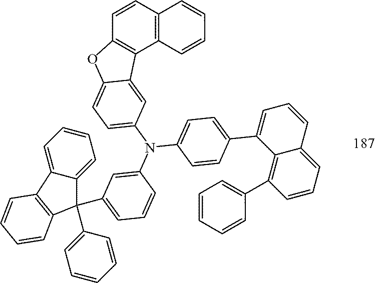 [SMILES: c1ccc(-c2cccc3cccc(-c4ccc(N(c5ccc6c(c5)C5(c7ccccc7-c7ccccc75)c5ccccc5-6)c5ccc6oc7ccc8ccccc8c7c6c5)cc4)c23)cc1]

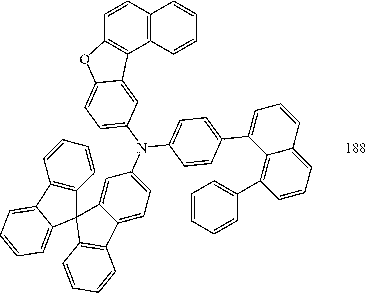 [SMILES: c1ccc(-c2cccc3cccc(-c4ccc(N(c5cccc6c5-c5ccccc5C65c6ccccc6-c6ccccc65)c5cccc6oc7ccc8ccccc8c7c56)cc4)c23)cc1]

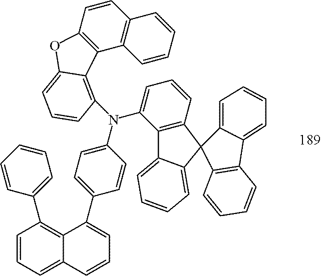 [SMILES: c1ccc(-c2cccc3cccc(-c4ccc(N(c5ccc6c(c5)-c5ccccc5C6(c5ccccc5)c5ccccc5)c5cccc6oc7ccc8ccccc8c7c56)cc4)c23)cc1]

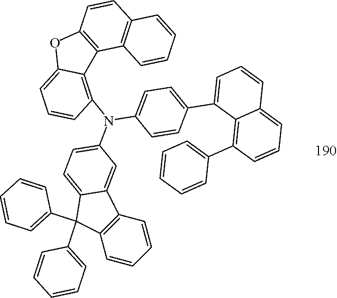 [SMILES: c1ccc(-c2cccc3cccc(-c4ccc(N(c5ccccc5C5(c6ccccc6)c6ccccc6-c6ccccc65)c5cccc6oc7ccc8ccccc8c7c56)cc4)c23)cc1]

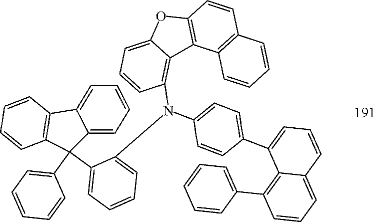 [SMILES: c1ccc(-c2cccc3cccc(-c4ccc(N(c5ccc6c(c5)-c5ccccc5C65c6ccccc6-c6ccccc65)c5cccc6oc7ccc8ccccc8c7c56)cc4)c23)cc1]

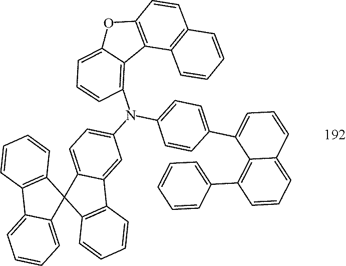 [SMILES: c1ccc(-c2cccc3cccc(-c4ccc(N(c5cccc6c5C5(c7ccccc7-c7ccccc75)c5ccccc5-6)c5cccc6oc7ccc8ccccc8c7c56)cc4)c23)cc1]

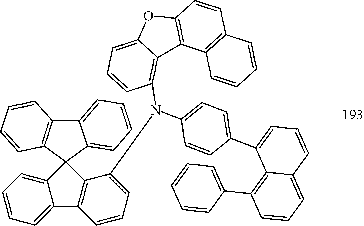 [SMILES: c1ccc(-c2ccc(-c3ccc(N(c4ccc(-c5cccc6cccc(-c7ccccc7)c56)cc4)c4cccc5oc6ccc7ccccc7c6c45)cc3)cc2)cc1]

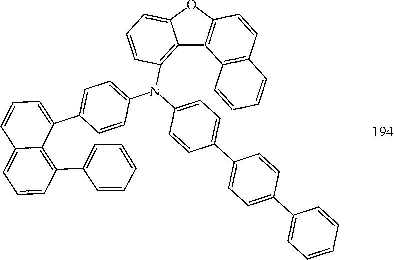 [SMILES: c1ccc(-c2cccc(-c3ccc(N(c4ccc(-c5cccc6cccc(-c7ccccc7)c56)cc4)c4cccc5oc6ccc7ccccc7c6c45)cc3)c2)cc1]

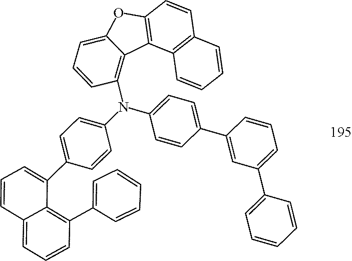 [SMILES: c1ccc(-c2cc(-c3ccccc3)cc(N(c3ccc(-c4cccc5cccc(-c6ccccc6)c45)cc3)c3cccc4oc5ccc6ccccc6c5c34)c2)cc1]

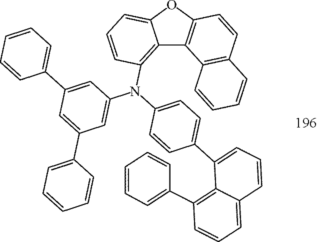 [SMILES: c1ccc(-c2ccc(N(c3ccc(-c4cccc5cccc(-c6ccccc6)c45)cc3)c3cccc4oc5ccc6ccccc6c5c34)c(-c3ccccc3)c2)cc1]

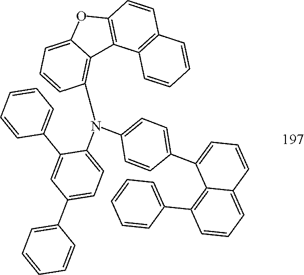 [SMILES: c1ccc(-c2ccc(-c3cccc(N(c4ccc(-c5cccc6cccc(-c7ccccc7)c56)cc4)c4cccc5oc6ccc7ccccc7c6c45)c3)cc2)cc1]

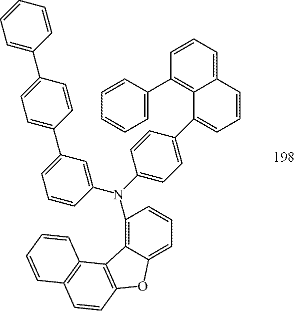 [SMILES: c1ccc(-c2ccc(-c3ccccc3N(c3ccc(-c4cccc5cccc(-c6ccccc6)c45)cc3)c3cccc4oc5ccc6ccccc6c5c34)cc2)cc1]

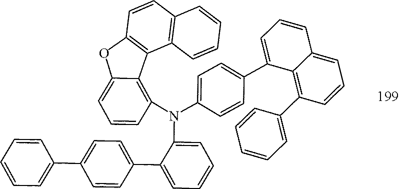 [SMILES: c1ccc(-c2cccc3cccc(-c4ccc(N(c5cc6ccccc6c6ccccc56)c5cc6ccccc6c6c5oc5ccccc56)cc4)c23)cc1]

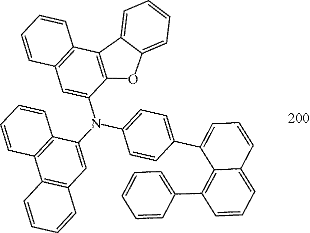 [SMILES: c1ccc(-c2cccc3cccc(-c4ccc(N(c5ccc6c(ccc7ccccc76)c5)c5cc6oc7ccccc7c6c6ccccc56)cc4)c23)cc1]

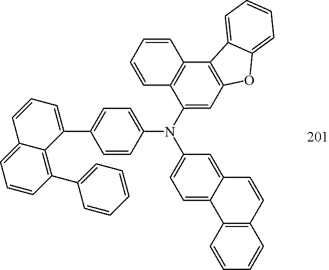 [SMILES: c1ccc(-c2cccc3cccc(-c4ccc(N(c5ccc(-c6cccc7ccccc67)cc5)c5cccc6c5ccc5oc7ccccc7c56)cc4)c23)cc1]

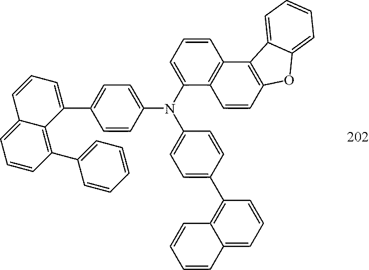 [SMILES: c1ccc(-c2ccc(N(c3ccc(-c4cccc5cccc(-c6ccccc6)c45)cc3)c3ccc4c(ccc5oc6ccccc6c54)c3)c3ccccc23)cc1]

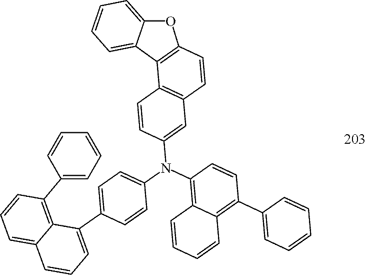 [SMILES: c1ccc(-c2cccc3cccc(-c4ccc(N(c5ccc(-c6ccc7ccccc7c6)cc5)c5ccc6ccc7oc8ccccc8c7c6c5)cc4)c23)cc1]

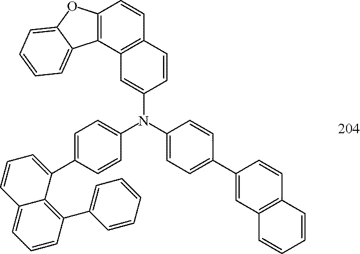 [SMILES: c1ccc(-c2ccc3cc(N(c4ccc(-c5cccc6cccc(-c7ccccc7)c56)cc4)c4cccc5ccc6oc7ccccc7c6c45)ccc3c2)cc1]

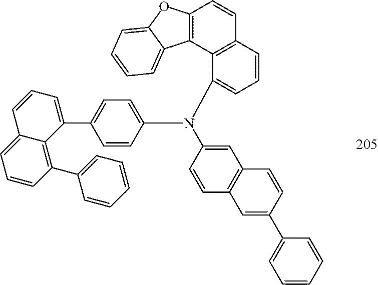 [SMILES: c1ccc(-c2cccc3cccc(-c4ccc(N(c5ccc(-c6ccc(-c7cccc8ccccc78)cc6)cc5)c5cc6ccccc6c6c5oc5ccccc56)cc4)c23)cc1]

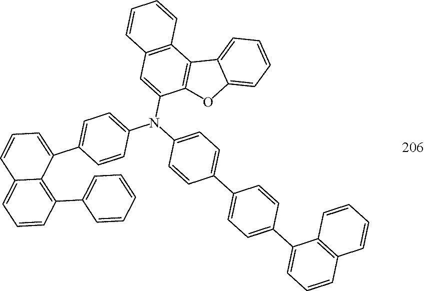 [SMILES: c1ccc(-c2cccc3cccc(-c4ccc(N(c5ccc(-c6ccc7c(c6)oc6ccccc67)cc5)c5cc6oc7ccccc7c6c6ccccc56)cc4)c23)cc1]

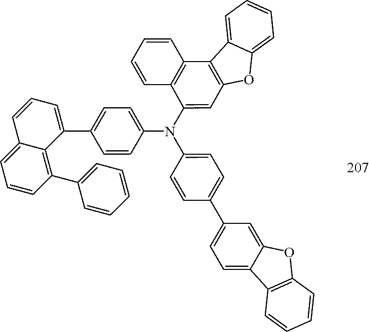 [SMILES: c1ccc(-c2cccc3cccc(-c4ccc(N(c5ccc(-c6ccc7oc8ccccc8c7c6)cc5)c5cccc6c5ccc5oc7ccccc7c56)cc4)c23)cc1]

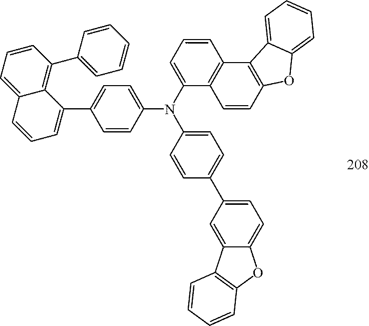 [SMILES: c1ccc(-c2cccc3cccc(-c4ccc(N(c5ccc(-c6cccc7oc8ccccc8c67)cc5)c5ccc6c(ccc7oc8ccccc8c76)c5)cc4)c23)cc1]

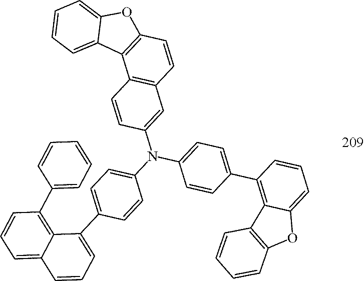 [SMILES: c1ccc(-c2cccc3cccc(-c4ccc(N(c5ccc(-c6cccc7c6oc6ccccc67)cc5)c5cc6oc7ccccc7c6c6ccccc56)cc4)c23)cc1]

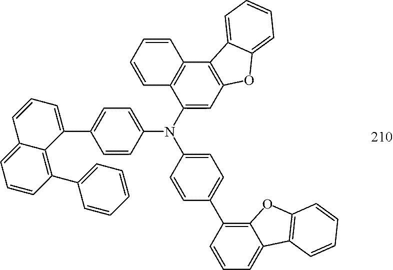 [SMILES: c1ccc(-c2cccc3cccc(-c4ccc(N(c5ccc(-c6ccc7c(c6)sc6ccccc67)cc5)c5cc6ccccc6c6c5oc5ccccc56)cc4)c23)cc1]

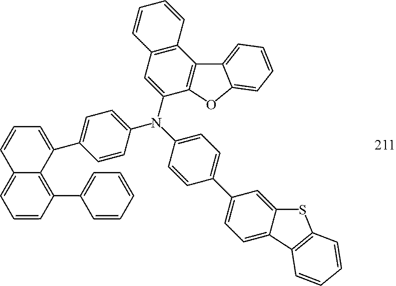 [SMILES: c1ccc(-c2cccc3cccc(-c4ccc(N(c5ccc(-c6ccc7sc8ccccc8c7c6)cc5)c5cccc6ccc7oc8ccccc8c7c56)cc4)c23)cc1]

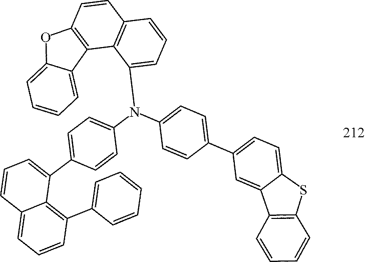 [SMILES: c1ccc(-c2cccc3cccc(-c4ccc(N(c5ccc(-c6cccc7sc8ccccc8c67)cc5)c5ccc6ccc7oc8ccccc8c7c6c5)cc4)c23)cc1]

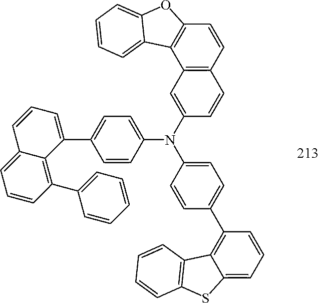 [SMILES: c1ccc(-c2cccc3cccc(-c4ccc(N(c5ccc(-c6cccc7c6sc6ccccc67)cc5)c5ccc6c(ccc7oc8ccccc8c76)c5)cc4)c23)cc1]

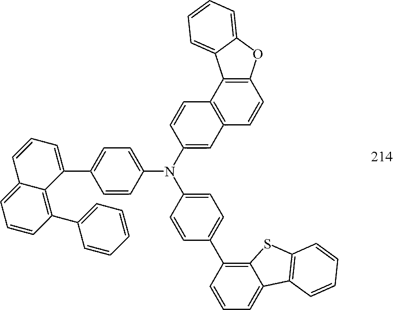 [SMILES: c1ccc(-c2cccc3cccc(-c4ccc(N(c5cccc(-c6cccc7oc8ccccc8c67)c5)c5cccc6c5ccc5oc7ccccc7c56)cc4)c23)cc1]

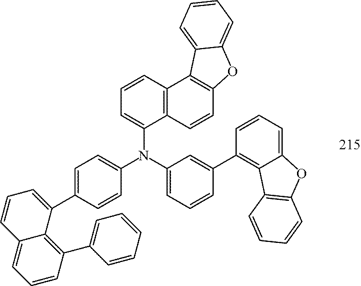 [SMILES: c1ccc(-c2cccc3cccc(-c4ccc(N(c5cccc(-c6cccc7c6oc6ccccc67)c5)c5cc6oc7ccccc7c6c6ccccc56)cc4)c23)cc1]

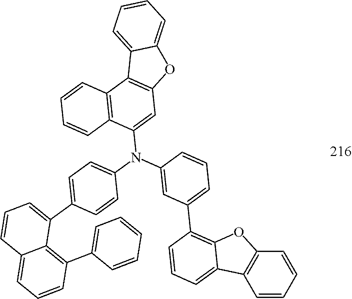 [SMILES: c1ccc(-c2cccc3cccc(-c4ccc(N(c5cccc(-c6cccc7sc8ccccc8c67)c5)c5cc6ccccc6c6c5oc5ccccc56)cc4)c23)cc1]

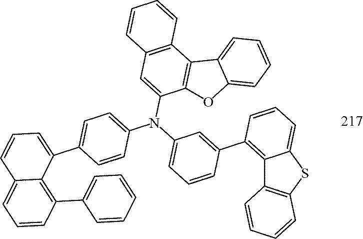 [SMILES: CC1(C)c2ccccc2-c2cc(-c3ccc(N(c4ccc(-c5cccc6cccc(-c7ccccc7)c56)cc4)c4cccc5ccc6oc7ccccc7c6c45)cc3)ccc21]

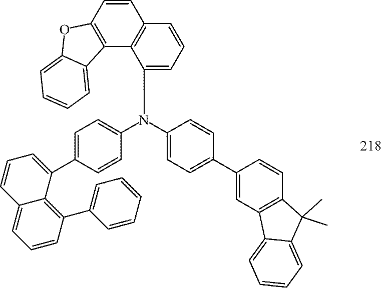 [SMILES: CC1(C)c2ccccc2-c2c(-c3ccc(N(c4ccc(-c5cccc6cccc(-c7ccccc7)c56)cc4)c4ccc5ccc6oc7ccccc7c6c5c4)cc3)cccc21]

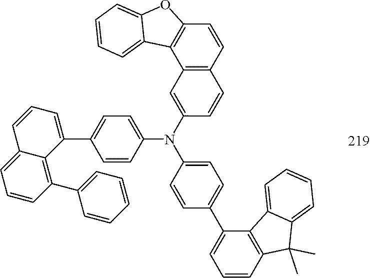 [SMILES: c1ccc(-c2cccc3cccc(-c4ccc(N(c5cccc(-c6cccc7cccc(-c8ccccc8)c67)c5)c5cccc6c5ccc5oc7ccccc7c56)cc4)c23)cc1]

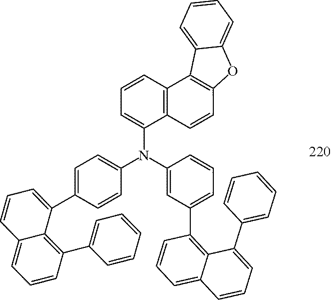 [SMILES: c1ccc(-c2cccc3cccc(-c4ccc(N(c5ccc6c(ccc7oc8ccccc8c76)c5)c5ccccc5-c5cccc6cccc(-c7ccccc7)c56)cc4)c23)cc1]

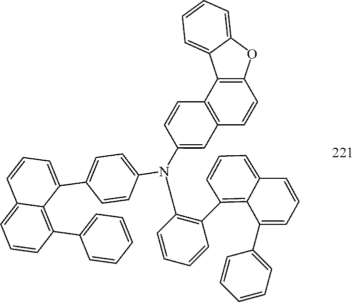 [SMILES: c1ccc(-c2ccc(N(c3cccc(-c4cccc5cccc(-c6ccccc6)c45)c3)c3cccc4c3oc3ccc5ccccc5c34)cc2)cc1]

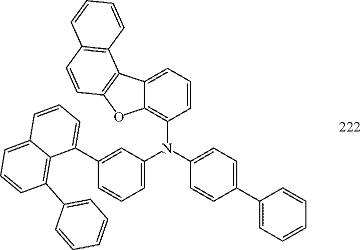 [SMILES: c1ccc(-c2cccc3cccc(-c4cccc(N(c5cccc6ccccc56)c5cccc6c5oc5ccc7ccccc7c56)c4)c23)cc1]

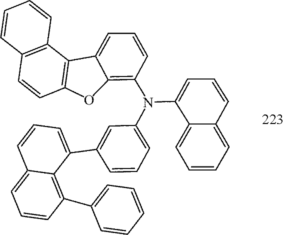 [SMILES: c1ccc(-c2cccc3cccc(-c4cccc(N(c5ccc6oc7ccccc7c6c5)c5cccc6c5oc5ccc7ccccc7c56)c4)c23)cc1]

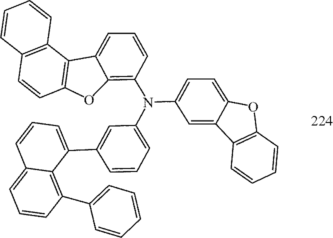 [SMILES: c1ccc(-c2cccc3cccc(-c4cccc(N(c5ccc6c(c5)oc5ccc7ccccc7c56)c5ccc6c(c5)sc5ccccc56)c4)c23)cc1]

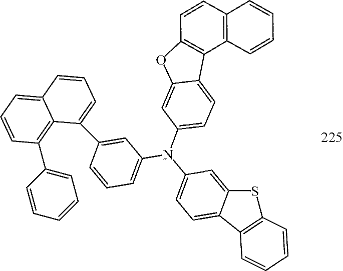 [SMILES: CC1(C)c2ccccc2-c2ccc(N(c3cccc(-c4cccc5cccc(-c6ccccc6)c45)c3)c3ccc4c(c3)oc3ccc5ccccc5c34)cc21]

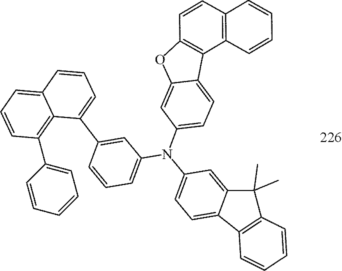 [SMILES: c1ccc(-c2cccc3cccc(-c4cccc(N(c5ccc6c(c5)oc5ccc7ccccc7c56)c5ccc6c7ccccc7n(-c7ccccc7)c6c5)c4)c23)cc1]

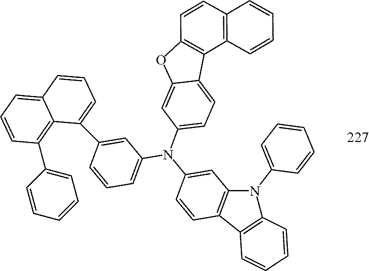 [SMILES: c1ccc(-c2cccc(-c3ccc(N(c4cccc(-c5cccc6cccc(-c7ccccc7)c56)c4)c4cccc5oc6ccc7ccccc7c6c45)cc3)c2)cc1]

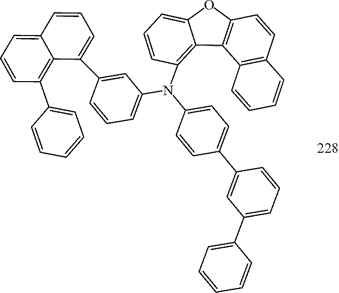 [SMILES: c1ccc(-c2ccc(N(c3ccccc3-c3cccc4cccc(-c5ccccc5)c34)c3cccc4c3oc3ccc5ccccc5c34)cc2)cc1]

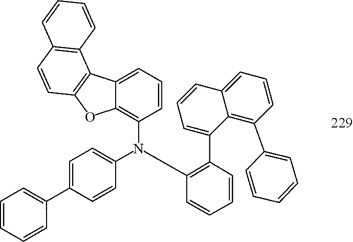 [SMILES: c1ccc(-c2cccc3cccc(-c4ccccc4N(c4cccc5ccccc45)c4cccc5c4oc4ccc6ccccc6c45)c23)cc1]

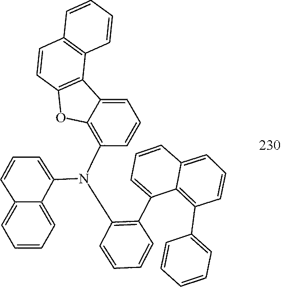 [SMILES: c1ccc(-c2cccc3cccc(-c4ccccc4N(c4ccc5oc6ccccc6c5c4)c4cccc5c4oc4ccc6ccccc6c45)c23)cc1]

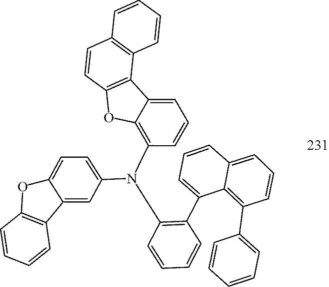 [SMILES: c1ccc(-c2cccc3cccc(-c4ccccc4N(c4ccc5c(c4)oc4ccc6ccccc6c45)c4ccc5c(c4)sc4ccccc45)c23)cc1]

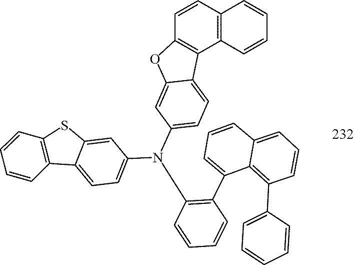 [SMILES: CC1(C)c2ccccc2-c2ccc(N(c3ccc4c(c3)oc3ccc5ccccc5c34)c3ccccc3-c3cccc4cccc(-c5ccccc5)c34)cc21]

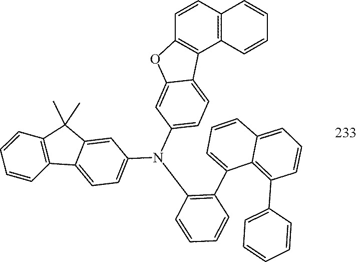 [SMILES: c1ccc(-c2cccc3cccc(-c4ccccc4N(c4ccc5c(c4)oc4ccc6ccccc6c45)c4ccc5c6ccccc6n(-c6ccccc6)c5c4)c23)cc1]

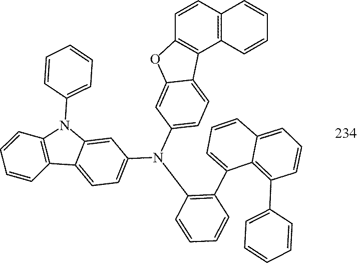 [SMILES: c1ccc(-c2cccc(-c3ccc(N(c4ccccc4-c4cccc5cccc(-c6ccccc6)c45)c4cccc5oc6ccc7ccccc7c6c45)cc3)c2)cc1]

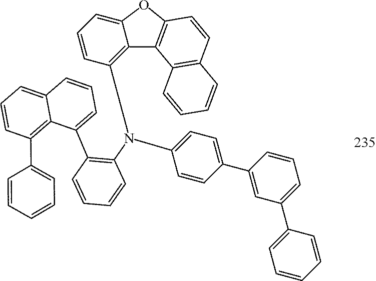 [SMILES: c1ccc(-c2cccc3cccc(-c4ccc(N(c5ccccc5)c5cccc6c7c8ccccc8ccc7n(-c7ccccc7)c56)cc4)c23)cc1]

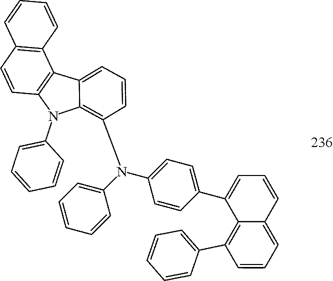 [SMILES: c1ccc(-c2ccc(N(c3ccc(-c4cccc5cccc(-c6ccccc6)c45)cc3)c3cccc4c5c6ccccc6ccc5n(-c5ccccc5)c34)cc2)cc1]

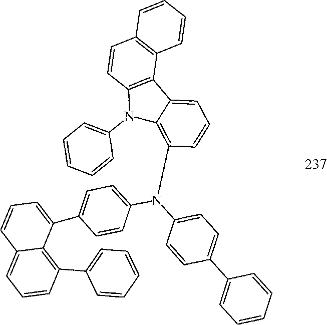 [SMILES: c1ccc(-c2cccc(N(c3ccc(-c4cccc5cccc(-c6ccccc6)c45)cc3)c3cccc4c5c6ccccc6ccc5n(-c5ccccc5)c34)c2)cc1]

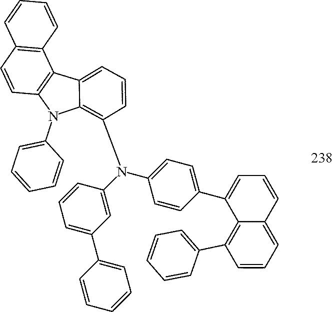 [SMILES: c1ccc(-c2ccccc2N(c2ccc(-c3cccc4cccc(-c5ccccc5)c34)cc2)c2cccc3c4c5ccccc5ccc4n(-c4ccccc4)c23)cc1]

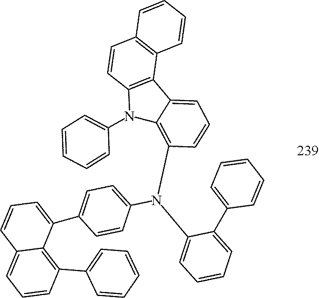 [SMILES: c1ccc(-c2cccc3cccc(-c4ccc(N(c5cccc6ccccc56)c5cccc6c7c8ccccc8ccc7n(-c7ccccc7)c56)cc4)c23)cc1]

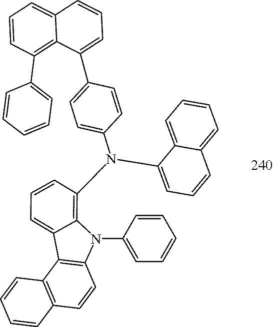 [SMILES: c1ccc(-c2cccc3cccc(-c4ccc(N(c5ccc6ccccc6c5)c5cccc6c7c8ccccc8ccc7n(-c7ccccc7)c56)cc4)c23)cc1]

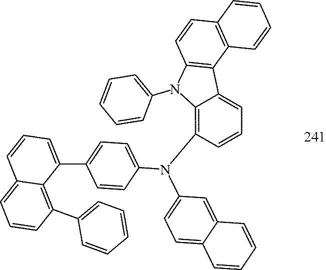 [SMILES: c1ccc(-c2cccc3cccc(-c4ccc(N(c5ccc6c(c5)oc5ccccc56)c5cccc6c7cc8ccccc8cc7n(-c7ccccc7)c56)cc4)c23)cc1]

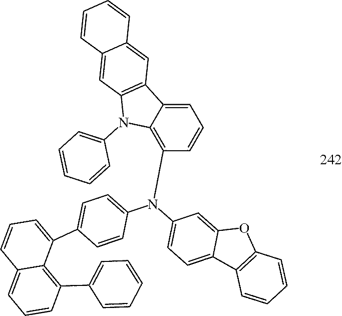 [SMILES: c1ccc(-c2cccc3cccc(-c4ccc(N(c5ccc6oc7ccccc7c6c5)c5cccc6c7cc8ccccc8cc7n(-c7ccccc7)c56)cc4)c23)cc1]

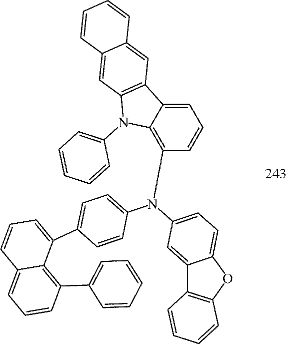 [SMILES: c1ccc(-c2cccc3cccc(-c4ccc(N(c5cccc6oc7ccccc7c56)c5cccc6c7cc8ccccc8cc7n(-c7ccccc7)c56)cc4)c23)cc1]

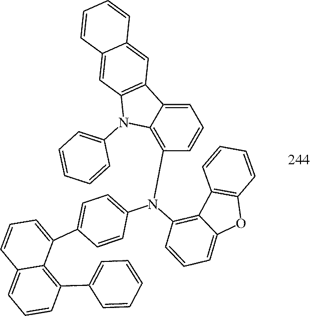 [SMILES: c1ccc(-c2cccc3cccc(-c4ccc(N(c5cccc6c5oc5ccccc56)c5cccc6c7cc8ccccc8cc7n(-c7ccccc7)c56)cc4)c23)cc1]

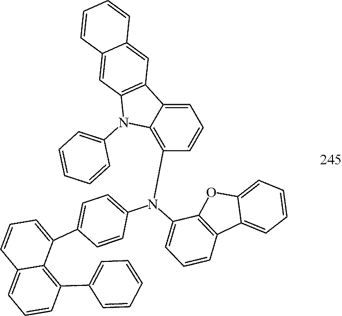 [SMILES: c1ccc(-c2cccc3cccc(-c4ccc(N(c5ccc6c(c5)sc5ccccc56)c5ccc6c7c8ccccc8ccc7n(-c7ccccc7)c6c5)cc4)c23)cc1]

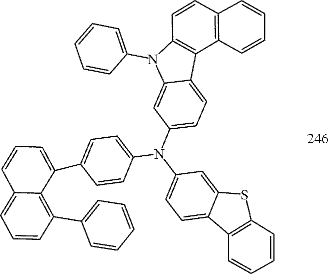 [SMILES: c1ccc(-c2cccc3cccc(-c4ccc(N(c5ccc6sc7ccccc7c6c5)c5ccc6c7c8ccccc8ccc7n(-c7ccccc7)c6c5)cc4)c23)cc1]

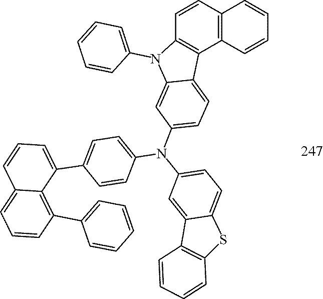 [SMILES: c1ccc(-c2cccc3cccc(-c4ccc(N(c5ccc6c7c8ccccc8ccc7n(-c7ccccc7)c6c5)c5cccc6sc7ccccc7c56)cc4)c23)cc1]

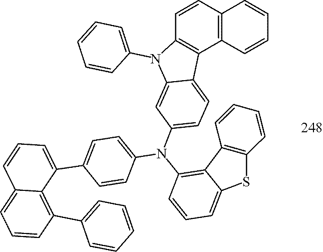 [SMILES: c1ccc(-c2cccc3cccc(-c4ccc(N(c5ccc6c7c8ccccc8ccc7n(-c7ccccc7)c6c5)c5cccc6c5sc5ccccc56)cc4)c23)cc1]

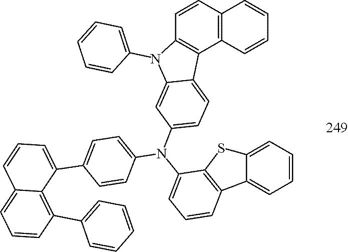 [SMILES: CC1(C)c2ccccc2-c2ccc(N(c3ccc(-c4cccc5cccc(-c6ccccc6)c45)cc3)c3ccc4c5ccc6ccccc6c5n(-c5ccccc5)c4c3)cc21]

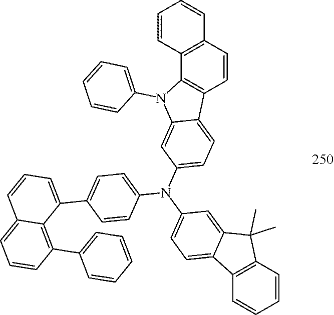 [SMILES: CC1(C)c2ccccc2-c2cc(N(c3ccc(-c4cccc5cccc(-c6ccccc6)c45)cc3)c3ccc4c5ccc6ccccc6c5n(-c5ccccc5)c4c3)ccc21]

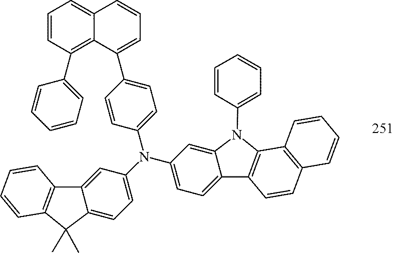 [SMILES: CC1(C)c2ccccc2-c2c(N(c3ccc(-c4cccc5cccc(-c6ccccc6)c45)cc3)c3ccc4c5ccc6ccccc6c5n(-c5ccccc5)c4c3)cccc21]

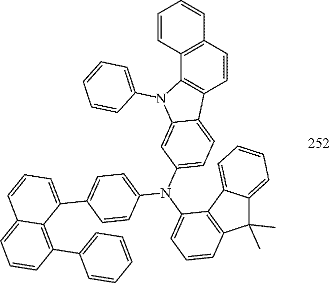 [SMILES: CC1(C)c2ccccc2-c2cccc(N(c3ccc(-c4cccc5cccc(-c6ccccc6)c45)cc3)c3ccc4c5ccc6ccccc6c5n(-c5ccccc5)c4c3)c21]

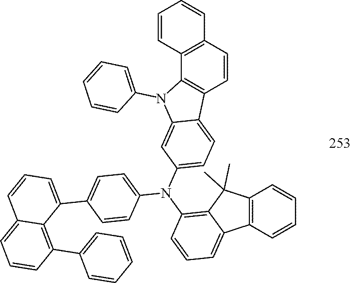 [SMILES: CC1(C)c2ccccc2-c2cccc(N(c3ccc(-c4cccc5cccc(-c6ccccc6)c45)cc3)c3ccc4c5c6ccccc6ccc5n(-c5ccccc5)c4c3)c21]

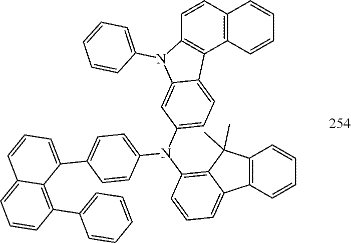 [SMILES: c1ccc(-c2cccc3cccc(-c4ccc(N(c5ccc6c7ccccc7n(-c7ccccc7)c6c5)c5ccc6c7c8ccccc8ccc7n(-c7ccccc7)c6c5)cc4)c23)cc1]

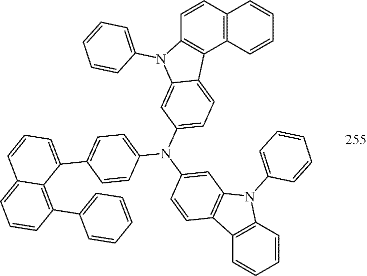 [SMILES: c1ccc(-c2cccc3cccc(-c4ccc(N(c5ccc6c(c5)c5ccccc5n6-c5ccccc5)c5ccc6c7c8ccccc8ccc7n(-c7ccccc7)c6c5)cc4)c23)cc1]

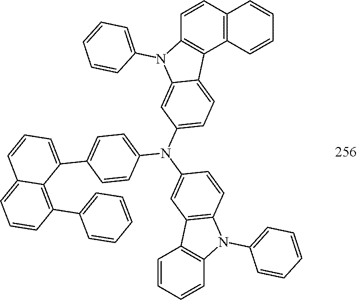 [SMILES: c1ccc(-c2cccc3cccc(-c4ccc(N(c5ccc(-n6c7ccccc7c7ccccc76)cc5)c5ccc6c(c5)c5c7ccccc7ccc5n6-c5ccccc5)cc4)c23)cc1]

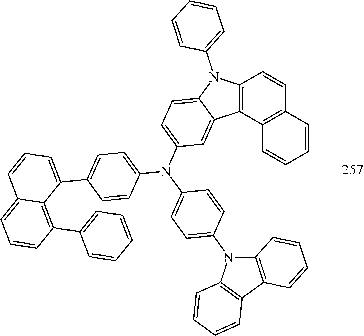 [SMILES: c1ccc(-c2cccc3cccc(-c4ccc(N(c5cccc(-n6c7ccccc7c7ccccc76)c5)c5ccc6c(c5)c5c7ccccc7ccc5n6-c5ccccc5)cc4)c23)cc1]

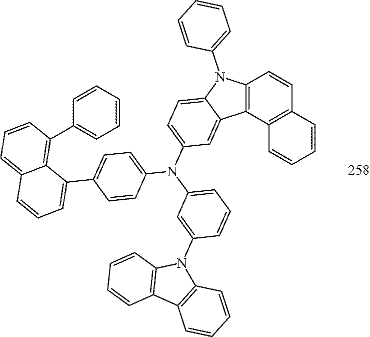 [SMILES: c1ccc(-c2cccc3cccc(-c4ccc(N(c5ccc6c(c5)c5c7ccccc7ccc5n6-c5ccccc5)c5ccccc5-n5c6ccccc6c6ccccc65)cc4)c23)cc1]

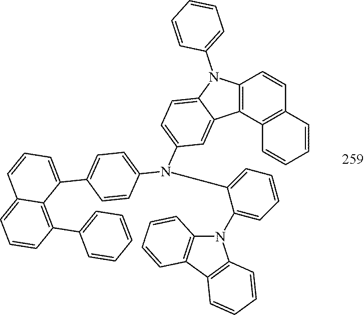 [SMILES: c1ccc(-c2cccc3cccc(-c4ccc(N(c5ccc6c(c5)c5c7ccccc7ccc5n6-c5ccccc5)c5cccc6c5c5ccccc5n6-c5ccccc5)cc4)c23)cc1]

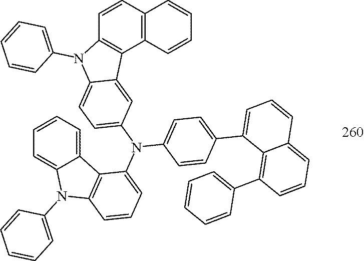 [SMILES: c1ccc(-c2cccc3cccc(-c4ccc(N(c5ccc6c(c5)c5c7ccccc7ccc5n6-c5ccccc5)c5cccc6c7ccccc7n(-c7ccccc7)c56)cc4)c23)cc1]

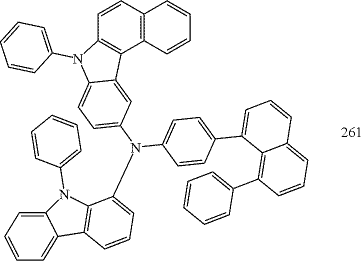 [SMILES: c1ccc(-c2cccc3cccc(-c4ccc(N(c5ccc6c(c5)C(c5ccccc5)(c5ccccc5)c5ccccc5-6)c5ccc6c(c5)c5c7ccccc7ccc5n6-c5ccccc5)cc4)c23)cc1]

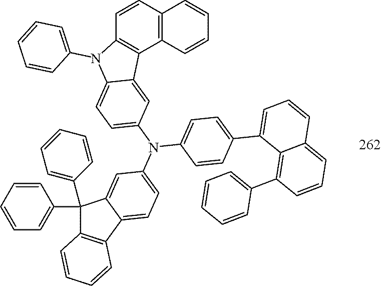 [SMILES: c1ccc(-c2cccc3cccc(-c4ccc(N(c5ccc6c(c5)c5c7ccccc7ccc5n6-c5ccccc5)c5cccc6c5-c5ccccc5C6(c5ccccc5)c5ccccc5)cc4)c23)cc1]

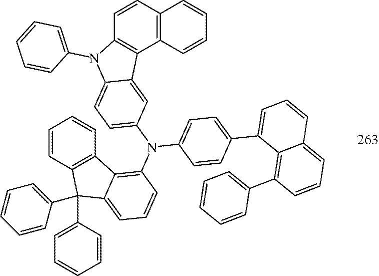 [SMILES: c1ccc(-c2cccc3cccc(-c4ccc(N(c5ccc(C6(c7ccccc7)c7ccccc7-c7ccccc76)cc5)c5ccc6c(c5)c5c7ccccc7ccc5n6-c5ccccc5)cc4)c23)cc1]

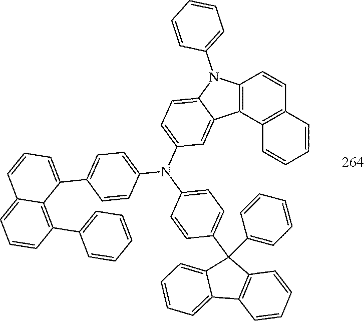 [SMILES: c1ccc(-c2cccc3cccc(-c4ccc(N(c5cccc(C6(c7ccccc7)c7ccccc7-c7ccccc76)c5)c5ccc6c(c5)c5c7ccccc7ccc5n6-c5ccccc5)cc4)c23)cc1]

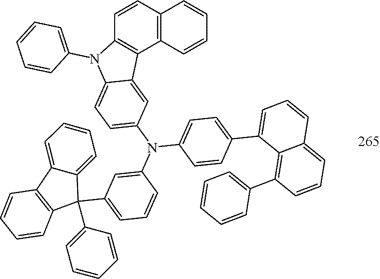 [SMILES: c1ccc(-c2cccc3cccc(-c4ccc(N(c5ccc6c(c5)C5(c7ccccc7-c7ccccc75)c5ccccc5-6)c5ccc6c(c5)c5c7ccccc7ccc5n6-c5ccccc5)cc4)c23)cc1]

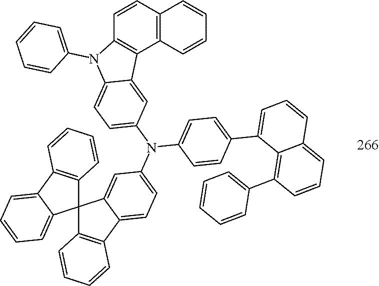 [SMILES: c1ccc(-c2cccc3cccc(-c4ccc(N(c5cccc6c5-c5ccccc5C65c6ccccc6-c6ccccc65)c5cccc6c5c5c7ccccc7ccc5n6-c5ccccc5)cc4)c23)cc1]

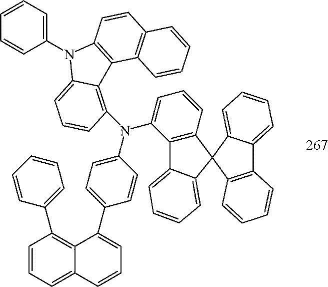 [SMILES: c1ccc(-c2cccc3cccc(-c4ccc(N(c5ccc6c(c5)-c5ccccc5C6(c5ccccc5)c5ccccc5)c5cccc6c5c5c7ccccc7ccc5n6-c5ccccc5)cc4)c23)cc1]

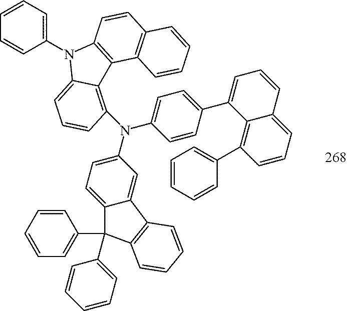 [SMILES: c1ccc(-c2cccc3cccc(-c4ccc(N(c5ccccc5C5(c6ccccc6)c6ccccc6-c6ccccc65)c5cccc6c5c5c7ccccc7ccc5n6-c5ccccc5)cc4)c23)cc1]

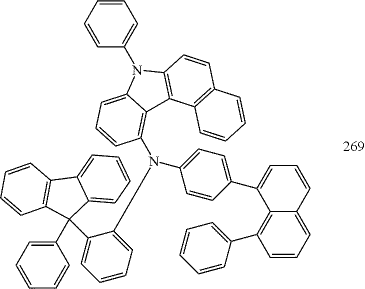 [SMILES: c1ccc(-c2cccc3cccc(-c4ccc(N(c5ccc6c(c5)-c5ccccc5C65c6ccccc6-c6ccccc65)c5cccc6c5c5c7ccccc7ccc5n6-c5ccccc5)cc4)c23)cc1]

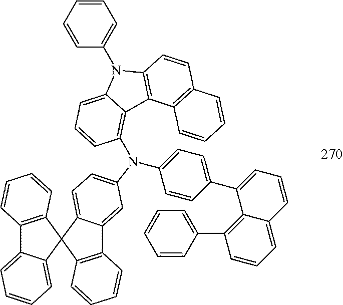 [SMILES: c1ccc(-c2cccc3cccc(-c4ccc(N(c5cccc6c5C5(c7ccccc7-c7ccccc75)c5ccccc5-6)c5cccc6c5c5c7ccccc7ccc5n6-c5ccccc5)cc4)c23)cc1]

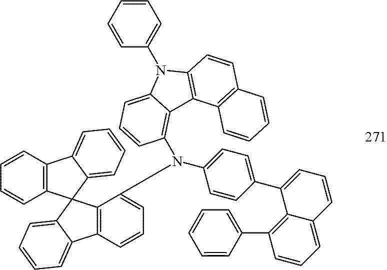 [SMILES: c1ccc(-c2ccc(-c3ccc(N(c4ccc(-c5cccc6cccc(-c7ccccc7)c56)cc4)c4cccc5c4c4c6ccccc6ccc4n5-c4ccccc4)cc3)cc2)cc1]

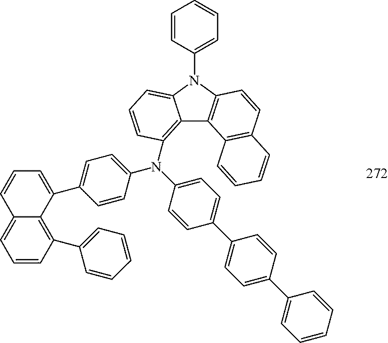 [SMILES: c1ccc(-c2cccc(-c3ccc(N(c4ccc(-c5cccc6cccc(-c7ccccc7)c56)cc4)c4cccc5c4c4c6ccccc6ccc4n5-c4ccccc4)cc3)c2)cc1]

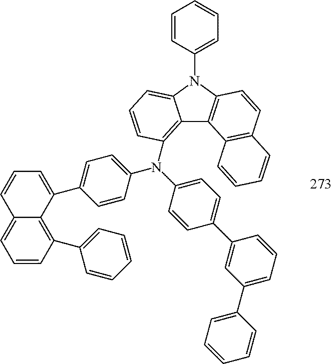 [SMILES: c1ccc(-c2cc(-c3ccccc3)cc(N(c3ccc(-c4cccc5cccc(-c6ccccc6)c45)cc3)c3cccc4c3c3c5ccccc5ccc3n4-c3ccccc3)c2)cc1]

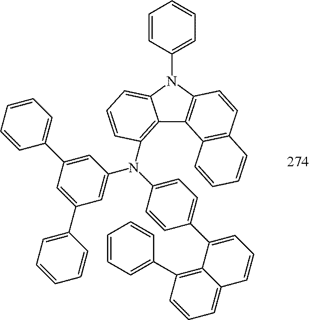 [SMILES: c1ccc(-c2ccc(N(c3ccc(-c4cccc5cccc(-c6ccccc6)c45)cc3)c3cccc4c3c3c5ccccc5ccc3n4-c3ccccc3)c(-c3ccccc3)c2)cc1]

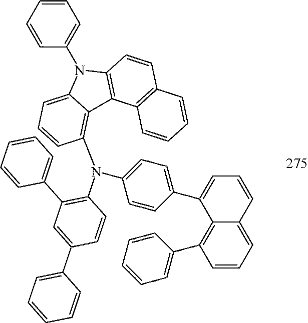 [SMILES: c1ccc(-c2ccc(-c3cccc(N(c4ccc(-c5cccc6cccc(-c7ccccc7)c56)cc4)c4cccc5c4c4c6ccccc6ccc4n5-c4ccccc4)c3)cc2)cc1]

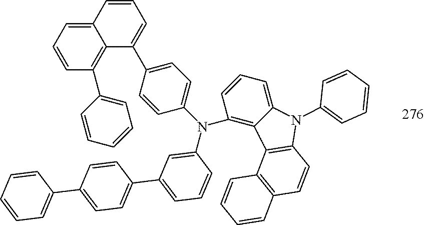 [SMILES: c1ccc(-c2ccc(-c3ccccc3N(c3ccc(-c4cccc5cccc(-c6ccccc6)c45)cc3)c3cccc4c3c3c5ccccc5ccc3n4-c3ccccc3)cc2)cc1]

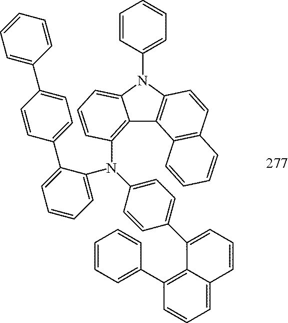 [SMILES: c1ccc(-c2cccc3cccc(-c4ccc(N(c5cc6ccccc6c6ccccc56)c5cc6ccccc6c6c7ccccc7n(-c7ccccc7)c56)cc4)c23)cc1]

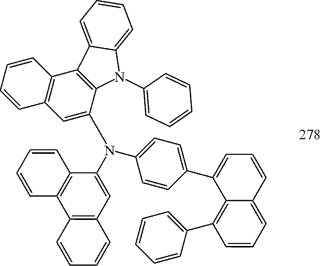 [SMILES: c1ccc(-c2cccc3cccc(-c4ccc(N(c5ccc6c(ccc7ccccc76)c5)c5cc6c(c7ccccc57)c5ccccc5n6-c5ccccc5)cc4)c23)cc1]

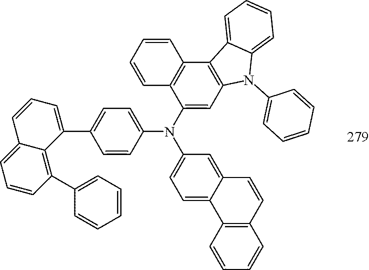 [SMILES: c1ccc(-c2cccc3cccc(-c4ccc(N(c5ccc(-c6cccc7ccccc67)cc5)c5cccc6c5ccc5c6c6ccccc6n5-c5ccccc5)cc4)c23)cc1]

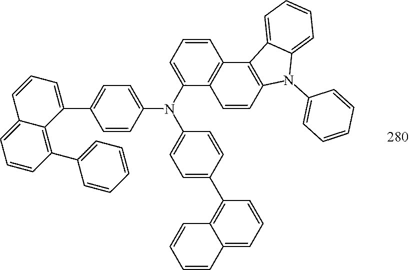 [SMILES: c1ccc(-c2ccc(N(c3ccc(-c4cccc5cccc(-c6ccccc6)c45)cc3)c3ccc4c(ccc5c4c4ccccc4n5-c4ccccc4)c3)c3ccccc23)cc1]

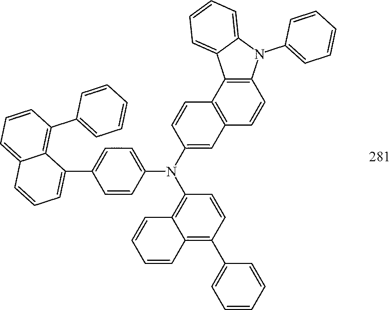 [SMILES: c1ccc(-c2cccc3cccc(-c4ccc(N(c5ccc(-c6ccc7ccccc7c6)cc5)c5ccc6ccc7c(c6c5)c5ccccc5n7-c5ccccc5)cc4)c23)cc1]

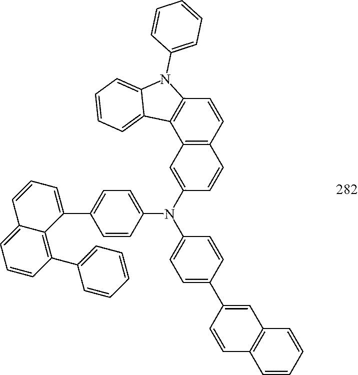 [SMILES: c1ccc(-c2ccc3cc(N(c4ccc(-c5cccc6cccc(-c7ccccc7)c56)cc4)c4cccc5ccc6c(c7ccccc7n6-c6ccccc6)c45)ccc3c2)cc1]

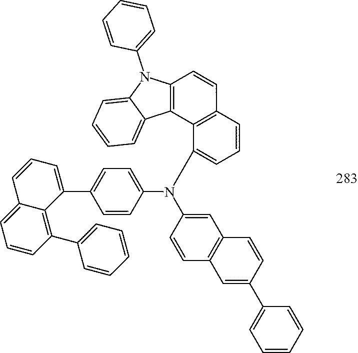 [SMILES: c1ccc(-c2cccc3cccc(-c4ccc(N(c5ccc(-c6ccc(-c7cccc8ccccc78)cc6)cc5)c5cc6ccccc6c6c7ccccc7n(-c7ccccc7)c56)cc4)c23)cc1]

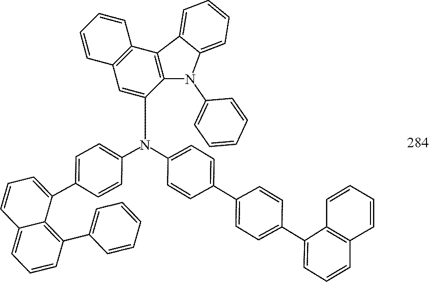 [SMILES: c1ccc(-c2cccc3cccc(-c4ccc(N(c5ccc(-c6ccc7c(c6)oc6ccccc67)cc5)c5cc6c(c7ccccc57)c5ccccc5n6-c5ccccc5)cc4)c23)cc1]

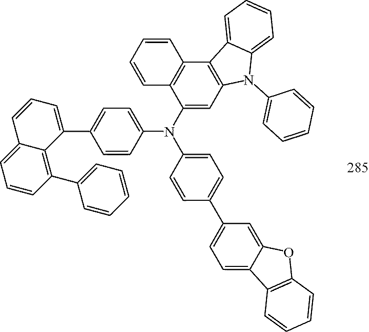 [SMILES: c1ccc(-c2cccc3cccc(-c4ccc(N(c5ccc(-c6ccc7oc8ccccc8c7c6)cc5)c5cccc6c5ccc5c6c6ccccc6n5-c5ccccc5)cc4)c23)cc1]

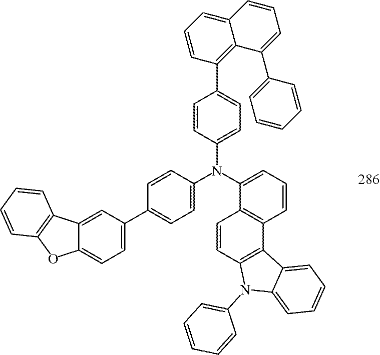 [SMILES: c1ccc(-c2cccc3cccc(-c4ccc(N(c5ccc(-c6cccc7oc8ccccc8c67)cc5)c5ccc6c(ccc7c6c6ccccc6n7-c6ccccc6)c5)cc4)c23)cc1]

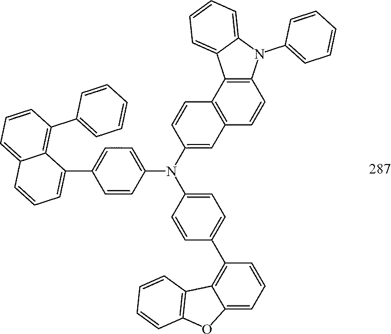 [SMILES: c1ccc(-c2cccc3cccc(-c4ccc(N(c5ccc(-c6cccc7c6oc6ccccc67)cc5)c5cc6c(c7ccccc57)c5ccccc5n6-c5ccccc5)cc4)c23)cc1]

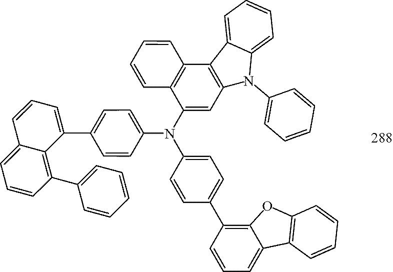 [SMILES: c1ccc(-c2cccc3cccc(-c4ccc(N(c5ccc(-c6ccc7c(c6)sc6ccccc67)cc5)c5cc6ccccc6c6c7ccccc7n(-c7ccccc7)c56)cc4)c23)cc1]

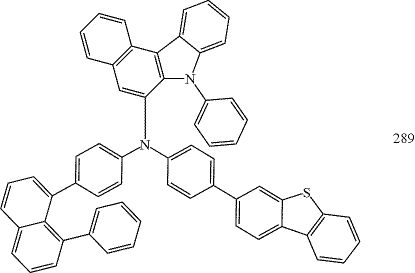 [SMILES: c1ccc(-c2cccc3cccc(-c4ccc(N(c5ccc(-c6ccc7sc8ccccc8c7c6)cc5)c5ccccc5-n5c6ccccc6c6c7ccccc7ccc65)cc4)c23)cc1]

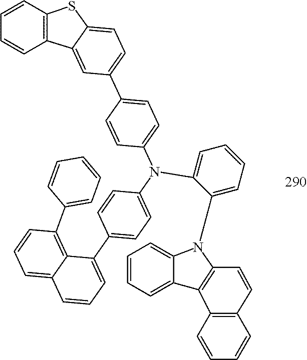 [SMILES: c1ccc(-c2cccc3cccc(-c4ccc(N(c5ccc(-c6cccc7sc8ccccc8c67)cc5)c5ccc6ccc7c(c6c5)c5ccccc5n7-c5ccccc5)cc4)c23)cc1]

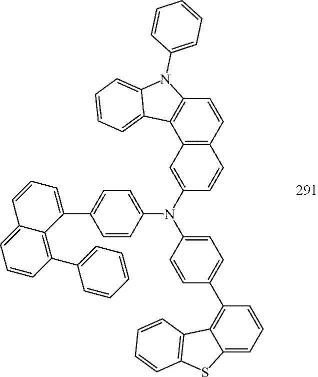 [SMILES: c1ccc(-c2cccc3cccc(-c4ccc(N(c5ccc(-c6cccc7c6sc6ccccc67)cc5)c5ccc6c(ccc7c6c6ccccc6n7-c6ccccc6)c5)cc4)c23)cc1]

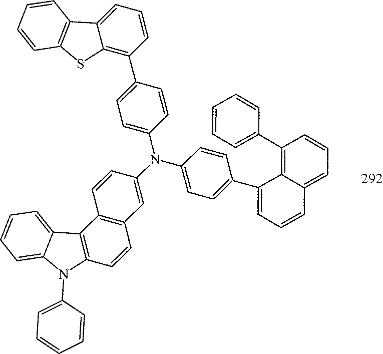 [SMILES: c1ccc(-c2cccc3cccc(-c4ccc(N(c5cccc(-c6cccc7oc8ccccc8c67)c5)c5cccc6c5ccc5c6c6ccccc6n5-c5ccccc5)cc4)c23)cc1]

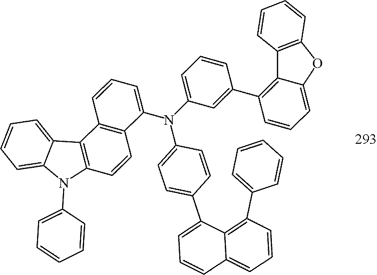 [SMILES: c1ccc(-c2cccc3cccc(-c4ccc(N(c5cccc(-c6cccc7c6oc6ccccc67)c5)c5cc6c(c7ccccc57)c5ccccc5n6-c5ccccc5)cc4)c23)cc1]

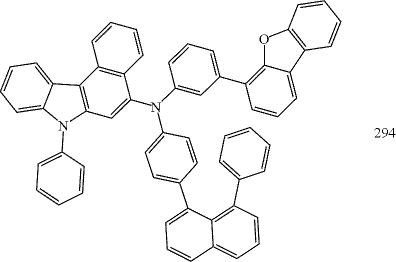 [SMILES: c1ccc(-c2cccc3cccc(-c4ccc(N(c5cccc(-c6cccc7sc8ccccc8c67)c5)c5cc6ccccc6c6c7ccccc7n(-c7ccccc7)c56)cc4)c23)cc1]

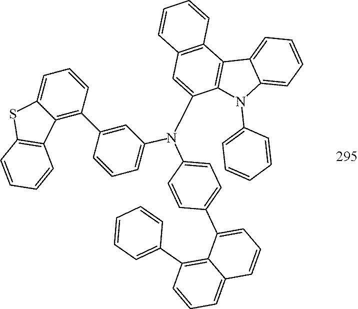 [SMILES: CC1(C)c2ccccc2-c2cc(-c3ccc(N(c4ccc(-c5cccc6cccc(-c7ccccc7)c56)cc4)c4cccc5ccc6c(c7ccccc7n6-c6ccccc6)c45)cc3)ccc21]

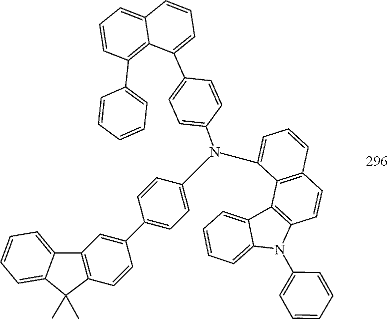 [SMILES: CC1(C)c2ccccc2-c2c(-c3ccc(N(c4ccc(-c5cccc6cccc(-c7ccccc7)c56)cc4)c4ccc5ccc6c(c5c4)c4ccccc4n6-c4ccccc4)cc3)cccc21]

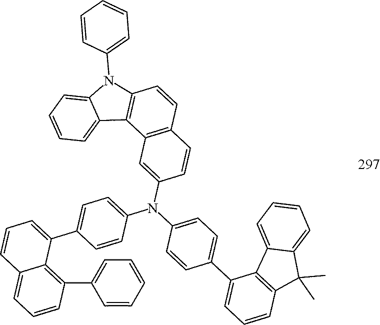 [SMILES: c1ccc(-c2cccc3cccc(-c4ccc(N(c5cccc(-c6cccc7cccc(-c8ccccc8)c67)c5)c5cccc(-n6c7ccccc7c7c8ccccc8ccc76)c5)cc4)c23)cc1]

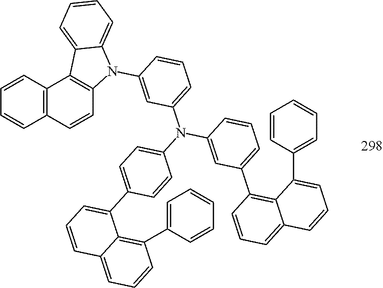 [SMILES: c1ccc(-c2cccc3cccc(-c4ccc(N(c5ccc(-n6c7ccccc7c7c8ccccc8ccc76)cc5)c5ccccc5-c5cccc6cccc(-c7ccccc7)c56)cc4)c23)cc1]

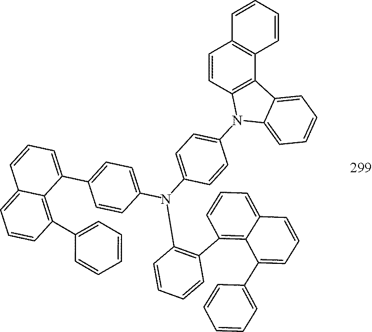 [SMILES: c1ccc(-c2ccc(N(c3cccc(-c4cccc5cccc(-c6ccccc6)c45)c3)c3cccc4c5c6ccccc6ccc5n(-c5ccccc5)c34)cc2)cc1]

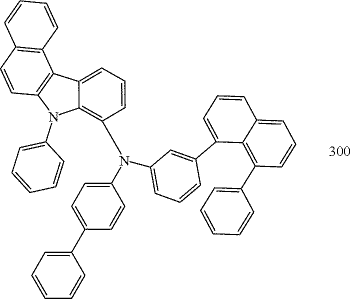 [SMILES: c1ccc(-c2cccc3cccc(-c4cccc(N(c5cccc6ccccc56)c5cccc6c7c8ccccc8ccc7n(-c7ccccc7)c56)c4)c23)cc1]

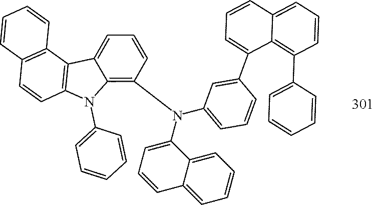 [SMILES: c1ccc(-c2cccc3cccc(-c4cccc(N(c5ccc6oc7ccccc7c6c5)c5cccc6c7c8ccccc8ccc7n(-c7ccccc7)c56)c4)c23)cc1]

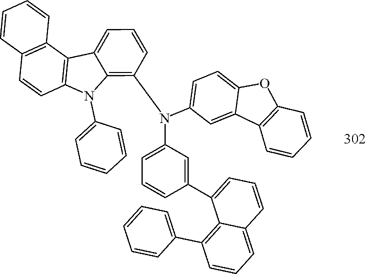 [SMILES: c1ccc(-c2cccc3cccc(-c4cccc(N(c5ccc6c(c5)sc5ccccc56)c5ccc6c7c8ccccc8ccc7n(-c7ccccc7)c6c5)c4)c23)cc1]

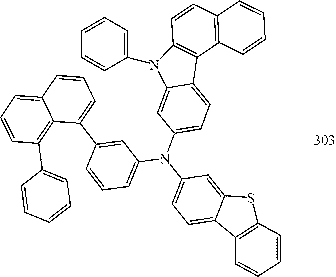 [SMILES: CC1(C)c2ccccc2-c2ccc(N(c3cccc(-c4cccc5cccc(-c6ccccc6)c45)c3)c3ccc4c5c6ccccc6ccc5n(-c5ccccc5)c4c3)cc21]

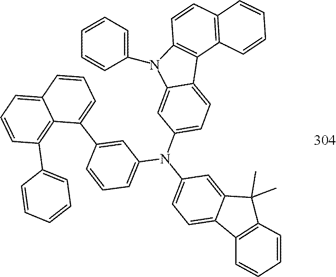 [SMILES: c1ccc(-c2cccc3cccc(-c4cccc(N(c5ccc6c7ccccc7n(-c7ccccc7)c6c5)c5ccc6c7c8ccccc8ccc7n(-c7ccccc7)c6c5)c4)c23)cc1]

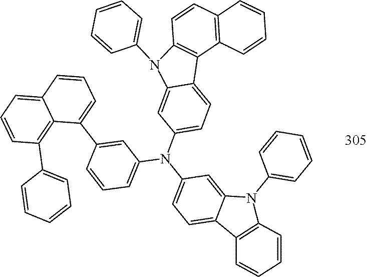 [SMILES: c1ccc(-c2cccc(-c3ccc(N(c4cccc(-c5cccc6cccc(-c7ccccc7)c56)c4)c4cccc5c4c4c6ccccc6ccc4n5-c4ccccc4)cc3)c2)cc1]

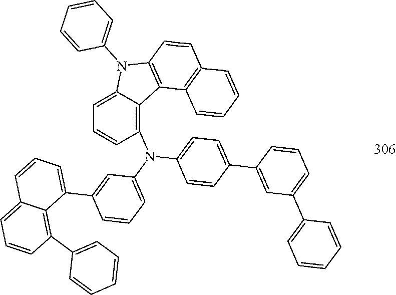 [SMILES: c1ccc(-c2ccc(N(c3ccccc3-c3cccc4cccc(-c5ccccc5)c34)c3cccc4c5c6ccccc6ccc5n(-c5ccccc5)c34)cc2)cc1]

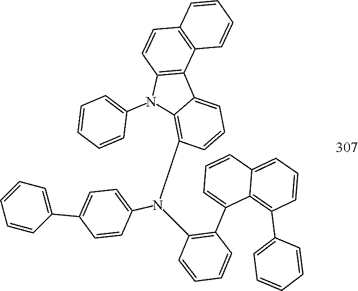 [SMILES: c1ccc(-c2cccc3cccc(-c4ccccc4N(c4cccc5ccccc45)c4cccc5c6c7ccccc7ccc6n(-c6ccccc6)c45)c23)cc1]

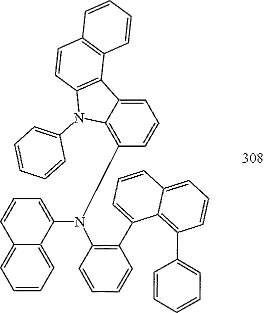 [SMILES: c1ccc(-c2cccc3cccc(-c4ccccc4N(c4ccc5oc6ccccc6c5c4)c4cccc5c6c7ccccc7ccc6n(-c6ccccc6)c45)c23)cc1]

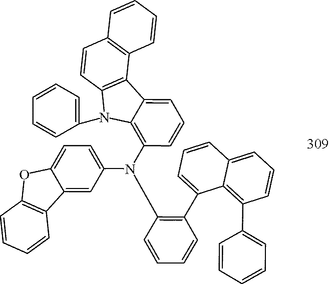 [SMILES: c1ccc(-c2cccc3cccc(-c4ccccc4N(c4ccc5c(c4)sc4ccccc45)c4ccc5c6c7ccccc7ccc6n(-c6ccccc6)c5c4)c23)cc1]

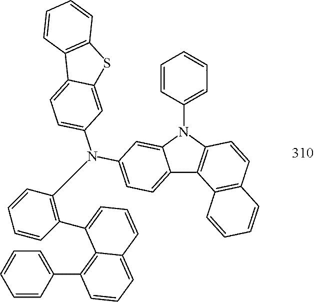 [SMILES: c1ccc(-c2ccc(N(c3ccccc3-c3cccc4cccc(-c5ccccc5)c34)c3cccc4c5c6ccccc6ccc5n(-c5ccccc5)c34)cc2)cc1]

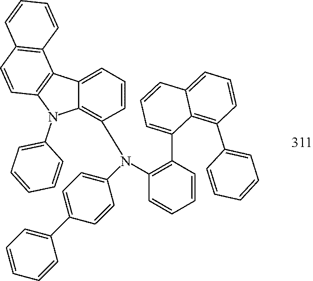 [SMILES: c1ccc(-c2cccc3cccc(-c4ccccc4N(c4ccc5c6ccccc6n(-c6ccccc6)c5c4)c4ccc5c6c7ccccc7ccc6n(-c6ccccc6)c5c4)c23)cc1]

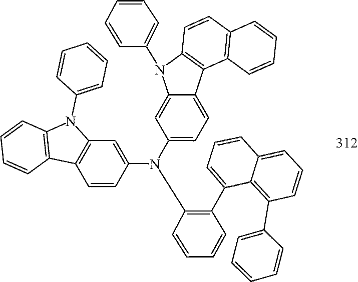 [SMILES: c1ccc(-c2cccc(-c3ccc(N(c4ccccc4-c4cccc5cccc(-c6ccccc6)c45)c4cccc5c4c4c6ccccc6ccc4n5-c4ccccc4)cc3)c2)cc1]

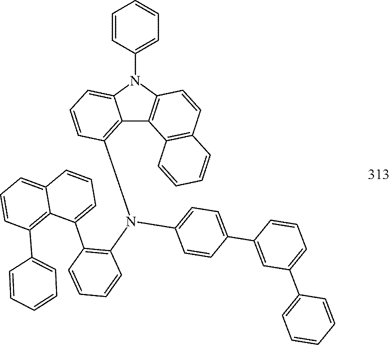 [SMILES: c1ccc(-c2cccc3cccc(-c4ccc(N(c5ccccc5)c5cccc6c5[Si](c5ccccc5)(c5ccccc5)c5ccc7ccccc7c5-6)cc4)c23)cc1]

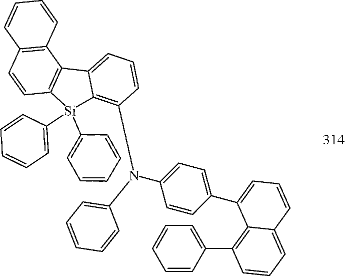 [SMILES: c1ccc(-c2ccc(N(c3ccc(-c4cccc5cccc(-c6ccccc6)c45)cc3)c3cccc4c3[Si](c3ccccc3)(c3ccccc3)c3ccc5ccccc5c3-4)cc2)cc1]

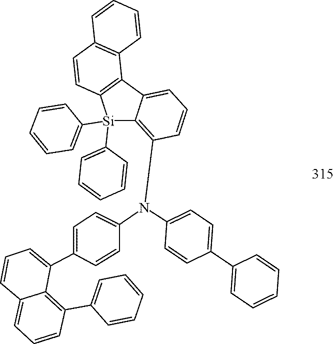 [SMILES: c1ccc(-c2cccc(N(c3ccc(-c4cccc5cccc(-c6ccccc6)c45)cc3)c3cccc4c3[Si](c3ccccc3)(c3ccccc3)c3ccc5ccccc5c3-4)c2)cc1]

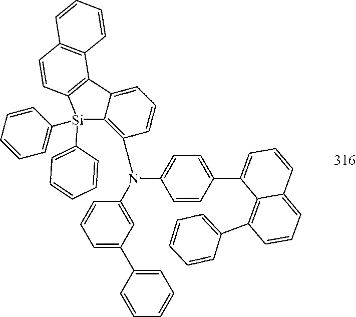 [SMILES: c1ccc(-c2cccc3cccc(-c4ccc(N(c5ccc6c(c5)oc5ccccc56)c5cccc6c5[Si](c5ccccc5)(c5ccccc5)c5ccc7ccccc7c5-6)cc4)c23)cc1]

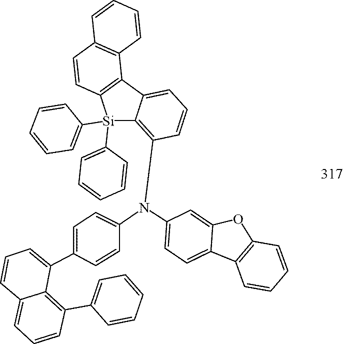 [SMILES: c1ccc(-c2cccc3cccc(-c4ccc(N(c5ccc6c(c5)[Si](c5ccccc5)(c5ccccc5)c5ccc7ccccc7c5-6)c5cccc6c5sc5ccccc56)cc4)c23)cc1]

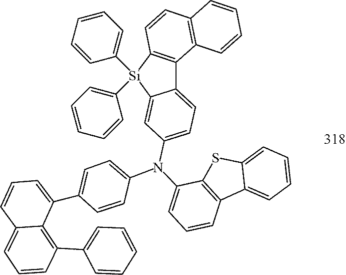 [SMILES: CC1(C)c2ccccc2-c2ccc(N(c3ccc(-c4cccc5cccc(-c6ccccc6)c45)cc3)c3ccc4c(c3)[Si](c3ccccc3)(c3ccccc3)c3ccc5ccccc5c3-4)cc21]

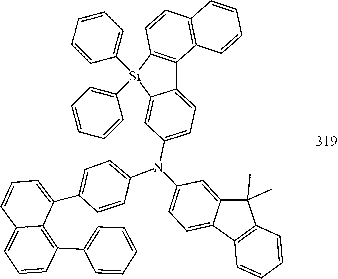 [SMILES: c1ccc(-c2ccc(-c3ccc(N(c4ccc(-c5cccc6cccc(-c7ccccc7)c56)cc4)c4cccc5c4-c4c(ccc6ccccc46)[Si]5(c4ccccc4)c4ccccc4)cc3)cc2)cc1]

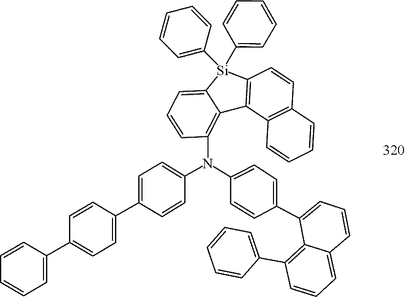 [SMILES: c1ccc(-c2cccc3cccc(-c4ccc(N(c5ccc6c(c5)-c5c(ccc7ccccc57)[Si]6(c5ccccc5)c5ccccc5)c5cccc6c5-c5ccccc5C6(c5ccccc5)c5ccccc5)cc4)c23)cc1]

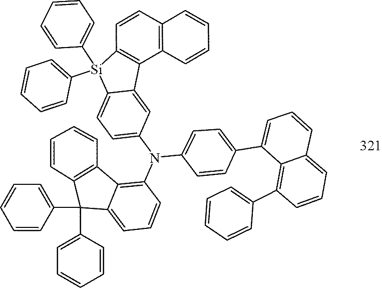 [SMILES: [2H]c1c([2H])c([2H])c(N(c2ccc(-c3cccc4cccc(-c5ccccc5)c34)cc2)c2cccc3c2C(C)(C)c2ccc4ccccc4c2-3)c([2H])c1[2H]]

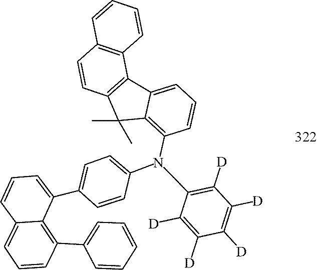 [SMILES: CC1(C)c2ccc3ccccc3c2-c2cccc(N(c3ccc(-c4ccc(F)cc4)cc3)c3ccc(-c4cccc5cccc(-c6ccccc6)c45)cc3)c21]

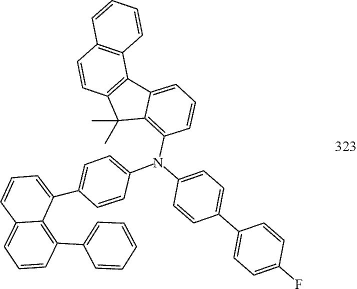

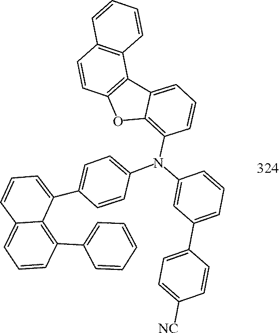 [SMILES: Cc1cccc2c1-c1ccc(N(c3ccc(-c4cccc5cccc(-c6ccccc6)c45)cc3)c3ccc4c(c3)oc3ccc5ccccc5c34)cc1C2(C)C]

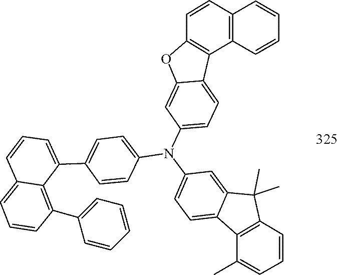 [SMILES: CCc1ccc(N(c2ccc(-c3cccc4cccc(-c5ccccc5)c34)cc2)c2cccc3c4c5ccccc5ccc4n(-c4ccccc4)c23)cc1]

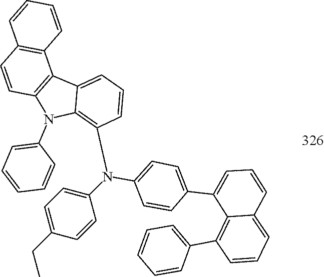 [SMILES: CC(C)(C)c1ccc(-c2ccc(N(c3ccc(-c4cccc5cccc(-c6ccccc6)c45)cc3)c3cccc4c5c6ccccc6ccc5n(-c5ccccc5)c34)cc2)cc1]

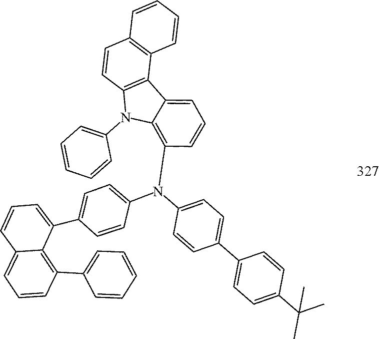 [SMILES: CC(C)c1ccc(-c2cccc(N(c3ccc(-c4cccc5cccc(-c6ccccc6)c45)cc3)c3cccc4c3sc3ccc5ccccc5c34)c2)cc1]

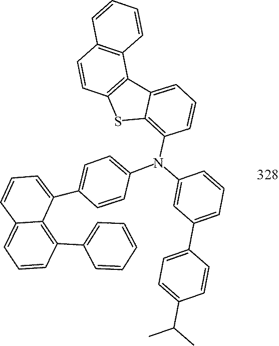 [SMILES: C[Si](C)(C)c1ccc(-c2ccc(N(c3cccc(-c4cccc5cccc(-c6ccccc6)c45)c3)c3cccc4c3sc3ccc5ccccc5c34)cc2)cc1]

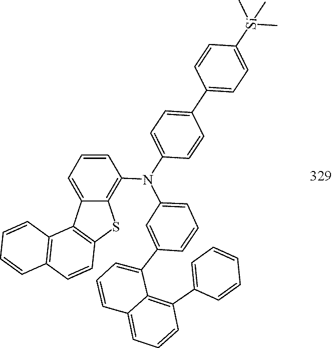 [SMILES: CC1(C)c2ccc3ccccc3c2-c2cccc(N(c3ccc(-c4cccc5cccc(-c6ccccc6)c45)cc3)c3cccc4c(-c5ccccc5)cccc34)c21]

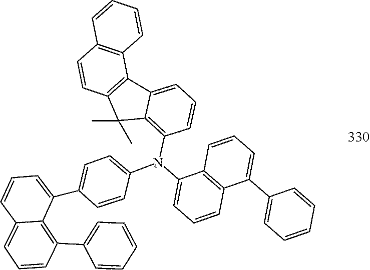 [SMILES: CC1(C)c2ccc3ccccc3c2-c2cccc(N(c3ccc(-c4cccc5cccc(-c6ccccc6)c45)cc3)c3ccc4c(-c5ccccc5)cccc4c3)c21]

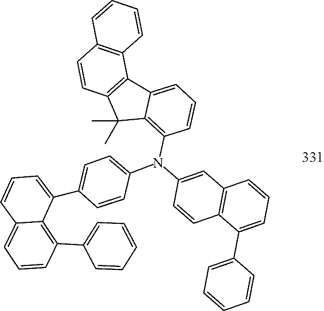 [SMILES: c1ccc(-c2ccc3ccccc3c2N(c2ccc(-c3cccc4cccc(-c5ccccc5)c34)cc2)c2cccc3c2oc2ccc4ccccc4c23)cc1]

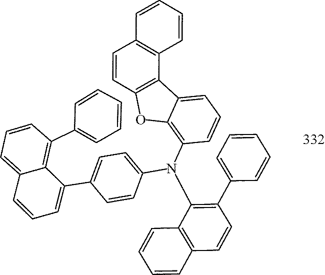 [SMILES: c1ccc(-c2cc3ccccc3cc2N(c2ccc(-c3cccc4cccc(-c5ccccc5)c34)cc2)c2ccc3c(c2)oc2ccc4ccccc4c23)cc1]

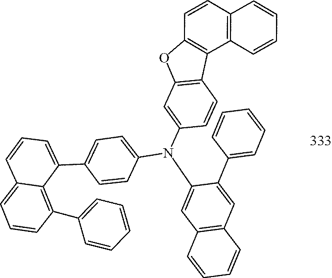 [SMILES: c1ccc(-c2cc(N(c3ccc(-c4cccc5cccc(-c6ccccc6)c45)cc3)c3cccc4c5c6ccccc6ccc5n(-c5ccccc5)c34)cc3ccccc23)cc1]

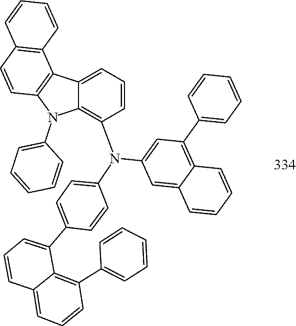 [SMILES: c1ccc(-c2ccc3cccc(N(c4ccc(-c5cccc6cccc(-c7ccccc7)c56)cc4)c4cccc5c6c7ccccc7ccc6n(-c6ccccc6)c45)c3c2)cc1]

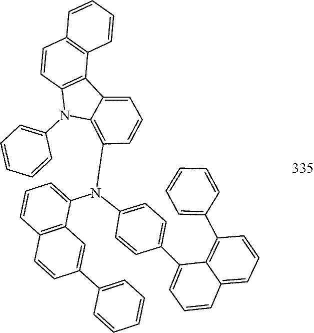 [SMILES: c1ccc(-c2ccccc2-c2cccc(N(c3ccc(-c4cccc5cccc(-c6ccccc6)c45)cc3)c3cccc4c3sc3ccc5ccccc5c34)c2)cc1]

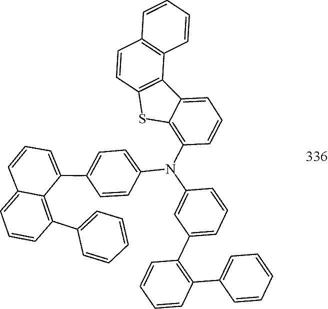 [SMILES: c1ccc(-c2ccccc2-c2ccccc2N(c2cccc(-c3cccc4cccc(-c5ccccc5)c34)c2)c2cccc3c2sc2ccc4ccccc4c23)cc1]

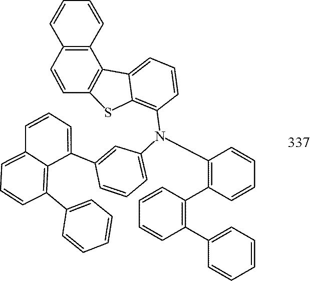 [SMILES: CC1(C)c2ccc3ccccc3c2-c2cccc(N(c3ccc(-c4cccc5cccc(-c6ccccc6)c45)cc3)c3cccc(-c4cccc(-c5ccccc5)c4)c3)c21]

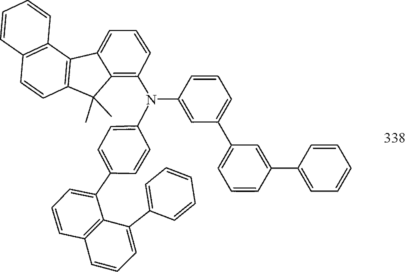 [SMILES: CC1(C)c2cc(N(c3ccc(-c4ccccc4)cc3)c3ccc(-c4cccc5cccc(-c6ccccc6)c45)cc3)ccc2-c2cc3ccccc3cc21]

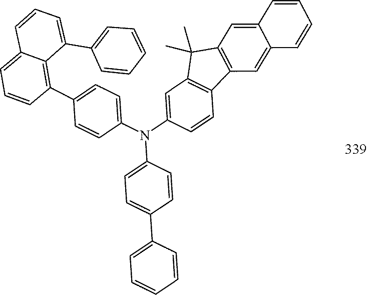 [SMILES: CC1(C)c2ccc(N(c3ccc(-c4cccc5cccc(-c6ccccc6)c45)cc3)c3cccc4ccccc34)cc2-c2cc3ccccc3cc21]

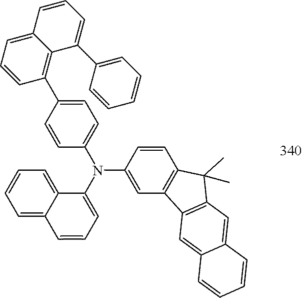 [SMILES: CC1(C)c2ccccc2-c2ccc(N(c3ccc(-c4cccc5cccc(-c6ccccc6)c45)cc3)c3cccc4c3-c3cc5ccccc5cc3C4(C)C)cc21]

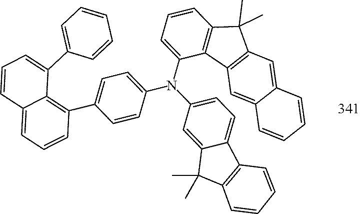 [SMILES: CC1(C)c2ccccc2-c2cc3ccccc3c(N(c3ccc(-c4cccc5cccc(-c6ccccc6)c45)cc3)c3cccc(-c4ccccc4)c3)c21]

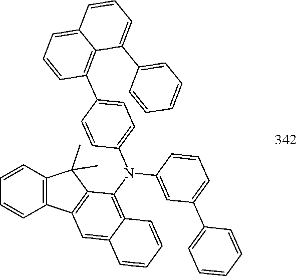 [SMILES: CC1(C)c2ccccc2-c2cc3ccc(N(c4cccc(-c5cccc6cccc(-c7ccccc7)c56)c4)c4ccc5c(c4)sc4ccccc45)cc3cc21]

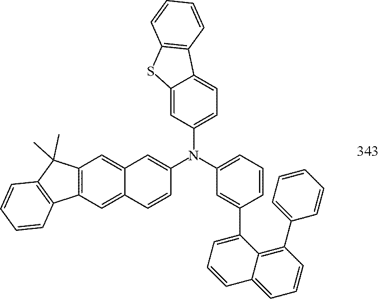 [SMILES: CC1(C)c2ccccc2-c2cc3cc(N(c4cccc(-c5cccc6cccc(-c7ccccc7)c56)c4)c4ccc5oc6ccccc6c5c4)ccc3cc21]

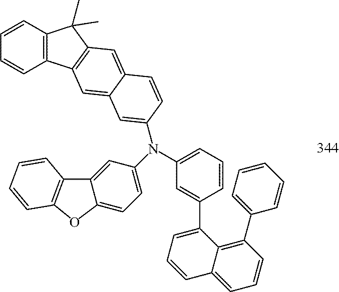 [SMILES: CC1(C)c2ccccc2-c2cc3c(N(c4ccc(-c5cccc6ccccc56)cc4)c4cccc(-c5cccc6cccc(-c7ccccc7)c56)c4)cccc3cc21]

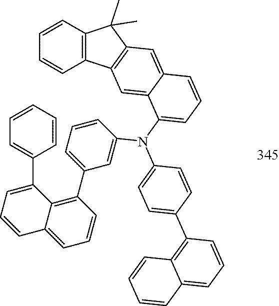 [SMILES: CC1(C)c2ccccc2-c2c1cc1ccccc1c2N(c1cccc(-c2cccc3cccc(-c4ccccc4)c23)c1)c1ccc2c3ccccc3n(-c3ccccc3)c2c1]

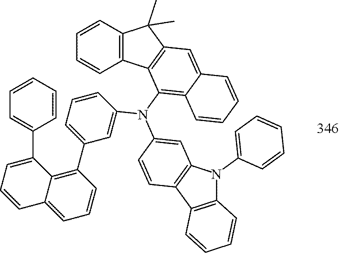 [SMILES: CC1(C)c2ccccc2-c2cc3cccc(N(c4cccc(-c5cccc6cccc(-c7ccccc7)c56)c4)c4cccc5c4oc4ccccc45)c3cc21]

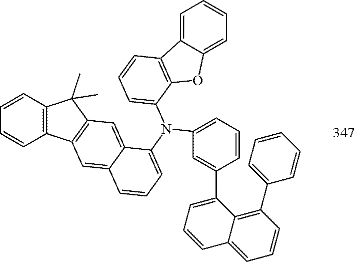 [SMILES: c1ccc(-c2cccc3cccc(-c4ccc(N(c5ccccc5)c5cccc6c5sc5c7ccccc7ccc65)cc4)c23)cc1]

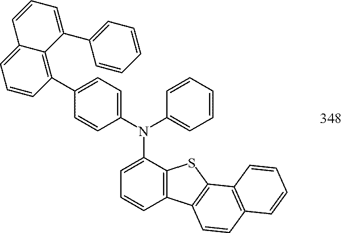 [SMILES: c1ccc(-c2ccc(N(c3ccc(-c4cccc5cccc(-c6ccccc6)c45)cc3)c3ccc4sc5c6ccccc6ccc5c4c3)cc2)cc1]

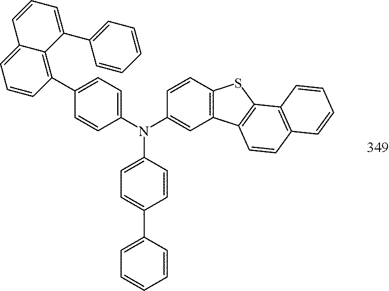 [SMILES: c1ccc(-c2cccc3cccc(-c4ccc(N(c5ccc6oc7ccccc7c6c5)c5cccc6sc7c8ccccc8ccc7c56)cc4)c23)cc1]

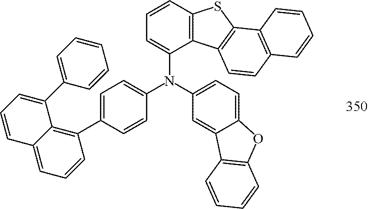 [SMILES: c1ccc(-c2cccc3cccc(-c4ccc(N(c5ccc(-c6ccc7ccccc7c6)cc5)c5cc6ccccc6c6sc7ccccc7c56)cc4)c23)cc1]

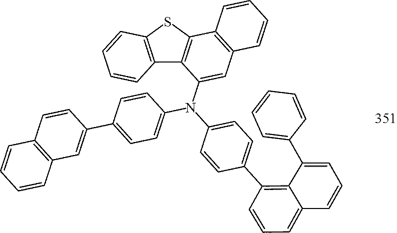 [SMILES: c1ccc(-c2cccc3cccc(-c4ccccc4N(c4cccc5ccccc45)c4cc5c6ccccc6sc5c5ccccc45)c23)cc1]

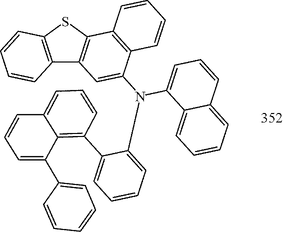 [SMILES: CC1(C)c2ccccc2-c2ccc(N(c3ccccc3-c3cccc4cccc(-c5ccccc5)c34)c3cccc4c3ccc3c5ccccc5sc43)cc21]

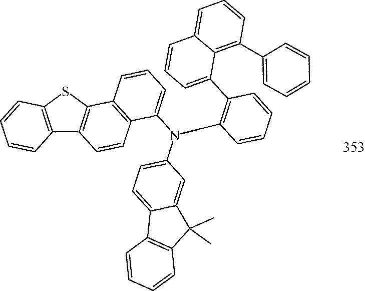 [SMILES: c1ccc(-c2cccc3cccc(-c4ccccc4N(c4ccc5c(ccc6c7ccccc7sc56)c4)c4ccc5oc6ccccc6c5c4)c23)cc1]

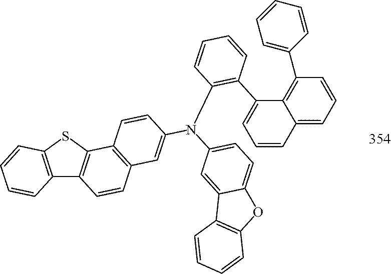 [SMILES: c1ccc(-c2cccc(N(c3ccc4ccc5c6ccccc6sc5c4c3)c3ccccc3-c3cccc4cccc(-c5ccccc5)c34)c2)cc1]

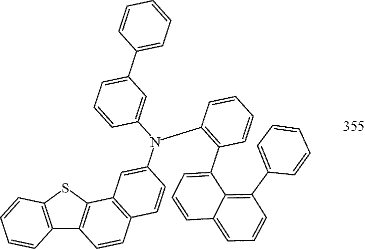 [SMILES: c1ccc(-c2ccc(N(c3ccccc3-c3cccc4cccc(-c5ccccc5)c34)c3cccc4ccc5c6ccccc6sc5c34)cc2)cc1]

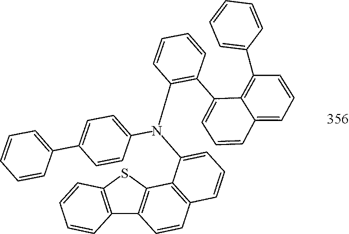 [SMILES: c1ccc(-c2cccc(N(c3ccc4ccc5c6ccccc6sc5c4c3)c3ccccc3-c3cccc4cccc(-c5ccccc5)c34)c2)cc1]

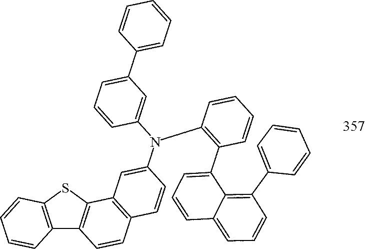 [SMILES: c1ccc(-c2cccc3cccc(-c4ccccc4N(c4ccc5c(ccc6c7ccccc7sc56)c4)c4ccc5oc6ccccc6c5c4)c23)cc1]

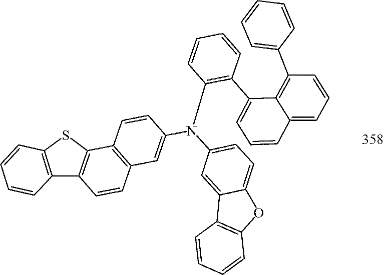 [SMILES: CC1(C)c2ccccc2-c2ccc(N(c3ccccc3-c3cccc4cccc(-c5ccccc5)c34)c3cccc4c3ccc3c5ccccc5sc43)cc21]

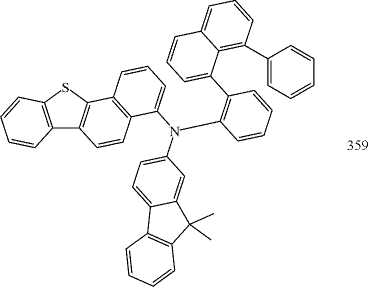 [SMILES: c1ccc(-c2cccc3cccc(-c4ccccc4N(c4cccc5ccccc45)c4cc5c6ccccc6sc5c5ccccc45)c23)cc1]

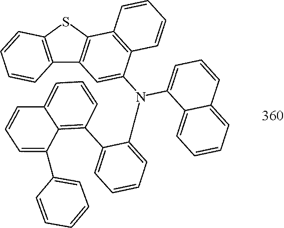 [SMILES: c1ccc(-c2cccc3cccc(-c4ccc(N(c5ccc6c(c5)sc5cc7ccccc7cc56)c5ccc6oc7ccccc7c6c5)cc4)c23)cc1]

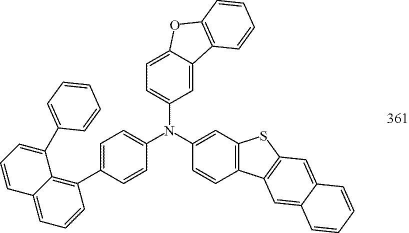 [SMILES: CC1(C)c2ccccc2-c2ccc(N(c3ccc(-c4cccc5cccc(-c6ccccc6)c45)cc3)c3ccc4sc5cc6ccccc6cc5c4c3)cc21]

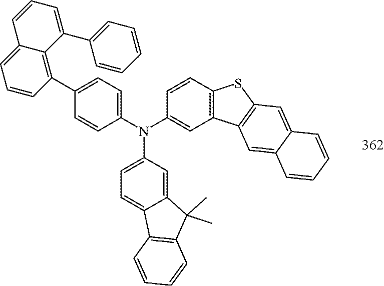 [SMILES: c1ccc(-c2cccc3cccc(-c4ccc(N(c5ccccc5)c5cccc6cc7sc8ccccc8c7cc56)cc4)c23)cc1]

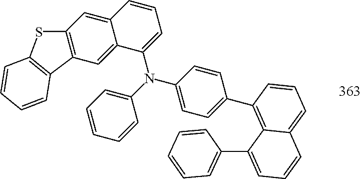 [SMILES: c1ccc(-c2cccc3cccc(-c4ccc(N(c5ccccc5)c5c6ccccc6cc6c5sc5ccccc56)cc4)c23)cc1]

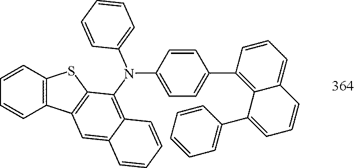 [SMILES: C1=CC2Sc3cc4c(N(c5ccc(-c6cccc7cccc(-c8ccccc8)c67)cc5)c5ccc(-n6c7ccccc7c7ccccc76)cc5)cccc4cc3C2C=C1]

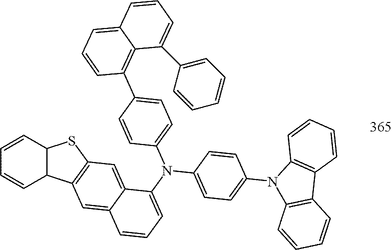 [SMILES: c1ccc(-c2cccc3cccc(-c4ccc(N(c5ccc6cc7c(cc6c5)sc5ccccc57)c5cccc6oc7ccccc7c56)cc4)c23)cc1]

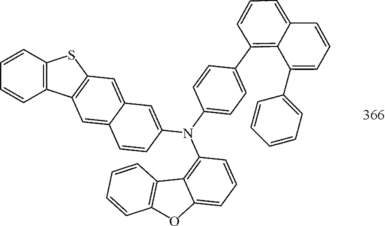 [SMILES: c1ccc(-c2cc(-c3ccccc3)cc(N(c3ccc(-c4cccc5cccc(-c6ccccc6)c45)cc3)c3ccc4cc5sc6ccccc6c5cc4c3)c2)cc1]

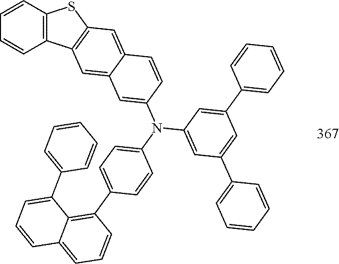 [SMILES: c1ccc(-c2cccc3cccc(-c4ccc(N(c5ccccc5)c5cccc6sc7cc8ccccc8cc7c56)cc4)c23)cc1]

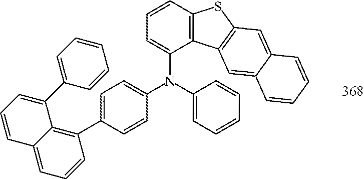 [SMILES: c1ccc(-c2cccc3cccc(-c4ccc(N(c5ccc(-c6ccc7c(c6)oc6ccccc67)cc5)c5c6ccccc6cc6sc7ccccc7c56)cc4)c23)cc1]

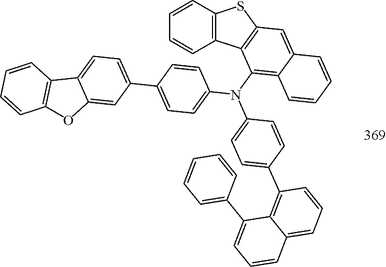 [SMILES: c1ccc(-c2cccc3cccc(-c4ccc(N(c5ccc6c(c5)sc5ccccc56)c5cccc6c5oc5c7ccccc7ccc65)cc4)c23)cc1]

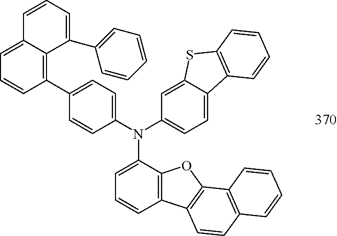 [SMILES: c1ccc(-c2cccc3cccc(-c4ccc(N(c5ccc6oc7ccccc7c6c5)c5ccc6oc7c8ccccc8ccc7c6c5)cc4)c23)cc1]

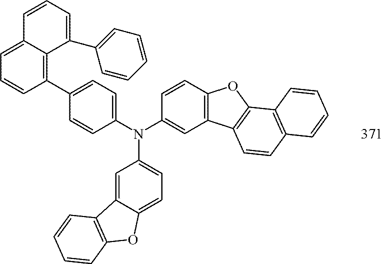 [SMILES: c1ccc(-c2cccc3cccc(-c4ccc(N(c5ccc(-c6cccc7ccccc67)cc5)c5cccc6oc7c8ccccc8ccc7c56)cc4)c23)cc1]

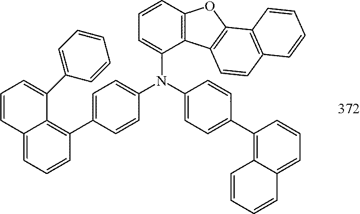 [SMILES: c1ccc(-c2ccc(N(c3ccc(-c4cccc5cccc(-c6ccccc6)c45)cc3)c3cc4ccccc4c4oc5ccccc5c34)cc2)cc1]

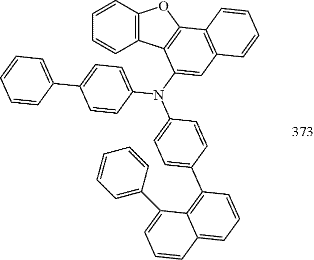 [SMILES: c1ccc(-c2cccc(N(c3ccc(-c4cccc5cccc(-c6ccccc6)c45)cc3)c3cc4c5ccccc5oc4c4ccccc34)c2)cc1]

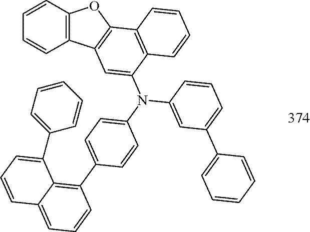 [SMILES: c1ccc(-c2cccc3cccc(-c4ccc(N(c5ccc(-n6c7ccccc7c7ccccc76)cc5)c5cccc6c5ccc5c7ccccc7oc65)cc4)c23)cc1]

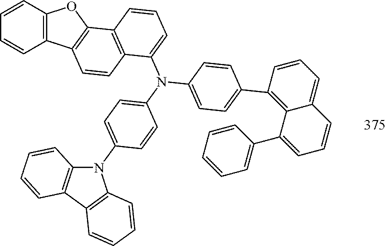 [SMILES: c1ccc(-c2cccc3cccc(-c4ccc(N(c5ccc6c(ccc7c8ccccc8oc67)c5)c5cccc6oc7ccccc7c56)cc4)c23)cc1]

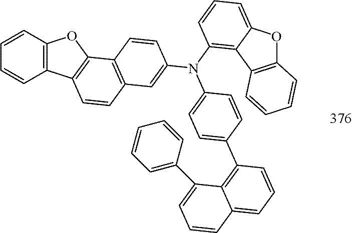 [SMILES: c1ccc(-c2cccc3cccc(-c4ccc(N(c5ccc6sc7ccccc7c6c5)c5ccc6ccc7c8ccccc8oc7c6c5)cc4)c23)cc1]

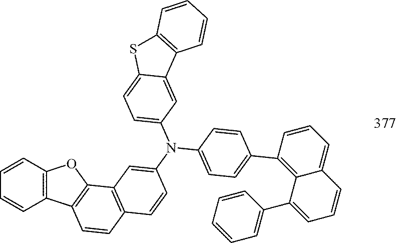 [SMILES: c1ccc(-c2cccc3cccc(-c4ccc(N(c5ccccc5)c5cccc6ccc7c8ccccc8oc7c56)cc4)c23)cc1]

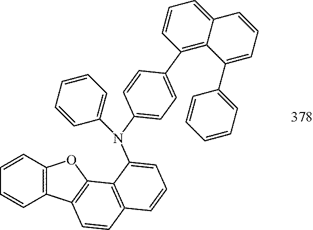 [SMILES: CC1(C)c2ccccc2-c2ccc(N(c3ccc(-c4cccc5cccc(-c6ccccc6)c45)cc3)c3ccc4c(c3)oc3cc5ccccc5cc34)cc21]

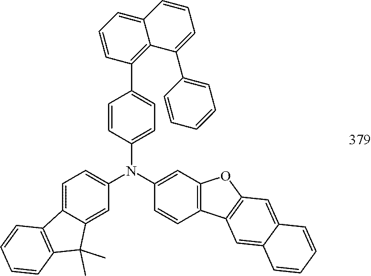 [SMILES: c1ccc(-c2cccc(N(c3ccc(-c4cccc5cccc(-c6ccccc6)c45)cc3)c3ccc4oc5cc6ccccc6cc5c4c3)c2)cc1]

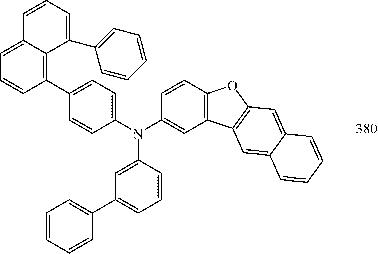 [SMILES: c1ccc(-c2cccc3cccc(-c4ccc(N(c5ccc6sc7ccccc7c6c5)c5cccc6oc7cc8ccccc8cc7c56)cc4)c23)cc1]

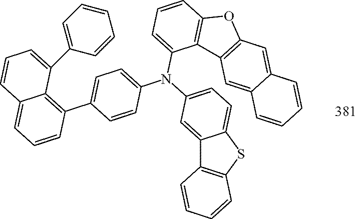 [SMILES: c1ccc(-c2cccc3cccc(-c4ccc(N(c5ccccc5)c5c6ccccc6cc6oc7ccccc7c56)cc4)c23)cc1]

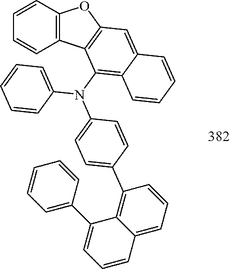 [SMILES: c1ccc(-c2ccc(N(c3ccc(-c4cccc5cccc(-c6ccccc6)c45)cc3)c3cccc4cc5oc6ccccc6c5cc34)cc2)cc1]

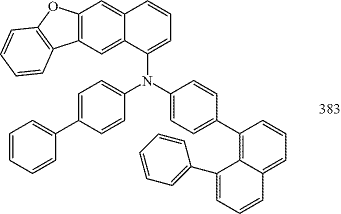 [SMILES: c1ccc(-c2cccc(N(c3ccc(-c4cccc5cccc(-c6ccccc6)c45)cc3)c3ccc4cc5oc6ccccc6c5cc4c3)c2)cc1]

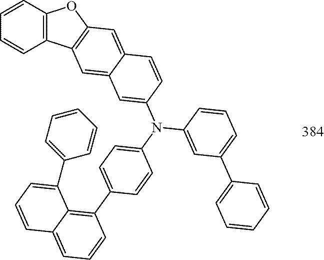 [SMILES: c1ccc(-c2cccc3cccc(-c4ccc(N(c5ccc6cc7c(cc6c5)oc5ccccc57)c5cccc6oc7ccccc7c56)cc4)c23)cc1]

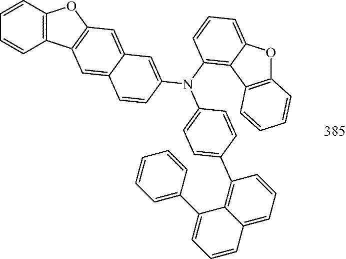 [SMILES: c1ccc(-c2cccc3cccc(-c4ccc(N(c5ccc6sc7ccccc7c6c5)c5cccc6cc7c(cc56)oc5ccccc57)cc4)c23)cc1]

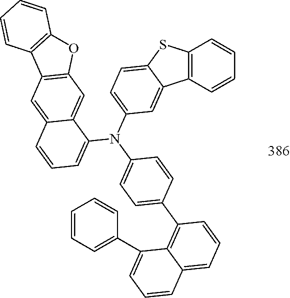 [SMILES: c1ccc(-c2cccc3cccc(-c4ccc(N(c5ccc(-n6c7ccccc7c7ccccc76)cc5)c5c6ccccc6cc6c5oc5ccccc56)cc4)c23)cc1]

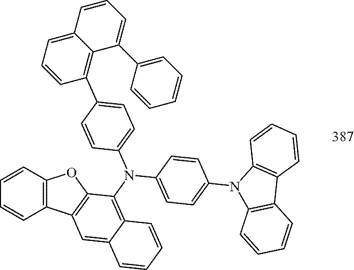 [SMILES: c1ccc(-c2cccc3cccc(-c4ccc(N(c5ccc6sc7ccccc7c6c5)c5ccc6c7cc8ccccc8cc7n(-c7ccccc7)c6c5)cc4)c23)cc1]

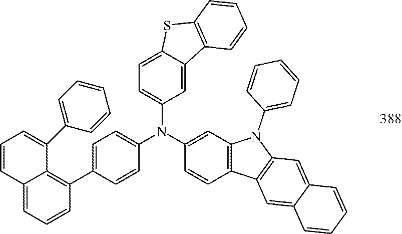 [SMILES: c1ccc(-c2ccc(-c3ccc(N(c4ccc(-c5cccc6cccc(-c7ccccc7)c56)cc4)c4ccc5c(c4)c4cc6ccccc6cc4n5-c4ccccc4)cc3)cc2)cc1]

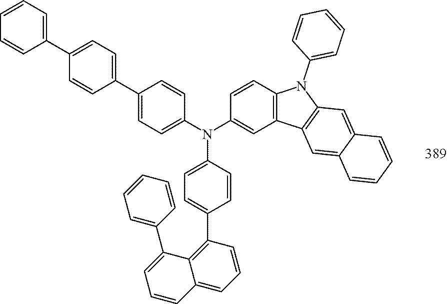 [SMILES: c1ccc(-c2cccc3cccc(-c4ccc(N(c5ccc(-c6cccc7sc8ccccc8c67)cc5)c5cccc6c5c5cc7ccccc7cc5n6-c5ccccc5)cc4)c23)cc1]

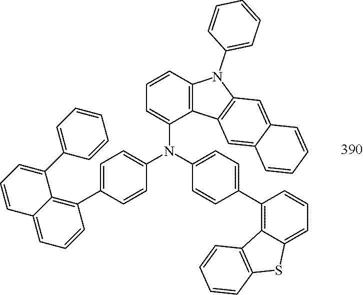 [SMILES: c1ccc(-c2cccc3cccc(-c4ccc(N(c5cc6ccccc6c6ccccc56)c5c6ccccc6cc6c5c5ccccc5n6-c5ccccc5)cc4)c23)cc1]

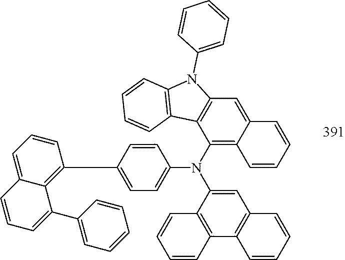 [SMILES: c1ccc(-c2cccc3cccc(-c4ccc(N(c5ccc6c(c5)oc5ccccc56)c5cccc6cc7c(cc56)c5ccccc5n7-c5ccccc5)cc4)c23)cc1]

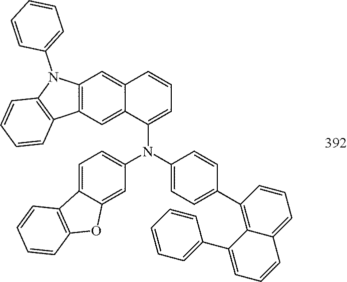 [SMILES: c1ccc(-c2ccc(N(c3ccc(-c4cccc5cccc(-c6ccccc6)c45)cc3)c3ccc4cc5c(cc4c3)c3ccccc3n5-c3ccccc3)cc2)cc1]

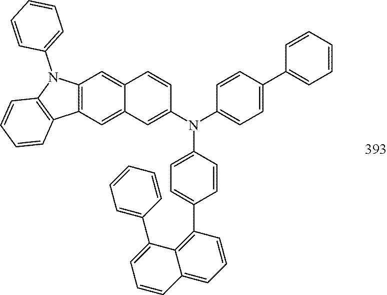 [SMILES: c1ccc(-c2cccc(N(c3ccc(-c4cccc5cccc(-c6ccccc6)c45)cc3)c3ccc4cc5c6ccccc6n(-c6ccccc6)c5cc4c3)c2)cc1]

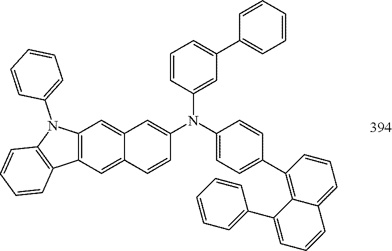 [SMILES: c1ccc(-c2cccc3cccc(-c4ccc(N(c5cccc6ccccc56)c5cccc6cc7c8ccccc8n(-c8ccccc8)c7cc56)cc4)c23)cc1]

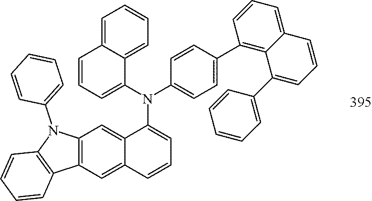 [SMILES: c1ccc(-c2cccc3cccc(-c4ccc(N(c5ccccc5)c5ccccc5-n5c6ccccc6c6cc7ccccc7cc65)cc4)c23)cc1]

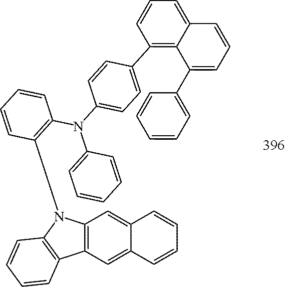 [SMILES: c1ccc(-c2cccc3cccc(-c4ccc(N(c5cccc(-n6c7ccccc7c7cc8ccccc8cc76)c5)c5ccc6oc7ccccc7c6c5)cc4)c23)cc1]

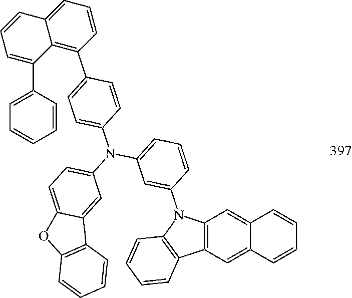 [SMILES: c1ccc(-c2cccc3cccc(-c4ccc(N(c5ccc(-n6c7ccccc7c7cc8ccccc8cc76)cc5)c5ccc6c(c5)sc5ccccc56)cc4)c23)cc1]

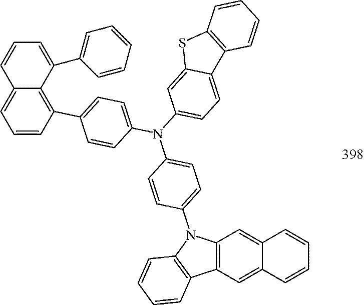 [SMILES: CC1(C)c2ccccc2-c2ccc(N(c3ccc(-c4cccc5cccc(-c6ccccc6)c45)cc3)c3c4ccccc4cc4c5ccccc5n(-c5ccccc5)c34)cc21]

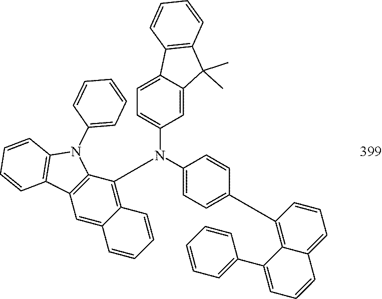 [SMILES: c1ccc(-c2cccc3cccc(-c4ccc(N(c5ccccc5)c5cccc6c7ccc8ccccc8c7n(-c7ccccc7)c56)cc4)c23)cc1]

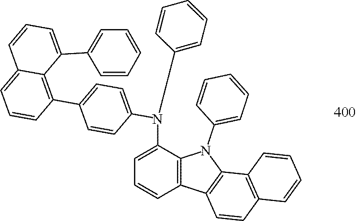 [SMILES: c1ccc(-c2cccc3cccc(-c4ccc(N(c5ccc6ccccc6c5)c5ccc6c(c5)c5ccc7ccccc7c5n6-c5ccccc5)cc4)c23)cc1]

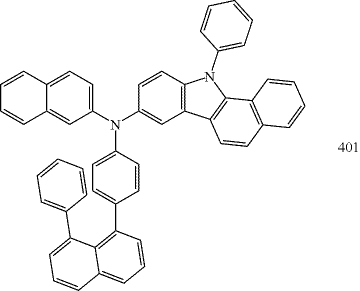 [SMILES: c1ccc(-c2ccc(N(c3ccc(-c4cccc5cccc(-c6ccccc6)c45)cc3)c3cccc4c3c3ccc5ccccc5c3n4-c3ccccc3)cc2)cc1]

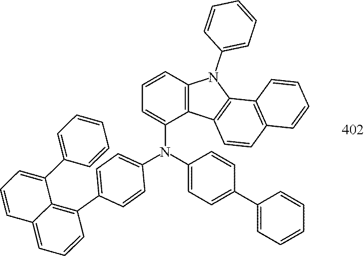 [SMILES: c1ccc(-c2cccc3cccc(-c4ccc(N(c5ccc6c(c5)sc5ccccc56)c5cc6ccccc6c6c5c5ccccc5n6-c5ccccc5)cc4)c23)cc1]

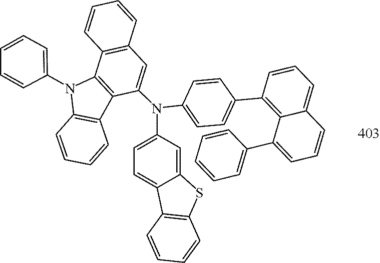 [SMILES: c1ccc(-c2ccc(N(c3ccc(-c4cccc5cccc(-c6ccccc6)c45)cc3)c3cc4c5ccccc5n(-c5ccccc5)c4c4ccccc34)cc2)cc1]

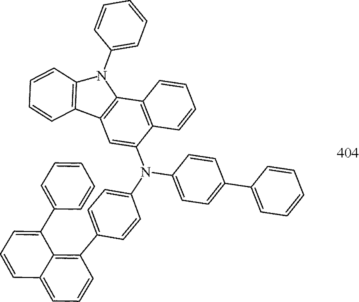 [SMILES: c1ccc(-c2cccc3cccc(-c4ccc(N(c5ccc6oc7ccccc7c6c5)c5cccc6c5ccc5c7ccccc7n(-c7ccccc7)c65)cc4)c23)cc1]

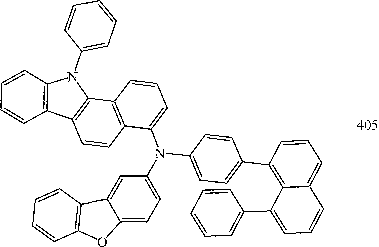 [SMILES: c1ccc(-c2cccc3cccc(-c4ccc(N(c5ccc6c(ccc7c8ccccc8n(-c8ccccc8)c67)c5)c5ccc6sc7ccccc7c6c5)cc4)c23)cc1]

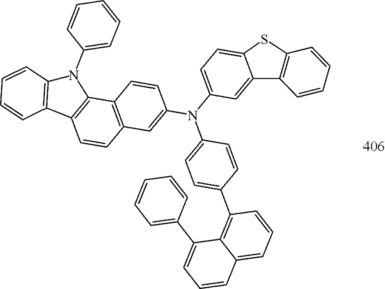 [SMILES: CC1(C)c2ccccc2-c2ccc(N(c3ccc(-c4cccc5cccc(-c6ccccc6)c45)cc3)c3ccc4ccc5c6ccccc6n(-c6ccccc6)c5c4c3)cc21]

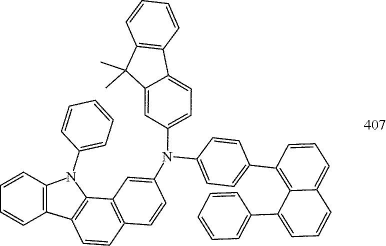 [SMILES: c1ccc(-c2cccc3cccc(-c4ccc(N(c5cccc6ccccc56)c5cccc6ccc7c8ccccc8n(-c8ccccc8)c7c56)cc4)c23)cc1]

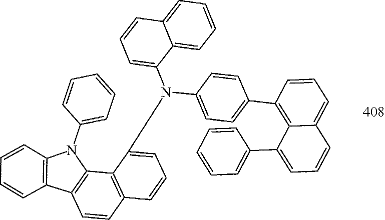 [SMILES: c1ccc(-c2cccc(N(c3ccc(-c4cccc5cccc(-c6ccccc6)c45)cc3)c3ccc(-n4c5ccccc5c5ccc6ccccc6c54)cc3)c2)cc1]

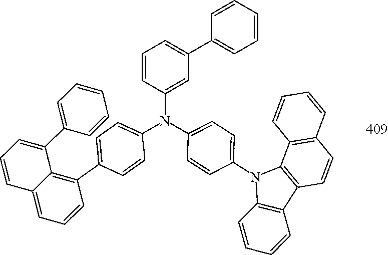 [SMILES: c1ccc(-c2cccc3cccc(-c4ccc(N(c5ccc(-c6cccc7ccccc67)cc5)c5cccc(-n6c7ccccc7c7ccc8ccccc8c76)c5)cc4)c23)cc1]

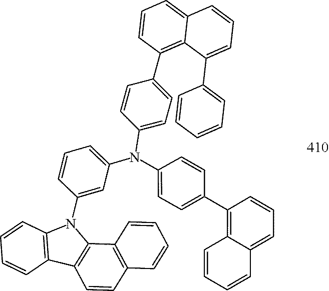 [SMILES: c1ccc(-c2cccc3cccc(-c4ccc(N(c5ccccc5-n5c6ccccc6c6ccc7ccccc7c65)c5cc6ccccc6c6ccccc56)cc4)c23)cc1]

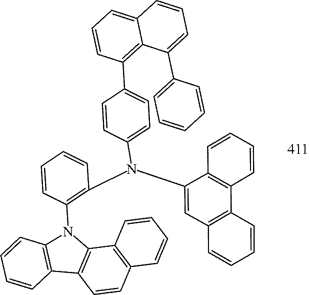 [SMILES: c1ccc(-c2cccc3cccc(-c4ccc(N(c5ccc(C6(c7ccccc7)c7ccccc7-c7ccccc76)cc5)c5cc6ccccc6c6c5[Si](c5ccccc5)(c5ccccc5)c5ccccc5-6)cc4)c23)cc1]

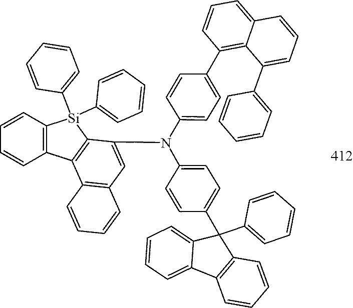 [SMILES: CC1(C)c2ccccc2-c2ccc(N(c3ccc(-c4cccc5cccc(-c6ccccc6)c45)cc3)c3cc4c(c5ccccc35)-c3ccccc3[Si]4(c3ccccc3)c3ccccc3)cc21]

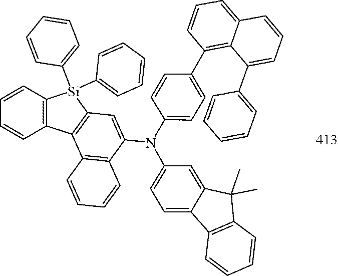 [SMILES: c1ccc(-c2cccc3cccc(-c4ccc(N(c5cccc6c7c(ccc56)[Si](c5ccccc5)(c5ccccc5)c5ccccc5-7)c5cccc6c5sc5ccccc56)cc4)c23)cc1]

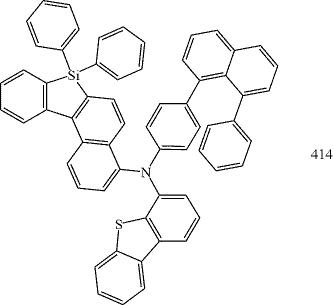 [SMILES: c1ccc(-c2cccc3cccc(-c4ccc(N(c5cccc(-n6c7ccccc7c7ccccc76)c5)c5ccc6c7c(ccc6c5)[Si](c5ccccc5)(c5ccccc5)c5ccccc5-7)cc4)c23)cc1]

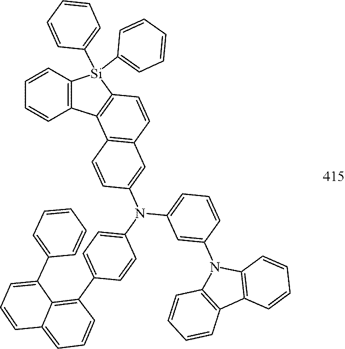 [SMILES: c1ccc(-c2cccc3cccc(-c4ccc(N(c5ccccc5)c5cccc6ccc7c(c56)-c5ccccc5[Si]7(c5ccccc5)c5ccccc5)cc4)c23)cc1]

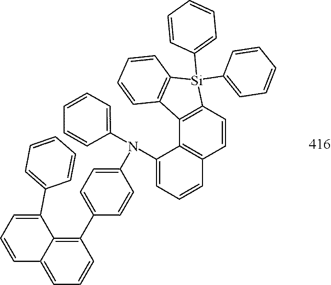 [SMILES: c1ccc(-c2cccc3cccc(-c4ccc(N(c5ccccc5)c5ccc6ccc7c(c6c5)-c5ccccc5[Si]7(c5ccccc5)c5ccccc5)cc4)c23)cc1]

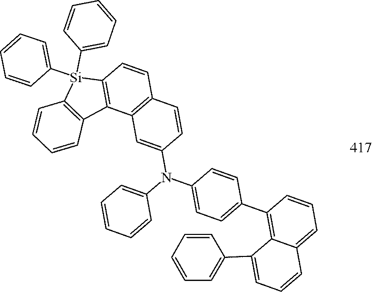 [SMILES: c1ccc(-c2cccc3cccc(-c4ccc(N(c5ccccc5)c5cccc6c5[Si](c5ccccc5)(c5ccccc5)c5cc7ccccc7cc5-6)cc4)c23)cc1]

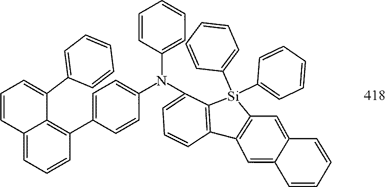 [SMILES: c1ccc(-c2cccc3cccc(-c4ccc(N(c5ccccc5)c5ccc6c(c5)[Si](c5ccccc5)(c5ccccc5)c5cc7ccccc7cc5-6)cc4)c23)cc1]

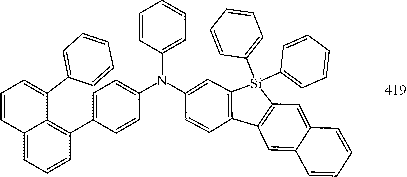 [SMILES: c1ccc(-c2cccc3cccc(-c4ccc(N(c5ccc6c(c5)-c5cc7ccccc7cc5[Si]6(c5ccccc5)c5ccccc5)c5cccc6ccccc56)cc4)c23)cc1]

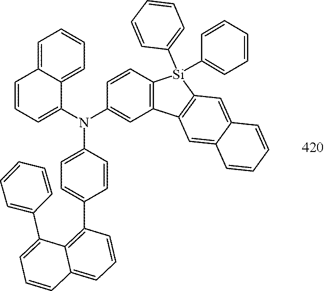 [SMILES: c1ccc(-c2cccc3cccc(-c4ccc(N(c5cccc6c5-c5cc7ccccc7cc5[Si]6(c5ccccc5)c5ccccc5)c5cccc6ccccc56)cc4)c23)cc1]

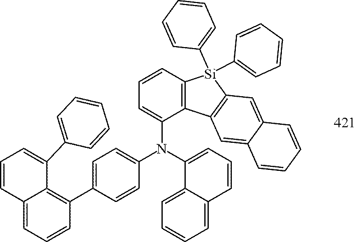 [SMILES: c1ccc(-c2cccc3cccc(-c4ccc(N(c5ccccc5)c5c6c(cc7ccccc57)[Si](c5ccccc5)(c5ccccc5)c5ccccc5-6)cc4)c23)cc1]

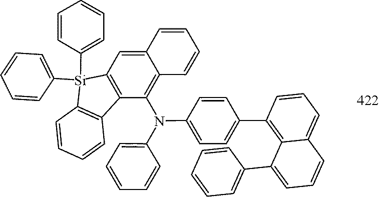 [SMILES: c1ccc(-c2cccc3cccc(-c4ccc(N(c5ccc6ccccc6c5)c5cccc6cc7c(cc56)-c5ccccc5[Si]7(c5ccccc5)c5ccccc5)cc4)c23)cc1]

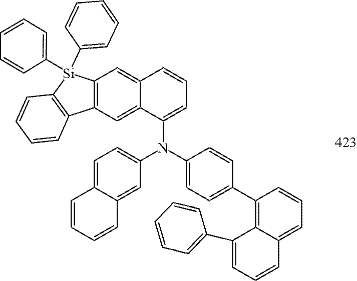 [SMILES: c1ccc(-c2ccc(N(c3ccc(-c4cccc5cccc(-c6ccccc6)c45)cc3)c3c4c(cc5ccccc35)-c3ccccc3[Si]4(c3ccccc3)c3ccccc3)cc2)cc1]

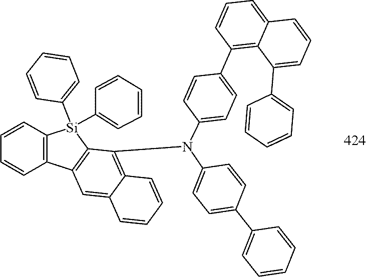 [SMILES: c1ccc(-c2cccc3cccc(-c4ccc(N(c5ccccc5)c5cccc6cc7c(cc56)[Si](c5ccccc5)(c5ccccc5)c5ccccc5-7)cc4)c23)cc1]

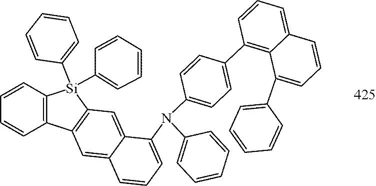 [SMILES: c1ccc(-c2ccc(N(c3ccc(-c4cccc5cccc(-c6ccccc6)c45)cc3)c3ccc4cc5c(cc4c3)[Si](c3ccccc3)(c3ccccc3)c3ccccc3-5)cc2)cc1]

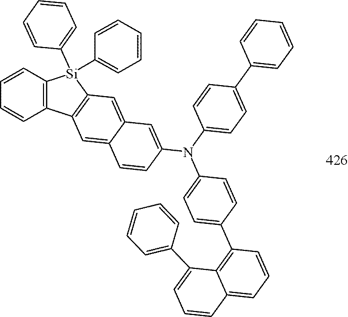 [SMILES: c1ccc(-c2ccc(N(c3ccc(-c4cccc5cccc(-c6ccccc6)c45)cc3)c3ccc4cc5c(cc4c3)-c3ccccc3[Si]5(c3ccccc3)c3ccccc3)cc2)cc1]

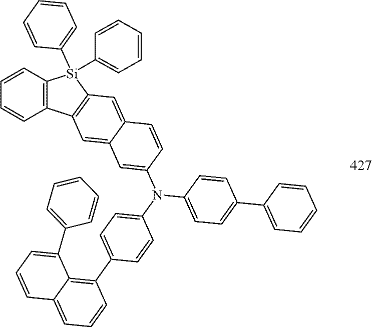 [SMILES: CC1(C)c2c(cccc2N(c2cccc(-c3cccc4cccc(-c5ccccc5)c34)c2)c2ccc3c(c2)oc2ccccc23)-c2ccc3ccccc3c21]

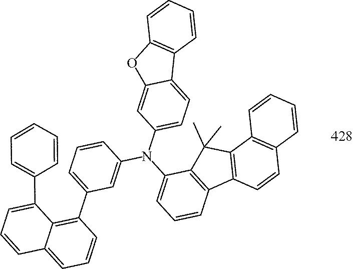 [SMILES: CC1(C)c2ccc(N(c3ccccc3-c3cccc4cccc(-c5ccccc5)c34)c3cccc4c3sc3ccccc34)cc2-c2ccc3ccccc3c21]

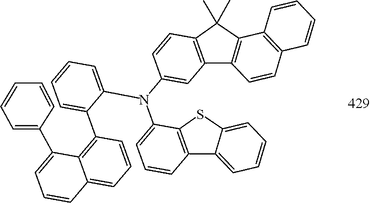 [SMILES: CC1(C)c2cccc(N(c3cccc(-c4cccc5c4C(c4ccccc4)CC=C5)c3)c3ccc(-c4ccccc4)c4ccccc34)c2-c2ccc3ccccc3c21]

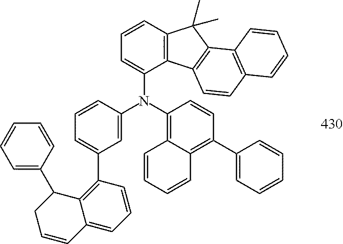 [SMILES: CC1(C)c2ccccc2-c2c(N(c3ccc(-c4cccc5cccc(-c6ccccc6)c45)cc3)c3ccc4c(c3)C3(c5ccccc5-c5ccccc53)c3ccccc3-4)cc3ccccc3c21]

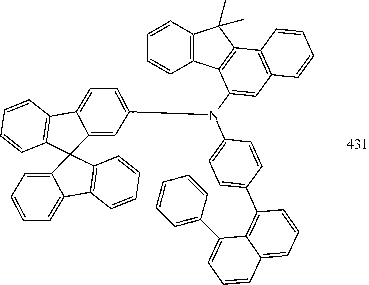 [SMILES: CC1(C)c2ccccc2-c2cc(N(c3ccc(-c4cccc5cccc(-c6ccccc6)c45)cc3)c3cccc(C4(c5ccccc5)c5ccccc5-c5ccccc54)c3)c3ccccc3c21]

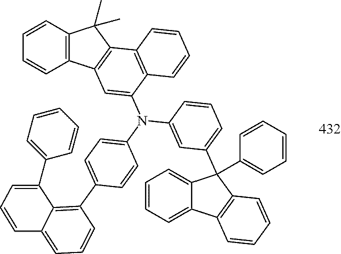 [SMILES: CC1(C)c2ccccc2-c2ccc3c(N(c4ccc(-c5cccc6cccc(-c7ccccc7)c56)cc4)c4cccc5c4-c4ccccc4C54c5ccccc5-c5ccccc54)cccc3c21]

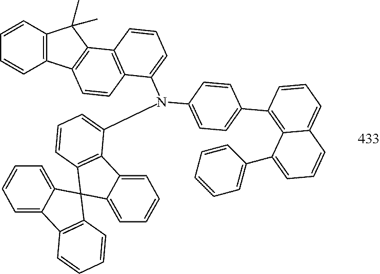 [SMILES: CC1(C)c2ccccc2-c2ccc3cc(N(c4ccc(-c5cccc6cccc(-c7ccccc7)c56)cc4)c4cccc5c4-c4ccccc4C5(c4ccccc4)c4ccccc4)ccc3c21]

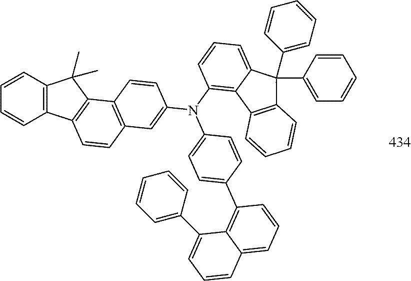 [SMILES: CC1(C)c2ccccc2-c2ccc3ccc(N(c4ccc(-c5cccc6cccc(-c7ccccc7)c56)cc4)c4ccc(C5(c6ccccc6)c6ccccc6-c6ccccc65)cc4)cc3c21]

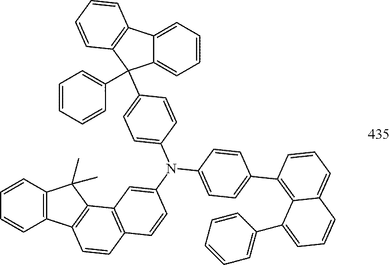 [SMILES: c1ccc(-c2ccc(N(c3ccc(-c4cccc5cccc(-c6ccccc6)c45)cc3)c3cccc4c3[Si](c3ccccc3)(c3ccccc3)c3c-4ccc4ccccc34)cc2)cc1]

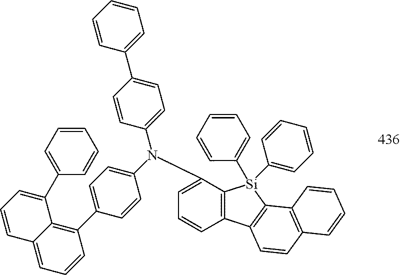 [SMILES: c1ccc(-c2ccc(N(c3ccc(-c4cccc5cccc(-c6ccccc6)c45)cc3)c3ccc4c(c3)[Si](c3ccccc3)(c3ccccc3)c3c-4ccc4ccccc34)cc2)cc1]

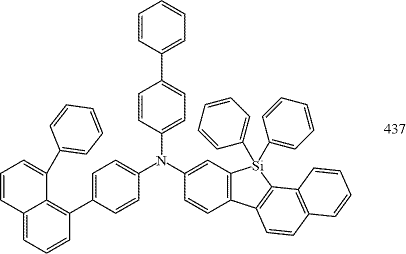 [SMILES: c1ccc(-c2ccc(N(c3ccc(-c4cccc5cccc(-c6ccccc6)c45)cc3)c3ccc4c(c3)-c3ccc5ccccc5c3[Si]4(c3ccccc3)c3ccccc3)cc2)cc1]

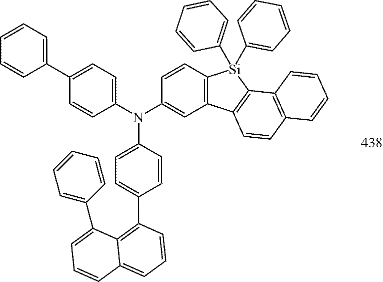 [SMILES: c1ccc(-c2cccc3cccc(-c4ccc(N(c5ccccc5)c5cccc6c5-c5ccc7ccccc7c5[Si]6(c5ccccc5)c5ccccc5)cc4)c23)cc1]

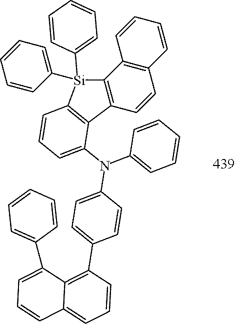 [SMILES: c1ccc(-c2cccc3cccc(-c4ccc(N(c5ccccc5)c5cc6ccccc6c6c5-c5ccccc5[Si]6(c5ccccc5)c5ccccc5)cc4)c23)cc1]

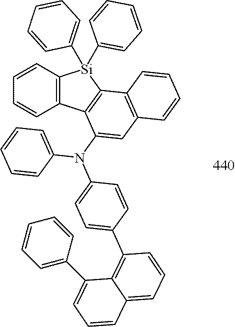 [SMILES: c1ccc(-c2cccc3cccc(-c4ccc(N(c5ccccc5)c5cc6c(c7ccccc57)[Si](c5ccccc5)(c5ccccc5)c5ccccc5-6)cc4)c23)cc1]

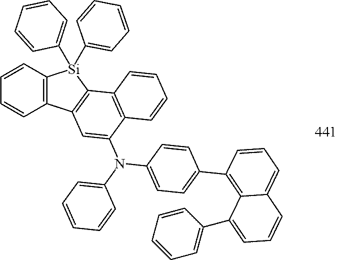 [SMILES: c1ccc(-c2ccc(N(c3ccc(-c4cccc5cccc(-c6ccccc6)c45)cc3)c3cc4c(c5ccccc35)[Si](c3ccccc3)(c3ccccc3)c3ccccc3-4)cc2)cc1]

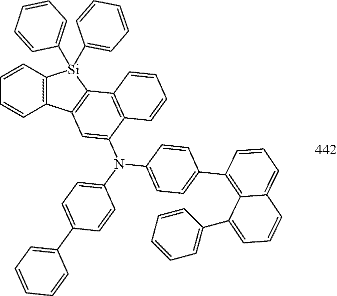 [SMILES: C1=Cc2cccc(-c3ccc(N(c4ccc(-c5ccccc5)cc4)c4ccc5c6c(ccc5c4)-c4ccccc4[Si]6(c4ccccc4)c4ccccc4)cc3)c2C(c2ccccc2)C1]

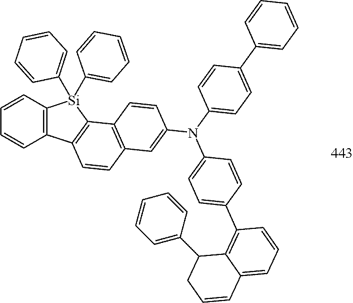 [SMILES: c1ccc(-c2ccc(N(c3ccc(-c4cccc5cccc(-c6ccccc6)c45)cc3)c3ccc4ccc5c(c4c3)[Si](c3ccccc3)(c3ccccc3)c3ccccc3-5)cc2)cc1]

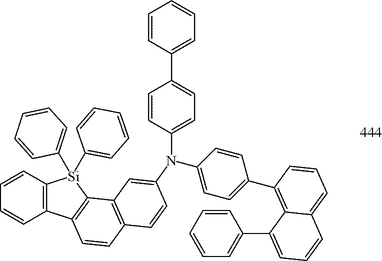 [SMILES: c1ccc(-c2cccc3cccc(-c4ccc(N(c5ccccc5)c5cccc6ccc7c(c56)[Si](c5ccccc5)(c5ccccc5)c5ccccc5-7)cc4)c23)cc1]

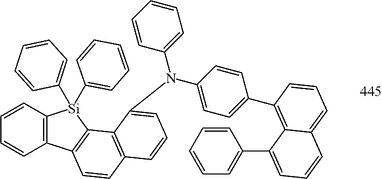 [SMILES: [2H]c1cccc2c3c(ccc12)-c1cc(N(c2ccccc2-c2cccc4cccc(-c5ccccc5)c24)c2cccc4c2sc2ccccc24)ccc1C31CCCCC1]

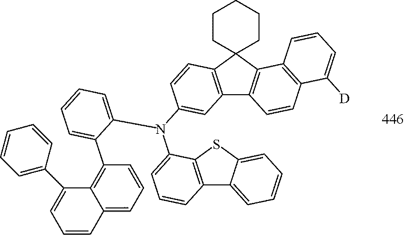 [SMILES: CCc1cc2c(c3ccccc13)-c1c(N(c3ccc(-c4cccc5cccc(-c6ccccc6)c45)cc3)c3cccc(-c4ccc(-c5ccccc5)cc4)c3)cccc1C21c2ccccc2-c2ccccc21]

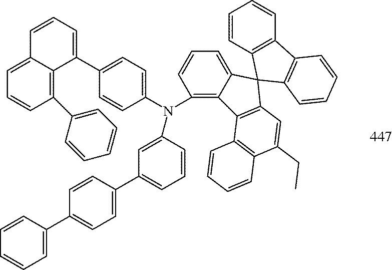

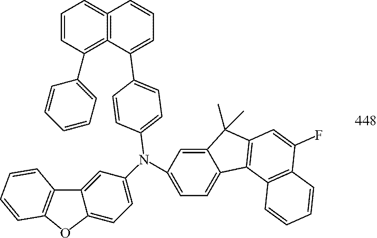 [SMILES: CC(C)(C)c1cc2sc3c(N(c4ccc(-c5cccc6cccc(-c7ccccc7)c56)cc4)c4ccccc4-c4ccccc4)cccc3c2c2ccccc12]

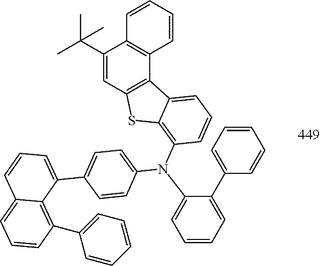 [SMILES: C[Si](C)(C)c1ccc2cc3oc4c(N(c5ccc(-c6cccc7cccc(-c8ccccc8)c67)cc5)c5cccc6ccccc56)cccc4c3cc2c1]

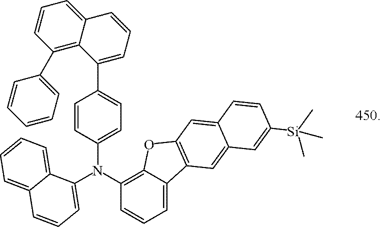

15. An electronic component comprising: an anode, a cathode, and a functional layer arranged between the anode and the cathode, wherein the functional layer comprises the organic compound according to claim 1; and
preferably, the functional layer comprises an electron blocking layer, and the electron blocking layer comprises the organic compound.

16. The electronic component according to claim 15, wherein the electronic component is an organic light-emitting device or a photoelectric conversion device.

17. An electronic device comprising the electronic component according to claim 15.

18. An electronic device comprising the electronic component according to claim 16.
